SEQUENCE LISTING (68 sequences)

SEQ ID NO: 1 - protein, 19 aa, Foot-and-mouth disease virus
LLNFDLLKLAGDVESNPGP

SEQ ID NO: 2 - protein, 19 aa, Foot-and-mouth disease virus
TLNFDLLKLAGDVESNPGP

SEQ ID NO: 3 - protein, 14 aa, Foot-and-mouth disease virus
LLKLAGDVESNPGP

SEQ ID NO: 4 - protein, 17 aa, Foot-and-mouth disease virus
NFDLLKLAGDVESNPGP

SEQ ID NO: 5 - protein, 20 aa, Foot-and-mouth disease virus
QLLNFDLLKLAGDVESNPGP

SEQ ID NO: 6 - protein, 24 aa, Foot-and-mouth disease virus
APVKQTLNFDLLKLAGDVESNPGP

SEQ ID NO: 7 - protein, 58 aa, Foot-and-mouth disease virus
VTELLYRMKRAETYCPRPLLAIHPTEARHKQKIVAPVKQTLNFDLLKLAGDVESNPGP

SEQ ID NO: 8 - protein, 40 aa, Foot-and-mouth disease virus
LLAIHPTEARHKQKIVAPVKQTLNFDLLKLAGDVESNPGP

SEQ ID NO: 9 - protein, 33 aa, Foot-and-mouth disease virus
EARHKQKIVAPVKQTLNFDLLKLAGDVESNPGP

SEQ ID NO: 10 - protein, 19 aa, Foot-and-mouth disease virus
NFDLLKLAGDVESNPGPFF

SEQ ID NO: 11 - protein, 25 aa, Encephalomyocarditis virus
GIFNAHYAGYFADLLIHDIETNPGP

SEQ ID NO: 12 - protein, 25 aa, Encephalomyocarditis virus
RIFNAHYAGYFADLLIHDIETNPGP

SEQ ID NO: 13 - protein, 25 aa, Encephalomyocarditis virus
HVFETHYAGYFADLLIHDVETNPGP

SEQ ID NO: 14 - protein, 25 aa, Theiler′s encephalomyelitis virus
KAVRGYHADYYKQRLIHDVEMNPGP

SEQ ID NO: 15 - protein, 25 aa, Theiler′s encephalomyelitis virus
RAVRAYHADYYKQRLIHDVEMNPGP

SEQ ID NO: 16 - protein, 25 aa, Theiler′s encephalomyelitis virus
KAVRGYHADYYRQRLIHDVETNPGP

SEQ ID NO: 17 - protein, 19 aa, Foot-and-mouth disease virus
LTNFDLLKLAGDVESNPGP

SEQ ID NO: 18 - protein, 19 aa, Foot-and-mouth disease virus
LLNFDLLKLAGDMESNPGP

SEQ ID NO: 19 - protein, 19 aa, Foot-and-mouth disease virus
MCNFDLLKLAGDVESNPGP

SEQ ID NO: 20 - protein, 19 aa, Equine rhinitis A virus
CTNYALLKLAGDVESNPGP

SEQ ID NO: 21 - protein, 20 aa, Equine rhinitis A virus
GATNFSLLKLAGDVELNPGP

SEQ ID NO: 22 - protein, 22 aa, Porcine teschovirus
GPGATNFSLLKQAGDVEENPGP

SEQ ID NO: 23 - protein, 20 aa, Drosophila C virus
EAARQMLLLLSGDVETNPGP

SEQ ID NO: 24 - protein, 20 aa, Cricket paralysis virus
FLRKRTQLLMSGDVESNPGP

SEQ ID NO: 25 - protein, 20 aa, Acute bee paralysis virus
GSWTDILLLLSGDVETNPGP

SEQ ID NO: 26 - protein, 20 aa, Thosea asigna virus
RAEGRGSLLTCGDVEENPGP

SEQ ID NO: 27 - protein, 20 aa, Infectious flacherie virus
TRAEIEDELIRAGIESNPGP

SEQ ID NO: 28 - protein, 20 aa, Bovine rotavirus
SKFQIDRILISGDIELNPGP

SEQ ID NO: 29 - protein, 20 aa, Porcine rotavirus
AKFQIDKILISGDVELNPGP

SEQ ID NO: 30 - protein, 20 aa, Human rotavirus
SKFQIDKILISGDIELNPGP

SEQ ID NO: 31 - protein, 20 aa, Trypanosoma brucei
SSIIRTKMLVSGDVEENPGP

SEQ ID NO: 32 - protein, 20 aa, Trypanosoma cruzi
CDAQRQKLLLSGDIEQNPGP

SEQ ID NO: 33 - protein, 5 aa, Artificial Sequence
  Description of Artificial Sequence Synthetic peptide
  MOD_RES (2): Variable amino acid
RXKRR

SEQ ID NO: 34 - protein, 5 aa, Artificial Sequence
  Description of Artificial Sequence Synthetic peptide
IEDGR

SEQ ID NO: 35 - protein, 10 aa, Artificial Sequence
  Description of Artificial Sequence Synthetic peptide
LAGFATVAQA

SEQ ID NO: 36 - protein, 6 aa, Artificial Sequence
  Description of Artificial Sequence Synthetic peptide
LVPRGS

SEQ ID NO: 37 - protein, 5 aa, Artificial Sequence
  Description of Artificial Sequence Synthetic peptide
  MOD_RES (1): Met, Leu or Ile
  MOD_RES (2): Variable amino acid
  MOD_RES (5): Variable amino acid
XXGGX

SEQ ID NO: 38 - protein, 5 aa, Artificial Sequence
  Description of Artificial Sequence Synthetic peptide
  MOD_RES (1): Met, Leu or Ile
  MOD_RES (2): Variable amino acid
  MOD_RES (4): Variable amino acid
XXGXG

SEQ ID NO: 39 - DNA, 34 bp, Artificial Sequence
  Description of Artificial Sequence Synthetic oligonucleotide
tacttatgactcgtactattgttattcatccagg

SEQ ID NO: 40 - RNA, 7 nt, Artificial Sequence
  Description of Artificial Sequence Synthetic oligonucleotide
  modified_base (2): a, c, t, g, unknown or other
ynyuray

SEQ ID NO: 41 - DNA, 128 bp, Human adenovirus
taatttactaagttacaaagctaatgtcaccactaactgctttactcgctgcttgcaaaacaaattcaaaaagttagcattataattagaataggatttaaaccccccggtcatttcctgctcaatac

SEQ ID NO: 42 - DNA, 32 bp, Artificial Sequence
  Description of Artificial Sequence Synthetic oligonucleotide
tacttatagtaatctaattcctgctctctcag

SEQ ID NO: 43 - DNA, 273 bp, Homo sapiens
catccggacaaagcctgcgcgcgccccgccccgccattggccgtaccgccccgcgccgccgccccatctcgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcgatgatatca

SEQ ID NO: 44 - DNA, 461 bp, Foot-and-mouth disease virus
agcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattaatgaccct

SEQ ID NO: 45 - DNA, 34 bp, Artificial Sequence; identical to SEQ ID NO: 39
  Description of Artificial Sequence Synthetic oligonucleotide

SEQ ID NO: 46 - DNA, 846 bp, Homo sapiens
atggctatgatggaggtccaggggggacccagcctgggacagacctgcgtgctgatcgtgatctttacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttaatagacatggaccatgaagccagttttttcggggcctttttagttggctaa

SEQ ID NO: 47 - DNA, 9587 bp, Artificial Sequence
  Description of Artificial Sequence Synthetic CP1524 sequence
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 48 - DNA, 3855 bp, Artificial Sequence
  Description of Artificial Sequence Synthetic CP1606 sequence
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtagctagcgggagggaggtcctggtctagacattcagagcttgtagaatttctgcatccaggtcagaagaatgtccacttccccaagggctttggtcagagctgcttctacgtccaactgtttgaatgctctccggaatagcagaaaccgcctgtgtgcactgtctctgatggaaaacatctcattttcttgactgggttgcagttgtgacacgatgagaacaaagttgttggccagagtagagaatgacttcagagtcctgacttcaactgttctattgtggtggtttttgaaaacagttttcaagtagaactccagcagggtgtggacaaggtaacagctctcagcatccgagacgttctgcagaacctcctgctgcagcagccgggcactcgtgatgttatcctgagcttgcatagtgtctttcacagcccagaaggcttcccacagtttctggggaacaacccccttcacttggcagggcccaaagtggaattcttggccctgggcccctgatacctggctccagagaagcagggtaaaacccaggcaagggagcacaaccatctgcatttgagaggctgtcgccagcaaaggagggcagaagggtctggctaaagtccacaggctttgcagcctctgttgaaaattcatgatggccctcctaccgcctggatgaataacaatagtacgagtcataagtaattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaaaaagggcgaattctgcagatatccatcacactggcggccgctcgagcatgcatctagaggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 49 - DNA, 36264 bp, Human adenovirus
cagggtaatcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgggatctctgcaggaatttgatatcaagcttatcgataccgtcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgctagcggcgcgccgtttcatccggacaaagcctgcgcgcgccccgccccgccattggccgtaccgccccgcgccgccgccccatctcgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcgatgatatcagatcaaacgatatcaccggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaattaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgattaccctgttat

SEQ ID NO: 50 - DNA, 37613 bp, Human adenovirus
  modified_base (22433): a, c, t, g, unknown or other
  modified_base (22443): a, c, t, g, unknown or other
cagggtaatcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgggatctctgcaggaatttgatatcaagcttatcgataccgtcgaaacttgtttattgcagcttataatggttacaaataaagcaatagcatcacaaatttcacaaataaagcatttttttcactgcattctagttgtggtttgtccaaactcatcaatgtatcttatcatgtctggatccgctagcggcgcgccgtttcatccggacaaagcctgcgcgcgccccgccccgccattggccgtaccgccccgcgccgccgccccatctcgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcgatgatatcagatcaaacgatatcaccggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataagcgatcgcagcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaatangtgaccggangtcggcacctttcctttgcaattaatgaccctgtatacgccaccatggctatgatggaggtccaggggggacccagcctgggacagacctgcgtgctgatcgtgatctttacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttaatagacatggaccatgaagccagttttttcggggcctttttagttggctaagtatacttcgaatgcatgcgatcgcagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaattaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgattaccctgttat

SEQ ID NO: 51 - DNA, 37155 bp, Human adenovirus
agggtaatcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgggatctctgcaggaatttgatatcaagcttatcgataccgtcgaaacttgtttattgcagcttataatggttacaaataaagcaatagcatcacaaatttcacaaataaagcatttttttcactgcattctagttgtggtttgtccaaactcatcaatgtatcttatcatgtctggatccgctagcggcgcgccgtttcatccggacaaagcctgcgcgcgccccgccccgccattggccgtaccgccccgcgccgccgccccatctcgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcgatgatatcagatcaaacgatatcaccggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataattacttatgactcgtactattgttattcatccaggcggtaggagggccatcatggctatgatggaggtccaggggggacccagcctgggacagacctgcgtgctgatcgtgatctttacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttaatagacatggaccatgaagccagttttttcggggcctttttagttggctaagctagctactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaattaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgattaccct

SEQ ID NO: 52 - DNA, 20 bp, Artificial Sequence
  Description of Artificial Sequence Synthetic primer
taccggggtacccaactcca

SEQ ID NO: 53 - DNA, 18 bp, Artificial Sequence
  Description of Artificial Sequence Synthetic primer
gacgcggcctgtccggcc

SEQ ID NO: 54 - DNA, 55 bp, Artificial Sequence
  Description of Artificial Sequence Synthetic primer
tacttatgactcgtactattgttattcatccaggcggtaggagggccatcatgaa

SEQ ID NO: 55 - DNA, 43 bp, Artificial Sequence
  Description of Artificial Sequence Synthetic primer
cctttattgaaagtgtctctagtagctagcgggagggaggtcc

SEQ ID NO: 56 - DNA, 24 bp, Artificial Sequence
  Description of Artificial Sequence Synthetic primer
tactagagacactttcaataaagg

SEQ ID NO: 57 - DNA, 21 bp, Artificial Sequence
  Description of Artificial Sequence Synthetic primer
gttaacatggttctgcagagc

SEQ ID NO: 58 - DNA, 30 bp, Artificial Sequence
  Description of Artificial Sequence Synthetic primer
ggctcgtccatgggatccacctcaaaagtc

SEQ ID NO: 59 - DNA, 20 bp, Artificial Sequence
  Description of Artificial Sequence Synthetic primer
ggatcccatggacgagccca

SEQ ID NO: 60 - DNA, 34 bp, Artificial Sequence
  Description of Artificial Sequence Synthetic primer
cttattaccggggtacccaactcctcgagtattt

SEQ ID NO: 61 - DNA, 38 bp, Artificial Sequence
  Description of Artificial Sequence Synthetic primer
tacaagtatacgccaccatggctatgatggaggtccag

SEQ ID NO: 62 - DNA, 32 bp, Artificial Sequence
  Description of Artificial Sequence Synthetic primer
ttgtagtatacttagccaactaaaaaggcccc

SEQ ID NO: 63 - protein, 4 aa, Artificial Sequence
  Description of Artificial Sequence Synthetic peptide
RAKR

SEQ ID NO: 64 - protein, 4 aa, Artificial Sequence
  Description of Artificial Sequence Synthetic peptide
RKKR

SEQ ID NO: 65 - protein, 4 aa, Artificial Sequence
  Description of Artificial Sequence Synthetic peptide
RKRR

SEQ ID NO: 66 - protein, 4 aa, Artificial Sequence
  Description of Artificial Sequence Synthetic peptide
RRKR

SEQ ID NO: 67 - protein, 4 aa, Artificial Sequence
  Description of Artificial Sequence Synthetic peptide
RRRR

SEQ ID NO: 68 - DNA, 32 bp, Artificial Sequence
  Description of Artificial Sequence Synthetic oligonucleotide
tacttatagtgaaaacgttcctgctctcacag

(19) United States
(12) Patent Application Publication
(10) Pub. No.: US 2006/0292682 A1
(43) Pub. Date: Dec. 28, 2006

(54) Addition of transgenes into adenoviral vectors

(76) Inventors: Lynda K. Hawkins, Brookline, MA (US); Seshidar Reddy Police, Chester Springs, PA (US)

Correspondence Address: DLA PIPER RUDNICK GRAY CARY US LLP;Patent Group, 1200 Nineteenth Street, N.W., Washington, DC, 20036-2412, US

(21) Appl. No.: 11/181,850

(22) Filed: Jul. 15, 2005

Related U.S. Application Data
(60) Provisional application No. 60/589,804, filed on Jul. 22, 2004.
(60) Provisional application No. 60/660,542, filed on Mar. 11, 2005.

(51) Int. Cl. C12N 15/861; C12N 7/00

(52) U.S. Cl. 435/235.1; 435/456; 977/802

(57) ABSTRACT

Adenoviral vectors are provided that contain a transgene inserted in the viral genome at a novel site such that one or more of native processing, regulatory signals or heterologous transcription signals such as branch point sites, splice acceptor sites, internal ribosome entry sites, self-processing cleavage sites and/or polyA signals contribute to transgene expression.

This application claims priority from U.S. Provisional Application Ser. No. 60/589,804, filed Jul. 22, 2004 and U.S. Provisional Application Ser. No. 60/660,542, filed Mar. 11, 2005.

### BACKGROUND OF THE INVENTION

1. Field of the Invention

The invention relates to adenoviral vectors comprising transgenes inserted into the genome at various locations, such that transgene expression relies on either native adenoviral processing and/or regulatory signals or heterologous transcription signals, and methods for making the same.

2. Background of the Technology

Adenoviral vectors have been utilized extensively to deliver and express genes in mammalian cells, in vitro, ex vivo and in vivo. Adenovirus vectors offer many advantages for gene delivery: wild type adenoviruses cause only minor illness in healthy individuals, they are easy to propagate to high titers, they can infect most cell types regardless of their growth state, they can deliver nucleic acids encoding proteins of interest to cells, and in their most recent embodiments they can be engineered to replicate selectively in certain cells and can be targeted by incorporating or attaching a ligand to capsid proteins.

There are two main types of adenoviral vectors, replication-incompetent (replication defective) and replication-competent. Replication defective vectors traditionally lack one or more genes essential for replication. These replication incompetent viruses are propagated on cells that complement the essential gene(s) which are lacking. Replication competent adenoviral vectors traditionally do not lack any of the adenovirus genes essential for replication. One type of replication competent adenovirus replicates selectively in certain cells. For example, a large number of various cell- and tissue-specific replication competent adenovirus vectors, which preferentially replicate in (and thus destroy) certain cell types, have been described. One method of constructing a cell-specific replication competent virus is to place a heterologous transcriptional regulatory element (TRE) in operative linkage with an essential adenoviral coding sequence. Another method is to delete or inactivate an adenoviral coding sequence that when deleted or inactivated results in preferential replication of the virus in a certain cell-type, for example tumor cells.

Adenoviral vectors are limited by the size of their genome (Bett et al, J Virol 67:5911-5921, 1993). This in turn limits the amount of heterologous DNA that may be inserted into the vector and therefore limits the amount and or length of heterologous coding sequences that maybe incorporated into the adenovirus genome. Therefore, replication defective viruses are capable of the largest heterologous DNA insertions, but are still limited by the size of the adenovirus genomic DNA and the amount of adenoviral DNA deleted.

In the case of replication competent viruses, they are limited not only because they contain the majority of adenoviral coding sequences, but they are limited in the number of insertion sites for heterologous DNA. Traditionally, heterologous insertions of DNA have been inserted in place of an adenovirus E3 coding sequence(s) in replication competent viruses (WO 02/067861, WO 01/02540). This limits the size of the heterologous DNA insertion to a slightly larger size than that of the E3 deletion. Also, for a particular application it may not be desired or advantageous to delete an E3 coding sequence(s) or to insert the heterologous DNA in the E3 region.

Adenoviral infection is divided into early and late phases, separated by viral DNA replication. Viral genes are expressed before (early phase) and/or after (late phase) viral DNA replication. In general, early genes encode proteins that prepare the cell for viral propagation and late proteins are capsid proteins or are involved in packaging and assembly of virions. One method that has been used extensively to express transgenes is to insert an expression cassette into the viral genome in such a way that the expression is constitutive and not regulated by the virus itself. In such cases, typically the transgene is inserted in place of a non-essential gene or in place of an essential gene, wherein the essential gene is complemented by a cell line used for propagation of the viral vector.

In adenoviruses, late transcription initiates predominantly at the major late promoter (MLP) after the initiation of DNA replication. At late time points, transcription from the MLP accounts for approximately 30% of the total RNA synthesis. In all adenoviruses, the primary transcript initiated from the MLP extends towards the right for about 28 kb and terminates at a position close to 99 map units in the viral genome. Each region is characterized by its own polyadenylation signal sequence and usually consists of several differentially spliced messages. The primary transcripts are cleaved and become polyadenylated at one of the five locations along the genome, generating five families (L1-L5) of mRNAs, each with 3′ co termini. After the selection of the 3′ end, the primary transcript is processed by splicing in such a way that each mature RNA gains a common set of three short 5′ leader segments called tripartite leader (TPL) sequences derived from 16.8, 19.8 and 26.9 map units. Each of the five families expresses more than one protein with the exception of L5 in some adenovirus serotypes. Ad 40 and 41 express more than one protein from the L5 region. The mRNAs within a family differ from each other by positions where the tripartite leader is spliced into a splice acceptor site located upstream of an open reading frame (ORF) in a late region of the genome. For example, four types of mRNAs are produced from the L2 region and all have common 3′ ends. Although the messages are polycistronic, only the ORF present at the 5′ end is thought to be translated in native Ad transcripts. Fuerer et al. (Gene Therapy 11: 142-151 (2004)) describes inserting a coding sequence for cytosine deaminase (CD) in a replication competent virus using at least two methods. One method relies on the use of the encephalomyocarditis virus internal ribosome entry site (IRES) to convert the L5 transcript into a bicistronic mRNA. Fuerer et al. also describe a method that makes use of the splice acceptor site sequence from the Ad41 long fiber gene to splice the CD cassette onto the tripartite leader exons of the major late transcript.

### SUMMARY OF THE INVENTION

The invention relates to a novel gene delivery system using the adenovirus (Ad) genome as a gene delivery and/or expression vehicle. The adenovirus genome may be either replication competent or incompetent (defective).

The invention provides adenoviral vectors wherein transgenes are inserted into the adenoviral genome at various locations, e.g., upstream or downstream of existing genes, eliminating the need for the addition of large regulatory sequences such as promoters, allowing heterologous DNA (a transgene) to be inserted into the Ad genome, in a location and manner effective to take advantage of and/or utilize native adenoviral processing.

In some aspects of the invention, transcription signals (e.g. branch point sites, splice acceptor sites, IRES, self-processing cleavage sites and/or polyA signals) are added to the adenoviral vectors of the invention. The addition of genes with processing signals such as branch point sequences along with splice acceptor sequences takes advantage of the mRNA processing and expression system utilized by native adenovirus.

### BRIEF DESCRIPTION OF THE FIGURES

FIG. 1 is a schematic depiction of the native genome organization of Ad5.

FIG. 2 is a schematic depiction of the native Ad5 transcription units.

FIG. 3 is a schematic depiction of the L1 region of Ad5.

FIG. 4 is a schematic depiction of the L2 region of Ad5.

FIG. 5 is a schematic depiction of the L3 region of Ad5.

FIG. 6 is a schematic depiction of the E3 transcription units.

FIG. 7 is a schematic depiction of the E3A region. The star denotes 192 base pairs in Ad5 that are frequently deleted from adenoviral vectors.

FIG. 8 is a schematic depiction of the E3B region.

FIG. 9 is a schematic depiction of an exemplary embodiment of the invention wherein an L3 region insertion site is shown with an example of a chimeric adenoviral leader region, wherein a transgene is inserted between the hexon and 23K protease coding sequences. The transgene is operatively linked to (i.e. utilizes for expression) the native splice acceptor site for the 23K mRNA which is located about 95 bases upstream of the hexon stop codon in Ad5. A heterologous splice acceptor site is inserted between the transgene coding sequence and the 23K coding sequence and the heterologous splice acceptor site is operatively linked to the 23K coding sequence. This allows four different mRNAs to be transcribed from the L3 region. In this example, the native L3 polyadenylation sequence is left intact and is utilized by all four mRNAs of the chimeric L3 region.

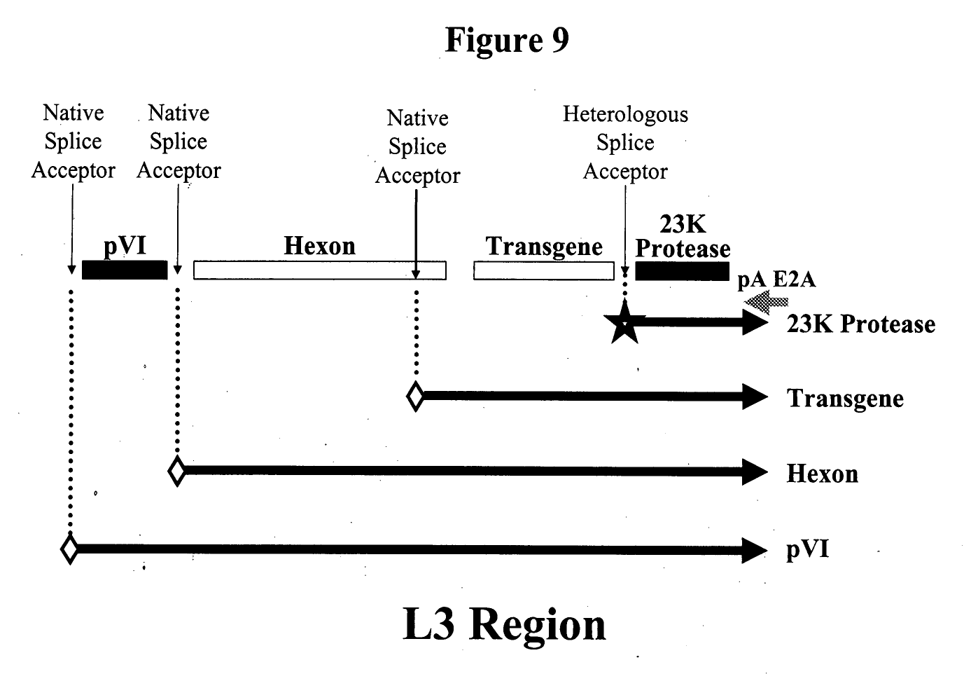

FIG. 10 is a schematic depiction of an exemplary embodiment of the invention wherein an L3 region insertion site is shown with an example of a chimeric adenoviral leader region. In this case, a transgene is inserted between the 23K protease coding sequence and the L3 polyadenylation sequence in the L3 region. The transgene is operatively linked to (i.e. utilizes for expression) a 2A-like sequence or IRES. The 2A-like sequence or IRES is also operatively linked to the 23K coding sequence. Similar to the native L3 region, this chimeric adenoviral L3 region will result in production of three L3 mRNAs. In this case, all three of the L3 mRNAs will contain an IRES (or 2A-like sequence) and therefore translation of all three of these mRNAs will result in the expression of the transgene. For this example, the native L3 polyadenylation sequence is left intact and is utilized by all three mRNAs of the chimeric L3 region.

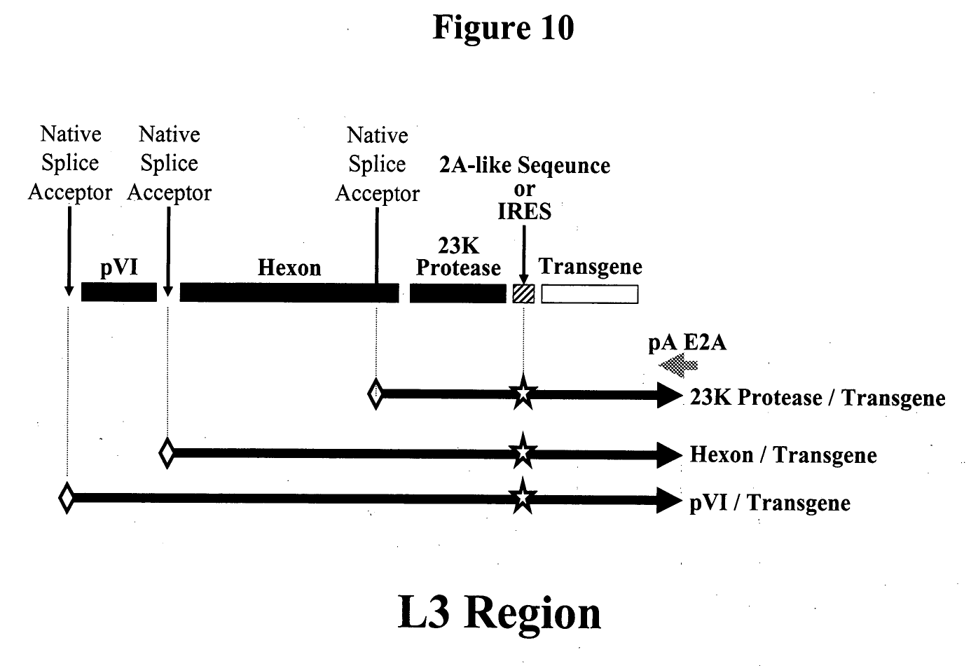

FIG. 11 provides a schematic depiction of OV-1160 comprising in the 5′ to 3′ direction: the E2F-1 promoter operatively linked to E1A; and in the L3 region: pVI, hexon, a splice acceptor and the TRAIL coding sequence. The vector carries the packaging signal in the native location and carries a polyadenylation signal upstream of the E2F-1 promoter to inhibit transcriptional read-through from the LITR.

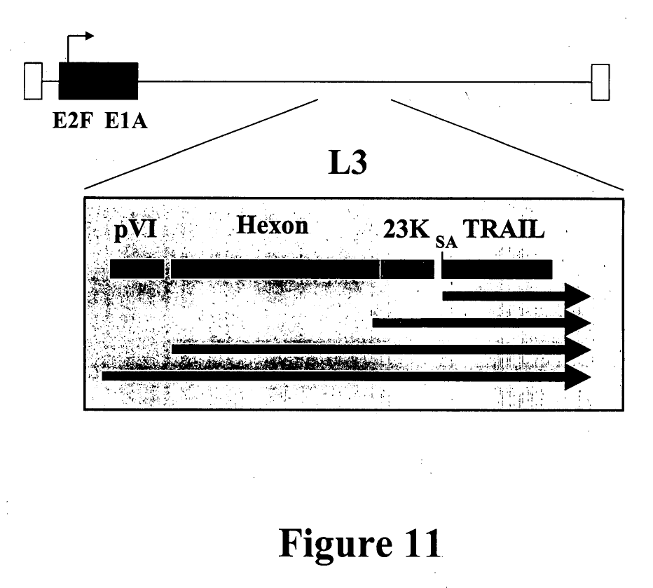

FIG. 12 provides a schematic depiction of OV-1164 comprising in the 5′ to 3′ direction: the E2F-1 promoter operatively linked to E1A; and in the L3 region: pVI, hexon, an IRES, the TRAIL coding sequence and the 23K gene. The vector carries the packaging signal in the native location and carries a polyadenylation signal upstream of the E2F-1 promoter to inhibit transcriptional read-through from the LITR.

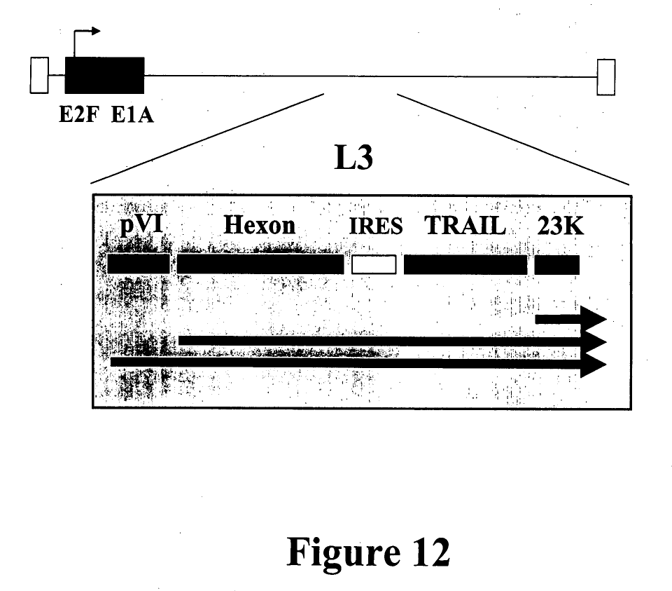

### DETAILED DESCRIPTION OF THE INVENTION

The present invention provides recombinant adenoviral vectors which rely on the use of existing (native) transcriptional and/or translational regulatory elements for the expression of one or more transgenes. In one example, the major late promoter is used for expression of a transgene during the late phase of infection by insertion of one or more transgenes into a late region. In another example, this strategy is used to insert transgenes into an adenoviral early region. A transgene may be inserted in operative linkage with any endogenous adenoviral promoter, so long as the open reading frames (ORF) of essential adenoviral genes and native adenoviral processing signals, such as endogenous polyadenylation and transcriptional signals are not disturbed and the virus can effectively replicate.

In addition to a transgene, a branch point, splice acceptor site, or other similar sequence, may be included in an adenoviral vector of the invention to allow correct processing of the transcript. The quantity and kinetics of transgene expression can be manipulated by altering the particular sequence of the processing signals, such as splicing efficiency, translation initiation efficiency or the efficiency of the signals, so that the desired kinetics are obtained. Multiple transgenes may be included in the same vector, either in the same or different regions of the genome. Other endogenous adenoviral genes may be deleted, to achieve a desired effect or to allow for the inclusion of longer sequences of exogenous DNA.

In one aspect, the present invention “restores transcription signals” to a adenoviral vector. By “restoring transcription signals” it is meant that a non-native transcription signal is used to perform a similar function or to affect transcription in a manner similar to that of the native transcription signal. For example, in the L5 region the fiber stop codon is embedded in the L5 polyA signal. Insertion of a transgene downstream of the fiber coding region will usually include a mutation that disrupts the function of the native L5 polyA site, but maintains the fiber stop codon. Therefore, the polyA site must be restored for correct processing of the L5 message. This entails adding a polyA signal to the L5 region. This polyA signal may be the same sequence as the one that was disrupted or may be a non-native or heterologous polyA signal. In an exemplary embodiment where a transgene is inserted downstream of the fiber coding region, the polyA signal is inserted downstream of the transgene coding region. In a different embodiment of the invention where a transgene is inserted between existing adenoviral coding sequences, the restoration/addition of an additional splice acceptor site is typically employed. For example, the transgene may use an adenoviral native splice site that would have been the native splice site for the next adenoviral coding sequence downstream of the transgene coding sequence. In this case, a splice site has to be restored for the next adenoviral coding sequence downstream of the transgene coding sequence. This “restored” splice site could be the same sequence as the native sequence now utilized for splicing upstream of the transgene sequence or it could be a heterologous splice site. Different splice sites are known in the art to have different splicing efficiencies. Therefore, one skilled in the art can choose splice sites with different efficiencies as desired for a particular application.

The advantages provided by the present invention are several-fold: a) by utilizing existing expression signals, larger size transgene(s) may be incorporated in a size-limited vector; b) the known and predictable expression patterns of adenoviral genes allows for design of adenoviral vectors with desired transgene expression kinetics, such as expression level and timing, wherein expression is tailored dependent upon the nature of the transgene; c) the adenoviral vectors of the invention take advantage of native regulation systems that are already in place in the adenoviral genome; d) the adenoviral vectors of the invention include introduced regulatory sequences such as self-processing cleavage sites, branch point sequences and splice acceptor sequences which are small compared to other elements typically used for transgene expression; and e) the selection of heterologous transcription factors can be used to further regulate expression. Numerous transgenes and/or transgene categories exist that can be transgene candidates, as further described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis“(M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

### DEFINITIONS

Unless otherwise indicated, all terms used herein have the same meaning as they would to one skilled in the art and the practice of the present invention will employ, conventional techniques of microbiology and recombinant DNA technology, which are within the knowledge of those of skill of the art.

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring DNA into cells either in vitro or in vivo. For purposes of the present invention, these terms refer to adenoviruses, including recombinant adenoviruses formed when an adenoviral vector of the invention is encapsulated in an adenovirus capsid.

An “adenovirus vector” or “adenoviral vector” (used interchangeably) as referred to herein is a polynucleotide construct, which is replication competent or replication incompetent (defective).

Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG to immunologically “mask” the antigenicity and/or increase half-life, or conjugated to a nonviral protein. Hence, the terms “adenovirus vector” or “adenoviral vector” as used herein include adenovirus or adenoviral particles.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. Preferably, a vector of the invention comprises DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides. A nucleic acid sequence is “operatively linked” or “operably linked” when it is placed into a functional relationship with another nucleic acid sequence. For example, a promoter or regulatory DNA sequence is said to be “operatively linked” or “operably linked” to a DNA sequence that codes for an RNA and/or a protein if the two sequences situated such that the promoter or regulatory DNA sequence affects the expression level of the coding or structural DNA sequence. Operatively linked means that the DNA sequences being linked are generally contiguous and, where necessary to join two protein coding regions, contiguous and in the same reading frame. However, since enhancers generally function when separated from the promoter by several kilobases and intronic sequences may be of variable length, some polynucleotide elements may be operatively linked but not contiguous.

The term “coding region”, as used herein, refers to a region of a nucleic acid that contains the coding sequence. The coding region may contain other regions from the corresponding gene including introns. The term “coding sequence” (CDS) refers to the nucleic acid sequence containing the codons that encode a protein. The coding sequence generally begins with a translation start codon (e.g. ATG) and ends with a translation stop codon. Sequences said to be upstream of a coding sequence are 5′ to the translational start codon and sequences downstream of a CDS are 3′ of the translational stop codon.

The term “ORF” means open reading frame.

The term “gene” refers to a defined region that is located within a genome and that, in addition to the aforementioned coding sequence, comprises other, primarily regulatory, nucleic acid sequences responsible for the control of expression, i.e., transcription and translation of the coding portion. A gene may also comprise other 5′ and 3′ untranslated sequences and termination sequences. Depending on the source of the gene, further elements that may be present are, for example, introns.

The terms “heterologous” and “exogenous” as used herein with reference to nucleic acid molecules such as promoters and gene coding sequences, refer to sequences that originate from a source foreign (non-native) to a particular virus or host cell or, if from the same source, are modified from their original form. Thus, a heterologous gene in a virus or cell includes a gene that is endogenous to the particular virus or cell but has been modified through, for example, codon optimization. The terms also include non-naturally occurring multiple copies of a naturally occurring nucleic acid sequence. Thus, the terms refer to a nucleic acid segment that is foreign or heterologous to the virus or cell, or homologous to the virus or cell but in a position within the host viral or cellular genome in which it is not ordinarily found.

The term “native” refers to a gene that is present in the genome of the wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

The terms “complement” and “complementary” refer to two nucleotide sequences that comprise antiparallel nucleotide sequences capable of pairing with one another upon formation of hydrogen bonds between the complementary base residues in the antiparallel nucleotide sequences. The term “recombinant” as used herein with reference to nucleic acid molecules refers to a combination of nucleic acid molecules that are joined together using recombinant DNA technology into a progeny nucleic acid molecule. As used herein with reference to viruses, cells, and organisms, the terms “recombinant,” “transformed,” and “transgenic” refer to a host virus, cell, or organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Recombinant viruses, cells, and organisms are understood to encompass not only the end product of a transformation process, but also recombinant progeny thereof. A “non-transformed,” “non-transgenic,” or “non-recombinant” host refers to a wildtype virus, cell, or organism that does not contain the heterologous nucleic acid molecule.

“Regulatory elements” are sequences involved in controlling the expression of a nucleotide sequence. Regulatory elements include promoters, enhancers, and termination signals. They also typically encompass sequences required for proper translation of the nucleotide sequence.

The term “promoter” refers to an untranslated DNA sequence usually located upstream of the coding region that contains the binding site for RNA polymerase II and initiates transcription of the DNA. The promoter region may also include other elements that act as regulators of gene expression. The term “minimal promoter” refers to a promoter element, particularly a TATA element that is inactive or has greatly reduced promoter activity in the absence of upstream activation elements.

The term “enhancer” within the meaning of the invention may be any genetic element, e.g., a nucleotide sequence that increases transcription of a coding sequence operatively linked to a promoter to an extent greater than the transcription activation effected by the promoter itself when operatively linked to the coding sequence, i.e. it increases transcription from the promoter.

A “chimeric adenoviral region” is defined as a portion of an adenoviral vector that contains both a portion of a native adenovirus genome and a portion of a heterologous DNA sequence. For example, a “chimeric adenoviral leader region” comprises at least a portion of a native adenoviral leader region, such as L1, L2, L3, L4, L5, and further comprises a heterologous DNA sequence. In one embodiment, the heterologous DNA sequence comprises a transgene CDS. In another embodiment, the heterologous DNA sequence comprises a transgene CDS, a branch site and a splice acceptor site.

The terms “transcriptional regulation elements” and “translational regulation elements” are those elements that affect transcription and/or translation of nucleic acids. These elements include, but are not limited to, splice donor and acceptor sites, translation stop and start codons, and adenylation signals.

As used herein, a “transcriptional response element” or “transcriptional regulatory element”, or “TRE” is a polynucleotide sequence, preferably a DNA sequence, comprising one or more enhancer(s) and/or promoter(s) and/or promoter elements such as a transcriptional regulatory protein response sequence or sequences, which increases transcription of an operatively linked polynucleotide in a host cell that allows a TRE to function.

“Under transcriptional control” is a term well understood in the art and indicates that transcription of a polynucleotide sequence, usually a DNA sequence, depends on its being operatively linked to an element which contributes to the initiation of, or promotes, transcription.

The term “vector”, as used herein, refers to a nucleic acid construct designed for transfer between different host cells. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector. Any vector for use in gene introduction can basically be used as a “vector” into which the DNA having the desired sequence is to be introduced. Plasmid vectors will find use in practicing the present invention. The term vector as it applies to the present invention is used to describe a recombinant vector, e.g., a plasmid or viral vector (including a replication defective or replication competent virus). The terms “vector,” “polynucleotide vector,““polynucleotide vector construct,” “nucleic acid vector construct,” and “vector construct” are used interchangeably herein to mean any nucleic acid construct for gene transfer, as understood by one skilled in the art.

The term “replication defective” as used herein relative to a viral vector of the invention means the viral vector cannot further replicate and package its genomes. For example, when the cell of a subject are infected with an adenoviral vector that has the entire E1 and the E4 coding region deleted or inactivated, the heterologous transgene is expressed in the patient's cells if the transgene is transcriptionally active in the cell. However, due to the fact that the patient's cells lack the Ad E1 and E4 coding sequences, the Ad vector is replication defective and viral particles cannot be formed in these cells.

The term “replication competent” means the vector can replicate in particular cell types (“target cells”), e.g., cancer cells and preferentially effect cytolysis of those cells. Specific replication competent viral vectors have been developed for which selective replication in cancer cells preferentially destroys those cells. Various cell-specific replication competent adenovirus constructs, which preferentially replicate in (and thus destroy) certain cell types. Such viral vectors may be referred to as “oncolytic viruses” or “oncolytic vectors“and may be considered to be “cytolytic” or “cytopathic” and to effect “selective cytolysis” of target cells. Examples of “replication competent” or “oncolytic” viral vectors are described in, for example PCT Publication Nos. WO98/39466, WO95/19434, WO97/01358, WO98/39467, WO98/39465, WO01/72994, WO 04/009790, WO 00/15820, WO 98/14593, WO 00/46355, WO 02/067861, WO 98/39464, WO 98/13508, WO 20004/009790; U.S. Provisional Application Ser. Nos. 60/511,812, 60/423,203 and U.S. Patent Publication No. US 2001/0053352, each of which is expressly incorporated by reference herein.

The terms “replication conditional viruses”, “preferentially replicating viruses”, “specifically replicating viruses” and “selectively replicating viruses” are terms that are used interchangeably and are replication competent viral vectors and particles that preferentially replicate in certain types of cells or tissues but to a lesser degree or not at all in other types. In one embodiment of the invention, the viral vector and/or particle selectively replicates in tumor cells and or abnormally proliferating tissue, such as solid tumors and other neoplasms. Such viruses may be referred to as “oncolytic viruses” or “oncolytic vectors” and may be considered to be “cytolytic” or “cytopathic” and to effect “selective cytolysis” of target cells. “Preferential replication” and “selective replication” and “specific replication” may be used interchangeably and mean that the virus replicates more in a target cell than in a non-target cell. The virus replicates at a higher rate in target cells than non target cells, e.g. at least about 3-fold higher, at least about 10-fold higher, at least about 50-fold higher, and in some instances at least about 100-fold, 400-fold, 500-fold, 1000-fold or even 1×10⁶ higher. In one embodiment, the virus replicates only in the target cells (that is, does not replicate at all or replicates at a very low level in non-target cells).

The term “plasmid” as used herein refers to a DNA molecule that is capable of autonomous replication within a host cell, either extrachromosomally or as part of the host cell chromosome(s). The starting plasmids herein are commercially available, are publicly available on an unrestricted basis, or can be constructed from such available plasmids as disclosed herein and/or in accordance with published procedures. In certain instances, as will be apparent to the ordinarily skilled artisan, other plasmids known in the art may be used interchangeable with plasmids described herein.

The term “expression” refers to the transcription and/or translation of an endogenous gene, transgene or coding region in a cell.

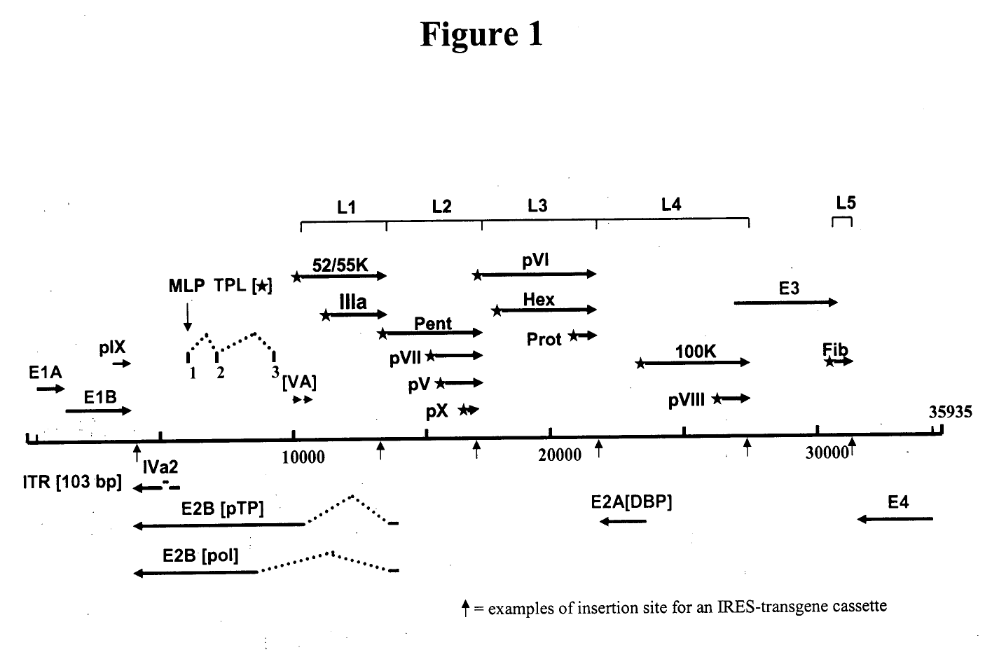

A “polyadenylation signal sequence” is a recognition region for endonuclease cleavage of a RNA transcript that is followed by a polyadenylation consensus sequence AATAAA. A polyadenylation signal sequence provides a “polyA site”, i.e. a site on a RNA transcript to which adenine residues will be added by post-transcriptional polyadenylation. Generally, a polyadenylation signal sequence includes a core poly(A) signal that consists of two recognition elements flanking a cleavage-polyadenylation site (e.g., FIG. 1 of WO 02/067861 and WO 02/068627). The choice of a suitable polyadenylation signal sequence will consider the strength of the polyadenylation signal sequence, as completion of polyadenylation process correlates with poly(A) site strength (Chao et al., Molecular and Cellular Biology, 1999, 19:5588-5600). For example, the strong SV40 late poly(A) site is committed to cleavage more rapidly than the weaker SV40 early poly(A) site. The person skilled in the art will consider choosing a stronger polyadenylation signal sequence if desired. In principle, any polyadenylation signal sequence may be useful for the purposes of the present invention. However, in some embodiments of this invention the termination signal sequence is the SV40 late polyadenylation signal sequence or the SV40 early polyadenylation signal sequence. Usually, the termination signal sequence is isolated from its genetic source or synthetically constructed and inserted into a vector of the invention at a suitable position.

A “multicistronic transcript” refers to a mRNA molecule that contains more than one protein coding region, or cistron. A mRNA comprising two coding regions is denoted a “bicistronic transcript.” The “5′-proximal” coding region or cistron is the coding region whose translation initiation codon (usually AUG) is closest to the 5′-end of a multicistronic mRNA molecule. A “5′-distal” coding region or cistron is one whose translation initiation codon (usually AUG) is not the closest initiation codon to the 5′ end of the mRNA. The terms “5′-distal” and “downstream” are used synonymously to refer to coding regions that are not adjacent to the 5′ end of a mRNA molecule.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene (Jackson R J, Howell M T, Kaminski A (1990) Trends Biochem Sci 15(12):477-83) and Jackson R J and Kaminski, A. (1995) RNA 1(10):985-1000). The present invention encompasses the use of any IRES element, which is able to promote direct internal ribosome entry to the initiation codon of a cistron. PCT publication WO 01/55369 describes examples of IRES sequences including synthetic sequences and these sequences may also be used according to the present invention. “Under translational control of an IRES” as used herein means that translation is associated with the IRES and proceeds in a cap-independent manner. Examples of “IRES” known in the art include, but are not limited, to IRES obtainable from picornavirus (Jackson et al., 1990, Trends Biochem Sci 15(12):477-483); and IRES obtainable from viral or cellular mRNA sources, such as for example, immunoglobulin heavy-chain binding protein (BiP), the vascular endothelial growth factor (VEGF) (Huez et al. (1998) Mol. Cell. Biol. 18(11):6178-6190), the fibroblast growth factor 2, and insulin-like growth factor, the translational initiation factor eIF4G, yeast transcription factors TFIID and HAP4. IRES have also been reported in different viruses such as cardiovirus, rhinovirus, aphthovirus, HCV, Friend murine leukemia virus (FrMLV) and Moloney murine leukemia virus (MoMLV). As used herein, “IRES” encompasses functional variations of IRES sequences as long as the variation is able to promote direct internal ribosome entry to the initiation codon of a cistron. In some embodiments, the IRES is mammalian. In other embodiments, the IRES is viral or protozoan. In one embodiment, the IRES is obtainable from encephelomycarditis virus (ECMV) (commercially available from Novogen, Duke et al. (1992) J. Virol 66(3): 1602-1609). In another illustrative embodiment disclosed herein, the IRES is from VEGF. Examples of IRES sequences are described in U.S. Pat. No. 6,692,736.

A “self-processing cleavage site” or “self-processing cleavage sequence” as referred to herein is a DNA or amino acid sequence, wherein upon translation, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, e.g., a 2A site, sequence or domain. A 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly, 2001). Alternatively, a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

The term “splice acceptor site” or “3′ splice acceptor site” (hereinafter referred to as a 3′ SAS) is a sequence that may be engineered into different locations of the wild type Ad5 genome. Introduction of the site provides a means to facilitate the expression of heterologous stretches of DNA (i.e. transgenes) by utilizing the endogenous alternative splicing mechanisms of adenoviruses. An exemplary 3′ SAS site is: TACTTATGACTCGTACTATTGTTATTCATCCAG↓G (SEQ ID NO: 45) where the underlined bases are the consensus branch site (which is variable), A refers to a branch point, which is highly conserved and the down arrow refers to the splice site (such that sequences 5′ to (before) the arrow are cleaved out, and sequences after the arrow are part of the exon).

The terms “branch point” and “branch point sequence” are used interchangeably and refer to a nucleotide sequence involved in splicing and are understood in the art to be a recognition signal for the site of lariat formation. When referring to a DNA vector, a branch point sequence is the DNA sequence that codes for the RNA branch point sequence.

As used herein, “transgene” refers to a polynucleotide that can be expressed, via recombinant techniques, in a non-native environment or heterologous cell under appropriate conditions. In the present invention, the transgene coding region is inserted in a viral vector. In one embodiment, the viral vector is an adenoviral vector. The transgene may be derived from the same type of cell in which it is to be expressed, but introduced from an exogenous source, modified as compared to a corresponding native form and/or expressed from a non-native site, or it may be derived from a heterologous cell. “Transgene” is synonymous with “exogenous gene”, “foreign gene”, “heterologous coding sequence” and “heterologous gene”. In the context of a vector for use in practicing the present invention, a “heterologous polynucleotide” or “heterologous gene” or “transgene” is any polynucleotide or gene that is not present in the corresponding wild-type vector or virus. The transgene coding sequence may be a sequence found in nature that codes for a certain protein. The transgene coding sequence may alternatively be a non-natural coding sequence. For example, one skilled in the art can readily recode a coding sequence to optimize the codons for expression in a certain species using a codon usage chart. In one embodiment, the recoded sequence still codes for the same amino acid sequence as a natural coding sequence for the transgene. Examples of preferred transgenes for inclusion in the vectors of the invention, are provided herein. A transgene may be a therapeutic gene. A transgene does not necessarily code for a protein.

As used herein, a “therapeutic” gene refers to a transgene that, when expressed, confers a beneficial effect on a cell, tissue or mammal in which the gene is expressed. Examples of beneficial effects include amelioration of a sign or symptom of a condition or disease, prevention or inhibition of a condition or disease, or conferral of a desired characteristic. Numerous examples of therapeutic genes are known in the art, a number of which are further described below.

In the context of a vector for use in practicing the present invention, a “heterologous” sequence or element is one which is not associated with or derived from the corresponding wild-type vector or virus.

In the context of a vector for use in practicing the present invention, an “endogenous” sequence or element is native to or derived from the corresponding wild-type vector or virus. “Replication” and “propagation” are used interchangeably and refer to the ability of a viral vector of the invention to reproduce or proliferate. These terms are well understood in the art. For purposes of this invention, replication involves production of virus proteins and is generally directed to reproduction of virus. Replication can be measured using assays standard in the art and described herein, such as a virus yield assay, burst assay or plaque assay. “Replication” and “propagation” include any activity directly or indirectly involved in the process of virus manufacture, including, but not limited to, viral gene expression; production of viral proteins, replication of nucleic acids or other components; packaging of viral components into complete viruses and cell lysis.

As used herein, a “packaging cell” is a cell that is able to package adenoviral genomes or modified genomes to produce viral particles. It can provide a missing gene product or its equivalent. Thus, packaging cells can provide complementing functions for the genes deleted in an adenoviral genome and are able to package the adenoviral genomes into the adenovirus particle. The production of such particles requires that the genome be replicated and that those proteins necessary for assembling an infectious virus are produced. The particles also can require certain proteins necessary for the maturation of the viral particle. Such proteins can be provided by the vector or by the packaging cell.

“Producer cells” for viral vectors are well known in the art. A producer cell is a cell in which the adenoviral vector is delivered and the adenoviral vector is replicated and packaged into virions. If the viral vector has an essential gene deleted or inactivated, then the producer cell complements for the inactivated gene. Examples of adenoviral vector producer cells are PerC.6 (Fallaux et al. Hum Gene Ther. Sep. 1, 1998; 9(13):1909-17) and 293 cells (Graham et al. J Gen Virol. 1977 July; 36(1):59-74). In the case of selectively replicating viruses, producer cells may be of a cell type in which the virus selectively replicates. Alternatively or in addition, the producer cell may express the genes that are selectively controlled or inactivated in the viral vector.

The term “HeLa-S3” means the human cervical tumor-derived cell line available from American Type Culture Collection (ATCC, Manassas, Va.) and designated as ATCC number CCL-2.2. HeLa-S3 is a clonal derivative of the parent HeLa line (ATCC CCL-2). HeLa-S3 was cloned in 1955 by T. T. Puck et al. (J. Exp. Med. 103: 273-284 (1956)).

An “individual” is a vertebrate, a mammal, or a human. Mammals include, but are not limited to, farm animals, sport animals, rodents, primates, and pets. A “host cell” includes an individual cell or cell culture which can be or has been a recipient of an adenoviral vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with an adenoviral vector of this invention.

As used herein, “cytotoxicity” is a term well understood in the art and refers to a state in which a cell's usual biochemical or biological activities are compromised (i.e., inhibited). These activities include, but are not limited to, metabolism; cellular replication; DNA replication; transcription; translation; uptake of molecules. “Cytotoxicity” includes cell death and/or cytolysis. Assays are known in the art which indicate cytotoxicity, such as dye exclusion, 3H-thymidine uptake, and plaque assays.

As used herein, the terms “neoplastic cells”, “neoplasia”, “tumor”, “tumor cells”, “carcinoma”, “carcinoma cells”, “cancer” and “cancer cells”, (used interchangeably) refer to cells which exhibit relatively autonomous growth, so that they exhibit an aberrant growth phenotype characterized by a significant loss of control of cell proliferation. Neoplastic cells can be malignant or benign.

Adenoviral Vectors

As used herein, the terms “adenovirus” and “adenoviral particle” are used to include any and all viruses that may be categorized as an adenovirus, including any adenovirus that infects a human or an animal, including all known and later discovered groups, subgroups, and serotypes. Thus, as used herein, “adenovirus” (“Ad”) and “adenovirus particle” refer to the virus itself or derivatives thereof and cover all serotypes and subtypes and both naturally occurring and recombinant forms, except where indicated otherwise. Such adenoviruses may be wildtype or may be modified in various ways known in the art or as disclosed herein. Such modifications include changes to the adenovirus genome that are packaged in the particle in order to make an infectious virus. Such modifications also include deletions known in the art, such as deletions in one or more of the adenoviral genes that are essential for replicion, e.g., the E1a, E1b, E2a, E2b, E3, or E4 coding regions. The term “gene essential for replication” refers to a nucleic acid sequence whose transcription is required for a viral vector to replicate in a target cell. For example, in an adenoviral vector of the invention, a gene essential for replication may be selected from the group consisting of the E1a, E1b, E2a, E2b, and E4 genes. The terms also include replication-specific adenoviruses; that is, viruses that preferentially replicate in certain types of cells or tissues but to a lesser degree or not at all in other types of cells or tissues. Such viruses are sometimes referred to as “cytolytic” or “cytopathic” viruses (or vectors), and, if they have such an effect on neoplastic cells, are referred to as “oncolytic” viruses (or vectors).

Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG to immunologically “mask” the antigenicity and/or increase half-life, or conjugated to a nonviral protein.

In the context of adenoviral vectors, the term “5′” is used interchangeably with “upstream” and means in the direction of the left inverted terminal repeat (ITR). In the context of adenoviral vectors, the term “3′” is used interchangeably with “downstream” and means in the direction of the right ITR.

Standard systems for generating adenoviral vectors for expression of inserted sequences are known in the art and are available from commercial sources, for example the Adeno-X expression system from Clontech (Clontechniques (January 2000) p. 10-12).

The adenoviral vectors of the invention include replication defective and replication competent vectors. A replication defective vector does not replicate, or does so at very low levels, in the target cell. In one embodiment, a replication defective vector has at least one coding region in E1a, E1b, E2a, E2b or E4 inactivated, usually by deleting or mutating, part or all of the coding region. Methods for propagating these vectors are well known in the art.

The present invention contemplates the use of all adenoviral serotypes to construct adenoviral vectors and virus particles according to the present invention. Adenoviral stocks that can be employed according to the invention include any adenovirus serotype. Adenovirus serotypes 1 through 47 are currently available from American Type Culture Collection (ATCC, Manassas, Va.), and the invention includes any other serotype of adenovirus available from any source. The adenoviruses that can be employed according to the invention may be of human or non-human origin. For instance, an adenovirus can be of subgroup A (e.g., serotypes 12, 18, 31), subgroup B (e.g., serotypes 3, 7, 11, 14, 16, 21, 34, 35), subgroup C (e.g., serotypes 1, 2, 5, 6), subgroup D (e.g., serotypes 8, 9, 10, 13, 15, 17, 19, 20, 22-30, 32, 33, 36-39, 42-47), subgroup E (serotype 4), subgroup F (serotype 40, 41), or any other adenoviral serotype. Throughout the specification reference is made to specific nucleotides in adenovirus type 5. Based on knowledge generally available to those skilled in the art, one can determine the corresponding nucleotides in other serotypes and therefore construct similar adenoviral vectors in adenovirus serotypes other than serotype 5. In one preferred embodiment, the adenoviral nucleic acid backbone is derived from adenovirus serotype 2 (Ad2), 5 (Ad5) or 35 (Ad35), or a chimeric adenovirus backbone comprising a combination of a portion of adenovirus serotype 2 (Ad2) or 5 (Ad5) with a portion of adenovirus serotype 35 (Ad35).

The DNA and protein sequences of Adenovirus serotypes 2 and 5 can be found in GenBank under Accession Number NC_{—}001405 (Ad2) and AY339865 (Ad5), both of which are incorporated herein in their entirety. Along with the complete genome DNA sequence, the GenBank entries include useful details such as references, location of splicing signals, polyadenylation sites, TATA signals, introns, start and stop codons for each identified gene, protein sequence, cDNA for each gene, and a list of sequence variations that exist throughout the literature. Also, of special interest with regards to the present invention, the mRNA structures for each region can be deduced from the indicated splicing site and polyadenylation cleavage site for each gene or region and the reference list of relevant publications in these GenBank records.

The following references provide details of adenovirus gene locations, gene splicing, locations of transcription elements (e.g. splice sites, polyadenylation signal sequences): Nevins and Chen-Kiang, Adv Virus Res. 1981; 26:1-35.; Akusjarvi and Stevenin, Curr Topics Microbiol Immunology 272:253-86 (2003); Prescott and Falck-Pedersen, Mol Cell Biol 1994 14:4682-4693; Muhlemann et al. 1995 J Virol 69:7324-7327; Nevins and Wilson, Nature. Mar. 12, 1981; 290(5802):113-8; Prescott and Falck-Pederson, J Biol Chem 267:8175; 1992; Larsson, Svensson, and Akusjarvi J Mol Biol 225:287; 1992; Farley, Brown, and Leppard, J Virol 78: 1782; 2004. Adenovirus encodes genes and processing signals for genes on both strands (upper and lower) of its genome.

Care is taken when making any changes that there are no undesired changes on the complimentary strand. Also, in some cases, transgenes are relatively small and therefore multiple transgenes can potentially be inserted into one adenoviral vector genome. Transgenes can be essentially inserted anywhere between the Ad genes as long as essential genes and processing signals on both DNA strands are kept intact. Alternatively, in some situations, it may be desirable to alter existing genes and/or signals to change viral gene expression to suit the purpose of the vector. Also, it may be necessary to add branch point and splice acceptor sites or other processing signals along with the transgene(s) for proper processing of the newly introduced mRNA for the new CDS.

In one preferred aspect, the adenoviral vector is replication-competent or replication conditional. Such vectors are able to replicate in a target cell. Replication competent viruses include wild-type viruses and viruses engineered to replicate in target cells. These include vectors designed to replicate specifically or preferentially in one type of target cell as compared to another. The target cell can be of a certain cell type, tissue type or have a certain cell status. Replication competent adenoviral vectors wherein replication is dependent upon cell type, tissue type or cell status are further described below.

In one embodiment, an adenoviral vector of the invention comprises one or more adenoviral genes essential for replication under transcriptional control of a heterologous transcriptional regulatory element (TRE) which confers selective replication on the adenovirus. The adenoviral gene essential for replication is an early gene, selected from the group consisting of E1A, E1B, E2a, E2b and E4. In a further embodiment, one or more additional TREs is operatively linked to one or more adenoviral genes essential for replication or a transgene, e.g., a therapeutic gene.

The adenoviral E1B 19-kDa region refers to the genomic region of the adenovirus E1B gene encoding the E1B 19-kDa product. According to wild-type Ad5, the E1B 19-kDa region is a 261 bp region located between nucleotide (nt) 1714 and nt 2244. The E1B 19-kDa region has been described in, for example, Rao et al., Proc. Natl. Acad. Sci. USA, 89:7742-7746. In one embodiment, the present invention encompasses deletion of part or all of the E1B 19-kDa region as well as embodiments wherein the E1B 19-kDa region is mutated, as long as the deletion or mutation lessens or eliminates the inhibition of apoptosis associated with E1B-19 kDa.

In an embodiment of the invention, adenovirus vectors replicate preferentially in carcinoma target cells, which replication preference is indicated by comparing the level of replication (e.g., cytolysis or cell killing and/or titer) in carcinoma target cells to the level of replication in non-target, non-carcinoma cells, normal or control cells. Comparison of the titer of a particular adenovirus in a target cell to the titer of the adenovirus in a non-target (or “TRE inactive” cell) indicates that the replication preference is enhanced in target cells and/or depressed in non-target cells.

An adenovirus vector may further include one or more heterologous TREs, which may or may not be operatively linked to the same gene. For example a cell type-specific, cell status-specific or tissue-type specific TRE, (all described herein as forms of “cell-specific” or “target cell specific” TREs) may be juxtaposed to a second TRE of the same or a different type. “Juxtaposed” means a first TRE and a second TRE transcriptionally control the same gene. For these embodiments, the more than one TRE may be in any of a number of configurations, including, but not limited to, (a) next to each other (i.e., abutting); (b) both 5′ to the gene that is transcriptionally controlled (wherein the TREs may have intervening sequences between them); (c) one TRE may be 5′ to the gene and the other TRE 3′ to the gene.

The viral vectors of this invention can be prepared using recombinant techniques that are standard in the art. Methods of modifying replication-competent or replication-incompetent viral vectors are well known in the art and are described herein and in publications cited herein. Various methods for cloning transgenes and desired transcriptional elements into adenovirus are described herein and are standard and well known in the art. The transgene and desired transcriptional elements are cloned into various sites, as described herein, in the adenoviral vector genome. For example, there are various plasmids in the art that contain the different portions of the adenovirus genome, including plasmids that contain the entire adenovirus genome. The construction of these plasmids is also well described in the art (e.g. US20030104625). Once a site is selected for transgene(s) insertion an appropriate plasmid can be used to perform the modifications. Then the modifications may be introduced into a full-length adenoviral vector genome by, for example homologous recombination or in vitro ligation. The homologous recombination may take place in a mammalian cell (e.g. PerC6) or in a bacterial cell (e.g. E. Coli, see WO9617070). Manipulation of the viral vector genome can alternatively or in addition include well known molecular biology methods including, but not limited to, polymerase chain reaction (PCR), PCR-SOEing, restriction digests. If homologous recombination is employed, the two plasmids typically share at least about 500 bp of sequence overlap, although smaller regions of overlap will recombine, but usually with lower efficiencies. Each plasmid, as desired, may be independently manipulated, followed by cotransfection in a competent host, providing complementing genes as appropriate for propagation of the adenoviral vector. Plasmids are generally introduced into a suitable host cell (e.g. 293, PerC.6, Hela-S3 cells) using appropriate means of transduction, such as cationic liposomes or calcium phosphate. Alternatively, in vitro ligation of the right and left-hand portions of the adenovirus genome can also be used to construct recombinant adenovirus derivative containing all the replication-essential portions of adenovirus genome. Berkner et al. (1983) Nucleic Acid Research 11: 6003-6020; Bridge et al. (1989) J. Virol. 63: 631-638.

For convenience, plasmids are available that provide the necessary portions of adenovirus. Plasmid pXC.1 (McKinnon (1982) Gene 19:33-42) contains the wild-type left-hand end of Ad5. pBHG10 (Bett et al. (1994); Microbix Biosystems Inc., Toronto) provides the right-hand end of Ad5, with a deletion in E3. Deletion in E3 provides more room in the viral vector to insert heterologous sequences. The gene for E3 is located on the opposite strand from E4 (r-strand). pBHG11 provides an even larger E3 deletion, an additional 0.3 kb is deleted (Bett et al. (1994). Alternatively, the use of pBHGE3 (Microbix Biosystems, Inc.) provides the right hand end of Ad5, with a full-length of E3.

Methods of packaging polynucleotides into adenovirus particles are known in the art and are also described in PCT/US98/04080. The preferred packaging cells are those that have been designed to limit homologous recombination that could lead to wildtype adenoviral particles. Cells that may be used to produce the adenoviral particles of the invention include the human embryonic kidney cell line 293 (Graham et al., J Gen. Virol. 36:59-72 (1977)), the human embryonic retinoblast cell line PER.C6 (U.S. Pat. Nos. 5,994,128 and 6,033,908; Fallaux et al., Hum. Gene Ther. 9: 1909-1917 (1998)), and the human cervical tumor-derived cell line HeLa-S3 (PCT Application No. US 04/11855). The viral vectors may be delivered to the target cell in a variety of ways, including, but not limited to, liposomes, general transfection methods that are well known in the art (such as calcium phosphate precipitation or electroporation), direct injection, and intravenous infusion. The means of delivery will depend in large part on the particular vector (including its form) as well as the type and location of the target cells (i.e., whether the cells are in vitro or in vivo).

The invention further provides a recombinant adenovirus particle comprising a recombinant viral vector according to the invention. In one embodiment, a capsid protein of the adenovirus particle comprises a targeting ligand. In one approach, the capsid protein is a fiber protein or pIX. In one aspect, the capsid protein is a fiber protein and the ligand is in the C terminus (carboxyl end) or HI loop of the fiber protein. The adenoviral vector particle may also include other mutations to the fiber protein. In an additional embodiment, the virus is targeted by replacing the fiber knob with a fiber knob from another adenovirus serotype. Examples of these mutations include, but are not limited to those described in U.S. application Ser. No. 10/403,337; US Application Publication No. 2004/0002060; PCT Publication Nos. WO 98/07877; WO 99/39734; WO 00/67576; WO 01/92299; and U.S. Pat. Nos. 5,543,328; 5,731,190; 5,756,086; 5,770,442; 5,846,782; 5,962,311; 5,922,315; 6,057,155; 6,127,525; 6,153,435; 6,455,314; 6,555,368 and 6,683,170 and Wu et al. (J Virol. Jul. 1, 2003; 77(13):7225-7235). These include, but are not limited to, mutations that decrease binding of the viral vector particle to a particular cell type or more than one cell type, enhance the binding of the viral vector particle to a particular cell type or more than one cell type and/or reduce the immune response to the adenoviral vector particle in a mammal.

The adenoviral major late transcription unit (MLTU) is divided into five regions in Group C Adenoviruses. Each region is characterized by its own polyadenylation signal sequence and usually consists of several differentially spliced messages. All late messages are spliced at their 5′ end to the tripartite leader (TPL). A description of each region follows.

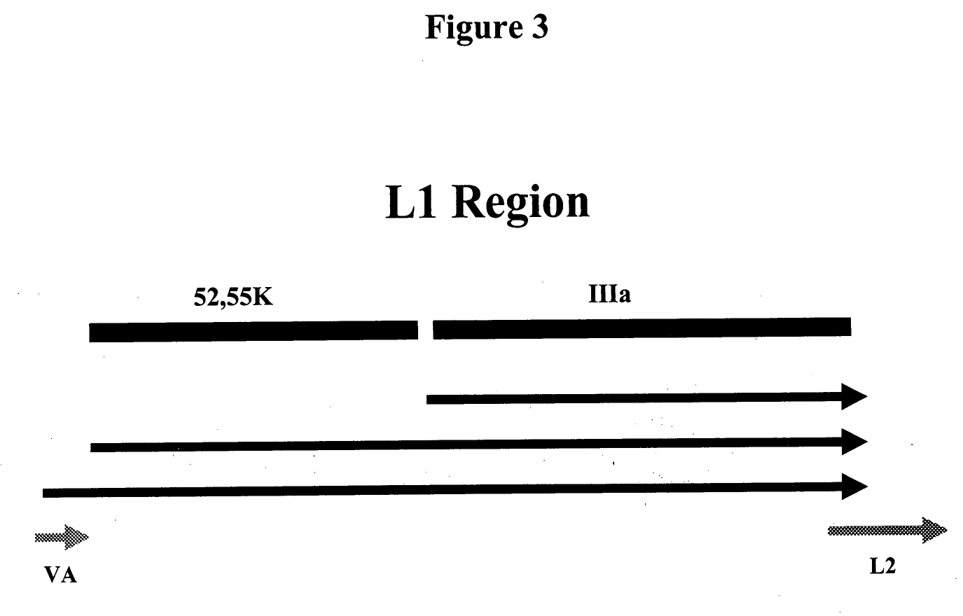

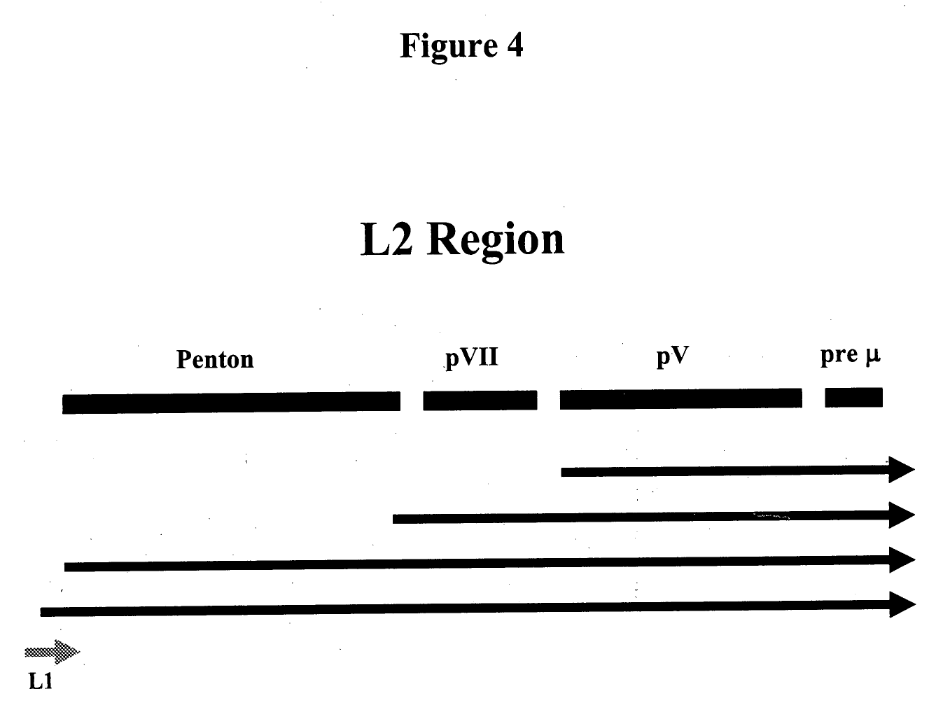

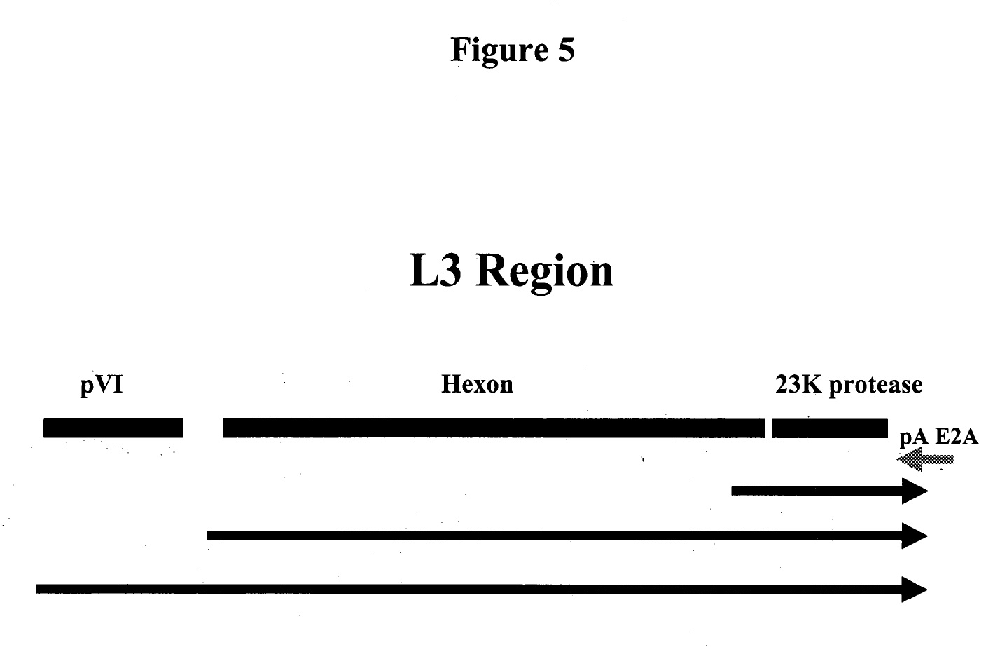

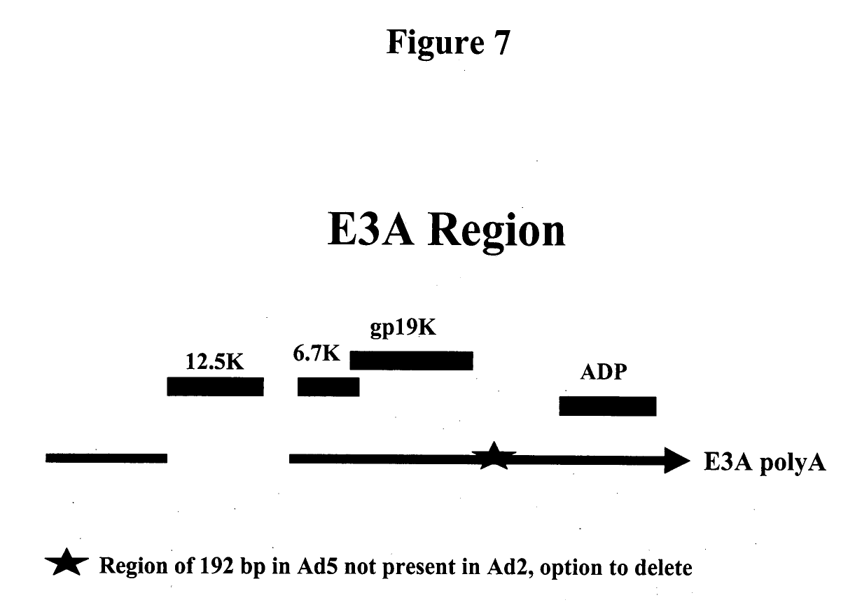

In exemplary embodiments of the present invention, a transgene is inserted in at least one of the following locations: A) L1 region, insert between the 52, 55K and IIIa CDS or after the pIIIa CDS and upstream of the polyadenylation signal sequence of L1 region (FIG. 3); B) L2 region: inserted between either the CDS for pV and Mu, or pVII and pV, and/or after penton and before pVII; FIG. 4); C) L3 region: inserted upstream of pVI, or between pVI and hexon, or between hexon and 23K, or after the 23K gene and upstream of the polyadenylation signal sequence FIGS. 5-7); D) L4 region: may be inserted upstream of the polyadenylation signal sequence of the L4 region using a similar strategy as described for the other regions. However, there is a significant overlap between L4 and E2 transcription units and processing signals. Therefore, one skilled in the art will insert a transgene in a way that does not significantly disrupt the other transcription units and processing signals, unless of course disruption is desired. E) L5 region: Only one protein is encoded in the Ad5 L5 region, the fiber gene. The stop codon is embedded in the polyadenylation signal sequence. Transgenes inserted into L5, are either upstream of the fiber CDS or, if inserted downstream, a polyadenylation signal sequence will be reconstructed/added and the native polyadenylation signal sequence mutated to maintain a stop codon for the fiber CDS and inactivate the polyadenylation function. F) E3 region: Transgenes are inserted without deletions in the E3 region. For example, transgene insertion sites include sites upstream or downstream of E3 genes. G) Other transcription units that exist in the Ad genome, such as intermediate regions encoding pIX and IVa2, can be used in an analogous manner. H) A new transcription unit can be added to the genome. For example, a new late region may be added to the late transcription unit, making a “L6 region”.

In one embodiment, the transgene is expressed as part of one of the L1, L2, L3, L4, or L5 transcripts.

In one embodiment, the transgene in the vector utilizes (i.e is operatively linked to) the same polyA signal used by at least one of the viral mRNAs. For example, when inserting the transgene in a major late transcription unit, the method for inserting the transgene does not create a new or 6th leader sequence. The same polyA signal may be a native Ad polyA or a heterologous polyA signal.

Modifications of sequences near a polyadenylation signal sequence may influence the efficiency of the cleavage. Therefore modifications to these regions may increase, decrease or result in equivalent cleavage efficiency. One skilled in the art can consider the desired timing of expression and amount of expression of the transgene when selecting an insertion site.

In one embodiment of the invention, an IRES or self-processing cleavage sequence, is operatively linked to a transgene that is inserted upstream of a polyadenylation signal sequence and downstream of an adenoviral coding sequence. The polyadenylation signal sequence is usually an adenoviral polyadenylation signal sequence, but can be a heterologous polyadenylation signal sequence. In one embodiment, said heterologous polyadenylation signal sequence replaces the function of an adenoviral polyadenylation signal sequence. In one embodiment of the invention, the IRES or self-processing cleavage sequence and transgene are inserted into an adenovirus transcription unit or leader sequence (e.g. L1, L2, L3, L4) that codes for more than one protein (e.g. due to differential splicing). This embodiment gives the advantage that the IRES or self-processing cleavage sequence will be functional with each mRNA from said transcript or leader region. In other words, each mRNA from said transcription unit should operatively code and express the transgene in addition to the adenoviral coding sequence (CDS). In one example, an IRES or self-processing cleavage sequence followed by a transgene is inserted upstream of the L2 polyadenylation signal, all four types of mRNAs produced from the L2 region would contain the inserted sequences and thus all four types of mRNAs would express the transgene. Without being bound by theory, it is believed that the closer the IRES or self-processing cleavage sequence and transgene are placed to the polyadenylation signal, the higher the expression level of the transgene. One skilled in the art can consider the desired timing of expression and amount of expression of the transgene when selecting an insertion site. For example, if expression of transgenes at an intermediate time in relation to the viral replication cycle is desired, then an IRES or self-processing cleavage sequence transgene cassette may also be inserted upstream of the polyA signal of either pIX or IVa2. Various types of IRESs and self-processing cleavage sequences are available and each one varies in its efficiency to mediate translation. One skilled in the art can choose a particular IRES or self-processing cleavage sequence with a known efficiency for tailored transgene expression efficiency. When adding an IRES or self-processing cleavage sequence and transgene, care must be taken not to disrupt other adenovirus sequences including those coded for on the other strand (i.e. anti-sense strand) unless disruption of certain adenovirus gene expression is desired. Due to the extensive characterization of adenoviral gene expression, one skilled in the art can determine sites that will and will not disrupt adenoviral gene expression.

L1 Region

The L1 region includes the coding regions for 52/55K and IIIa, as shown in FIGS. 1-3. Both genes have only one base upstream of the ATG after the splice acceptor site. In all late regions, the mRNAs encoded in each region are attached to the tripartite leader at their splice acceptor site. The presence and location of splicing enhancer sequences (25 base pair sequences) between 52/55K and IIIa coding regions has been identified. In addition, there is a splice repressor region directly upstream of the splice enhancer. Upstream of the L1 region are the VA RNA coding regions and the region that encodes E2B, which is present on the complementary strand. In fact, VA RNA II ends about 10 base pairs from the splice acceptor site for 52/55K mRNA. The splice acceptor site for the downstream L2 region is upstream of the L1 polyadenylation signal sequence. These overlapping features should be considered and usually will be left intact when inserting transgenes into the L1 region. L1 mRNA begins to be synthesized during the early phase of infection, although only detectable levels of the 52/55K gene are made. After entering the late phase, the IIIa coding region is translated and the level of 52/55K translation is much lower. Again the mechanism of this shift in regulation is due to differential splice site usage which is affected by entry into the late phase of infection.

Transgene(s) can potentially be placed between the two L1 coding regions, or upstream of the 52/55K coding region, or immediately downstream of the IIIa coding region and upstream of the L1 polyadenylation signal sequence. In one embodiment, a branch point sequence and splice acceptor site is added for the transgene and/or downstream for the adenoviral coding region. In another embodiment, a self-processing cleavage site is added downstream of and operatively linked to the IIIa region and then the self-processing cleavage site is operably linked to a transgene located downstream of the self-processing cleavage site. Also, when designing insertions downstream of the IIIa coding region, care must be taken to avoid disruption of the L1 polyadenylation signal sequence and the L2 splice acceptor site. Alternatively, these signals can be restored or altered to change the protein expression, if desired. It is thought that transgenes inserted upstream and downstream of 52/55K will be expressed in the early phase and transgenes inserted downstream of IIIa will be expressed during the late phase of infection, similar to the viral proteins. This may be altered depending on, for example, the disposition of the splice enhancer and repressor regions.

In another embodiment an IRES operatively linked to a transgene is inserted downstream of the IIIa coding region and upstream of (i.e. operatively linked to) the L1 polyadenylation signal sequence or a heterologous polyadenylation signal sequence.

L2 Region

The L2 region encodes four identified coding regions: penton (aka III), proVII (aka pVII), pV, and pX (sometimes called pV precursor or “mu”), as shown in FIGS. 1-2 and 4. Penton has a splice acceptor site 2 bases upstream of its start codon. The splice site for proVII mRNA is embedded in the penton coding region and its start codon is 7 base pairs downstream of the stop codon for penton. The stop codon for proVII, the splice site for pV mRNA, and start codon for pV are separated by 46 and 23 base pairs, respectively. The pX, and its ATG start codon is 124 bases downstream of the pV stop codon. The polyadenylation signal sequence for the L2 region is 26 bases after the pX stop codon, and cleavage takes place about 12 bases after the polyA signal.

Options for transgene insertion include, but are not limited to: a) inserting after pV, or pX CDSs and upstream of a L2 polyadenylation signal sequence, b) inserting downstream of a splice acceptor site for pV mRNA and upstream of the pV CDS, and c) inserting between a penton stop codon and a pVII start codon. When the transgene insertion method and expression relies on alternative splicing, it is desirable to have a splice acceptor site for each coding region, be it an existing or an exogenously added sequence. All L2 mRNA messages will usually use the L2 polyadenylation signal, but in some embodiments a non-native polyA signal may be utilized.

A transgene may also be expressed from these regions of the L2 region using a self-processing cleavage site. In one embodiment, the self-processing cleavage site is operatively linked to the 3′ end of the Ad CDS (e.g. penton, proVII, pV, and pX) and the transgene CDS is operatively linked to the 3′ end of the self-processing cleavage site. In an alternative embodiment, the transgene is inserted up stream of an Ad CDS, wherein the self-processing cleavage site is operatively linked to the 3′ end of the transgene and the Ad CDS is operatively linked to the 3′ end of the self-processing cleavage site. In this case, the transgene is inserted with the proper transcriptional elements (as described herein) so that it is transcribed from the vector. This may include inserting the transgene so it is operatively linked to an Ad splice site and branch point. In one embodiment, the Ad splice site and branch point are the ones linked in the native virus to the downstream Ad CDS. In another embodiment, the transgene is operatively linked to a splice site and branch point wherein either one or both are heterologous.

In another embodiment an IRES operatively linked to a transgene is inserted downstream of the pV coding region and upstream of (i.e. operatively linked to) the L2 polyA signal or a heterologous polyadenylation signal sequence.

L3 Region

The L3 region contains coding regions for pVI, hexon (II), and 23K (viral protease), as shown in FIGS. 5-7. The splice acceptor site for the pVI mRNA is downstream of the L2 polyadenylation site and one base upstream of the pVI start codon. The stop codon for pVI is about 50 bases upstream of the splice acceptor site for the hexon message, and the hexon start codon is about 35 bases from its mRNA splice acceptor site. The splice acceptor site for the 23K mRNA is contained in the coding sequence for hexon, about 95 bases upstream of the hexon stop codon. The start codon for 23K is 32 bases downstream of the hexon stop codon. The stop codon for 23K is about 25 bases upstream of the L3 polyA signal. The L3 polyadenylation region overlaps with the polyadenylation signal for E2A located on the complementary strand. In most cases, it is desirable to keep the polyadenylation signal sequence on both strands intact for efficient processing.

There are several options for transgene insertions into the L3 region: a) upstream of the pVI coding region, b) between pVI and hexon coding region, c) between the hexon and 23K coding region, and d) at the end of 23K CDS. Note that the L3 polyA signal is very strong and hexon mRNA is very abundantly expressed. It is predicted that transgenes inserted into L3 will also be expressed abundantly. Also, for insertions between the hexon and 23K coding regions, it may be desirable to use the splice acceptor site within the hexon coding region (normally used for the 23K mRNA) for the transgene mRNA and add a new branch point and splice acceptor site for synthesis of the 23K message. In another embodiment, the transgene is linked to the hexon CDS with a self-processing cleavage site. In another embodiment an IRES operatively linked to a transgene is inserted downstream of the 23K coding region and upstream of (i.e. operatively linked to) the L3 polyA signal or a heterologous polyadenylation signal sequence.

L4 Region

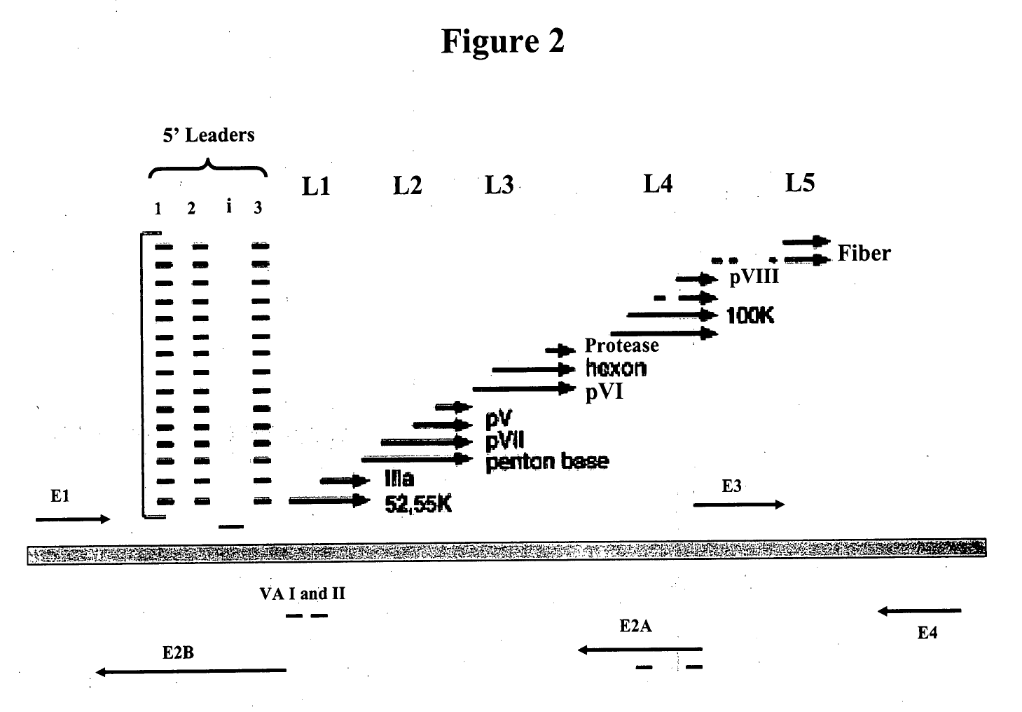

The L4 region contains coding regions for the 100K, 33K, and pVIII proteins, as shown in FIGS. 1 and 2. A portion of the CDS for 33K overlaps the CDS for 100K. Although there are non-coding base pairs between these two genes, it is not a desirable location to insert a transgene since this may disrupt the intron for 33K or alter its usage. The pVIII CDS and the polyA region overlaps the E2 and E3 promoters. On the complementary strand are the leader sequences for the E2A and E2B transcription units. Therefore when utilizing this region for transgene insertion, the designer should be aware of this. It is likely that any insertions or disruptions in this region may alter or disrupt expression of essential genes in the E2, E3, and/or L4 region.

In another embodiment an IRES or self processing cleavage site is operatively linked to a transgene is inserted downstream of the 100K coding region and upstream of (i.e. operatively linked to) the L4 polyA signal or a heterologous polyadenylation signal sequence. In this case, the E3 12.5K CDS will likely be interrupted since its CDS overlaps the L4 polyA sequence. However, some mutants that have this gene deleted have not shown a change in phenotype in vitro, so this location/method for transgene insertion can lead to a functional adenoviral vector.

L5 Region

The L5 region in Ad5 encodes only the fiber protein as shown in FIGS. 1 and 2. The splice acceptor site for fiber mRNA is 2 nucleotides (nts) upstream of the fiber start codon. The fiber stop codon is embedded in the L5 polyA signal. Transgenes can be inserted either upstream or downstream of the fiber coding region. However, for downstream insertions, the native L5 polyadenylation signal sequence is modified so the sequence no longer provides a polyadenylation function. In one embodiment, a splice acceptor site and a transgene are inserted for synthesis of the mRNA. A polyadenylation signal is then restored downstream of the inserted transgene for use by the altered L5 region. In another embodiment, a self-processing cleavage site is operatively linked to the downstream end of the fiber CDS and a transgene CDS is inserted downstream of and operatively linked to the self-processing cleavage site.

Additional Late Region

Another approach to inserting transgenes into a viral vector, such as Ad is to insert an additional late region. This can be done, for example, by inserting just downstream of an existing region or transcription unit (such as L3, for example), a cassette consisting of a branch point and a splice acceptor site, a transgene(s), and a polyadenylation signal (and any other necessary signals). This cassette could also consist of an IRES or self processing cleavage site operatively linked to a second transgene. The second transgene could code for the same or a different protein as the first transgene. In the case of the same protein, it is preferred that the coding sequence of one of the transgenes be “recoded”. In other words, use different codons to code for the same amino acids. This is done to reduce the amount of homology between the two transgenes at the DNA level, thus reducing or eliminating homologous recombination between the two transgenes.

E3 Region

The E3 region in adenovirus encodes the genes for the 12.5K, 6.7K, gp19K, ADP, RID-alpha, RID-beta and 14.7K identified proteins. In one embodiment, a transgene is inserted downstream of the gp19K coding region and upstream of the ADP CDS. Interestingly, there are 128 bps in the Ad5 virus located between the gp19K and ADP coding regions (base pairs 29209-29336 in Ad genome accession number AY339865; SEQ ID NO:41).

In one embodiment of the invention, these 128 bps or the majority of these 128 bps are deleted from an Ad5 vector. In one embodiment, the vector contains the gp19K and/or ADP coding regions and is deleted for these 128 bps in an Ad5 vector. Ad2 virus does not contain these 128 bps or similar sequences in this region. In another embodiment, a transgene is inserted after the 14.7K coding region and upstream of the E3B polyadenylation signal sequence. In another embodiment, a transgene is inserted after the ADP coding region. However the ADP stop codon is embedded in the E3A polyA site. Therefore, the polyA site would be modified so the sequence no longer provides a polyadenylation function, but still contains a stop codon in frame with the ADP coding sequence. A transgene can be added (e.g. along with a splice acceptor site or self-processing cleavage site) downstream of the ADP CDS. Then a polyadenylation signal sequence is inserted downstream of the transgene coding region. The inserted polyadenylation signal sequence may be the native E3A polyA site or another polyA site (e.g. SV40 polyA site).

The E3 coding regions are not required for viral replication. Therefore, the E3 coding regions are not necessary in an adenoviral vector. As a result, viral vectors of the invention may have one or more E3 coding regions deleted or alternatively, may contain all of the E3 coding regions. In embodiments of the invention, a E3 14.7, 14.5 or 10.4k coding regions or combinations thereof are retained in the adenoviral vector. In one embodiment of the invention, the transgene is not inserted in place of an E3 CDS. For example, the transgene is inserted between two native E3 CDSs, wherein said two native coding sequences are adjacent to each other in the native virus and do not have another E3 CDS located between them. In some embodiments, adenoviral vectors of the invention contain a heterologous splice acceptor site operatively linked to a transgene, which is transcribed as part of the E3 transcription unit. For example, a heterologous splice acceptor site is operatively linked to a transgene and both are inserted into the E3 region.

In another embodiment, a self-processing cleavage site operatively linked to a transgene CDS is inserted so the self-processing cleavage site is also operatively linked to an adenoviral E3 CDS. The transgene CDS may be downstream of the self-processing cleavage site, with the E3 CDS being upstream of the self-processing cleavage site. In another embodiment, the E3 CDS may be downstream of the self-processing cleavage site, with the transgene CDS being upstream of the self-processing cleavage site.

In another embodiment an IRES operatively linked to a transgene is inserted downstream of the 14.7K coding region and upstream of (i.e. operatively linked to) the E3B polyA signal or a heterologous polyadenylation signal sequence. In one embodiment, an IRES operatively linked to a transgene is inserted downstream of the ADP coding region and upstream of (i.e. operatively linked to) the E3A polyA signal or a heterologous polyadenylation signal sequence. The E3 region codes for proteins that are not necessary for viral replication and therefore are dispensable for certain type of and uses of adenoviral vectors. Therefore, in another embodiment one or more E3 coding sequences are deleted or mutated and the heterologous DNA comprised of an IRES operatively linked to a transgene is inserted between the E3A or E3B polyadenylation signal sequence and the E3 coding sequence present immediately 5′ to the E3A or E3B polyadenylation signal sequence, respectively.

Any of the methods for transgene insertions described herein may be combined with any other method of transgene insertion described herein or elsewhere as long as the sites of insertion are compatible. For example, a first transgene operatively linked to a splice acceptor site and a branch point may be inserted in a transcriptional unit. The inserted first transgene may then be operatively linked to a self-processing cleavage site inserted downstream of said first transgene and a second transgene is then inserted downstream of and operatively linked to the self-processing cleavage site. It is appreciated that one skilled in the art can rely on the teachings herein and insert transgenes using combinations of the disclosed transgene insertion and expression methods, all of which methods and insertion sites are encompassed by the present invention.

If the adenoviral vector DNA is to be packaged into a viral virion, then care must be taken not to exceed the packaging capacity of the virus. For example, for Ad5 when the genomic DNA is larger than about 105% of the size of the wild-type Ad5 genome, the packaging efficiency greatly decreases (Bett et al. Virol. 1993 October; 67(10):5911-21). However, Ad5 vectors of larger sizes have been reported to be stable.

For the purposes of simplicity, the descriptions hereinabove, unless otherwise stated, refer to Ad serotype 5. One skilled in the art can readily deduce without undue experimentation equivalent insertion sites for transgenes in other adenoviruses or other viruses.

Depending on the properties of the transgene(s), it may be desirable to express it during the early or late phase of infection; additionally, it may be desirable to have either high or low levels of expression. For example, in some cases, there may be a preference for expression during the late phase of infection following viral DNA replication. If the vector is an oncolytic Ad vector with a tumor-specific promoter driving expression of early genes, then late expression allows an extra regulation mechanism because late gene expression requires DNA replication. If the transgene is inserted in a late transcript, expression will be specific since viral replication will be specific for the target cells if the Ad vector is designed to replicate selectively.

Another mechanism that can be used to help regulate expression of transgenes or change expression of viral genes, is to modify the sequence of the added elements (e.g. branch point sequences and/or splice acceptor sites or self-processing cleavage sites) as compared to a consensus sequence. This changes the efficiency of usage. For example, in the case of a splice acceptor site, if high expression levels are desired, one would use sequences very close to a consensus splice acceptor sequence. A consensus splice acceptor site sequence was determined to be (T/C)₈, N, C/T, A, G, G (Mount, Nucleic Acids Res 10:459; 1982). Mutational analysis has been performed that changes the efficiency of cleavage (Roscigno, et al. J Biol Chem 268: 11222; 1993; Lee et al. Gene Therapy 11: 94; 2004). It should be noted that the general accepted consensus sequence definitions and usage can and do change after the virus goes into late phase (Akusjarvi and Svevenin, Curr Top Microbiol Immunol 272: 253; 2003; Nevins and Wilson, Nature 290:113; 1981; Akusjarvi and Persson, J Virol 38: 469; 1981). For example, non-consensus splice acceptor site usage is enhanced during the late phase of infection (Muhlemann et al. J Virol 69: 7324; 1995). Most of these changes are due to the effect that viral proteins have on the cellular machinery for transcription and translation.

In addition to the splice acceptor site, a branch point sequence may be included in the adenoviral vectors of the invention. A branch point sequence is necessary for efficient splicing of an intron (Vandenbroucke et al., BMC Genomics 3: 13; 2002; Hall et al., PNAS 85:704-708; 1988; Harris et al. Nucl Acid Res 18(10) 3015-3019 1990). The distance of the branch point to the splice acceptor site also influences the efficiency of splicing and is usually, but not always, located 10-50 and even more frequently 18-37 base pairs upstream of the splice acceptor site (Vandenbroucke et al., 2002; Hall et al. 1988; Harris et al. 1990). The consensus sequence of a branch point sequence is believed to be YNYURAY (SEQ ID NO:40) (where Y is a pyrimidine, N is any nucleotide, and R is a purine) and the underlined A is invariant (Liao et al., Virology 323:131; 2004). Any deviations in this sequence may result in changes in efficiency of usage of the branch point and splice acceptor site.

In one embodiment of the invention, the sequence of the branch point plus splice acceptor sequence is: TACTTAT GACTCGTACTATTGTTATTCATCC AG↓G (SEQ ID NO:39) The underlined sequence is the branch point sequence and the arrow indicates the location of the splice site according to splicing rules. Since the rules governing the consensus sequence are not invariant, other similar sequences that conform to the rules can be used. In one embodiment, a branch point plus splice acceptor site is used from another Ad serotype. In other words, the branch point plus splice acceptor site is hetereologous, i.e., not from the native adenovirus that the Ad vector is based on, but is from a different Ad serotype.

Other optional sequences can be added that change the efficiency of splicing and/or expression of the transgene as desired. For example, cis-acting elements present in the exon have been identified that can enhance or suppress splicing. These sequences are called exonic splicing enhancer (ESE) or exonic splicing suppressor (ESS) (reviewed in Zheng, J Biomed Sci 11:278; 2004). Although there are not consensus sequences for these elements, many examples of ESSs and ESEs have been identified in other viral and non-viral organisms and these may be used. One skilled in the art is able to survey the literature and data and choose appropriate sequences.

In summary, the present invention provides methods for inserting transgene coding regions in specific regions of the viral vector genome. The methods take advantage of known viral transcription elements and the mechanisms for expression of Ad genes, reduce the size of the DNA sequence for transgene expression that is inserted into the Ad genome since no additional promoter is necessary and the regulation signals encompass a smaller size DNA fragment, provide flexibility in temporal regulation of the transgene (e.g. early versus late stage of infection; early versus intermediate stage of infection), and provide techniques to regulate the amount of transgene expressed. For example, a higher amount of transgene can be expressed by inserting the transgene into a transcript that is expressed normally at high levels and/or by operatively linking a high efficiency splice acceptor site to the transgene coding region. Expression levels are also affected by how close the regulating signals are to their consensus sequences; changes can be made to tailor expression as desired.

In some cases expression of a transgene may inhibit the life cycle of a replication competent virus. In this case the transgene may be inserted in a way that the transgene is only or mostly expressed at the late stages of infection (after viral DNA replication). For example, the transgene may be inserted, according to the present invention, in L3. For some transgenes, it may be desired to express the transgene early in the viral life cycle. For example, the transgene may be inserted in any of the early regions (e.g. E3) or into the upstream L1 region.

Transcriptional Regulatory Elements (TREs)

Transcriptional regulatory element (TREs), as well as methods for their identification, isolation, characterization, genetic manipulation and use for regulation of operatively linked coding sequences, are known in the art. A TRE can be derived from the transcriptional regulatory sequence of a single gene, sequences from different genes can be combined to produce a functional TRE, or a TRE can be synthetically generated (e.g. the CTP4 promoter).

A TRE can be tissue-specific, tumor-specific, developmental stage-specific, cell status specific, etc., depending on the type of cell present in the target tissue or tumor. Such TREs are collectively referred to herein as tissue-specific or target cell-specific. As described in more detail below, a target cell-specific TRE can comprise any number of configurations, including, but not limited to, a target cell-specific promoter and target cell-specific enhancer; a heterologous promoter and a target cell-specific enhancer; a target cell-specific promoter and a heterologous enhancer; a heterologous promoter and a heterologous enhancer; and multimers of the foregoing. The promoter and enhancer components of a target cell-specific TRE may be in any orientation and/or distance from the coding sequence of interest, as long as the desired target cell-specific transcriptional activity is obtained.

Transcriptional activation can be measured in a number of ways known in the art (and described in more detail below), but is generally measured by detection and/or quantitation of mRNA or the protein product of the coding sequence under control of (i.e., operably linked to) the target cell-specific TRE.

As further discussed herein, a target cell-specific TRE can be of varying lengths, and of varying sequence composition. A target cell-specific TRE is preferentially functional in a limited population (or type) of cells, e.g., prostate cells, liver cells, melanoma cells, etc. Accordingly, in some embodiments, the TRE used is preferentially functional in any of the following tissue types: prostate; liver; breast; urothelial (bladder); colon; lung; ovarian; pancreas; stomach; uterine, etc.

As is readily appreciated by one skilled in the art, a TRE is a polynucleotide sequence, and, as such, can exhibit function over a variety of sequence permutations. Methods of nucleotide substitution, addition, and deletion are known in the art, and readily-available functional assays (such as the CAT or luciferase reporter gene assay) allow one of ordinary skill to determine whether a sequence variant exhibits requisite cell-specific transcription regulatory function. Hence, functionally preserved variants of TREs, comprising nucleic acid substitutions, additions, and/or deletions, can be used in the vectors disclosed herein. Accordingly, variant TREs retain function in the target cell but need not exhibit maximal function. In fact, maximal transcriptional activation activity of a TRE may not always be necessary to achieve a desired result, and the level of induction afforded by a fragment of a TRE may be sufficient for certain applications. For example, if used for treatment or palliation of a disease state, less-than-maximal responsiveness may be sufficient if, for example, the target cells are not especially virulent and/or the extent of disease is relatively confined.

Certain base modifications may result in enhanced expression levels and/or cell-specificity. For example, nucleic acid sequence deletions or additions within a TRE can move transcription regulatory protein binding sites closer or farther away from each other than they exist in their normal configuration, or rotate them so they are on opposite sides of the DNA helix, thereby altering spatial relationship among TRE-bound transcription factors, resulting in a decrease or increase in transcription, as is known in the art. Thus, while not wishing to be bound by theory, the present disclosure contemplates the possibility that certain modifications of a TRE will result in modulated expression levels as directed by the TRE, including enhanced cell-specificity. Achievement of enhanced expression levels may be especially desirable in the case of more aggressive forms of neoplastic growth, and/or when a more rapid and/or aggressive pattern of cell killing is warranted (for example, in an immunocompromised subject).

A TRE for use in the present vectors may or may not comprise a silencer. The presence of a silencer (i.e., a negative regulatory element known in the art) can assist in shutting off transcription (and thus replication) in non-target cells. Thus, presence of a silencer can confer enhanced cell-specific vector replication by more effectively preventing replication in non-target cells. Alternatively, lack of a silencer may stimulate replication in target cells, thus conferring enhanced target cell-specificity.

Transcriptional activity directed by a TRE (including both inhibition and enhancement) can be measured in a number of ways known in the art, but is generally measured by detection and/or quantitation of mRNA and/or of a protein product encoded by the sequence under control of (i.e., operably linked to) a TRE.

As discussed herein, a TRE can be of varying lengths, and of varying sequence composition. The size of a heterologous TRE will be determined in part by the capacity of the viral vector, which in turn depends upon the contemplated form of the vector. Generally minimal sizes are preferred for TREs, as this provides potential room for insertion of other sequences which may be desirable, such as transgenes and/or additional regulatory sequences. In a preferred embodiment, such an additional regulatory sequence is an IRES, a self-processing cleavage sequence such as a 2A or 2A-like sequence, a splicing sequence or a branch site.

By way of example, an adenoviral vector can be packaged with extra sequences totaling up to about 105% of the genome size, or approximately 1.8 kb, without requiring deletion of viral sequences. If non-essential sequences are removed from the adenovirus genome, an additional 4.6 kb of insert can be tolerated (i.e., for a total insertion capacity of about 6.4 kb).

In the case of replication-competent adenoviral vectors, in order to minimize non-specific replication, endogenous (adenovirus) TREs (i.e., the native E1A and/or E1B promoter) are preferably removed from the vector. Besides facilitating target cell-specific replication, removal of endogenous TREs also provides greater insert capacity in a vector, which is of special concern if an adenoviral vector is to be packaged within a virus particle. Even more importantly, deletion of endogenous TREs prevents the possibility of a recombination event whereby a heterologous TRE is deleted and the endogenous TRE assumes transcriptional control of its respective adenovirus coding sequences (thus allowing non-specific replication). In one embodiment, an adenoviral vector is constructed such that the endogenous transcription control sequences of one or more adenoviral genes are deleted and replaced by one or more heterologous TREs. However, endogenous TREs can be maintained in the adenovirus vector(s), provided that sufficient cell-specific replication preference is preserved. These embodiments are constructed by inserting heterologous TREs between an endogenous TRE and a gene coding segment required for replication. Requisite cell-specific replication preference is determined by conducting assays that compare replication of the adenovirus vector in a cell which allows function of the heterologous TREs with replication in a cell which does not.

Generally, a TRE will increase replication of a vector in a target cell by at least about 2-fold, preferably at least about 5-fold, preferably at least about 10-fold more preferably at least about 20-fold, more preferably at least about 50-fold, more preferably at least about 100-fold, more preferably at least about 200-fold, even more preferably at least about 400- to about 500-fold, even more preferably at least about 1000-fold, compared to basal levels of replication in the absence of a TRE. The acceptable differential can be determined empirically (by measurement of mRNA levels using, for example, RNA blot assays, RNase protection assays or other assays known in the art) and will depend upon the anticipated use of the vector and/or the desired result.

Adenoviral vectors directed at specific target cells can be generated using TREs that are preferentially functional in a target cell. In one embodiment of the present invention, a target cell-specific or cell status-specific, heterologous TRE is tumor cell-specific. A vector can comprise a single tumor cell-specific TRE or multiple heterologous TREs which are tumor cell-specific and functional in the same cell. In another embodiment, a vector comprises one or more heterologous TREs which are tumor cell-specific and additionally comprises one or more heterologous TREs which are tissue specific, whereby all TREs are functional in the same cell.

In a preferred embodiment for the oncolytic adenovirus platform, bicistronic or multicistronic cassettes containing an IRES or a self processing cleavage sequence such as a 2A or 2A-like sequence comprise adenoviral early viral genes (E1A, E1B, E2, E3, and/and or E4) or genes expressed later in the viral life cycle (fiber, penton, and hexon).

In certain instances, it may be desirable to enhance the degree and/or rate of cytotoxic activity, due to, for example, the relatively refractory nature or particular aggressiveness of the cancerous target cell. An example of a viral gene that contributes to cytotoxicity includes, but is not limited to, the adenovirus death protein (ADP) gene. In another embodiment disclosed herein, the adenovirus comprises the adenovirus E1B gene which has a deletion in or of its endogenous promoter. In other embodiments disclosed herein, the 19-kDa region of E1B is deleted.

To provide enhanced cytotoxicity to target cells, one or more transgenes having a cytotoxic effect may be present in the vector. Additionally, or alternatively, an adenovirus gene that contributes to cytotoxicity and/or cell death, such as the adenovirus death protein (ADP) gene, can be included in the vector, optionally under the selective transcriptional control of a heterologous TRE and optionally under the translational control of an IRES or a self-processing cleavage sequence, such as a 2A or 2A-like sequence. This could be accomplished by coupling the target cell-specific cytotoxic activity with cell-specific expression of, a heterologous gene or transgene.

Any of a number of heterologous therapeutic genes or transgenes may be included in the replication competent viral vectors of the invention, as further described below.

Typically, the aforementioned bicistronic or multicistronic cassettes are placed under the control of a transcriptional response element, generally a cell type or cell status associated transcriptional regulatory element that is preferentially expressed in cancer or tumor cells. Accordingly, the therapeutic gene included in a given construct will vary dependent upon the type of target cell.

As is known in the art, activity of TREs can be inducible. Inducible TREs generally exhibit low activity in the absence of inducer, and are up-regulated in the presence of an inducer. Inducers include, for example, nucleic acids, polypeptides, small molecules, organic compounds and/or environmental conditions such as temperature, pressure or hypoxia. Inducible TREs may be preferred when expression is desired only at certain times or at certain locations, or when it is desirable to titrate the level of expression using an inducing agent. For example, transcriptional activity from the PSE-TRE, PB-TRE and hKLK2-TRE is inducible by androgen, as described herein and in PCT/US98/04080, expressly incorporated by reference herein. Accordingly, in one embodiment of the present invention, the adenovirus vector comprises an inducible heterologous TRE.

A TRE as used in the present invention can be present in a variety of configurations. A TRE can comprise multimers. For example, a TRE can comprise a tandem series of at least two, at least three, at least four, or at least five target cell-specific TREs. These multimers may also contain heterologous promoter and/or enhancer sequences.

Alternatively, a TRE can comprise one or more promoter regions along with one or more enhancer regions. TRE multimers can also comprise promoter and/or enhancer sequences from different genes. The promoter and enhancer components of a TRE can be in any orientation with respect to each other and can be in any orientation and/or any distance from the coding sequence of interest, as long as the desired cell-specific transcriptional activity is obtained.

As used herein, a TRE derived from a specific gene is referred to by the gene from which it was derived and is a polynucleotide sequence which regulates transcription of an operably linked polynucleotide sequence in a host cell that expresses the gene. For example, as used herein, a “human glandular kallikrein transcriptional regulatory element”, or “hKLK2-TRE” is a polynucleotide sequence, preferably a DNA sequence, which increases transcription of an operably linked polynucleotide sequence in a host cell that allows an hKLK2-TRE to function, such as a cell (preferably a mammalian cell, even more preferably a human cell) that expresses androgen receptor, such as a prostate cell. An hKLK2-TRE is thus responsive to the binding of androgen receptor and comprises at least a portion of an hKLK2 promoter and/or an hKLK2 enhancer (i.e., the ARE or androgen receptor binding site). A human glandular kallikrein enhancer and adenoviral vectors comprising the enhancer are described in WO99/06576, expressly incorporated by reference herein.

As used herein, a “probasin (PB) transcriptional regulatory element”, or “PB-TRE” is a polynucleotide sequence, preferably a DNA sequence, which selectively increases transcription of an operably-linked polynucleotide sequence in a host cell that allows a PB-TRE to function, such as a cell (preferably a mammalian cell, more preferably a human cell, even more preferably a prostate cell) that expresses androgen receptor. A PB-TRE is thus responsive to the binding of androgen receptor and comprises at least a portion of a PB promoter and/or a PB enhancer (i.e., the ARE or androgen receptor binding site). Adenovirus vectors specific for cells expressing androgen are described in WO98/39466, expressly incorporated by reference herein.

As used herein, a “prostate-specific antigen (PSA) transcriptional regulatory element”, or “PSA-TRE”, or “PSE-TRE” is a polynucleotide sequence, preferably a DNA sequence, which selectively increases transcription of an operably linked polynucleotide sequence in a host cell that allows a PSA-TRE to function, such as a cell (preferably a mammalian cell, more preferably a human cell, even more preferably a prostate cell) that expresses androgen receptor. A PSA-TRE is thus responsive to the binding of androgen receptor and comprises at least a portion of a PSA promoter and/or a PSA enhancer (i.e., the ARE or androgen receptor binding site). A tissue-specific enhancer active in prostate and use in adenoviral vectors is described in WO 95/19434 and WO 97/01358, each of which is expressly incorporated by reference herein.

As used herein, a “carcinoembryonic antigen (CEA) transcriptional regulatory element”, or “CEA-TRE” is a polynucleotide sequence, preferably a DNA sequence, which selectively increases transcription of an operably linked polynucleotide sequence in a host cell that allows a CEA-TRE to function, such as a cell (preferably a mammalian cell, even more preferably a human cell) that expresses CEA. The CEA-TRE is responsive to transcription factors and/or co-factor(s) associated with CEA-producing cells and comprises at least a portion of the CEA promoter and/or enhancer. Adenovirus vectors specific for cells expressing carcinoembryonic antigen are described in WO 98/39467, expressly incorporated by reference herein.

As used herein, an “alpha-fetoprotein (AFP) transcriptional regulatory element”, or “AFP-TRE” is a polynucleotide sequence, preferably a DNA sequence, which selectively increases transcription (of an operably linked polynucleotide sequence) in a host cell that allows an AFP-TRE to function, such as a cell (preferably a mammalian cell, even more preferably a human cell) that expresses AFP. The AFP-TRE is responsive to transcription factors and/or co-factor(s) associated with AFP-producing cells and comprises at least a portion of the AFP promoter and/or enhancer. Adenovirus vectors specific for cells expressing alpha fetoprotein are described in WO 98/39465, expressly incorporated by reference herein.

As used herein, an “a mucin gene (MUC) transcriptional regulatory element”, or “MUC1-TRE” is a polynucleotide sequence, preferably a DNA sequence, which selectively increases transcription (of an operably-linked polynucleotide sequence) in a host cell that allows a MUC1-TRE to function, such as a cell (preferably a mammalian cell, even more preferably a human cell) that expresses MUC1. The MUC1-TRE is responsive to transcription factors and/or co-factor(s) associated with MUC1-producing cells and comprises at least a portion of the MUC1 promoter and/or enhancer.

As used herein, a “urothelial cell-specific transcriptional response element”, or “urothelial cell-specific TRE” is polynucleotide sequence, preferably a DNA sequence, which increases transcription of an operably linked polynucleotide sequence in a host cell that allows a urothelial-specific TRE to function, i.e., a target cell. A variety of urothelial cell-specific TREs are known, are responsive to cellular proteins (transcription factors and/or co-factor(s)) associated with urothelial cells, and comprise at least a portion of a urothelial-specific promoter and/or a urothelial-specific enhancer. Exemplary urothelial cell specific transcriptional regulatory sequences include a human or rodent uroplakin (UP), e.g., UPI, UPII, UPIII and the like. Human urothelial cell specific uroplakin transcriptional regulatory sequences and adenoviral vectors comprising the same are described in WO 01/72994, expressly incorporated by reference herein.

As used herein, a “melanocyte cell-specific transcriptional response element”, or “melanocyte cell-specific TRE” is a polynucleotide sequence, preferably a DNA sequence, which increases transcription of an operably linked polynucleotide sequence in a host cell that allows a melanocyte-specific TRE to function, i.e., a target cell. A variety of melanocyte cell-specific TREs are known, are responsive to cellular proteins (transcription factors and/or co-factor(s)) associated with melanocyte cells, and comprise at least a portion of a melanocyte-specific promoter and/or a melanocyte-specific enhancer. Methods are described herein for measuring the activity of a melanocyte cell-specific TRE and thus for determining whether a given cell allows a melanocyte cell-specific TRE to function. Examples of a melanocyte-specific TRE for use in practicing the invention include but are not limited to a TRE derived from the 5′ flanking region of a tyrosinase gene, a tyrosinase related protein-1 gene, a TRE derived from the 5′-flanking region of a tyrosinase related protein-2 gene, a TRE derived from the 5′ flanking region of a MART-1 gene or a TRE derived from the 5′-flanking region of a gene which is aberrantly expressed in melanoma.

In another aspect, the invention provides adenoviral vectors comprising a metastatic colon cancer specific TRE derived from a PRL-3 gene operably linked to a gene essential for adenovirus replication or a transgene. As used herein, a “metastatic colon cancer specific TRE derived from a PRL-3 gene” or a “PRL-3 TRE” is a polynucleotide sequence, preferably a DNA sequence, which selectively increases transcription of an operably linked polynucleotide sequence in a host cell that allows a PRL-3 TRE to function, such as a cell (preferably a mammalian cell, more preferably a human cell, even more preferably a metastatic colon cancer cell). The metastatic colon cancer-specific TRE may comprise one or more regulatory sequences, e.g. enhancers, promoters, transcription factor binding sites and the like, which may be derived from the same or different genes. In one preferred aspect, the PRL-3 TRE comprises a PRL-3 promoter. The PRL-3 protein tyrosine phosphatase gene has been found to be specifically expressed at a high level in metastatic colon cancers (Saha et al. (2001) Science 294:1343). Originally identified as a member of a group of up-regulated genes in a metastatic colon cancer library, identified by the serial analysis of gene expression (SAGE), the PRL-3 gene was confirmed to be elevated in only the metastases, not the primary cancer or pre-malignant adenomas. Replication competent adenoviral vectors comprising PRL-3 transcriptional regulatory sequences are described in WO 20004/009790. Examples of relevant sequences are presented as a 0.6 kb and 1 kb sequence upstream of the translational start codon for the PRL-3 gene (identified as SEQ ID NO:1 and SEQ ID NO:2 in WO 20004/009790).

In another aspect, the invention provides adenoviral vectors comprising a liver cancer specific TREs derived from the CRG-L2 gene operably linked to a gene essential for adenovirus replication or a transgene. As used herein, a “liver cancer specific TREs derived from the CRG-L2 gene” or a “CRG-L2 TRE” is a polynucleotide sequence, preferably a DNA sequence, which selectively increases transcription of an operably linked polynucleotide sequence in a host cell that allows a CRG-L2 to function, such as a cell (preferably a mammalian cell, more preferably a human cell, even more preferably a hepatocellular carcinoma cell). The hepatocellular carcinoma specific TRE may comprise one or more regulatory sequences, e.g. enhancers, promoters, transcription factor binding sites and the like, which may be derived from the same or different genes. In one preferred aspect, the CRG-L2 TRE may be derived from the 0.8 kb sequence upstream of the translational start codon for the CRG-L2 gene, or from a 0.7 kb sequence contained within the 0.8 kb sequence (residues 119-803); or from an EcoRI to NcoI fragment derived from the 0.8 kb sequence, as described in U.S. Provisional Application Ser. No. 60/511,812, expressly incorporated by reference herein.

In another aspect, the invention provides adenoviral vectors comprising an EBV-specific transcriptional regulatory element (TRE) operably linked to a gene essential for adenovirus replication or a transgene. In one aspect, the EBV specific TRE is derived from a sequence upstream of the translational start codon for the LMP1, LMP2A or LMP2B genes, as further described in U.S. Provisional Application Ser. No. 60/423,203, expressly incorporated by reference herein. The EBV-specific TRE may comprise one or more regulatory sequences, e.g. enhancers, promoters, transcription factor binding sites and the like, which may be derived from the same or different genes.

In yet another aspect, the invention provides adenoviral vectors comprising a hypoxia-responsive element (“HRE”) operably linked to a gene essential for adenovirus replication or a transgene. HRE is a transcriptional regulatory element comprising a binding site for the transcriptional complex HIF-1, or hypoxia inducible factor-1, which interacts with a in the regulatory regions of several genes, including vascular endothelial growth factor, and several genes encoding glycolytic enzymes, including enolase-1. Accordingly, in one embodiment, an adenovirus vector comprises an adenovirus gene, preferably an adenoviral gene essential for replication, under transcriptional control of a cell status-specific TRE such as a HRE, as further described in WO 00/15820, expressly incorporated by reference herein.

In yet another aspect, the invention provides adenoviral vectors comprising a “telomerase promoter” or “TERT promoter” operably linked to a gene essential for adenovirus replication or a transgene. The term “telomerase promoter” or “TERT promoter” as used herein refers to a native TERT promoter and functional fragments, mutations and derivatives thereof. The TERT promoter does not have to be the full-length or wild type promoter. One skilled in the art knows how to derive fragments from a TERT promoter and test them for the desired selectivity. A TERT promoter fragment of the present invention has promoter activity selective for tumor cells, i.e. drives tumor selective expression of an operatively linked coding sequence. In one embodiment, the TERT promoter of the invention is a mammalian TERT promoter. In another embodiment, the mammalian TERT promoter is a human TERT (hTERT) promoter. See, e.g., WO 98/14593 and WO 00/46355 for exemplary TERT promoters that find utility in the compositions and methods of the present invention.

In yet another aspect, the invention provides adenoviral vectors comprising an “E2F promoter” operably linked to a gene essential for adenovirus replication or a transgene. The term “E2F promoter” as used herein refers to a native E2F promoter and functional fragments, mutations and derivatives thereof. The E2F promoter does not have to be the full-length or wild type promoter. One skilled in the art knows how to derive fragments from an E2F promoter and test them for the desired selectivity. An E2F promoter fragment of the present invention has promoter activity selective for tumor cells, i.e. drives tumor selective expression of an operatively linked coding sequence. The term “tumor selective promoter activity” as used herein means that the promoter activity of a promoter fragment of the present invention in tumor cells is higher than in non-tumor cell types. An E2F-responsive promoter has at least one E2F binding site. In one embodiment, the E2F-responsive promoter is a mammalian E2F promoter. In another embodiment, it is a human E2F promoter. For example, the E2F promoter may be the human E2F-1 promoter. Further, the human E2F-1 promoter may be, for example, a E2F-1 promoter having the sequence as described in SEQ ID NO:43. A number of examples of E2F promoters are known in the art (e.g. Parr et al. Nature Medicine 1997:3(10) 1145-1149, WO 02/067861, US20010053352 and WO 98/13508). E2F responsive promoters typically share common features such as Sp I and/or ATT7 sites in proximity to their E2F site(s), which are frequently located near the transcription start site, and lack of a recognizable TATA box. E2F-responsive promoters include E2F promoters such as the E2F-1 promoter, dihydrofolate reductase (DHFR) promoter, DNA polymerase A (DPA) promoter, c-myc promoter and the B-myb promoter. The E2F-1 promoter contains four E2F sites that act as transcriptional repressor elements in serum-starved cells. In one embodiment, an E2F-responsive promoter has at least two E2F sites. In another embodiment, an E2F promoter is operatively linked to the adenovirus E1a region. In a further embodiment, an E2F promoter is operatively linked to the adenovirus E1b region. In yet a further embodiment, an E2F promoter is operatively linked to the adenovirus E4 region.

In one embodiment of the invention, the recombinant viral vectors of the present invention selectively replicate in and lyse Rb-pathway defective cells. In one embodiment, the E2F promoter of the invention is a mammalian E2F promoter. In another embodiment, the mammalian E2F promoter is a human E2F promoter, for example a human E2F promoter which comprises or consists essentially of SEQ ID NO:43. Embodiments of the invention include adenoviral vectors comprising an E2F promoter wherein the E2F promoter comprises a nucleotide sequence selected from the group consisting of: (a) the nucleotide sequence shown in SEQ ID NO:43; (b) a fragment of the nucleotide sequence shown in SEQ ID NO: 43, wherein the fragment has tumor selective promoter activity; (c) a nucleotide sequence having at least 90, 91, 92, 93, 94, 95, 96, 97, 98, 99% or more % identity over its entire length to the nucleotide sequence shown in SEQ ID NO:43 wherein the nucleotide sequence has tumor selective promoter activity; and (d) a nucleotide sequence having a full-length complement that hybridizes under stringent conditions to the sequence shown in SEQ ID NO:43, wherein the nucleotide sequence has tumor selective promoter activity. In another embodiment of the invention, the E2F promoter comprises nucleotides 7 to 270 of SEQ ID NO:43. In another embodiment of the invention, the E2F promoter comprises nucleotides 7 to 270 of SEQ ID NO:43, wherein nucleotide 75 of SEQ ID NO:43 is a T instead of a C.

In other embodiments, a E2F promoter according to the present invention has at least 80, 85, 87, 89, 90, 91, 92, 93, 94, 95, 96, 97, 98, 99% or more sequence identity to the nucleotide sequence shown in SEQ ID NO:43, when compared and aligned for maximum correspondence, as measured using one of the following sequence comparison algorithms or by visual inspection. In one embodiment, the given % sequence identity exists over a region of the sequences that is at least about 50 nucleotides in length. In another embodiment, the given % sequence identity exists over a region of at least about 100 nucleotides in length. In another embodiment, the given % sequence identity exists over a region of at least about 200 nucleotides in length. In another embodiment, the given % sequence identity exists over the entire length of the sequence.

The E2F-responsive promoter does not have to be the full-length or wild type promoter, but should have a tumor-selectivity of at least 3-fold, at least 5-fold, at least 10-fold, at least 20-fold, at least 30-fold, at least 50-fold, at least 100-fold or even at least 300-fold. Tumor-selectivity can be determined by a number of assays using known techniques, such as the techniques employed in WO 02/067861, Example 4, for example RT-PCR or a comparison of replication in selected cell types.

Without being bound by theory, the selectivity of E2F-responsive promoters (hereinafter sometimes referred to as E2F promoters) is reported to be based on the derepression of the E2F promoter/transactivator in Rb-pathway defective tumor cells. In quiescent cells, E2F binds to the tumor suppressor protein pRB in ternary complexes.

The protein urokinase plasminogen activator (uPA) and its cell surface receptor, urokinase plasminogen activator receptor (uPAR), are expressed in many of the most frequently-occurring neoplasms and appear to represent important proteins in cancer metastasis. Both proteins are implicated in breast, colon, prostate, liver, renal, lung and ovarian cancer. Sequence elements that regulate uPA and uPAR transcription have been extensively studied. (Riccio et al. (1985) Nucleic Acids Res. 13:2759-2771; Cannio et al. (1991) Nucleic Acids Res. 19:2303-2308; See also, WO 98/39464).

Heterologous TRE(s) Operatively Linked to Essential Ad Coding Regions

For manipulation of the early genes, the transcription start site of Ad5 E1A is at 498 and the ATG start site of the E1A coding segment is at 560 in the virus genome. This region can be used for insertion of a heterologous TRE. FIG. 1 depicts the native genome organization of Ad5 and FIG. 2 depicts the native Ad5 transcription units.

A restriction site may be introduced by employing polymerase chain reaction (PCR), where the primer that is employed may be limited to the Ad5 genome, or may involve a portion of the plasmid carrying the Ad5 genomic DNA. For example, where pBR322 is used, the primers may use the EcoRI site in the pBR322 backbone and the XbaI site at nt 1339 of Ad5. By carrying out the PCR in two steps, where overlapping primers at the center of the region introduce a nucleotide sequence change resulting in a unique restriction site, one can provide for insertion of a heterologous TRE at that site.

A similar strategy may also be used for insertion of a heterologous TRE element in operative linkage to E1B. The E1B promoter of Ad5 consists of a single high-affinity recognition site for Sp1 and a TATA box. This region extends from Ad5 nt 1636 to 1701. By insertion of a cell-specific heterologous TRE in this region, one can provide for cell-specific transcription of the E1B gene. By employing the left-hand region modified with the cell-specific response element regulating E1A, as the template for introducing a heterologous TRE to regulate E1B, the resulting adenovirus vector will be dependent upon the cell-specific transcription factors for expression of both E1A and E1B. In some embodiments, part or all of the 19-kDa region of E1B is deleted.

Similarly, a heterologous TRE can be inserted upstream of the E2 gene to make its expression cell-specific. The E2 early promoter, mapping in Ad5 from about 27050-27150, consists of a major and a minor transcription initiation site, the latter accounting for about 5% of the E2 transcripts, two non-canonical TATA boxes, two E2F transcription factor binding sites and an ATF transcription factor binding site (for a detailed review of the E2 promoter architecture see Swaminathan et al., Curr. Topics in Micro. and Immunol. (1995) 199 (part 3):177-194.

The E2 late promoter overlaps with the coding sequences of a gene encoded by the counterstrand and is therefore not amenable for genetic manipulation. However, the E2 early promoter overlaps only for a few base pairs with sequences coding for a 33 kD protein on the counterstrand. Notably, the SpeI restriction site (Ad5 position 27082) is part of the stop codon for the above mentioned 33 kD protein and conveniently separates the major E2 early transcription initiation site and TATA-binding protein site from the upstream transcription factor binding sites E2F and ATF. Therefore, insertion of a heterologous TRE having SpeI ends into the SpeI site would disrupt the endogenous E2 early promoter of Ad5 and should allow cell-specific expression of E2 transcripts. For E4, one must use the right hand portion of the adenovirus genome. The E4 transcription start site is predominantly at about nt 35605 for Ad5, the TATA box at about nt 35631 and the first AUG/CUG of ORF I is at about nt 35532. Virtanen et al. (1984) J. Virol. 51: 822-831. Using any of the above strategies for the other genes, a heterologous TRE may be introduced upstream from the transcription start site. For the construction of full-length adenovirus with a heterologous TRE inserted in the E4 region, the co-transfection and homologous recombination may be performed in W162 cells (Weinberg et al. (1983) Proc. Natl. Acad. Sci. 80:5383-5386) which provide E4 proteins in trans to complement defects in synthesis of these proteins.

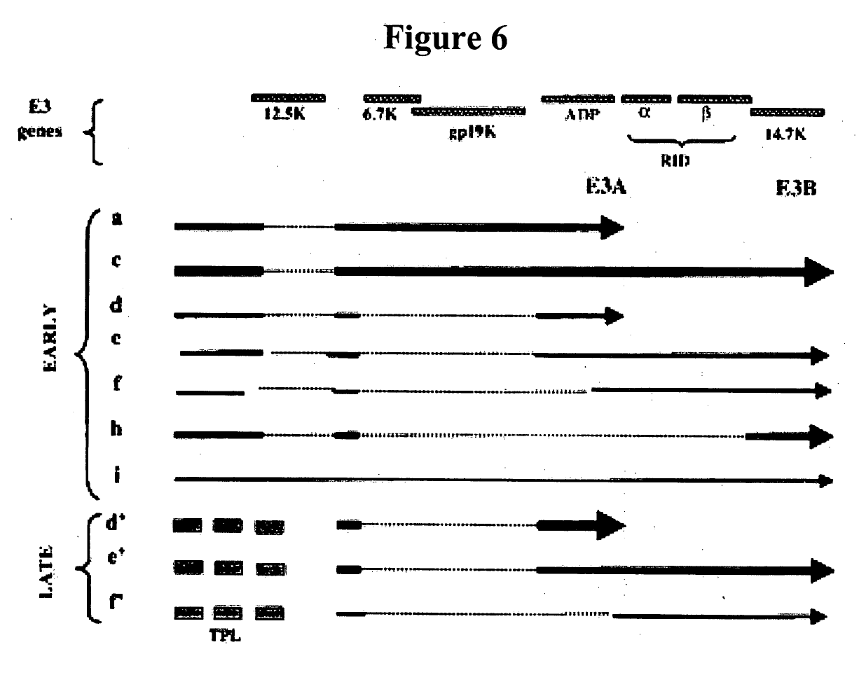

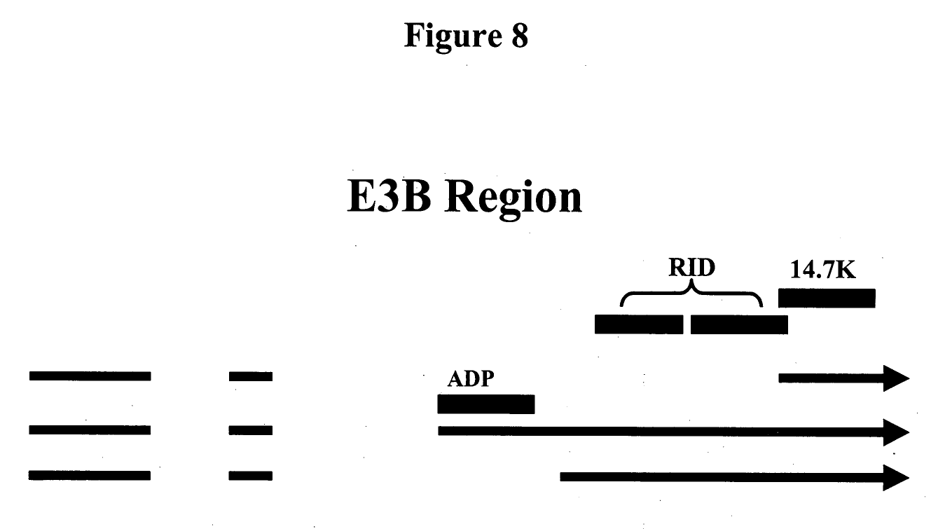

An “E3 region” (used interchangeably with “E3”) is a term well understood in the art and means the region of the adenoviral genome that encodes the E3 gene products. The E3 region has been described in various publications, including, for example, Wold et al. (1995) Curr. Topics Microbiol. Immunol. 199:237-274. A “portion” of the E3 region means less than the entire E3 region, and as such includes polynucleotide deletions as well as polynucleotides encoding one or more polypeptide products of the E3 region. See FIGS. 6, 7 and 8.

Adenoviral constructs containing an E3 region can be generated wherein homologous recombination between an E3-containing adenoviral plasmid, for example, BHGE3 (Microbix Biosystems Inc., Toronto) and a non-E3-containing adenoviral plasmid, is carried out.

Alternatively, an adenoviral vector comprising an E3 region can be introduced into cells, for example 293 cells, along with an adenoviral construct or an adenoviral plasmid construct, where they can undergo homologous recombination to yield adenovirus containing an E3 region. In this case, the E3-containing adenoviral vector and the adenoviral construct or plasmid construct contain complementary regions of adenovirus, for example, one contains the left-hand and the other contains the right-hand region, with sufficient sequence overlap as to allow homologous recombination.

Alternatively, an E3-containing adenoviral vector of the invention can be constructed using other conventional methods including standard recombinant methods (e.g., using restriction nucleases and/or PCR), chemical synthesis, or a combination of any of these. Further, deletions of portions of the E3 region can be created using standard techniques of molecular biology.

In some embodiments, the adenovirus death protein (ADP), encoded within the E3 region, is maintained in an adenovirus vector. The ADP gene, under control of the major late promoter (MLP), appears to code for a protein (ADP) that is important in expediting host cell lysis. Tollefson et al. (1996) J. Virol. 70(4):2296; Tollefson et al. (1992) J. Virol. 66(6):3633. Thus, adenoviral vectors containing the ADP gene may render the adenoviral vector more potent, making possible more effective treatment and/or a lower dosage requirement.

Accordingly in one embodiment, the invention provides adenovirus vectors in which an adenovirus gene is under transcriptional control of a first TRE and a polynucleotide sequence encoding an ADP under control of a second TRE element, and wherein preferably the adenovirus gene is essential for replication. The DNA sequence encoding ADP and the amino acid sequence of an ADP are publicly available. Briefly, an ADP coding sequence is obtained from Ad using techniques known in the art, such as PCR. Preferably, the Y leader (which is an important sequence for correct expression of late genes) is also obtained and ligated to the ADP coding sequence. The ADP coding sequence (with or without the Y leader) can then be introduced into the adenoviral genome, for example, in the E3 region (where the ADP coding sequence will be driven by the MLP). The ADP coding sequence could also be inserted in other locations of the adenovirus genome, such as the E4 region. Alternatively, the ADP coding sequence could be operatively linked to a different type of TRE, including, but not limited to, another viral TRE. In one embodiment, the vector of the invention had ADP operatively linked to its native TREs.

Internal Ribosome Entry Sites

To express two or more proteins from a single viral or non-viral vector, an internal ribosome entry site (IRES) sequence is commonly used to drive expression of the second, third, fourth gene, etc. The adenovirus vectors of the present invention may comprise one or more intergenic IRES elements, which link the translation of two or more coding sequences. Adenovirus vectors comprising an IRES linking two adenoviral coding regions are stable and may provide better specificity than vectors not containing an IRES. An adenovirus vector comprising an intergenic IRES rather than a second TRE may provide for additional space in the vector for inclusion of additional gene(s), e.g., a therapeutic gene. Examples of adenoviral vectors comprising an IRES are described in U.S. Pat. No. 6,692,736, expressly incorporated by reference herein. In one aspect of the invention, the viral vectors comprise at least one IRES within a multicistronic transcript, wherein production of the multicistronic transcript is regulated by a heterologous, cell type-, tissue type- or cell status-specific TRE. For adenovirus vectors comprising a second adenoviral coding region under control of an IRES, it is preferred that the endogenous promoter of the coding region under translational control of the IRES be deleted so that the endogenous promoter does not interfere with transcription of the second coding region. It is preferred that the second coding region be in frame with the IRES if the IRES contains an initiation codon. If an initiation codon, such as ATG, is present in the IRES, it is preferred that the initiation codon of the second coding sequence is removed and that the IRES and the second coding sequence are in frame. Alternatively, if the IRES does not contain an initiation codon or if the initiation codon is removed from the IRES, the initiation codon of the second coding region is used. In one embodiment, the adenovirus vectors comprise the adenovirus essential genes, E1A and E1B genes, under the transcriptional control of a heterologous TRE, and an IRES introduced between E1A and E1B. Thus, both E1A and E1B are under common transcriptional control, and translation of E1B coding region is obtained by virtue of the presence of the IRES. In one embodiment, E1A has its endogenous promoter deleted. In another embodiment, E1A has an endogenous enhancer deleted and in yet an additional embodiment, E1A has its endogenous promoter deleted and E1A enhancer deleted. In another embodiment, E1B has its endogenous promoter deleted. In yet further embodiments, E1B has a deletion of part or all of the 19-kDa region of E1B.

Insertion of an IRES into a vector is accomplished by methods and techniques that are known in the art and described herein supra, including but not limited to, restriction enzyme digestion, ligation, and PCR. A DNA copy of an IRES can be obtained by chemical synthesis, or by making a cDNA copy of, for example, a picornavirus IRES. See, for example, Duke et al. (1995) J. Virol. 66(3): 1602-9) for a description of the EMCV IRES and Huez et al. (1998), Mol. Cell. Biol. 18(11):6178-90) for a description of the VEGF IRES. The internal translation initiation sequence is inserted into a vector genome at a site such that it lies upstream of a 5′-distal coding region in a multicistronic mRNA. For example, in one embodiment of an adenovirus vector in which production of a bicistronic E1A-E1B mRNA is under the control of a heterologous TRE, the E1B promoter is deleted or inactivated, and an IRES sequence is placed between E 1A and E1B. In other embodiments, part or all of the 19-kDa region of E1B is deleted. IRES sequences of cardioviruses and certain aphthoviruses contain an AUG codon at the 3′ end of the IRES that serves as both a ribosome entry site and as a translation initiation site. Accordingly, this type of IRES is introduced into a vector so as to replace the translation initiation codon of the protein whose translation it regulates. However, in an IRES of the entero/rhinovirus class, the AUG at the 3′ end of the IRES is used for ribosome entry only, and translation is initiated at the next downstream AUG codon. Accordingly, if an entero/rhinovirus IRES is used in a vector for translational regulation of a downstream coding region, the AUG (or other translation initiation codon) of the downstream gene is retained in the vector construct.

In another embodiment, an IRES is operatively linked to a transgene inserted downstream of an adenovirus CDS that is not immediately upstream of a polyA signal. For example, the IRES transgene is not inserted after the furthest downstream Ad CDS in a leader sequence. For example, the IRES transgene cassette is operatively linked to a one of the following Ad CDSs: 52/55K, pV, penton, pVI, or hexon.

Multiple coding sequences can be linked with IRESs. In one embodiment, an Ad CDS is operatively linked by a first IRES to a first transgene and said first transgene is operatively linked by a second IRES to a second transgene. In one embodiment, the first and second transgenes encode for the same or different proteins. In the case of the same proteins, it is advantageous that the coding sequence of one of the transgenes be “recoded”. In other words, use different codons to code for the same amino acids. This is done to reduce the amount of homology between the two transgenes at the DNA level, thus reducing or eliminating homologous recombination between the two transgenes. Other embodiments include two adenovirus CDSs operatively linked by an IRES. This may accompany a deletion of adenoviral DNA sequences. For example, two adenoviral CDSs that are located in the same leader region and are adjacent to each other may be operatively linked by an IRES and a portion or all of the intervening Ad sequence may be deleted as long as the deletion does not disrupt other sequences or elements necessary for viral vector production, being especially mindful of the complementary strand. The deleted portion may be 1-5 nucleotides (nts), 6-15 nts, 16-25 nts, 26-35 nts, 36-40 nts, or greater than 40 nts. In one embodiment, a first transgene CDS is operatively linked by a first IRES to an Ad CDS and a second IRES operatively links a second transgene to said Ad CDS. Other embodiments include various combinations of Ad CDSs, and both Ad CDSs and transgene CDSs operatively linked with IRES and/or self-processing peptide sequences.

When using multiple IRES sequences in a vector, it is preferable that the two IRES sequences have minimal or no homology at the DNA level to reduce the frequency of homologous recombination.

Self-Processing Cleavage Sites or Sequences

In another aspect of the invention a “self-processing cleavage site” (e.g. 2A-like sequence) is utilized to express two polypeptides from one mRNA. A “self-processing cleavage site” or “self-processing cleavage sequence” is defined as a DNA or amino acid sequence, wherein upon translation, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, exemplified herein by a 2A site, sequence or domain. As used herein, a “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products. It has been reported that a 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly et al. J Gen Virol. 2001 May; 82(Pt 5):1013-25). Alternatively, it has also been reported that a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

Although the mechanism is not part of the invention, the activity of a 2A-like sequence may involve ribosomal skipping between codons which prevents formation of peptide bonds (de Felipe et al., Human Gene Therapy 11:1921-1931 (2000); Donnelly et al., J. Gen. Virol. 82:1013-1025 (2001); Donnelly et al. J Gen Virol. 2001 May; 82(Pt 5):1027-41); Szymczak et al. Nature Biotechnology 22:589-594 and 760 (2004), although it has been considered that the domain acts more like an autolytic enzyme (Ryan et al., Virol. 173:35-45 (1989)). Studies in which the Foot and Mouth Disease Virus (FMDV) 2A coding region was cloned into expression vectors and transfected into target cells showed FMDV 2A cleavage of artificial reporter polyproteins in wheat-germ lysate and transgenic tobacco plants (Halpin et al., U.S. Pat. No. 5,846,767; 1998 and Halpin et al., Plant J 17:453-459, 1999); Hs 683 human glioma cell line (de Felipe et al., Gene Therapy 6:198-208, 1999); hereinafter referred to as “de Felipe II”); rabbit reticulocyte lysate and human HTK-143 cells (Ryan et al., EMBO J. 13:928-933 (1994)); and insect cells (Roosien et al., J. Gen. Virol. 71:1703-1711, 1990). The FMDV 2A-mediated cleavage of a heterologous polyprotein has been shown for IL-12 (p40/p35 heterodimer; Chaplin et al., J. Interferon Cytokine Res. 19:235-241, 1999). The reference demonstrates that in transfected COS-7 cells, FMDV 2A mediated the cleavage of a p40-2A-p35 polyprotein into biologically functional subunits p40 and p35 having activities associated with IL-12.

The FMDV 2A sequence has been incorporated into retroviral vectors, alone or combined with different IRES sequences to construct bicistronic, tricistronic and tetracistronic vectors. The efficiency of 2A-mediated gene expression in animals was demonstrated by Furler et al. (Gene Ther. 2001 June; 8(11):864-73) using recombinant adeno-associated viral (AAV) vectors encoding a-synuclein and EGFP or Cu/Zn superoxide dismutase (SOD-1) and EGFP linked via the FMDV 2A sequence. EGFP and a-synuclein were expressed at substantially higher levels from vectors which included a 2A sequence relative to corresponding IRES-based vectors, while SOD-1 was expressed at comparable or slightly higher levels. Furler also demonstrated that the 2A sequence results in bicistronic gene expression in vivo after injection of 2A-containing AAV vectors into rat substantia nigra. Syzmczak et al. (Nature Biotechnology 22:589-594&760 (2004)) describe a retroviral vector with four coding regions linked with three 2A sequences.

For the present invention, the DNA sequence encoding a self-processing cleavage site is exemplified by viral sequences derived from a picornavirus, including but not limited to an entero-, rhino-, cardio-, aphtho- or Foot-and-Mouth Disease Virus (FMDV). In a preferred embodiment, the self-processing cleavage site coding sequence is derived from a FMDV. Self-processing cleavage sites include but are not limited to 2A and 2A-like sites, sequences or domains (Donnelly et al., J. Gen. Virol. 82:1027-1041 (2001)).

FMDV 2A is a polyprotein region, which functions in the FMDV genome to direct a single cleavage at its own C-terminus, thus functioning in cis. The FMDV 2A domain is typically reported to be about nineteen amino acids in length ((LLNFDLLKLAGDVESNPGP (SEQ ID NO:1); TLNFDLLKLAGDVESNPGP (SEQ ID NO:2); Ryan et al., J. Gen. Virol. 72:2727-2732 (1991)), however oligopeptides of as few as fourteen amino acid residues ((LLKLAGDVESNPGP (SEQ ID NO:3)) have also been shown to mediate cleavage at the 2A C-terminus in a fashion similar to its role in the native FMDV polyprotein processing. Variations of the 2A sequence have been studied for their ability to mediate efficient processing of polyproteins (Donnelly et al., J. Gen. Virol. 82:1027-1041 (2001)). Homologues and variant 2A sequences are included within the scope of the invention and include, but are not limited to, the sequences presented as SEQ ID NOs: 1-32.

In one embodiment, the FMDV 2A sequence included in a vector according to the invention encodes amino acid residues comprising the sequence presented as SEQ ID NO:1. Alternatively, a vector according to the invention may encode amino acid residues for other 2A-like regions as discussed in Donnelly et al., J. Gen. Virol. 82:1027-1041 (2001) and including, but not limited to, a 2A-like domain from picornavirus, insect virus, Type C rotavirus, trypanosome repeated sequences or the bacterium, Thermatoga maritima.

The invention contemplates the use of nucleic acid sequence variants that encode a self-processing cleavage site, such as a 2A or 2A-like polypeptide, and nucleic acid coding sequences that have a different codon for one or more of the amino acids relative to that of the parent (native) nucleotide. Such variants are specifically contemplated and encompassed by the present invention. Sequence variants of self-processing cleavage peptides and polypeptides are included within the scope of the invention as well.

In accordance with the present invention, also encompassed are sequence variants which encode self-processing cleavage polypeptides and polypeptides themselves that have 80, 85, 88, 89, 90, 91, 92, 93, 94, 95, 96, 97, 98, 99% or more sequence identity to the native sequence.

In one embodiment of the invention, a self-processing cleavage sequence (e.g. 2A or 2A-like sequence) is operably linked to an adenovirus protein coding region and a transgene. The adenovirus protein CDS may be upstream of the self-processing cleavage site, with the transgene being downstream. Alternatively, the transgene CDS may be upstream of the self-processing cleavage site, with the adenovirus protein CDS being downstream.

Multiple CDSs may be linked with self-processing cleavage sites. In one embodiment, an Ad CDS is operatively linked by a self-processing cleavage site to a first transgene and said first transgene is operatively linked by a self-processing cleavage site to a second transgene. In one embodiment, the first and second transgenes encodes for the same or different proteins. In the case of the same proteins, it is advantageous that the coding sequence of one of the transgenes be “recoded”. In other words, use different codons to code for the same amino acids. This is done to reduce the amount of homology between the two transgenes at the DNA level, thus reducing or eliminating homologous recombination between the two transgenes. Other embodiments include two Ad CDSs operatively linked by a self-processing cleavage site. This may accompany a deletion of adenoviral sequence. For example, two adenoviral CDSs that are located in the same leader region and are adjacent to each other may be operatively linked by a self-processing cleavage site and a portion or all of the intervening Ad sequence may be deleted as long the deletion does not disrupt other sequences or elements necessary for viral vector production, being especially mindful of the complementary strand. The deleted portion may be 1-5 nucleotides (nts), 6-15 nts, 16-25 nts, 26-35 nts, 36-40 nts, or greater than 40 nts.

In one embodiment, a first transgene CDS is operatively linked by a first self-processing cleavage site to an Ad CDS and the Ad CDS is operatively linked by a second self-processing cleavage site to a second transgene. Other embodiments include various combinations of Ad CDSs, and both Ad CDSs and transgene CDSs operatively linked with IRES and/or self-processing peptide sequences.

When using more than one self-processing peptide sequences in a vector, it is preferable that the self-processing peptide sequences have minimal or no homology at the DNA level to reduce the frequency of homologous recombination. For example, the self-processing peptide sequences may be derived from different sources wherein the multiple coding sequences for self-processing peptide sequences have minimal or no homology. In another embodiment, a coding sequence for a self-processing peptide sequence is recoded. In other words, different codons are used to code for the same amino acids of the self-processing peptide sequence. This is done to reduce the amount of homology between the more than one self-processing peptide coding sequences, thus reducing or eliminating homologous recombination between the two transgenes.

A self-processing peptide sequence is operatively linked to a CDS when the sequence encoding the self-processing peptide sequence is inserted in frame with the upstream and downstream CDS.

Removal of Self-Processing Peptide Sequences.

One concern associated with the use of self-processing peptides, such as a 2A or 2A-like sequence is that the C terminus of the expressed polypeptide contains amino acids derived from the self-processing peptide, i.e. 2A-derived amino acid residues. These amino acid residues are “foreign” to the host and may elicit an immune response when the recombinant protein is expressed in vivo or delivered in vivo following in vitro or ex vivo expression. In addition, if not removed, self-processing peptide-derived amino acid residues may interfere with protein function and/or alter protein conformation, resulting in a less than optimal expression level and/or reduced biological activity of the recombinant protein. In other words, depending on the application it may be advantageous that the resulting proteins not contain all of the 2A-derived amino acid residues.

The invention includes vectors, engineered such that an additional proteolytic cleavage site is provided between a first protein or polypeptide coding sequence (the first or 5′ ORF) and the self processing cleavage site as a means for removal of self processing cleavage site derived amino acid residues that are present in the expressed protein product.

Examples of additional proteolytic cleavage sites are furin cleavage sites with the consensus sequence RXK(R)R (SEQ ID NO:33), which can be cleaved by endogenous subtilisin-like proteases, such as furin and other serine proteases. As shown in Example 6 of U.S. Ser. No. 10/831304, the inventors have demonstrated that self processing 2A amino acid residues at the C terminus of a first expressed protein can be efficiently removed by introducing a furin cleavage site RAKR (SEQ ID NO:33) between the first polypeptide and a self processing 2A sequence. In addition, use of a plasmid containing a 2A sequence and a furin cleavage site adjacent to the 2A sequence resulting in a higher level of protein expression than achieved using a plasmid containing the 2A sequence alone. This improvement provides a further advantage in that when 2A amino acid residues are removed from the C-terminus of the protein, longer 2A- or 2A like sequences or other self-processing sequences can be used, as described in U.S. Ser. No. 10/831304, expressly incorporated by reference herein.

As detailed herein, the 2A peptide sequence provides a “cleavage” site that facilitates the generation of both chains of an immunoglobulin or other protein during the translation process. In one exemplary embodiment, the C-terminus of the first protein, for example the immunoglobulin heavy chain, contains approximately 13 amino acid residues which are derived from the 2A sequence itself. The number of residual amino acids is dependent upon the 2A sequence used. As set forth above and shown in the Examples, when a furin cleavage site sequence, e.g., RAKR, is inserted between the first protein and the 2A sequence, the 2A residues are removed from the C-terminus of the first protein. However, mass spectrum data indicates that the C-terminus of the first protein expressed from the RAKR-2A construct contains two additional amino acid residues, RA, derived from the furin cleavage site RAKR.

In one embodiment, the invention provides a method for removal of these residual amino acids and a composition for expression of the same. A number of novel constructs have been designed that provide for removal of these additional amino acids from the C-terminus of the protein. Furin cleavage occurs at the C-terminus of the cleavage site, which has the consensus sequence RXR(K)R, where X is any amino acid. In one aspect, the invention provides a means for removal of the newly exposed basic amino acid residues R or K from the C-terminus of the protein by use of an enzyme selected from a group of enzymes called carboxypeptidases (CPs), which include, but not limited to, carboxypeptidase D, E and H (CPD, CPE, CPH). Since CPs are able to remove basic amino acid residues at the C-terminus of a protein, all amino acid resides derived from a furin cleavage site which contain exclusively basic amino acids R or K, such as RKKR, RKRR, RRRR, etc, can be removed by a CP. A series of immunoglobulin expression constructs that contain a 2A sequence and a furin cleavage site and which have basic amino acid residues at the C terminus have been constructed to evaluate efficiency of cleavage and residue removal. An exemplary construct design is the following: H chain-furin (e.g, RKKR, RKRR, RRKR or RRRR)-2A-L chain or L chain-furin (e.g, RKKR, RKRR, RRKR or RRRR)-2A-H chain.

As will be apparent to those of skill in the art, there is a basic amino acid residue (K) at the C terminus of the immunoglobulin heavy (H) chain (rendering it subject to cleavage with carboxypeptidase), while the immunoglobulin light (L) chain, terminates with a non-basic amino acid C. In one preferred embodiment of the invention, an antibody expression construct comprising a furin site and a 2A sequence is provided wherein the immunoglobulin L chain is 5′ to the immunoglobulin H chain such that following translation, the additional furin amino acid residues are cleaved with carboxypeptidase.

It is often advantageous to produce therapeutic proteins, polypeptides, fragments or analogues thereof with fully human characteristics. These reagents avoid the undesired immune responses induced by proteins, polypeptides, fragments or analogues thereof originating from different species. To address possible host immune responses to amino acid residues derived from self-processing peptides, the coding sequence for a proteolytic cleavage site may be inserted (using standard methodology known in the art) between the coding sequence for a first protein and the coding sequence for a self-processing peptide so as to remove the self-processing peptide sequence from the expressed protein or polypeptide.

Any additional proteolytic cleavage site known in the art that can be expressed using recombinant DNA technology may be employed in practicing the invention. Exemplary additional proteolytic cleavage sites which can be inserted between a polypeptide or protein coding sequence and a self processing cleavage sequence include, but are not limited to a:
a). Furin consensus sequence or site: RXK(R)R (SEQ ID. NO:33);
b). Factor Xa cleavage sequence or site: IE(D)GR (SEQ ID NO:34);
c). Signal peptidase I cleavage sequence or site: e.g., LAGFATVAQA (SEQ ID. NO:35); and
d). Thrombin cleavage sequence or site: LVPRGS (SEQ ID NO:36).
e). Adenoviral consensus protease sequence or site (M,L,I)XGG/X (SEQ ID NO:37) and (M,L,I)XGX/G (SEQ ID NO:38) see Webster et al. J Gen Virol 70:3215-3223 (1989); Weber, Curr Top Microbiol Immunol 1991:227-235 (1995) and Balakirev et al. J of Virol 76:6323-6331 (2002)

In the case of an adenovirus protease sequence or site, the invention is not meant to be limited to the consensus sequences provided above. The invention contemplates the use of any adenoviral protease. In one embodiment, the adenoviral protease is from the same adenovirus serotype as from which the adenoviral vector genome is derived.

Transgenes

The vectors of the invention may include one or more transgenes. In this way, various genetic capabilities may be introduced into target cells. In one embodiment, the transgene encodes a selectable marker. In another embodiment, the transgene encodes a cytotoxic protein. These vectors encoding a cytotoxic protein may be used to eliminate certain cells in either an investigational setting or to achieve a therapeutic effect. For example, in certain instances, it may be desirable to enhance the degree of therapeutic efficacy by enhancing the rate of cytotoxic activity. This could be accomplished by coupling the cell-specific replicative cytotoxic activity with expression of, one or more metabolic enzymes such as HSV-tk, nitroreductase, cytochrome P450 or cytosine deaminase (CD) which render cells capable of metabolizing 5-fluorocytosine (5-FC) to the chemotherapeutic agent 5-fluorouracil (5-FU), carboxylesterase (CA), deoxycytidine kinase (dCK), purine nucleoside phosphorylase (PNP), carboxypeptidase G2 (CPG2; Niculescu-Duvaz et al. J Med Chem. May 6, 2004; 47(10):2651-2658), thymidine phosphorylase (TP), thymidine kinase (TK) or xanthine-guanine phosphoribosyl transferase (XGPRT). This type of transgene may also be used to confer a bystander effect.

Additional transgenes that may be introduced into a vector of the invention include a factor capable of initiating apoptosis, antisense or ribozymes, which among other capabilities may be directed to mRNAs encoding proteins essential for proliferation of the cells or a pathogen, such as structural proteins, transcription factors, polymerases, etc., viral or other pathogenic proteins, where the pathogen proliferates intracellularly, cytotoxic proteins, e.g., the chains of diphtheria, ricin, abrin, etc., genes that encode an engineered cytoplasmic variant of a nuclease (e.g., RNase A) or protease (e.g., trypsin, papain, proteinase K, carboxypeptidase, etc.), chemokines, such as MCP3 alpha or MIP-1, pore-forming proteins derived from viruses, bacteria, or mammalian cells, fusgenic genes, chemotherapy sensitizing genes and radiation sensitizing genes. Other genes of interest include cytokines, antigens, transmembrane proteins, and the like, such as IL-1, IL-2, IL-4, IL-5, IL-6, IL-10, IL-12, IL-18 or flt3, GM-CSF, G-CSF, M-CSF, IFN-α, -γ, -γ, TNF-α, -β, TGF-α, -β, NGF, MDA-7 (Melanoma differentiation associated gene-7, mda-7/interleukin-24), and the like. Further examples include, proapoptotic genes such as Fas, Bax, Caspase, TRAIL, Fas ligands, nitric oxide synthase (NOS) and the like; fusion genes which can lead to cell fusion or facilitate cell fusion such as V22, VSV and the like; tumor suppressor gene such as p53, RB, p16, p17, W9 and the like; genes associated with the cell cycle and genes which encode anti-angiogenic proteins such as endostatin, angiostatin and the like.

Other opportunities for specific genetic modification include T cells, such as tumor infiltrating lymphocytes (TILs), where the TILs may be modified to enhance expansion, enhance cytotoxicity, reduce response to proliferation inhibitors, enhance expression of lymphokines, etc. One may also wish to enhance target cell vulnerability by providing for expression of specific surface membrane proteins, e.g., B7, SV40 T antigen mutants, etc.

Although any gene or coding sequence of relevance can be used in the practice of the invention, certain genes, or fragments thereof, are particularly suitable. For example, coding regions encoding immunogenic polypeptides, toxins, immunotoxins and cytokines are useful in the practice of the invention. These coding regions include those hereinabove and additional coding regions include those that encode the following: proteins that stimulate interactions with immune cells such as B7, CD28, MHC class I, MHC class II, TAPs, tumor-associated antigens such as immunogenic sequences from MART-1, gp 100 (pmel-17), tyrosinase, tyrosinase-related protein 1, tyrosinase-related protein 2, melanocyte-stimulating hormone receptor, MAGE1, MAGE2, MAGE3, MAGE12, BAGE, GAGE, NY-ESO-1, -catenin, MUM-1, CDK-4, caspase 8, KIA 0205, HLA-A2R1701, a-fetoprotein, telomerase catalytic protein, G-250, MUC-1, carcinoembryonic protein, p53, Her2/neu, triosephosphate isomerase, CDC-27, LDLR-FUT, telomerase reverse transcriptase, PSMA, cDNAs of antibodies that block inhibitory signals (CTLA4 blockade), chemokines (MIP1, MIP3, CCR7 ligand, and calreticulin), anti-angiogenic genes include, but are not limited to, genes that encode METH-1, METH -2, TrpRS fragments, proliferin-related protein, prolactin fragment, PEDF, vasostatin, various fragments of extracellular matrix proteins and growth factor/cytokine inhibitors, various fragments of extracellular matrix proteins which include, but are not limited to, angiostatin, endostatin, kininostatin, fibrinogen-E fragment, thrombospondin, tumstatin, canstatin, restin, growth factor/cytokine inhibitors which include, but are not limited to, VEGF/VEGFR antagonist, sFlt-1, sFlk, sNRP1, murine Flt3 ligand (mFLT3L), angiopoietin/tie antagonist, sTie-2, chemokines (IP-10, PF-4, Gro-beta, IFN-gamma (Mig), IFN, FGF/FGFR antagonist (sFGFR), Ephrin/Eph antagonist (sEphB4 and sephrinB2), PDGF, TGF and IGF-1. Genes suitable for use in the practice of the invention can encode enzymes (such as, for example, urease, renin, thrombin, metalloproteases, nitric oxide synthase, superoxide dismutase, catalase and others known to those of skill in the art), enzyme inhibitors (such as, for example, alpha1-antitrypsin, antithrombin III, cellular or viral protease inhibitors, plasminogen activator inhibitor-1, tissue inhibitor of metalloproteases, etc.), the cystic fibrosis transmembrane conductance regulator (CFTR) protein, insulin, dystrophin, or a Major Histocompatibility Complex (MHC) antigen of class I or II. Also useful are genes encoding polypeptides that can modulate/regulate expression of corresponding genes, polypeptides capable of inhibiting a bacterial, parasitic or viral infection or its development (for example, antigenic polypeptides, antigenic epitopes, and transdominant protein variants inhibiting the action of a native protein by competition), apoptosis inducers or inhibitors (for example, Bax, Bc12, Bc1X and others known to those of skill in the art), cytostatic agents (e.g., p21, p16, Rb, etc.), apolipoproteins (e.g., ApoAI, ApoAIV, ApoE, etc.), oxygen radical scavengers, polypeptides having an anti-tumor effect, antibodies, toxins, immunotoxins, markers (e.g., beta-galactosidase, luciferase, etc.) or any other genes of interest that are recognized in the art as being useful for treatment or prevention of a clinical condition. Further transgenes include those coding for a polypeptide which inhibits cellular division or signal transduction, a tumor suppressor protein (such as, for example, p53, Rb, p73), a polypeptide which activates the host immune system, a tumor-associated antigen (e.g., MUC-1, BRCA-1, an HPV early or late antigen such as E6, E7, L1, L2, etc), optionally in combination with a cytokine.

TRAIL has been shown to induce apoptosis in a wide variety of transformed cell lines (Jeremias I et al, Eur J Immunol 1998, 28:143-152 and Walczak H et al., Nat Med 1999, 5:157-163). The physiological role of TRAIL appears to involve both the innate and adaptive immune responses (NK and T-cells regulation of virally infected and transformed cells). Although TRAIL expression is widespread, normal cells appear to be resistant to TRAIL-induced apoptosis allegedly due to the expression of intracellular proteins (bcl-2, IAPs, FLIP, etc. which mitigate the apoptosis signaling response.

Although the precise molecular mechanism of the anti-tumor specificity of TRAIL remains unclear at present, TRIAL was chosen as a transgene of interest due to a number of features of TRAIL that have been described in the literature and suggest that late expression of TRAIL by adenovirus should enhance cell killing. E1A has been described as able to enhance killing by TRAIL (E1A and TRAIL: Routes et al., J Immunol. Oct. 15, 2002; 165(8):4522-7); the E1B 19K and 55K proteins reportedly reduce the effects of TRAIL (E1B 19K and TRAIL: Routes et al., J Immunol. Oct. 15, 2000; 165(8):4522-7 and anti-apoptotic activity: Tollefson et al., J Virol. 2001 October; 75(19):8875-87); E3: 10.4K and 14.5K (RID) remove FAS and TRAIL receptors on the cell surface by inducing their degradation in lysosomes (RID, FAS and TRAIL: Tollefson et al., Nature. Apr. 16, 1998; 392(6677):726-30; Shisler et al., J Virol. 1997 November; 71(11):8299-306; Lichtenstein et al., J Virol. 2002 November; 76(22):11329-42; Tollefson et al., J Virol. 2001; E3: 14.7K inhibits apoptosis by TNF, Fas and TRAIL and binds to caspase 8 (E3 14.7K and TRAIL: Chen et al., J Biol. Chem. Mar 6, 1998; 273(10):5815-20 and Tollefson et al., J Virol. 2001) and E3: 6.7K prevents apoptosis by TRAIL and maintains ER Ca homeostasis (E3 6.7K and TRAIL/ER Ca2++ homeostasis: Benedict et al., J Biol. Chem. Feb. 2, 2001; 276(5):3270-8 and E3-6.7K protein of adenovirus/localization in the endoplasmic reticulum: Wilson-Rawls et al., Virology. 1993 July; 195(1):6-15).

The invention further comprises combinations of two or more transgenes with synergistic, complementary and/or non-overlapping toxicities and methods of action. In summary, the present invention provides methods for inserting transgene coding regions in specific regions of the viral vector genome. The methods take advantage of known viral transcription elements and the mechanisms for expression of Ad genes, reduce the size of the DNA sequence for transgene expression that is inserted into the Ad genome, since no additional promoter is necessary and the regulation signals encompass a smaller size DNA fragment, provide flexibility in temporal regulation of the transgene (e.g., early versus late stage of infection; early versus intermediate stage of infection), and provide techniques to regulate the amount of transgene expressed. For example, a higher amount of transgene can be expressed by inserting the transgene into a transcript that is expressed normally at high levels and/or by operatively linking a high efficiency splice acceptor site to the transgene coding region. Expression levels are also affected by how close the regulating signals are to their consensus sequences; changes can be made to tailor expression as desired.

In designing the adenoviral vectors of the invention the biological activity of the transgene is considered, e.g. in some cases it is advantageous that the transgene be inserted in the vector such that the transgene is only or mostly expressed at the late stages of infection (after viral DNA replication). For example, the transgene may be inserted, in L3, as further described herein. For some transgenes, it may be preferred to express the transgene early in the viral life cycle. In such cases, the transgene may be inserted in any of the early regions (for example, E3) or into the upstream L1 region.

Therapeutic Methods

An effective amount of a vector of the invention is administered to a mammal (e.g., a human) as a composition in a pharmaceutically acceptable excipient, which may include one or more of the following: a saline solution, a suitable buffer, preservatives, stabilizers, and the like. A vector of the invention may be administered in conjunction with suitable agents such as antiemetics. An effective amount is an amount sufficient to effect beneficial or desired results, including clinical efficacy. An effective amount can be administered in one or more administrations or doses. For purposes of this invention, an effective amount of vector is an amount that is sufficient to palliate, ameliorate, stabilize, reverse, slow or delay the progression of the disease state or alleviate one or more symptoms of the disease. The amount to be given will be determined by the condition of the individual, the extent of disease, the route of administration, how many doses will be administered, and the desired objective.

Delivery of vectors of the invention is often accomplished by either site-specific injection or intravenous injection. Site-specific injections of vector may include, for example, injection into tumors, as well as intraperitoneal, intrapleural, intrathecal, intra-arterial, subcutaneous injection, intradermal injection, intramuscular injection or topical application. These methods are easily accommodated in treatments using vector alone or a combination of vector and chemotherapeutic agent. The invention also contemplates the use of the vector to infect cells from a subject ex vivo. For example, cells are isolated from a mammal. The isolated cells may contain a mixture of tumor cells and non-tumor cells. The cells are infected with a virus that is replication competent and the virus specifically replicates in the tumor cells. Therefore, the tumor cells are eliminated and if desired the remaining non-tumor cells may be administered back to the same mammal or if desired to a different mammal.

If used as a packaged adenovirus, adenovirus vectors may be administered in an appropriate physiologically acceptable carrier at a dose of about 10⁴ to about 10¹⁴. If administered as a polynucleotide construct (i.e., not packaged as a virus) about 0.01 □g to about 1000 □g of an adenoviral vector can be administered. The exact dosage to be administered is dependent upon a variety of factors including the age, weight, and sex of the patient, and the size and severity of the tumor being treated. The adenoviral vector(s) may be administered one or more times, depending upon the intended use and the immune response potential of the host, and may also be administered as multiple, simultaneous injections. If an immune response is undesirable, the immune response may be diminished by employing a variety of immunosuppressants, or by employing a technique such as an immunoadsorption procedure (e.g., immunoapheresis) that removes adenovirus antibody from the blood, so as to permit repetitive administration, without a strong immune response. If packaged as another viral form, such as HSV, an amount to be administered is based on standard knowledge about that particular virus (which is readily obtainable from, for example, published literature) and can be determined empirically.

In one embodiment the host organism is a human patient. For human patients, if a therapeutic coding region is included in the vector, the therapeutic coding region may be of human origin although genes of closely related species that exhibit high homology and biologically identical or equivalent function in humans may be used if the gene does not produce an adverse immune reaction in the recipient. A therapeutically active amount of a nucleic acid sequence or a therapeutic gene is an amount effective at dosages and for a period of time necessary to achieve the desired result. This amount may vary according to various factors including but not limited to sex, age, weight of a subject, and the like.

Embodiments of the present invention include methods for the administration of combinations of a cancer-specific vector of the invention and a second anti-neoplastic therapy, which may include radiation, administration of an anti-neoplastic or chemotherapeutic agent, etc., to an individual with neoplasia, as detailed in U.S. Application 20030068307. The cancer-specific vector and chemotherapeutic agent may be administered simultaneously or sequentially, with various time intervals for sequential administration. In some embodiments, an effective amount of vector and an effective amount of at least one antineoplastic or chemotherpeutic agent are combined with a suitable excipient and/or buffer solutions and administered simultaneously from the same solution by any of the methods listed herein or those known in the art. This may be applicable when the chemotherapeutic agent does not compromise the viability and/or activity of the vector itself.

Where more than one chemotherapeutic agent is administered, the agents may be administered together in the same composition; sequentially in any order; or, alternatively, administered simultaneously in different compositions. If the agents are administered sequentially, administration may further comprise a time delay. Sequential administration may be in any order, and accordingly encompasses the administration of an effective amount of a vector first, followed by the administration of an effective amount of the chemotherapeutic agent. The interval between administration of the cancer-specific vector and chemotherapeutic agent may be in terms of at least (or, alternatively, less than) minutes, hours, or days. Sequential administration also encompasses administration of a chosen antineoplastic agent followed by the administration of the vector. The interval between administration may be in terms of at least (or, alternatively, less than) minutes, hours, or days.

Administration of the above-described methods may also include repeat doses or courses of a cancer-specific vector and chemotherapeutic agent depending, inter alia, upon the individual's response and the characteristics of the individual's disease. Repeat doses may be undertaken immediately following the first course of treatment (i.e., within one day), or after an interval of days, weeks or months to achieve and/or maintain suppression of tumor growth. A particular course of treatment according to the above-described methods, for example, combined cancer-specific vector and chemotherapy, may later be followed by a course of combined radiation and cancer-specific vector therapy.

Anti-neoplastic (chemotherapeutic) agents include those from each of the major classes of chemotherapeutics, including but not limited to: alkylating agents, alkaloids, antimetabolites, anti-tumor antibiotics, nitrosoureas, hormonal agonists/antagonists and analogs, immunomodulators, photosensitizers, enzymes and others. In some embodiments, the antineoplastic is an alkaloid, an antimetabolite, an antibiotic or an alkylating agent. In certain embodiments the antineoplastic agents include, for example, thiotepa, interferon alpha-2a, and the M-VAC combination (methotrexate-vinblastine, doxorubicin, cyclophosphamide). Preferred antineoplastic agents include, for example, 5-fluorouracil, cisplatin, 5-azacytidine, and gemcitabine. Particularly preferred embodiments include, but are not limited to, 5-fluorouracil, gemcitabine, doxorubicin, miroxantrone, mitomycin, dacarbazine, carmustine, vinblastine, lomustine, tamoxifen, docetaxel, paclitaxel or cisplatin. The specific choice of chemotherapeutic agent(s) is dependent upon, inter alia, the characteristics of the disease to be treated. These characteristics include, but are not limited to, location of the tumor, stage of the disease and the individual's response to previous treatments, if any.

In addition to the use of single antineoplastic agents in combination with a particular cancer-specific vector, the invention also includes the use of more than one agent in conjunction with the cancer-specific vector. These combinations of antineoplastics when used to treat neoplasia are often referred to as combination chemotherapy and are often part of a combined modality treatment which may also include surgery and/or radiation, depending on the characteristics of an individual's cancer. It is contemplated that the combined cancer-specific vector/chemotherapy of the present invention can also be used as part of a combined modality treatment program.

There are a variety of delivery methods for the administration of antineoplastic agents, which are well known in the art, including oral and parenteral methods. There are a number of drawbacks to oral administration for a large number of antineoplastic agents, including low bioavailability, irritation of the digestive tract and the necessity of remembering to administer complicated combinations of drugs. The majority of parenteral administration of antineoplastic agents is intravenously, as intramuscular and subcutaneous injection often leads to irritation or damage to the tissue. Regional variations of parenteral injections include intra-arterial, intravesical, intra-tumor, intrathecal, intrapleural, intraperitoneal and intracavity injections.

Delivery methods for chemotherapeutic agents include intravenous, intraparenteral and intraperitoneal methods as well as oral administration. Intravenous methods also include delivery through a vein of the extremities as well as including more site specific delivery, such as an intravenous drip into the portal vein. Other intraparenteral methods of delivery include direct injections of an antineoplastic solution, for example, subcutaneously, intracavity or intra-tumor.

Assessment of the efficacy of a particular treatment regimen may be determined by any of the techniques known in the art, including diagnostic methods such as imaging techniques, analysis of serum tumor markers, biopsy, the presence, absence or amelioration of tumor associated symptoms. It will be understood that a given treatment regime may be modified, as appropriate, to maximize efficacy.

In a further aspect of the invention, a pharmaceutical composition comprising the recombinant viral vectors and/or particles of the invention and a pharmaceutically acceptable carrier is provided. Such compositions, which can comprise an effective amount of cancer-specific vector and/or viral particles of the invention in a pharmaceutically acceptable carrier, are suitable for local or systemic administration to individuals in unit dosage forms, sterile parenteral solutions or suspensions, sterile non-parenteral solutions or oral solutions or suspensions, oil in water or water in oil emulsions and the like. Formulations for parenteral and non-parenteral drug delivery are known in the art. Compositions also include lyophilized and/or reconstituted forms of the cancer-specific vector or particles of the invention. Acceptable pharmaceutical carriers are, for example, saline solution, protamine sulfate (Elkins-Sinn, Inc., Cherry Hill, N.J.), water, aqueous buffers, such as phosphate buffers and Tris buffers, or Polybrene (Sigma Chemical, St. Louis Mo.) and phosphate-buffered saline and sucrose. The selection of a suitable pharmaceutical carrier is deemed to be apparent to those skilled in the art from the teachings contained herein. These solutions are sterile and generally free of particulate matter other than the desired cancer-specific vector. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients that enhance uptake of the cancer-specific vector by cells may be included.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the preferred embodiments.

### EXAMPLES

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

The following examples are offered by way of illustration and not by way of limitation. In the following examples, the sequence of the branch point plus splice acceptor sequence is: TACTTAT GACTCGTACTATTGTTATTCATCC AG↓G (SEQ ID NO:39) The underlined sequence is the branch point sequence and the arrow indicates the location of the splice site according to splicing rules. Since the rules governing the consensus sequence are not invariant, other similar sequences that conform to the rules may be used. Alternatively, a branch point plus splice acceptor site that exists in another Ad serotype can be used. The following examples pertain to exemplary adenoviral vectors derived from human Ad5 serotype. Similar constructs can be made from other adenoviruses.

### Example 1

### Constructing Ad Vectors and Propagating Ad Vector Virions Coding a Transgene(s)

The following examples describe inserting at least one transgene in vectors that contain either a portion of the adenovirus vector genome or the whole adenoviral vector genome and the inserted transgene. In the case of the vectors coding for the whole adenoviral vector genome and the inserted transgene, the vector is transfected into an adenoviral producer cell line and virus is propagated using standard techniques. Prior to transfecting, the vector may be digested with a restriction enzyme, which does not cut within the viral vector genome, but cuts the vector (e.g. plasmid) backbone.

Alternatively, the transgene is inserted into a vector that contains a portion of the viral genome. In this case, the portion of the vector containing part of the viral genome is cloned into a vector that contains the rest of the viral vector genome. Therefore creating a vector that encodes the complete viral vector. Next, the complete viral vector genome is transfected into a producer cell line and virus is propagated. In another method, the vector containing the transgene and portion of the viral genome is transfected into a producer cell line along with the appropriate fragment(s) of the viral vector genome, that as a result of homologous recombination, will form a complete viral vector genome, which will replicate and propagate in the producer cell lines.

The following references provide more details of viral vector construction and viral propagation: Ghosh-Choudhury et al. Gene. 1986;50(1-3):161-71; Toietta et al. Mol Ther. 2002 February; 5(2):204-10, incorporated by reference herein. This example describes inserting a transgene(s) in the various locations in the L2 region of Ad.

### Example 2

### Cloning Ad Vectors with Modifications and for Cloning Further Modifications in the L2 Region

For recombination using the bacterial system, it is an advantage to have a full length Ad genomic plasmid (of any specific modifications) that also contains at least one unique restriction site in the region that will be altered. In some examples below, the targeted region is the L2 region. However a unique site does not exist. Therefore, a plasmid was created that possesses these features by the following method. The BamHI to AscI region of Ad corresponding to bases 15672 to 21562 of wild type Ad was inserted into the cloning plasmid pNEB193 (New England Biolabs) and this plasmid is called CP1563. Site directed mutagenesis was performed to incorporate a unique SwaI site into position 16530 (base corresponding to Ad wild type) and this plasmid was designated CP 1564. A plasmid was made that contains the Ad5 sequences from 13259 to 21562, the BamHI to PmeI fragment, inserted into the cloning plasmid pMCS5 and is called cp1165. cp1165 was altered so that it no longer contains the AscI site that was present in the cloning vector and becomes CP1166. The SwaI-containing Ad fragment from CP1564 was removed by digestion with AscI and BamHI and inserted into AscI and BamHI-cut CP1566 and this plasmid is called CP1567. This is built back into any full length Ad genomic plasmid to generate a genomic plasmid that contains a unique SwaI site in the L2 region that can be used for cutting for recombination into the L2 region.

### Example 3

### Transgene Insertions in the L2 Region Between the pVII CDS and pV CDS

The following cloning steps are performed in a vector (e.g. plasmid) containing either the whole adenoviral vector genome (i.e. ITR to ITR) or a vector that contains the L2 region of the adenoviral genome or a relevant portion thereof. A transgene CDS, i.e., an open reading frame (ORF) or cDNA, was inserted downstream of the pVII CDS and upstream of the pV CDS. There is a splice acceptor sequence between these two genes already and it is used for the expression of pV; the associated branch point sequence has not been exactly mapped. The transgene CDS or cDNA is inserted upstream of the endogenous pV splice site and an additional branch point and splice acceptor site is inserted upstream of the transgene. These additional splicing sequences are used for transgene expression. The sequence for an exemplary splice acceptor for the L2 region differs from the one used for the L3 region. The new splice acceptor site was chosen according to the existing splice site for pVII: TACTTATAGTGAA AACG TTCCTGCTCTCACAG by conserving the strong bases to obtain TACTTATAGTAATCTAATTCCTGCTCTCTCAG (SEQ ID NO:42). Alternatively, an additional branch point and splice acceptor site operatively linked to the transgene CDS is inserted upstream of the native pV branch point and splice acceptor site.

### Example 4

### Transgene Insertions in the L2 Region Between the Penton CDS and pVII CDS

A transgene CDS is inserted downstream of the penton CDS and upstream of the pVII CDS. The presumed branch point and splice acceptor site for pVII is present in the coding region for penton. Therefore, the inserted transgene uses the endogenous splicing signals present in the penton CDS and an additional branch point and splice acceptor site is placed after the transgene CDS and upstream of (and operatively linked to) the pVII CDS to synthesize the mRNA for pVII gene.

### Example 5

### Transgene Insertions in the L2 Region Between the Mu CDS and L4 PolyA Signal

A transgene is inserted downstream of the Mu (aka pX) CDS and upstream of the L2 polyadenylation signal sequence. A branch point, splice acceptor site and transgene CDS are inserted downstream of the Mu CDS and upstream of the L2 polyadenylation signal sequence. The inserted branch point and splice acceptor site are operatively linked to transgene CDS

### Example 6

### Transgene Insertions in the L2 Region Downstream of the Mu CDS and L4 PolyA Signal

An IRES operatively linked to a transgene is inserted downstream of the Mu CDS and upstream of the L2 polyadenylation signal sequence.

### Example 7

### Cloning Splicing Elements and hIL-24 into the L3 Region Between 23K CDS and L3 PolyA

pCR2.1 Topo (Invitrogen, Carlsbad, Calif.) is cut with EcoRI and religated to remove one EcoRI site. This plasmid is then cut with BspHI, filled in with Klenow, and religated to remove the BspHI site. This plasmid is then cut with ApaI and RsrII, filled in with Klenow, and religated to remove excess sequences between these sites. The portion containing the ampicillin resistance gene is retained. This plasmid is designated CP1601. pBHGE3 (Microbix Biosystems, Toronto, Ontario, Canada) is cut with HindIII and XhoI and the HindIII/XhoI fragment containing nucleotides 18318 to 24795 of the wild-type Ad5 genome is ligated into the HindIII- and XhoI-cut fragment (vector backbone) of CP1601. The resulting plasmid is designated CP1603.

To insert the transgene human-IL24 (hIL-24, also known as MDA-7) downstream of the 23K CDS and upstream of the L3 polyadenylation signal sequence and to allow expression by a splicing mechanism, the following steps are performed: Using PCR SOEing (Horton et al. Biotechniques. 1990 May; 8(5):528-35) a fragment containing the branch point and splice acceptor site and the hIL-24 CDS is amplified. (The IL-24 CDS is obtained from Invivogen plasmid reference “porf-hil24”). The branch point and splice acceptor site are coded for in the PCR primers. The same branch point and splice acceptor site sequence outlined above is used. The PCR product is TA cloned into pCR2.1 Topo (Invitrogen, Carlsbad, Calif.) creating CP1602. Then the HindIII/XhoI region from CP1602 which contains the Ad and hIL-24 sequences, is ligated into HindIII/XhoI digested CP1601 resulting in plasmid CP1606 Rev (SEQ ID NO:48). The KpnI/SfiI fragment of CP1606 Rev is ligated into KpnI/SfiI-cut CP1524 to create CP1610. CP1610 is the shuttle plasmid for “shuttling” the modified L3 region in to a complete adenoviral vector genome. With this plasmid, any transgene can be inserted here after digestion of the transgene fragment (often generated by PCR with the appropriate ends) and CP1610 with BspHI and NheI. Generation of the Ad vector or virus is accomplished through homologous recombination of CP1606 Rev (SEQ ID NO:48) with an adenoviral vector genome backbone plasmid. The adenoviral vector genome backbone contains all of the other necessary adenoviral genes and regions of DNA that provide homology with the region to be inserted, thereby generating a vector that contains the complete sequence of the adenoviral vector genome including the modifications to the L3 region. The recombination can be performed in a strain of E. coli to generate a plasmid that contains the full length of Ad genome with the desired changes. This vector is then used to create adenoviral viral particles by transfection into mammalian cells and amplification of virus.

Other transgenes can be inserted into the L3 region just upstream of the L2 polyadenylation site using this same strategy. Alternatively, transgenes can be amplified with the restriction sites BspHI and NheI on their 5′ and 3′ ends, respectively, and inserted into CP1606 Rev (SEQ ID NO:48) and generation of the corresponding Ad virus can be done using the same strategy.

### Example 8

### Cloning mIL21 into the L3 Region Between the Hexon CDS and 23K CDS

Insertion of a murine interleukin-21 transgene downstream of the L3 hexon CDS and upstream of the 23K CDS utilizing splicing for expression is performed as follows. Using methods routinely employed by those of skill in the art, a splice acceptor site and a mIL21 cDNA (Invivogen reference: porf-mIL21) are inserted downstream of the hexon CDS and upstream of the 23K CDS. The branch point and splice acceptor site are coded for in PCR primers. The same branch point and splice acceptor site sequence outlined above is used. In this case, the branch point plus splice acceptor site is added to the 3′ end of the transgene because the normal 23K splicing signals are present in the ORF for hexon and, as such, upstream of where the transgene can be inserted. This strategy utilizes this endogenous (native viral) splicing sequence for the transgene and adds an additional branch point plus splice acceptor to be utilized for expression of the 23K CDS. PCR products containing fragments of the L3 region of the Ad5 genome (modified with restriction enzyme sites for cloning and/or the splice processing signals) and the hIL24 cDNA are digested with the appropriate restriction endnonucleases. The fragments are then ligated into a CP1601 equivalent vector to create CP1623. Then the BamHI/KpnI fragment of CP1623 is ligated into BamHI/KpnI-cut CP1524 (Example 7) to create CP1631, a shuttle in which the engineered cassette (the 3′ splice acceptor site preceded by the hIL24 cDNA) is inserted after the stop codon for Hexon and the start codon for 23K. CP1623 is the shuttle plasmid that can be used for other transgenes to be expressed in this manner. A complete adenoviral vector containing the inserted mIL21 transgene is attained through homologous recombination of CP1631 with the desired full length vector containing the desired adenoviral genome backbone. The resulting plasmid, CP1631, is then used to create the new Ad virus, OV1153, which will express the transgene.

### Example 9

### Cloning an IRES and hIL-24 into the L3 Region Between 23K CDS and the L3 PolyA Signal

A transgene such as hIL-24 (Invivogen reference: porf-hil24) can be inserted into the L3 region just upstream of the L3 polyadenylation site and downstream of all the L3 coding regions using an IRES for expression. The IRES and transgene are expected to be included in and translated from all the L3 mRNAs. Using PCR SOEing (Horton et al. Biotechniques. 1990 May; 8(5): 528-35), a PCR product is generated so that an IRES, such as that obtained from FMDV or ECMV, is operatively linked to a hIL-24 CDS and is inserted downstream of the 23K CDS and upstream of the L3 polyadenylation signal sequence. The PCR product is TA cloned into pCR2.1 creating CP1604. The HindIII/XhoI region from CP1604 which contains the Ad and hIL-24 sequences, is ligated into HindIII/XhoI digested CP1601-NotI resulting in CP1607. CP1601-NotI was made by cutting CP1601 with NotI, filling in the ends with Klenow, and then religated the plasmid so that the NotI site is destroyed. The BamHI/SfiI fragment of CP1607 is ligated into BamHI/SfiI-cut CP1603 to create the plasmid CP1609. CP1607 is the shuttle plasmid that can be used for other transgenes to be expressed in this manner. A complete adenoviral vector containing the inserted mFLT3L transgene is attained through homologous recombination of CP1609 with the desired full length adenoviral genome backbone plasmid. The resulting plasmid is then used to generate an Ad virus by transfection into mammalian cells.

### Example 10

### Construction of OV1165 Control Virus

OV1165 is a control virus comprising the E2F-1 promoter operatively linked to E1A. The vector carries the packaging signal in the native location and carries a polyadenylation signal upstream of the E2F-1 promoter to inhibit transcriptional read-through from the LITR. These sequences contained in OV1165 were derived from the plasmid pFLAr21pAE2Ff.

The full-length recombinant adenoviral genome (pFLAr21pAE2Ff) was generated as follows. First, a full-length plasmid, pFLAd5 was generated by combining the SmaI-linearized pAd5LtRtSmaI shuttle plasmid containing I-SceI restriction sites introduced by PCR with genomic DNA of Ad5 in E. coli resulting in pFLAd5 which contained the Ad5 genome bordered by I-SceI sites. Next, pFLAd5 was digested with SmaI and the fragments containing the left and right terminal fragments of Ad5 were gel purified and self-ligated to generate pAd5LtRtSmaI. The plasmid pAd5LtRtSmaI containing the left (1006 bp) and right terminal (582 bp) restriction fragments of Ad5 was digested with SmaI and combined with genomic viral DNA of Ar21pAE2f in E-coli to generate the full length plasmid pFLAr21pAE2Ff.

A plasmid called CP 1585 containing a full-length adenoviral-derived genome of the type described above (pFLAr21pAE2Ff) was generated as follows:

A 4648 bp SmaI fragment was isolated from the pFLAr21pAE2fF plasmid (pFLAr21 Sma shuttle) which contains the E2F promoter upstream of E1A. The BamHI site was removed from pFLAr21 Sma shuttle by digestion with Bam HI, filled in with klenow polymerase to destroy the site and religated. A 4646 bp plasmid product called pE2f LtRT shuttle was isolated. Plasmid CP 1585 (38998 bp) was generated by bacterial homologous recombination in BJ 5183 cells using CV802 viral DNA containing the wild-type Ad 5 genome and linearized pE2f LtRt Shuttle. The final product CP 1585 has the full-length recombinant adenoviral genome with the E2F-1 promoter operatively linked to E1A. CP 1585 also has a unique BamHI site (map site 21796) near the L4 region which can be used to facilitate gene insertion in the late region.

The OV 1165 virus was generated by lipofectamine transfection of A549 cells with the 36,246 base pair I-SceI fragment derived from CP 1585. OV 1165 was grown on A549 cells and scaled up to 5 roller bottles.

The virus was purified by CsCl centrifugation and formulated in ARCA buffer with a yield of 4.97 ml and a particle titer of 2.33×10¹² vp/ml (2.3×10¹² vp per roller bottle).

### Example 11

### Generation of L3 Vectors

Shuttle plasmids for use in generating vectors expressing transgenes from the Ad5 L3 region, utilizing alternative splicing mechanisms were constructed as follows:

Plasmids containing recombinant, full-length adenoviral genomes were generated using the BJ5183 bacterial recombination system.

Plasmids containing recombinant, full-length adenoviral genomes were generated using the BJ5183 bacterial recombination system. In this application of the system, modifications were made to the CP1606 and CP 1524 shuttle plasmids containing smaller fragments of the adenoviral genome.

L3 vectors were also generated by the BJ5183 bacterial recombination system. A large, linearized vector containing a full-length adenoviral genome in a plasmid backbone was co-transformed with a smaller fragment of DNA derived from a similar adenoviral genome into BJ5183 bacteria, a strain with functional recB, recC, sbcB, and sbcC genes. The smaller fragment contains significant stretches of nucleotide sequence homologous to the larger vector flanking the coding sequence for TRAIL. Co-transformation of the large, linearized vector and the smaller fragment of DNA into BJ5183 cells was used to promote homologous recombination of the smaller fragment into the larger, linearized vector. The resulting progeny plasmid contains a full-length, adenoviral-derived genome incorporating the TRAIL coding sequence (cDNA; SEQ ID NO: 46).

A fragment was excised from the final shuttle plasmid (CP1581, derived from CP1524) and co-transformed with a linearized plasmid containing the full-length, recombinant adenoviral genome (pFLAr21pAE2Ff). Progeny plasmids are derivatives of pFLAr21pAE2Ff. Prior to recombination, introduction of deliberate alterations to the Ad sequence contained in CP1524 were performed in a smaller base vectors, CP1606 Rev (SEQ ID NO:48). CP1524 (SEQ ID NO:47) and CP1606 Rev are further described below.

CP1524 (SEQ ID NO:47) is a base shuttle vector containing nucleotides 18318-24795 of the wild-type Ad5 genome in a pcDNA3.1+ (Invitrogen) derived plasmid backbone. The stretch of the Ad5 genome from nucleotides 18318-24795 contains a portion of the wt Ad5 L3 region.

CP1524 was constructed by using molecular cloning methods routinely employed by those of skill in the art. Initially, pcDNA3.1+ was digested with the restriction endonucleases BstZ17 I and Xba I (New England Biolabs). The resulting digested vector was treated with DNA Polymerase I (Klenow) (New England Biolabs) to fill in the single-stranded stretch of DNA created by Xba I digestion. The resulting “blunt” ends were ligated together to create a modified pcDNA3.1+ derived vector, CP1523, with excess sequence removed. The Ad5 sequence of CP1524 was derived from pBHGE3 (Microbix), a commercially available plasmid containing the wt Ad5 genome, with the exception of a large deletion in the E1 region. The region representing the wt Ad5 sequence from nucleotides 18318-24795 was excised from pBHGE3 with the restriction endonucleases HindIII and XhoI (New England Biolabs) and ligated into the similarly cut CP1523 to yield the progeny plasmid, CP1524.

### Example 12

### Cloning Splicing Elements into the L3 Region Between 23K CDS and L3 PolyA (CP1606 Rev Construction)

CP1606 Rev contains a modified segment of the wt Ad5 L3 region representing nucleotides 22181 to 23008 of the wt Ad5 genome in a truncated pCR2.1 Topo plasmid backbone (Invitrogen). Modifications to the region include a 3′ splice acceptor site (hereinafter referred to as 3′ SAS) preceding the hIL24 cDNA. The hIL24 cDNA is flanked by BspHI (compatible with NcoI) and NheI sites for replacement with a different cDNA sequence, e.g., TRAIL.

CP1606 Rev was constructed by standard molecular cloning methods. Initially, a PCR fragment was generated encompassing nucleotides 22181 to 23008 of the wt Ad5 genome, modified to include a 3′ SAS and the hIL24 cDNA between the stop codon for the wt Ad5 23K gene (hereinafter referred to as 23K) and the L3 polyA. The fragment was constructed by PCR splice overlap extension.

Briefly, 3 precursor PCR fragments were generated. Fragment PCRp 1618.95.1/2 contains the portion of the wt Ad5 genome from nucleotides 22181 to 22354 amplified from pBHGE3 with the primers 1618.95.1 (SEQ ID NO:52) and 1618.95.2 (SEQ ID NO: 56). PCRp 1618.95.1/2 incorporates a 3′ SAS (SEQ ID NO: 45) at its downstream end.

Fragment PCRp 1618.97.1 (SEQ ID NO:54)/1618.97.2 (SEQ ID NO:55) contains the hIL24 cDNA, amplified from pORF9-hIL24 (Invivogen) with the primers 1618.97.1 and 1618.97.2. PCRp 1618.97.1/2 incorporates a 3′ SAS and a portion of the wt Ad5 L3 region at its upstream and downstream ends, respectively.

Nucleotides 22355 to 23008 of the wt Ad5 genome were amplified with primers 1618.95.5 (SEQ ID NO:56) and 1618.95.6 (SEQ ID NO:53). Fragments PCRp 1618.95.1/2 and PCRp 1618.97.1/2 have overlapping ends. A mixture of the two fragments was placed in a PCR reaction with flanking primers 1618.95.1 (SEQ ID NO:52) and 1618.97.2 (SEQ ID NO:55). The resulting product, PCRp 1618.95.1/1618.97.2, joined the portion of the wt Ad5 genome from nucleotides 22181 to 22354 with a 3′ SAS and the hIL24 cDNA.

Likewise, fragments PCRp 1618.97.1/2 and PCRp 1618.95.5/6 have overlapping ends. A mixture of the two fragments was placed in a PCR reaction with flanking primers 1618.97.1 and 1618.95.6. The resulting product, PCRp 1618.97.1/1618.95.6, has a 3′ SAS and the hIL24 cDNA with the portion of the wt Ad5 genome from positions 22355 to 23008.

Therefore, PCRp 1618.95.1/1618.97.2 and PCRp 1618.97.1/1618.95.6 overlap at the engineered 3′ SAS and hIL24 cDNA. A mixture of the two fragments was placed in a PCR reaction with flanking primers 1618.95.1 (SEQ ID NO:52) and 1618.95.6 (SEQ ID NO:53). The resulting product, PCRp 1618.95.1/6, represents nucleotides 22181 to 23008 of the original wt Ad5 genome incorporating an engineered 3′ SAS and the hIL24 cDNA between the original positions 22354 and 22355 (so that the introduced features are between the stop codon for 23K and the L3 polyA site). PCRp 1618.95.1/6 was placed in the vector pCR2.1 Topo by Topo TA cloning resulting in plasmid CP1602.

To generate a base shuttle for the removal and insertion of transgenes into the modified 23K region described above, a plasmid backbone was constructed by modification of pCR2.1 Topo. Initially, pCR 2.1 Topo was digested with the restriction endonuclease EcoRI (New England Biolabs) and religated to remove one of the EcoRI sites and the excess nucleotide sequence between the original two sites, yielding pCR2.1 TopoEcoRI. In order to facilitate the downstream cloning of transgenes, the BspHI restriction site was removed from pCR2.1 TopoEcoRI by digestion with the restriction endonuclease BspHI (New England Biolabs), treatment with DNA Polymerase I (Klenow), followed by ligation of the treated vector to yield pCR2.1 Topo EcoRI-BspHI. To ease downstream cloning efforts, excess sequence was removed from pCR.21 Topo EcoRI-BspHI by digestion with the restriction endonucleases ApaI and RsrII (New England Biolabs), treatment with DNA Polymerase I (Klenow), followed by ligation of the treated vector to yield CP1601.

The final base shuttle, CP1606 Rev (SEQ ID NO:48), was constructed by excising the HindIII to XhoI fragment of CP1602 and placing it into a similarly cut CP1601 vector.

To generate a larger shuttle plasmid suitable for recombination with the linearized vector pFLAr21pAE2Ff, the Kpn I to SFiI (New England Biolabs) fragment was excised from CP1606 Rev and placed into a similarly cut CP1524 vector, resulting in plasmid CP1610.

### Example 13

### Generation of an Oncolytic Vector with a Splice Acceptor (SA) Site and TRAIL in the L3 region between 23K CDS and L3 PolyA (OV 1160)

In order to generate a recombinant derivative of pFLAr21 pAE2Ff containing the engineered 3′ SAS and TRAIL cDNA (Invivogen; reference pORF-TRAIL), two sub-clonings were carried out. Plasmid CP1580 was constructed by amplification of the TRAIL cDNA (SEQ ID NO: 46) from pORF-hTTRAIL by PCR. This fragment was subjected to restriction digest using NcoI-NheI sites and ligated into CP1606 Rev following digestion with BspHI-NheI. The BspHI site is compatible with the NcoI site. The CP1581 plasmid was constructed from the KpnI-SfiI fragment of CP1580 which contains TRAIL 3′ to the splice site of the 23K protein ligated into the CP1524 shuttle plasmid for use in recombination. Plasmid CP1582 was obtained following recombination of the XhoI-HindIII fragment of CP1581 with the full length pFLar21paE2f. Recombinant derivatives of pFLAR21pAE2Ff containing the engineered 3′ SAS (SEQ ID NO:45) and TRAIL cDNA (SEQ ID NO:46) were isolated and designated CP1582.

OV 1160 virus (SEQ ID NO:51) was generated by restriction digest of CP 1582 with I-SceI enzyme liberating the full length adenoviral sequence (containing the new splice acceptor site and the TRAIL).

### Example 14

### Generation of an Oncolytic Vector with an IRES and TRAIL in the L3 Region Between 23K CDS and L3 PolyA (OV 1164)

OV1164 (SEQ ID NO:50) has an IRES after the stop codon of the Hexon coding sequence. The plasmids involved in the generation of OV1164 are CP1590, CP1591 and CP1592 (full length).

The full length CP 1592 plasmid was generated following three sub-clonings. The assembly of the HexonFmdvTrail-containing plasmid was performed by PCR Soeing. In order to generate a fragment that would have an overlap sufficient to generate the next plasmid by recombination, 1 kb of sequence from Ad5 was included on both sides of that sequence. PCR SOEing was performed as follows:

(1) the Ad5 sequence from pBHG3 was amplified using 1706.83.1(SEQ ID NO:57)/1706.83.2 (SEQ ID NO:58); (2) HexFmdvTRAIL fragment from pmcsHexFmdvTRAIL was amplified using oligos 1706.95.1(SEQ ID NO:59)/1618.116.3 (SEQ ID NO:60): and (3) the fragments were assembled using the 1706.83.1 and 1618.116.3 cloned new fragment in PCRstopo.

(2) CP1590 is a PCR2.1 topo vector containing the PCR assembled fragment (ad5HexFmdvTRAILAd5). CP1591 was generated by recombination of the HpaI-XhoI frag (Ad5HexFmdvTRAILAd5) of CP1590 with CP1524 linearized with BamHI. The final full length CP1592 plasmid was generated by recombination of the XhoI-HindIII fragment of CP1591 with the pFLar21paE2f linearized with SgfI.

TRAIL was cloned in pmcs HexFMDV by amplification of TRAIL from pORF-hTRAIL with oligos 1619.144.1 (SEQ ID NO:61)/1619.144.3 (SEQ ID NO:62) using Expand polymerase. The amplified fragment was digested with BSTZ171 and ligated into CP1557 (pmcsHexFmdv bamHI-EcoRI) to generate pmcsHexFmdvTrail.

Virus was generated by restriction digest of CP 1592 with I-SceI enzyme liberating the full length adenoviral sequence (containing the IRES and TRAIL).

### Example 15

### Virus Production, Trail Expression and Biological Activity in Vitro

A549 cells (1-2×10e7 cells) were transferred from plates into a roller bottle (Falcon 35-3069 pleated surface roller bottle with vented cap) on day one in RPMI 1640 medium with 2 mM L-Glutamine and 10% FBS. On day 4, the cells were infected for 3-6 hours with 5-10 viral particles/cell. The cells were harvested at 72-96 hours post infection and stored at −80° C. until purification by CsCl centrifugation. A titer of 2.55 e12, 2.05 e12 and 2.11 e12 was obtained for OV 1160, lot 1; OV 1160, lot 2; and OV 1164, respectively (as determined by HPLC). The titer was determine by OD at 260 nm (viruses were diluted 1:10 and 1:20 in TE containing 0.1% of SDS, incubated 20 min at 56° C. and the absorbance was read at 260 and 280 nm on a spectrophotometer.

The virus yield was performed at 50 ppc for each viruses on A549, SW780 and Hela S3 cells. Infection was carried out for 4 hrs, then the cells were washed twice with PBS before adding 3 ml of fresh complete media. The cells were harvested at Day 3 (72 hours following transduction), freeze-thawed 3 times and analyzed. Table 1 shows the virus yield at Day 3.

The virus yield was determined at Day 3 (72 hours following transduction) and titrated using a Hexon Assay on 293 cells on Collagen-coated 12-well plates. Table 1 shows the virus yield at Day 3.

TABLE 1

| | | |
|---|---|---|
| total PFU | PFU/Cell | Fold Diff |

A549

| | | | |
|---|---|---|---|
| OV1165 | 3.50E+10 | 7.00E+04 | 1.00E+00 |
| OV1160#2 | 4.87E+09 | 9.74E+03 | 7.19E+00 |
| OV1160#1 | 4.27E+09 | 8.54E+03 | 8.20E+00 |
| OV1164 | 1.23E+09 | 2.46E+03 | 2.85E+01 |
| 802 | 5.83E+10 | 1.17E+05 | 6.00E−01 |

HelaS3

| | | | |
|---|---|---|---|
| OV1165 | 3.20E+10 | 6.40E+04 | 1.00E+00 |
| OV1160#2 | 2.45E+09 | 4.90E+03 | 1.31E+01 |
| OV1160#1 | 2.24E+09 | 4.48E+03 | 1.43E+01 |
| OV1164 | 5.50E+08 | 1.10E+03 | 5.82E+01 |
| 802 | 7.30E+10 | 1.46E+05 | 4.38E−01 |

SW780

| | | | |
|---|---|---|---|
| OV1165 | 4.55E+10 | 9.10E+04 | 1.00E+00 |
| OV1160#2 | 2.97E+09 | 5.94E+03 | 1.53E+01 |
| OV1160#1 | 2.98E+09 | 5.96E+03 | 1.53E+01 |
| OV1164 | n/a | | |
| 802 | 8.90E+10 | 1.78E+05 | 5.11E−01 |

The Hexon assay is a biological assay to measure, by immunostaining, the production of the adenovirus hexon protein in a given culture of cells. Briefly, cells are infected with serial dilutions of CVL or purified virus and incubated at 37° C. 48 hours post-infection, the cells are fixed with methanol then probed with an anti-Hexon primary antibody followed by a horseradish peroxidase (hereinafter referred to as HRP) conjugated secondary antibody. Hexon-producing cells are then visualized by exposing the fixed culture to a diaminobenzidine (hereinafter referred to as DAB) substrate which is converted, by the HRP of the secondary antibody, into a dark precipitate readily visualized under a microscope. The spots where the dark precipitate has formed are then visually scored. The infectious units per milliliter (hereinafter referred to as IU/mL) were calculated based on the spots scored per microscope field, the total number of fields in the culture dish, the volume of serially diluted virus used for infection, and the dilution factor at which the spots were scored:
IU/mL=[(scored spots per field)*(total fields)]/[(volume diluted virus)*(dilution factor)]

In this case, 293 cells were infected with serially diluted OV1160 (lot 1), OV1160 (lot 2), or OV1164. An anti-hexon primary antibody (Chemicon; MAB8043), a secondary antibody (Amersham Biosciences; NA931V) and DAB substrate (Pierce; 1856090) was used to carry out the assay. PBS (Mediatech; 1-031-CV) supplemented with 1% BSA (Boehringer Mannheim; 100-350) was used as diluent for antibodies and for all intermediate washing steps.

The EC50 of the OV1165, 2 stocks of OV1160, OV1164, OV802, a laboratory-derived wild-type Ad5 isolate (Yu et al., 1999) and Addl312 (an E1a deleted replication defective virus) was determined on a number of cell lines using an MTS assay at day 7. The results are provided in Table 2, below as the dose a which 50% of the cells are killed (reported as PPC).

TABLE 2

| Cells/virus | OV1165 | OV1160#1 | OV1160#2 | OV1164 | 802 | dl312 |
|---|---|---|---|---|---|---|
| A549 | 0.12 | 0.07 | 0.21 | 0.86 | 0.02 | 543 |
| lung carcinoma | | | | | | |
| SW780 | 6 | 2 | 2 | 52 | 0.63 | 895 |
| Transitional cell carcinoma | | | | | | |
| SKMel-28 | 133 | 148 | 230 | 1033 | 15 | 4787 |
| melanoma | | | | | | |
| HT29 | 147 | 90 | 106 | 784 | 5 | 4301 |
| Colorectal adenocarcinoma | | | | | | |
| H460 | 0.62 | 0.43 | 0.7 | 10 | 0.02 | 1862 |
| Lung carcinoma | | | | | | |
| DLD-1 | 64 | 28 | 34 | 525 | 18 | na |

Colorectal adenocarcinoma

Western Blot and ELISA Analysis for TRAIL Expression

Western blot analysis was performed using methods widely known in the art (e.g., Anton and Graham, J. Virology, 69, 4600-4606, 1995, Sambrook and Russell, supra). A first series of Western blots was performed using the CVL (Crude viral lysate) of OV1164 and OV1160 following infection of A549 cells. For OV1164, A549 cells were infected at 1000, 100 or 10 particles per cell (ppc) for 24, 48 and 72hrs and the viral particle (vp) count as determined by HPLC was 8.6e9 vp/ml.

Various volumes of untitered OV1160 CVL were also used to infect A549 cells for 72 hrs. Following infection A549 cells were harvested using a cell scraper, centrifuged at 14,000 for 3 min and the cell pellet from cells infected with OV1160 or OV1164 was resuspended in 200 ul of cold Western lysis buffer, incubated 30 min on ice and stored at −80° C. until use. The quantity of protein in each sample was evaluated using the Bradford protein assay. 10 or 30 ug/ml of total cellular protein from OV1160 or OV1164 infected cells was combined in a solution containing 100 mM DTT and loading dye, denatured 5 min at 85□ C. and loaded onto a 4-12% gradient Bis-Tris SDS-Page gel (Novex from Invitrogen).

A second series of Western blots was performed using purified OV1160, OV1164 and OV1165 virus to infect A549 cells. The infection of A549 cells was carried out at PPC of 10 and 100 and the supernatant was harvested at 6, 24 and 48 hrs post infection. The cells were collected at 24 and 48 hrs post-infection. 30ug of total protein or 30 ul of supernatant were loaded onto a 4-12% gradient Bis-Tris SDS-Page gel (Novex from Invitrogen). The gel was subjected to 100 Volts for 1.5 hrs in 1× MOPS Buffer, and then transferred onto a PDVF membrane for 1 hr at 400 mAmp in 1× MOPS buffer with 10% of MeOH. The first antibody used was a Goat IgG anti-human TRAIL polyclonal (R&D Systems; AF375). A goat anti-HRP secondary antibody (Santa Cruz Bio SC-2056) was used. The results were detected using a ECL-Plus kit (Amersham) following exposure on Biomax MS film (Kodak).

TRAIL protein was detected by Western blot in cell lysates from A549 cells infected for 24, 48 or 72 hrs. with OV1160 and OV164 at a PPC of 10, 100 and 1000. The 19 kDa cleaved portion of TRAIL (soluble form) was also detected in the supernatant of A549 cells infected for 48 hrs with OV1160 at a PPC of 100.

TRAIL expression was quantitated by ELISA and the bystander effect of TRAIL produced by virally infected cells was performed by evaluation of the degree of apoptosis (as evaluated in a bioassay which is further described below). The analysis was carried out in order to determine whether TRAIL was produced following viral infection and if it was biologically active.

A549 cells were infected at a PPC of 100 for 24 and 48 hrs, respectively, with OV1160, OV1164 and OV1165. Supernatants were harvested and frozen at −80° C. until an assay was performed to determine the amount of TRAIL in the supernatant by ELISA.

Conditioned medium was collected and assayed for sTRAIL expression using a sandwich ELISA. 96-well microtiter plates were coated with mouse anti-human TRAIL monoclonal antibody (BioSource) in 0.1M carbonate pH 9.6 buffer and incubated overnight at 4° C. The plates were washed extensively with PBS-0.05% Tween-20, and blocked with PBS-1% BSA-0.05% Tween-20 buffer for 1 hr. Recombinant human TRAIL protein (R&D Systems, Minneapolis, Minn.) was used for standard curves after serial dilutions. Samples were incubated in the wells for 1 hr, washed extensively, and incubated with goat anti-human TRAIL polyclonal antibody (R&D Systems, Minneapolis, Minn.) for 1 hr. After extensive washing, the samples were incubated with HRP-conjugated anti-goat IgG antibody (Sigma Chemical Co.) for 1 hr, washed again, and detected using Sure Blue TMB substrate (KPL, Gaithersburg, Md.) at an optical density of 450/650 nm. A standard curve was generated using recombinant Human TRAIL (R&D Systems; 375 TEC). The results of this assay is provided in Table 3A, below.

TABLE 3A

| | PPC100 | | | |
|---|---|---|---|---|
| | 24 hr | | Mean | TRAIL (ng/ml) |
| OV1160#1 | 0.093 | 0.11 | 0.1015 | 1.1 |
| OV1160#2 | 0.1 | 0.1 | 0.1 | 0.9 |
| OV1165 | 0.078 | 0.079 | 0.0785 | |
| OV1164 | | | | |

| | 48 hr | | Mean | TRAIL (ng/ml) |
|---|---|---|---|---|
| OV1160#1 | 0.192 | 0.191 | 0.1915 | 9.6 |
| OV1160#2 | 0.208 | 0.207 | 0.2075 | 13.3 |
| OV1165 | 0.079 | 0.08 | 0.0795 | |
| OV1164 | 0.082 | 0.08 | 0.081 | |

A second ELISA was performed on 6 cell lines (SW780, A549, DID-I, SKMel28, HT29, H460) infected at their respective EC50 (determine by MTS at day 7) with OV1160 or OV1165 for 40 hrs. This study was carried out to evaluate the amount of TRAIL produced by a number of different cell lines. The results shown in Table 3B indicate that SW780, A549 and HT460 cells infected with OV1160 produced the greatest amount of TRAIL.

TABLE 3B

| TRAIL (ng/ml) | | SW780 | A549 | DLD1 | SK-Mel28 | HT29 | H460 |
|---|---|---|---|---|---|---|---|
| mean | OV1165 | 0 | 0 | 0.201301 | 0 | 0 | 0 |
| | OV1160#1 | 10.048 | 110.3783 | 4.790433 | 1.839404 | 0.727786 | 13.83983 |
| | OV1160#2 | 5.921467 | 87.18996 | 5.231936 | 1.890064 | 0.616181 | 13.55111 |
| sd | | 0 | 0 | 0.284682 | 0 | 0 | 0 |
| | | 1.997327 | 7.356895 | 0.159734 | 0.365634 | 0.072695 | 0 |
| | | | 5.811357 | 1.379575 | 0.437278 | 0.04107 | 1.353553 |

The bystander effect of TRAIL produced by cells infected with OV1160, OV1164 and OV1165 was evaluated by transfer of supernatant derived from infected cells and transferred onto fresh non-infected cells. The degree of apoptosis was evaluated by DNA fragmentation using an ELISA kit (Cell death detection kit Elisa plus/Roche 1774425).

A first assay was performed on 100 ul of supernatant collected following infection of A549 cells at a PPC 100 with OV1165, OV 1164 or one of two lots of OV1160 for 48 hrs with and without a general caspase inhibitor (R&D systems: FMK001). The analysis was performed 16 hrs following addition of the supernatant. The results shown in Table 4 indicate that OV1160 and not OV1165 is capable of inducing apoptosis on SW780 cells and when a caspase inhibitor is included in the culture, the effect is decreased.

TABLE 4

| | SW780 | A549 |
|---|---|---|
| Cells alone | 0.35 | 0.24 |
| 1165 | 0.68 | 0.26 |
| 1165 + inhibitor | 1 | 0.3 |
| 1160 | 3 | 0.175 |
| 1160 + inhibitor | 0.56 | 0.15 |
| rTrail | 3.75 | 1.45 |
| Positive control from kit | 3.3 | |
| Incubation buffer | 0.21 | |
| ABTS (dye) | 0.18 | |

A second assay is performed using 6 cell lines (SW780, DLD-1, HT29, SkMel28, H460 and A549) infected with OV1165 and OV1160 at their respective EC50s as determined by MTS on day 7. Freshly harvested supernatant from each virus-infected cell line is added to the corresponding non-infected cells with and without caspase inhibitor for 40 hrs before performing the ELISA. As shown in Table 3B, the level of TRAIL produced was variable for different cell lines. This study is designed to evaluate the biological activity of TRAIL produced by a particular cell line.

### Example 16

### Anti-Tumor Efficacy in the Subcutaneous Human Bladder SW780 Xenograft Tumor Model

A study was performed to evaluate the anti-tumor efficacy of TRAIL-expressing (OV1160) and control (OV1165) virus using a human bladder TCC cell line, SW780. In this current study, xenograft tumors of SW780 cells in nude mice are treated by five intratumoral injections of OV1160 ad OV 1165, respectively. The parameters measured include survival, changes in tumor volume, body weight, as well as the extent of viral infection, TRAIL expression and degree of apoptosis in tumors.

Tumors are established by injecting 2×10⁶ SW780 cells subcutaneously into female mice (10 animals per group). Intratumoral virus treatment is initiated when the mean tumor volume reaches approximately 150 mm³. Starting on Study Day (SD 1), tumors are injected five times at a dose of 1×10e8, 1×10e9, or 1×10e10 virus particles (vp) per injection, every other day for a total of 5 administrations. Tumor volume [(W²×L)/2] is measured twice per week; body weight is measured once per week. Hexon-staining and TRAIL ELISA is performed at various time points post infection.

Brief Description of the Sequences

The following is a description of the sequences employed in practicing the invention.

LLNFDLLKLAGDVESNPGP (SEQ ID NO: 1)
TLNFDLLKLAGDVESNPGP (SEQ ID NO: 2);
LLKLAGDVESNPGP (SEQ ID NO: 3)
NFDLLKLAGDVESNPGP (SEQ ID NO: 4)
QLLNFDLLKLAGDVESNPGP (SEQ ID NO: 5)
APVKQTLNFDLLKLAGDVESNPGP (SEQ ID NO: 6).
VTELLYRMKRAETYCPRPLLAIHPTEARHKQKIVAPVKQTLNFDLLKLAGDVESNPGP
(SEQ ID NO: 7)
LLAIHPTEARHKQKIVAPVKQTLNFDLLKLAGDVESNPGP (SEQ ID NO: 8)
EARHKQKIVAPVKQTLNFDLLKLAGDVESNPGP (SEQ ID NO: 9)
NFDLLKLAGDVESNPGPFF (SEQ ID NO: 10)
GIFNAHYAGYFADLLIHDIETNPGP (SEQ ID NO: 11)
RIFNAHYAGYFADLLIHDIETNPGP (SEQ ID NO: 12)
HVFETHYAGYFADLLIHDVETNPGP (SEQ ID NO: 13)
KAVRGYHADYYKQRLIHDVEMNPGP (SEQ ID NO: 14)
RAVRAYHADYYKQRLIHDVEMNPGP (SEQ ID NO: 15)
KAVRGYHADYYRQRLIHDVETNPGP (SEQ ID NO: 16)
LTNFDLLKLAGDVESNPGP (SEQ ID NO: 17)
LLNFDLLKLAGDMESNPGP (SEQ ID NO: 18)
MCNFDLLKLAGDVESNPGP (SEQ ID NO: 19)
CTNYALLKLAGDVESNPGP (SEQ ID NO: 20)
GATNFSLLKLAGDVELNPGP (SEQ ID NO: 21)
GPGATNFSLLKQAGDVEENPGP (SEQ ID NO: 22)
EAARQMLLLLSGDVETNPGP (SEQ ID NO: 23)
FLRKRTQLLMSGDVESNPGP (SEQ ID NO: 24)
GSWTDILLLLSGDVETNPGP (SEQ ID NO: 25)
RAEGRGSLLTCGDVEENPGP (SEQ ID NO: 26)
TRAEIEDELIRAGIESNPGP (SEQ ID NO: 27)
SKFQIDRILISGDIELNPGP (SEQ ID NO: 28)
AKFQIDKILISGDVELNPGP (SEQ ID NO: 29)
SKFQIDKILISGDIELNPGP (SEQ ID NO: 30)
SSIIRTKMLVSGDVEENPGP (SEQ ID NO: 31)
CDAQRQKLLLSGDIEQNPGP (SEQ ID NO: 32)
SEQ ID NO: 33 IS A FURIN CONSENSUS SEQUENCE OR SITE: RXK(R)R
SEQ ID NO: 34 IS A FACTOR XA CLEAVAGE SEQUENCE OR SITE: IE(D)GR
SEQ ID NO: 35 IS A SIGNAL PEPTIDASE I CLEAVAGE SEQUENCE OR SITE:
LAGFATVAQA
SEQ ID NO: 36 IS A THROMBIN CLEAVAGE SEQUENCE OR SITE: LVPRGS
SEQ ID NO: 37 IS AN Adenoviral consensus protease sequence or site (M, L, I)XGG/X
SEQ ID NO: 38 IS AN Adenoviral consensus protease sequence or site (M, L, I)XGX/G
SEQ ID NO: 39 is an exemplary sequence for a branch point plus splice acceptor
SEQ ID NO: 40 is a branch point consensus sequence
SEQ ID NO: 41 is nucleotides 29209 to 29336 of GenBank AY339865
SEQ ID NO: 42 is an L2 region splice site
SEQ ID NO: 43 is the E2F PROMOTER sequence
SEQ ID NO: 44 is an FMDV (VIRUS STRAIN C) IRES Sequence
SEQ ID NO: 45 is a 3′ splice acceptor site or SAS
SEQ ID NO: 46 is the hTRAIL ORF (INVIVOGEN)
SEQ ID NO: 47 is the CP1524 SEQUENCE (9587 BASE PAIRS)
SEQ ID NO: 48 is the CP1606 REV SEQUENCE (3855 BASE PAIRS)
SEQ ID NO: 49 is the OV 1165 sequence
SEQ ID NO: 50 is the OV1164 sequence
SEQ ID NO: 51 is the OV1160 sequence
SEQ ID NO: 52 is the sequence for oligonucleotide primer 1618.95.1
SEQ ID NO: 53 is the sequence for oligonucleotide primer 1618.95.
SEQ ID NO: 54 is the sequence for oligonucleotide primer 1618.97.1
SEQ ID NO: 55 is the sequence for oligonucleotide primer 1618.97.2
SEQ ID NO: 56 is the sequence for oligonucleotide primer 1618.95.5
SEQ ID NO: 57 is the sequence for oligonucleotide primer 1706.83.1
SEQ ID NO: 58 is the sequence for oligonucleotide primer 1706.83.2
SEQ ID NO: 59 is the sequence for oligonucleotide primer 1706.95.1
SEQ ID NO: 60 is the sequence for oligonucleotide primer 1618.116.3
SEQ ID NO: 61 is the sequence for oligonucleotide primer 1619.144.1
SEQ ID NO: 62 is the sequence for oligonucleotide primer 1619.144.3

The following is a description of the sequences employed in practicing the invention.

LLNFDLLKLAGDVESNPGP (SEQ ID NO: 1)
TLNFDLLKLAGDVESNPGP (SEQ ID NO: 2);
LLKLAGDVESNPGP (SEQ ID NO: 3)
NFDLLKLAGDVESNPGP (SEQ ID NO: 4)
QLLNFDLLKLAGDVESNPGP (SEQ ID NO: 5)
APVKQTLNFDLLKLAGDVESNPGP (SEQ ID NO: 6).
VTELLYRMKRAETYCPRPLLAIHPTEARHKQKIVAPVKQTLNFDLLKLA
GDVESNPGP (SEQ ID NO: 7)
LLAIHPTEARHKQKIVAPVKQTLNFDLLKLAGDVESNPGP
(SEQ ID NO: 8)
EARHKQKIVAPVKQTLNFDLLKLAGDVESNPGP (SEQ ID NO: 9)
NFDLLKLAGDVESNPGPFF (SEQ ID NO: 10)
GIFNAHYAGYFADLLIHDIETNPGP (SEQ ID NO: 11)
RIFNAHYAGYFADLLIHDIETNPGP (SEQ ID NO: 12)
HVFETHYAGYFADLLIHDVETNPGP (SEQ ID NO: 13)
KAVRGYHADYYKQRLIHDVEMNPGP (SEQ ID NO: 14)
RAVRAYHADYYKQRLIHDVEMNPGP (SEQ ID NO: 15)
KAVRGYHADYYRQRLIHDVETNPGP (SEQ ID NO: 16)
LTNFDLLKLAGDVESNPGP (SEQ ID NO: 17)
LLNFDLLKLAGDMESNPGP (SEQ ID NO: 18)
MCNFDLLKLAGDVESNPGP (SEQ ID NO: 19)
CTNYALLKLAGDVESNPGP (SEQ ID NO: 20)
GATNFSLLKLAGDVELNPGP (SEQ ID NO: 21)
GPGATNFSLLKQAGDVEENPGP (SEQ ID NO: 22)
EAARQMLLLLSGDVETNPGP (SEQ ID NO: 23)
FLRKRTQLLMSGDVESNPGP (SEQ ID NO: 24)
GSWTDILLLLSGDVETNPGP (SEQ ID NO: 25)
RAEGRGSLLTCGDVEENPGP (SEQ ID NO: 26)
TRAEIEDELIRAGIESNPGP (SEQ ID NO: 27)
SKFQIDRILISGDIELNPGP (SEQ ID NO: 28)
AKFQIDKILISGDVELNPGP (SEQ ID NO: 29)
SKFQIDKILISGDIELNPGP (SEQ ID NO: 30)
SSIIRTKMLVSGDVEENPGP (SEQ ID NO: 31)
CDAQRQKLLLSGDIEQNPGP (SEQ ID NO: 32)
SEQ ID NO: 33 IS A FURIN CONSENSUS SEQUENCE OR
SITE: RXK(R)R
SEQ ID NO: 34 IS A FACTOR XA CLEAVAGE SEQUENCE OR
SITE: IE(D)GR
SEQ ID NO: 35 IS A SIGNAL PEPTIDASE I CLEAVAGE
SEQUENCE OR SITE:
LAGFATVAQA
SEQ ID NO: 36 IS A THROMBIN CLEAVAGE SEQUENCE OR
SITE: LVPRGS
SEQ ID NO: 37 IS AN Adenoviral consensus protease
sequence or site (M, L, I)XGG/X
SEQ ID NO: 38 IS AN Adenoviral consensus protease
sequence or site (M, L, I)XGX/G
SEQ ID NO: 39 is an exemplary sequence for a
branch point plus splice acceptor TACTTAT
GACTCGTACTATTGTTATTCATCC AG^{—}G
SEQ ID NO: 40 is a branch point consensus sequence
YNYURAY (where Y is a pyrimidine,
N is any nucleotide, and R is a purine)
SEQ ID NO: 41 is nucleotides 29209 to 29336 of
GenBank AY339865
TAATTTACTAAGTTACAAAGCTAATGTCACCACTAACTGCTTTACTCGC
TGCTTGCAAAACAAATTCAAAAAGTTAGCATTATAATTAGAATAGGATT
TAAACCCCCCGGTCATTTCCTGCTCAATAC
SEQ ID NO: 42 is an L2 region splice site
TAC TTAT AGT AAT CTA A TT CCT G CT CTC TC AG
SEQ ID NO: 43 is the E2F PROMOTER sequence
CATCCGGACAAAGCCTGCGCGCGCCCCGCCCCGCCATTGGCCGTACCGCC
CCGC
GCCGCCGCCCCATCTCGCCCCTCGCCGCCGGGTCCGGCGCGTTAAAGCCA
ATAG
GAACCGCCGCCGTTGTTCCCGTCACGGCCGGGGCAGCCAATTGTGGCGGC
GCTC
GGCGGCTCGTGGCTCTTTCGCGGCAAAAAGGATTTGGCGCGTAAAAGTGG
CGGG
GACTTTGCAGGCAGCGGCGGCCGGGGGCGGAGCGGGATCGAGCCCTCGAT
GATATCA
SEQ ID NO: 44 is an FMDV (VIRUS STRAIN C) IRES
Sequence
AGCAGGTTTCCCCAACTG
ACACAAAACGTGCAACTTGAAACTCCGCCTGGTCTTTCCAGGTCTAGAGG
GGTAACACTTTGTACTGCGT
TTGGCTCCACGCTCGATCCACTGGCGAGTGTTAGTAACAGCACTGTTGCT
TCGTAGCGGAGCATGACGGC
CGTGGGAACTCCTCCTTGGTAACAAGGACCCACGGGGCCAAAAGCCACGC
CCACACGGGCCCGTCATGTG
TGCAACCCCAGCACGGCGACTTTACTGCGAAACCCACTTTAAAGTGACAT
TGAAACTGGTACCCACACAC
TGGTGACAGGCTAAGGATGCCCTTCAGGTACCCCGAGGTAACACGCGACA
CTCGGGATCTGAGAAGGGGA
CTGGGGCTTCTATAAAAGCGCTCGGTTTAAAAAGCTTCTATGCCTGAATA
GGTGACCGGAGGTCGGCACC
TTTCCTTTGCAATTAATGACCCT
SEQ ID NO: 45 is a 3′ splice acceptor site
or SAS
TACTTATGACTCGTACTATTGTTATTCATCCAG G
SEQ ID NO: 46 is the hTRAIL ORF (INVIVOGEN)
(SEQ ID NO: 46)
ATGGCTATGATGGAGGTCCAGGGGGGACCCAGCCTGGGACAGACCTGCG
TGCTGATCGTGATCTTTACAGTG
CTCCTGCAGTCTCTCTGTGTGGCTGTAACTTACGTGTACTTTACCAACG
AGCTGAAGCAGATGCAGGACAAG
TACTCCAAAAGTGGCATTGCTTGTTTCTTAAAAGAAGATGACAGTTATT
GGGACCCCAATGACGAAGAGAGT
ATGAACAGCCCCTGCTGGCAAGTCAAGTGGCAACTCCGTCAGCTCGTTA
GAAAGATGATTTTGAGAACCTCT
GAGGAAACCATTTCTACAGTTCAAGAAAAGCAACAAAATATTTCTCCCC
TAGTGAGAGAAAGAGGTCCTCAG
AGAGTAGCAGCTCACATAACTGGGACCAGAGGAAGAAGCAACACATTGT
CTTCTCCAAACTCCAAGAATGAA
AAGGCTCTGGGCCGCAAAATAAACTCCTGGGAATCATCAAGGAGTGGGC
ATTCATTCCTGAGCAACTTGCAC
TTGAGGAATGGTGAACTGGTCATCCATGAAAAAGGGTTTTACTACATCT
ATTCCCAAACATACTTTCGATTT
CAGGAGGAAATAAAAGAAAACACAAAGAACGACAAACAAATGGTCCAAT
ATATTTACAAATACACAAGTTAT
CCTGACCCTATATTGTTGATGAAAAGTGCTAGAAATAGTTGTTGGTCTA
AAGATGCAGAATATGGACTCTAT
TCCATCTATCAAGGGGGAATATTTGAGCTTAAGGAAAATGACAGAATTT
TTGTTTCTGTAACAAATGAGCAC
TTAATAGACATGGACCATGAAGCCAGTTTTTTCGGGGCCTTTTTAGTTG
GCTAA
SEQ ID NO: 47 is the CP1524 SEQUENCE (9587 BASE
PAIRS)
GACGGATCGGGAGATCTCCCGATCCCCTATGGTGCACTCTCAGTACAAT
CTGCTCTGATGCCGCATAGTTAAG
CCAGTATCTGCTCCCTGCTTGTGTGTTGGAGGTCGCTGAGTAGTGCGCG
AGCAAAATTTAAGCTACAACAAGG
CAAGGCTTGACCGACAATTGCATGAAGAATCTGCTTAGGGTTAGGCGTT
TTGCGCTGCTTCGCGATGTACGGG
CCAGATATACGCGTTGACATTGATTATTGACTAGTTATTAATAGTAATC
AATTACGGGGTCATTAGTTCATAG
CCCATATATGGAGTTCCGCGTTACATAACTTACGGTAAATGGCCCGCCT
GGCTGACCGCCCAACGACCCCCGC
CCATTGACGTCAATAATGACGTATGTTCCCATAGTAACGCCAATAGGGA
CTTTCCATTGACGTCAATGGGTGG
AGTATTTACGGTAAACTGCCCACTTGGCAGTACATCAAGTGTATCATAT
GCCAAGTACGCCCCCTATTGACGT
CAATGACGGTAAATGGCCCGCCTGGCATTATGCCCAGTACATGACCTTA
TGGGACTTTCCTACTTGGCAGTAC
ATCTACGTATTAGTCATCGCTATTACCATGGTGATGCGGTTTTGGCAGT
ACATCAATGGGCGTGGATAGCGGT
TTGACTCACGGGGATTTCCAAGTCTCCACCCCATTGACGTCAATGGGAG
TTTGTTTTGGCACCAAAATCAACG
GGACTTTCCAAAATGTCGTAACAACTCCGCCCCATTGACGCAAATGGGC
GGTAGGCGTGTACGGTGGGAGGTC
TATATAAGCAGAGCTCTCTGGCTAACTAGAGAACCCACTGCTTACTGGC
TTATCGAAATTAATACGACTCACT
ATAGGGAGACCCAAGCTGGCTAGCGTTTAAACTTAAGCTTGATCCCCGC
CCTCCCGTAGAGGAGCCTCCACCG
GCCGTGGAGACAGTGTCTCCAGAGGGGCGTGGCGAAAAGCGTCCGCGCC
CCGACAGGGAAGAAACTCTGGTGA
CGCAAATAGACGAGCCTCCCTCGTACGAGGAGGCACTAAAGCAAGGCCT
GCCCACCACCCGTCCCATCGCGCC
CATGGCTACCGGAGTGCTGGGCCAGCACACACCCGTAACGCTGGACCTG
CCTCCCCCCGCCGACACCCAGCAG
AAACCTGTGCTGCCAGGCCCGACCGCCGTTGTTGTAACCCGTCCTAGCC
GCGCGTCCCTGCGCCGCGCCGCCA
GCGGTCCGCGATCGTTGCGGCCCGTAGCCAGTGGCAACTGGCAAAGCAC
ACTGAACAGCATCGTGGGTCTGGG
GGTGCAATCCCTGAAGCGCCGACGATGCTTCTGAATAGCTAACGTGTCG
TATGTGTGTCATGTATGCGTCCAT
GTCGCCGCCAGAGGAGCTGCTGAGCCGCCGCGCGCCCGCTTTCCAAGAT
GGCTACCCCTTCGATGATGCCGCA
GTGGTCTTACATGCACATCTCGGGCCAGGACGCCTCGGAGTACCTGAGC
CCCGGGCTGGTGCAGTTTGCCCGC
GCCACCGAGACGTACTTCAGCCTGAATAACAAGTTTAGAAACCCCACGG
TGGCGCCTACGCACGACGTGACCA
CAGACCGGTCCCAGCGTTTGACGCTGCGGTTCATCCCTGTGGACCGTGA
GGATACTGCGTACTCGTACAAGGC
GCGGTTCACCCTAGCTGTGGGTGATAACCGTGTGCTGGACATGGCTTCC
ACGTACTTTGACATCCGCGGCGTG
CTGGACAGGGGCCCTACTTTTAAGCCCTACTCTGGCACTGCCTACAACG
CCCTGGCTCCCAAGGGTGCCCCAA
ATCCTTGCGAATGGGATGAAGCTGCTACTGCTCTTGAAATAAACCTAGA
AGAAGAGGACGATGACAACGAAGA
CGAAGTAGACGAGCAAGCTGAGCAGCAAAAAACTCACGTATTTGGGCAG
GCGCCTTATTCTGGTATAAATATT
ACAAAGGAGGGTATTCAAATAGGTGTCGAAGGTCAAACACCTAAATATG
CCGATAAAACATTTCAACCTGAAC
CTCAAATAGGAGAATCTCAGTGGTACGAAACTGAAATTAATCATGCAGC
TGGGAGAGTCCTTAAAAAGACTAC
CCCAATGAAACCATGTTACGGTTCATATGCAAAACCCACAAATGAAAAT
GGAGGGCAAGGCATTCTTGTAAAG
CAACAAAATGGAAAGCTAGAAAGTCAAGTGGAAATGCAATTTTTCTCAA
CTACTGAGGCGACCGCAGGCAATG
GTGATAACTTGACTCCTAAAGTGGTATTGTACAGTGAAGATGTAGATAT
AGAAACCCCAGACACTCATATTTC
TTACATGCCCACTATTAAGGAAGGTAACTCACGAGAACTAATGGGCCAA
CAATCTATGCCCAACAGGCCTAAT
TACATTGCTTTTAGGGACAATTTTATTGGTCTAATGTATTACAACAGCA
CGGGTAATATGGGTGTTCTGGCGG
GCCAAGCATCGCAGTTGAATGCTGTTGTAGATTTGCAAGACAGAAACAC
AGAGCTTTCATACCAGCTTTTGCT
TGATTCCATTGGTGATAGAACCAGGTACTTTTCTATGTGGAATCAGGCT
GTTGACAGCTATGATCCAGATGTT
AGAATTATTGAAAATCATGGAACTGAAGATGAACTTCCAAATTACTGCT
TTCCACTGGGAGGTGTGATTAATA
CAGAGACTCTTACCAAGGTAAAACCTAAAACAGGTCAGGAAAATGGATG
GGAAAAAGATGCTACAGAATTTTC
AGATAAAAATGAAATAAGAGTTGGAAATAATTTTGCCATGGAAATCAAT
CTAAATGCCAACCTGTGGAGAAAT
TTCCTGTACTCCAACATAGCGCTGTATTTGCCCGACAAGCTAAAGTACA
GTCCTTCCAACGTAAAAATTTCTG
ATAACCCAAACACCTACGACTACATGAACAAGCGAGTGGTGGCTCCCGG
GTTAGTGGACTGCTACATTAACCT
TGGAGCACGCTGGTCCCTTGACTATATGGACAACGTCAACCCATTTAAC
CACCACCGCAATGCTGGCCTGCGC
TACCGCTCAATGTTGCTGGGCAATGGTCGCTATGTGCCCTTCCACATCC
AGGTGCCTCAGAAGTTCTTTGCCA
TTAAAAACCTCCTTCTCCTGCCGGGCTCATACACCTACGAGTGGAACTT
CAGGAAGGATGTTAACATGGTTCT
GCAGAGCTCCCTAGGAAATGACCTAAGGGTTGACGGAGCCAGCATTAAG
TTTGATAGCATTTGCCTTTACGCC
ACCTTCTTCCCCATGGCCCACAACACCGCCTCCACGCTTGAGGCCATGC
TTAGAAACGACACCAACGACCAGT
CCTTTAACGACTATCTCTCCGCCGCCAACATGCTCTACCCTATACCCGC
CAACGCTACCAACGTGCCCATATC
CATCCCCTCCCGCAACTGGGCGGCTTTCCGCGGCTGGGCCTTCACGCGC
CTTAAGACTAAGGAAACCCCATCA
CTGGGCTCGGGCTACGACCCTTATTACACCTACTCTGGCTCTATACCCT
ACCTAGATGGAACCTTTTACCTCA
ACCACACCTTTAAGAAGGTGGCCATTACCTTTGACTCTTCTGTCAGCTG
GCCTGGCAATGACCGCCTGCTTAC
CCCCAACGAGTTTGAAATTAAGCGCTCAGTTGACGGGGAGGGTTACAAC
GTTGCCCAGTGTAACATGACCAAA
GACTGGTTCCTGGTACAAATGCTAGCTAACTACAACATTGGCTACCAGG
GCTTCTATATCCCAGAGAGCTACA
AGGACCGCATGTACTCCTTCTTTAGAAACTTCCAGCCCATGAGCCGTCA
GGTGGTGGATGATACTAAATACAA
GGACTACCAACAGGTGGGCATCCTACACCAACACAACAACTCTGGATTT
GTTGGCTACCTTGCCCCCACCATG
CGCGAAGGACAGGCCTACCCTGCTAACTTCCCCTATCCGCTTATAGGCA
AGACCGCAGTTGACAGCATTACCC
AGAAAAAGTTTCTTTGCGATCGCACCCTTTGGCGCATCCCATTCTCCAG
TAACTTTATGTCCATGGGCGCACT
CACAGACCTGGGCCAAAACCTTCTCTACGCCAACTCCGCCCACGCGCTA
GACATGACTTTTGAGGTGGATCCC
ATGGACGAGCCCACCCTTCTTTATGTTTTGTTTGAAGTCTTTGACGTGG
TCCGTGTGCACCGGCCGCACCGCG
GCGTCATCGAAACCGTGTACCTGCGCACGCCCTTCTCGGCCGGCAACGC
CACAACATAAAGAAGCAAGCAACA
TCAACAACAGCTGCCGCCATGGGCTCCAGTGAGCAGGAACTGAAAGCCA
TTGTCAAAGATCTTGGTTGTGGGC
CATATTTTTTGGGCACCTATGACAAGCGCTTTCCAGGCTTTGTTTCTCC
ACACAAGCTCGCCTGCGCCATAGT
CAATACGGCCGGTCGCGAGACTGGGGGCGTACACTGGATGGCCTTTGCC
TGGAACCCGCACTCAAAAACATGC
TACCTCTTTGAGCCCTTTGGCTTTTCTGACCAGCGACTCAAGCAGGTTT
ACCAGTTTGAGTACGAGTCACTCC
TGCGCCGTAGCGCCATTGCTTCTTCCCCCGACCGCTGTATAACGCTGGA
AAAGTCCACCCAAAGCGTACAGGG
GCCCAACTCGGCCGCCTGTGGACTATTCTGCTGCATGTTTCTCCACGCC
TTTGCCAACTGGCCCCAAACTCCC
ATGGATCACAACCCCACCATGAACCTTATTACCGGGGTACCCAACTCCA
TGCTCAACAGTCCCCAGGTACAGC
CCACCCTGCGTCGCAACCAGGAACAGCTCTACAGCTTCCTGGAGCGCCA
CTCGCCCTACTTCCGCAGCCACAG
TGCGCAGATTAGGAGCGCCACTTCTTTTTGTCACTTGAAAAACATGTAA
AAATAATGTACTAGAGACACTTTC
AATAAAGGCAAATGCTTTTATTTGTACACTCTCGGGTGATTATTTACCC
CCACCCTTGCCGTCTGCGCCGTTT
AAAAATCAAAGGGGTTCTGCCGCGCATCGCTATGCGCCACTGGCAGGGA
CACGTTGCGATACTGGTGTTTAGT
GCTCCACTTAAACTCAGGCACAACCATCCGCGGCAGCTCGGTGAAGTTT
TCACTCCACAGGCTGCGCACCATC
ACCAACGCGTTTAGCAGGTCGGGCGCCGATATCTTGAAGTCGCAGTTGG
GGCCTCCGCCCTGCGCGCGCGAGT
TGCGATACACAGGGTTGCAGCACTGGAACACTATCAGCGCCGGGTGGTG
CACGCTGGCCAGCACGCTCTTGTC
GGAGATCAGATCCGCGTCCAGGTCCTCCGCGTTGCTCAGGGCGAACGGA
GTCAACTTTGGTAGCTGCCTTCCC
AAAAAGGGCGCGTGCCCAGGCTTTGAGTTGCACTCGCACCGTAGTGGCA
TCAAAAGGTGACCGTGCCCGGTCT
GGGCGTTAGGATACAGCGCCTGCATAAAAGCCTTGATCTGCTTAAAAGC
CACCTGAGCCTTTGCGCCTTCAGA
GAAGAACATGCCGCAAGACTTGCCGGAAAACTGATTGGCCGGACAGGCC
GCGTCGTGCACGCAGCACCTTGCG
TCGGTGTTGGAGATCTGCACCACATTTCGGCCCCACCGGTTCTTCACGA
TCTTGGCCTTGCTAGACTGCTCCT
TCAGCGCGCGCTGCCCGTTTTCGCTCGTCACATCCATTTCAATCACGTG
CTCCTTATTTATCATAATGCTTCC
GTGTAGACACTTAAGCTCGCCTTCGATCTCAGCGCAGCGGTGCAGCCAC
AACGCGCAGCCCGTGGGCTCGTGA
TGCTTGTAGGTCACCTCTGCAAACGACTGCAGGTACGCCTGCAGGAATC
GCCCCATCATCGTCACAAAGGTCT
TGTTGCTGGTGAAGGTCAGCTGCAACCCGCGGTGCTCCTCGTTCAGCCA
GGTCTTGCATACGGCCGCCAGAGC
TTCCACTTGGTCAGGCAGTAGTTTGAAGTTCGCCTTTAGATCGTTATCC
ACGTGGTACTTGTCCATCAGCGCG
CGCGCAGCCTCCATGCCCTTCTCCCACGCAGACACGATCGGCACACTCA
GCGGGTTCATCACCGTAATTTCAC
TTTCCGCTTCGCTGGGCTCTTCCTCTTCCTCTTGCGTCCGCATACCACG
CGCCACTGGGTCGTCTTCATTCAG
CCGCCGCACTGTGCGCTTACCTCCTTTGCCATGCTTGATTAGCACCGGT
GGGTTGCTGAAACCCACCATTTGT
AGCGCCACATCTTCTCTTTCTTCCTCGCTGTCCACGATTACCTCTGGTG
ATGGCGGGCGCTCGGGCTTGGGAG
AAGGGCGCTTCTTTTTCTTCTTGGGCGCAATGGCCAAATCCGCCGCCGA
GGTCGATGGCCGCGGGCTGGGTGT
GCGCGGCACCAGCGCGTCTTGTGATGAGTCTTCCTCGTCCTCGGACTCG
ATACGCCGCCTCATCCGCTTTTTT
GGGGGCGCCCGGGGAGGCGGCGGCGACGGGGACGGGGACGACACGTCCT
CCATGGTTGGGGGACGTCGCGCCG
CACCGCGTCCGCGCTCGGGGGTGGTTTCGCGCTGCTCCTCTTCCCGACT
GGCCATTTCCTTCTCCTATAGGCA
GAAAAAGATCATGGAGTCAGTCGAGAAGAAGGACAGCCTAACCGCCCCC
TCTGAGTTCGCCACCACCGCCTCC
ACCGATGCCGCCAACGCGCCTACCACCTTCCCCGTCGAGGCACCCCCGC
TTGAGGAGGAGGAAGTGATTATCG
AGCAGGACCCAGGTTTTGTAAGCGAAGACGACGAGGACCGCTCAGTACC
AACAGAGGATAAAAAGCAAGACCA
GGACAACGCAGAGGCAAACGAGGAACAAGTCGGGCGGGGGGACGAAAGG
CATGGCGACTACCTAGATGTGGGA
GACGACGTGCTGTTGAAGCATCTGCAGCGCCAGTGCGCCATTATCTGCG
ACGCGTTGCAAGAGCGCAGCGATG
TGCCCCTCGCCATAGCGGATGTCAGCCTTGCCTACGAACGCCACCTATT
CTCACCGCGCGTACCCCCCAAACG
CCAAGAAAACGGCACATGCGAGCCCAACCCGCGCCTCAACTTCTACCCC
GTATTTGCCGTGCCAGAGGTGCTT
GCCACCTATCACATCTTTTTCCAAAACTGCAAGATACCCCTATCCTGCC
GTGCCAACCGCAGCCGAGCGGACA
AGCAGCTGGCCTTGCGGCAGGGCGCTGTCATACCTGATATCGCCTCGCT
CAACGAAGTGCCAAAAATCTTTGA
GGGTCTTGGACGCGACGAGAAGCGCGCGGCAAACGCTCTGCAACAGGAA
AACAGCGAAAATGAAAGTCACTCT
GGAGTGTTGGTGGAACTCGAGTTACCGTCGACCTCTAGCTAGAGCTTGG
CGTAATCATGGTCATAGCTGTTTC
CTGTGTGAAATTGTTATCCGCTCACAATTCCACACAACATACGAGCCGG
AAGCATAAAGTGTAAAGCCTGGGG
TGCCTAATGAGTGAGCTAACTCACATTAATTGCGTTGCGCTCACTGCCC
GCTTTCCAGTCGGGAAACCTGTCG
TGCCAGCTGCATTAATGAATCGGCCAACGCGCGGGGAGAGGCGGTTTGC
GTATTGGGCGCTCTTCCGCTTCCT
CGCTCACTGACTCGCTGCGCTCGGTCGTTCGGCTGCGGCGAGCGGTATC
AGCTCACTCAAAGGCGGTAATACG
GTTATCCACAGAATCAGGGGATAACGCAGGAAAGAACATGTGAGCAAAA
GGCCAGCAAAAGGCCAGGAACCGTA
AAAAGGCCGCGTTGCTGGCGTTTTTCCATAGGCTCCGCCCCCCTGACGA
GCATCACAAAAATCGACGCTCAAGT
CAGAGGTGGCGAAACCCGACAGGACTATAAAGATACCAGGCGTTTCCCC
CTGGAAGCTCCCTCGTGCGCTCTCC
TGTTCCGACCCTGCCGCTTACCGGATACCTGTCCGCCTTTCTCCCTTCG
GGAAGCGTGGCGCTTTCTCATAGCT
CACGCTGTAGGTATCTCAGTTCGGTGTAGGTCGTTCGCTCCAAGCTGGG
CTGTGTGCACGAACCCCCCGTTCAG
CCCGACCGCTGCGCCTTATCCGGTAACTATCGTCTTGAGTCCAACCCGG
TAAGACACGACTTATCGCCACTGGC
AGCAGCCACTGGTAACAGGATTAGCAGAGCGAGGTATGTAGGCGGTGCT
ACAGAGTTCTTGAAGTGGTGGCCTA
ACTACGGCTACACTAGAAGAACAGTATTTGGTATCTGCGCTCTGCTGAA
GCCAGTTACCTTCGGAAAAAGAGTT
GGTAGCTCTTGATCCGGCAAACAAACCACCGCTGGTAGCGGTTTTTTTG
TTTGCAAGCAGCAGATTACGCGCAG
AAAAAAAGGATCTCAAGAAGATCCTTTGATCTTTTCTACGGGGTCTGAC
GCTCAGTGGAACGAAAACTCACGTT
AAGGGATTTTGGTCATGAGATTATCAAAAAGGATCTTCACCTAGATCCT
TTTAAATTAAAAATGAAGTTTTAAA
TCAATCTAAAGTATATATGAGTAAACTTGGTCTGACAGTTACCAATGCT
TAATCAGTGAGGCACCTATCTCAGC
GATCTGTCTATTTCGTTCATCCATAGTTGCCTGACTCCCCGTCGTGTAG
ATAACTACGATACGGGAGGGCTTAC
CATCTGGCCCCAGTGCTGCAATGATACCGCGAGACCCACGCTCACCGGC
TCCAGATTTATCAGCAATAAACCAG
CCAGCCGGAAGGGCCGAGCGCAGAAGTGGTCCTGCAACTTTATCCGCCT
CCATCCAGTCTATTAATTGTTGCCG
GGAAGCTAGAGTAAGTAGTTCGCCAGTTAATAGTTTGCGCAACGTTGTT
GCCATTGCTACAGGCATCGTGGTGT
CACGCTCGTCGTTTGGTATGGCTTCATTCAGCTCCGGTTCCCAACGATC
AAGGCGAGTTACATGATCCCCCATG
TTGTGCAAAAAAGCGGTTAGCTCCTTCGGTCCTCCGATCGTTGTCAGAA
GTAAGTTGGCCGCAGTGTTATCACT
CATGGTTATGGCAGCACTGCATAATTCTCTTACTGTCATGCCATCCGTA
AGATGCTTTTCTGTGACTGGTGAGT
ACTCAACCAAGTCATTCTGAGAATAGTGTATGCGGCGACCGAGTTGCTC
TTGCCCGGCGTCAATACGGGATAAT
ACCGCGCCACATAGCAGAACTTTAAAAGTGCTCATCATTGGAAAACGTT
CTTCGGGGCGAAAACTCTCAAGGAT
CTTACCGCTGTTGAGATCCAGTTCGATGTAACCCACTCGTGCACCCAAC
TGATCTTCAGCATCTTTTACTTTCA
CCAGCGTTTCTGGGTGAGCAAAAACAGGAAGGCAAAATGCCGCAAAAAA
GGGAATAAGGGCGACACGGAAATGT
TGAATACTCATACTCTTCCTTTTTCAATATTATTGAAGCATTTATCAGG
GTTATTGTCTCATGAGCGGATACAT
ATTTGAATGTATTTAGAAAAATAAACAAATAGGGGTTCCGCGCACATTT
CCCCGAAAAGTGCCACCTGACGTC
SEQ ID NO: 48 is the CP1606 REV SEQUENCE (3855
BASE PAIRS)
AGCGCCCAATACGCAAACCGCCTCTCCCCGCGCGTTGGCCGATTCATTA
ATGCAGCTGGCACGACAGGTTTCCCG
ACTGGAAAGCGGGCAGTGAGCGCAACGCAATTAATGTGAGTTAGCTCAC
TCATTAGGCACCCCAGGCTTTACACT
TTATGCTTCCGGCTCGTATGTTGTGTGGAATTGTGAGCGGATAACAATT
TCACACAGGAAACAGCTATGACCATG
ATTACGCCAAGCTTGGTACCGAGCTCGGATCCACTAGTAACGGCCGCCA
GTGTGCTGGAATTCGCCCTTGACGCG
GCCTGTCCGGCCAATCAGTTTTCCGGCAAGTCTTGCGGCATGTTCTTCT
CTGAAGGCGCAAAGGCTCAGGTGGCT
TTTAAGCAGATCAAGGCTTTTATGCAGGCGCTGTATCCTAACGCCCAGA
CCGGGCACGGTCACCTTTTGATGCCA
CTACGGTGCGAGTGCAACTCAAAGCCTGGGCACGCGCCCTTTTTGGGAA
GGCAGCTACCAAAGTTGACTCCGTTC
GCCCTGAGCAACGCGGAGGACCTGGACGCGGATCTGATCTCCGACAAGA
GCGTGCTGGCCAGCGTGCACCACCCG
GCGCTGATAGTGTTCCAGTGCTGCAACCCTGTGTATCGCAACTCGCGCG
CGCAGGGCGGAGGCCCCAACTGCGAC
TTCAAGATATCGGCGCCCGACCTGCTAAACGCGTTGGTGATGGTGCGCA
GCCTGTGGAGTGAAAACTTCACCGAG
CTGCCGCGGATGGTTGTGCCTGAGTTTAAGTGGAGCACTAAACACCAGT
ATCGCAACGTGTCCCTGCCAGTGGCG
CATAGCGATGCGCGGCAGAACCCCTTTGATTTTTAAACGGCGCAGACGG
CAAGGGTGGGGGTAAATAATCACCCG
AGAGTGTACAAATAAAAGCATTTGCCTTTATTGAAAGTGTCTCTAGTAG
CTAGCGGGAGGGAGGTCCTGGTCTAG
ACATTCAGAGCTTGTAGAATTTCTGCATCCAGGTCAGAAGAATGTCCAC
TTCCCCAAGGGCTTTGGTCAGAGCTG
CTTCTACGTCCAACTGTTTGAATGCTCTCCGGAATAGCAGAAACCGCCT
GTGTGCACTGTCTCTGATGGAAAACA
TCTCATTTTCTTGACTGGGTTGCAGTTGTGACACGATGAGAACAAAGTT
GTTGGCCAGAGTAGAGAATGACTTCA
GAGTCCTGACTTCAACTGTTCTATTGTGGTGGTTTTTGAAAACAGTTTT
CAAGTAGAACTCCAGCAGGGTGTGGA
CAAGGTAACAGCTCTCAGCATCCGAGACGTTCTGCAGAACCTCCTGCTG
CAGCAGCCGGGCACTCGTGATGTTAT
CCTGAGCTTGCATAGTGTCTTTCACAGCCCAGAAGGCTTCCCACAGTTT
CTGGGGAACAACCCCCTTCACTTGGC
AGGGCCCAAAGTGGAATTCTTGGCCCTGGGCCCCTGATACCTGGCTCCA
GAGAAGCAGGGTAAAACCCAGGCAAG
GGAGCACAACCATCTGCATTTGAGAGGCTGTCGCCAGCAAAGGAGGGCA
GAAGGGTCTGGCTAAAGTCCACAGGC
TTTGCAGCCTCTGTTGAAAATTCATGATGGCCCTCCTACCGCCTGGATG
AATAACAATAGTACGAGTCATAAGTA
ATTATTTTTACATGTTTTTCAAGTGACAAAAAGAAGTGGCGCTCCTAAT
CTGCGCACTGTGGCTGCGGAAGTAGG
GCGAGTGGCGCTCCAGGAAGCTGTAGAGCTGTTCCTGGTTGCGACGCAG
GGTGGGCTGTACCTGGGGACTGTTGA
GCATGGAGTTGGGTACCCCGGTAAAAAGGGCGAATTCTGCAGATATCCA
TCACACTGGCGGCCGCTCGAGCATGC
ATCTAGAGGACCGCTATCAGGACATAGCGTTGGCTACCCGTGATATTGC
TGAAGAGCTTGGCGGCGAATGGGCTG
ACCGCTTCCTCGTGCTTTACGGTATCGCCGCTCCCGATTCGCAGCGCAT
CGCCTTCTATCGCCTTCTTGACGAGT
TCTTCTGAATTGAAAAAGGAAGAGTATGAGTATTCAACATTTCCGTGTC
GCCCTTATTCCCTTTTTTGCGGCATT
TTGCCTTCCTGTTTTTGCTCACCCAGAAACGCTGGTGAAAGTAAAAGAT
GCTGAAGATCAGTTGGGTGCACGAGT
GGGTTACATCGAACTGGATCTCAACAGCGGTAAGATCCTTGAGAGTTTT
CGCCCCGAAGAACGTTTTCCAATGAT
GAGCACTTTTAAAGTTCTGCTATGTGGCGCGGTATTATCCCGTATTGAC
GCCGGGCAAGAGCAACTCGGTCGCCG
CATACACTATTCTCAGAATGACTTGGTTGAGTACTCACCAGTCACAGAA
AAGCATCTTACGGATGGCATGACAGT
AAGAGAATTATGCAGTGCTGCCATAACCATGAGTGATAACACTGCGGCC
AACTTACTTCTGACAACGATCGGAGG
ACCGAAGGAGCTAACCGCTTTTTTGCACAACATGGGGGATCATGTAACT
CGCCTTGATCGTTGGGAACCGGAGCT
GAATGAAGCCATACCAAACGACGAGCGTGACACCACGATGCCTGTAGCA
ATGGCAACAACGTTGCGCAAACTATT
AACTGGCGAACTACTTACTCTAGCTTCCCGGCAACAATTAATAGACTGG
ATGGAGGCGGATAAAGTTGCAGGACC
ACTTCTGCGCTCGGCCCTTCCGGCTGGCTGGTTTATTGCTGATAAATCT
GGAGCCGGTGAGCGTGGGTCTCGCGG
TATCATTGCAGCACTGGGGCCAGATGGTAAGCCCTCCCGTATCGTAGTT
ATCTACACGACGGGGAGTCAGGCAAC
TATGGATGAACGAAATAGACAGATCGCTGAGATAGGTGCCTCACTGATT
AAGCATTGGTAACTGTCAGACCAAGT
TTACTCATATATACTTTAGATTGATTTAAAACTTCATTTTTAATTTAAA
AGGATCTAGGTGAAGATCCTTTTTGA
TAATCTCATGCATGACCAAAATCCCTTAACGTGAGTTTTCGTTCCACTG
AGCGTCAGACCCCGTAGAAAAGATCA
AAGGATCTTCTTGAGATCCTTTTTTTCTGCGCGTAATCTGCTGCTTGCA
AACAAAAAAACCACCGCTACCAGCGG
TGGTTTGTTTGCCGGATCAAGAGCTACCAACTCTTTTTCCGAAGGTAAC
TGGCTTCAGCAGAGCGCAGATACCAA
ATACTGTTCTTCTAGTGTAGCCGTAGTTAGGCCACCACTTCAAGAACTC
TGTAGCACCGCCTACATACCTCGCTC
TGCTAATCCTGTTACCAGTGGCTGCTGCCAGTGGCGATAAGTCGTGTCT
TACCGGGTTGGACTCAAGACGATAGT
TACCGGATAAGGCGCAGCGGTCGGGCTGAACGGGGGGTTCGTGCACACA
GCCCAGCTTGGAGCGAACGACCTACA
CCGAACTGAGATACCTACAGCGTGAGCTATGAGAAAGCGCCACGCTTCC
CGAAGGGAGAAAGGCGGACAGGTATC
CGGTAAGCGGCAGGGTCGGAACAGGAGAGCGCACGAGGGAGCTTCCAGG
GGGAAACGCCTGGTATCTTTATAGTC
CTGTCGGGTTTCGCCACCTCTGACTTGAGCGTCGATTTTTGTGATGCTC
GTCAGGGGGGCGGAGCCTATGGAAAA
ACGCCAGCAACGCGGCCTTTTTACGGTTCCTGGCCTTTTGCTGGCCTTT
TGCTCACATGTTCTTTCCTGCGTTAT
CCCCTGATTCTGTGGATAACCGTATTACCGCCTTTGAGTGAGCTGATAC
CGCTCGCCGCAGCCGAACGACCGAGC
GCAGCGAGTCAGTGAGCGAGGAAGCGGAAG
SEQ ID NO: 49 is the sequence for OV 1165
CAGGGTAATCATCATCAATAATATACCTTATTTTGGATTGAAGCCAATA
TGATAATGAGG
GGGTGGAGTTTGTGACGTGGCGCGGGGCGTGGGAACGGGGCGGGTGACG
TAGTAGTGTGG
CGGAAGTGTGATGTTGCAAGTGTGGCGGAACACATGTAAGCGACGGATG
TGGCAAAAGTG
ACGTTTTTGGTGTGCGCCGGTGTACACAGGAAGTGACAATTTTCGCGCG
GTTTTAGGCGG
ATGTTGTAGTAAATTTGGGCGTAACCGAGTAAGATTTGGCCATTTTCGC
GGGAAAACTGA
ATAAGAGGAAGTGAAATCTGAATAATTTTGTGTTACTCATAGCGCGTAA
TATTTGTCTAG
GGCCGGGATCTCTGCAGGAATTTGATATCAAGCTTATCGATACCGTCGA
AACTTGTTTAT
TGCAGCTTATAATGGTTACAAATAAAGCAATAGCATCACAAATTTCACA
AATAAAGCATT
TTTTTCACTGCATTCTAGTTGTGGTTTGTCCAAACTCATCAATGTATCT
TATCATGTCTG
GATCGATCCGCTAGCGGCGCGCCGTTTCATCCGGACAAAGCCTGCGCGC
GCCCCGCCCCG
CCATTGGCCGTACCGCCCCGCGCCGCCGCCCCATCTCGCCCCTCGCCGC
CGGGTCCGGCG
CGTTAAAGCCAATAGGAACCGCCGCCGTTGTTCCCGTCACGGCCGGGGC
AGCCAATTGTG
GCGGCGCTCGGCGGCTCGTGGCTCTTTCGCGGCAAAAAGGATTTGGCGC
GTAAAAGTGGC
CGGGACTTTGCAGGCAGCGGCGGCCGGGGGCGGAGCGGGATCGAGCCCT
CGATGATATCA
GATCAAACGATATCACCGGTCGACTGAAAATGAGACATATTATCTGCCA
CGGAGGTGTTA
TTACCGAAGAAATGGCCGCCAGTCTTTTGGACCAGCTGATCGAAGAGGT
ACTGGCTGATA
ATCTTCCACCTCCTAGCCATTTTGAACCACCTACCCTTCACGAACTGTA
TGATTTAGACG
TGACGGCCCCCGAAGATCCCAACGAGGAGGCGGTTTCGCAGATTTTTCC
CGACTCTGTAA
TGTTGGCGGTGCAGGAAGGGATTGACTTACTCACTTTTCCGCCGGCGCC
CGGTTCTCCGG
AGCCGCCTCACCTTTCCCGGCAGCCCGAGCAGCCGGAGCAGAGAGCCTT
GGGTCCGGTTT
CTATGCCAAACCTTGTACCGGAGGTGATCGATCTTACCTGCCACGAGGC
TGGCTTTCCAC
CCAGTGACGACGAGGATGAAGAGGGTGAGGAGTTTGTGTTAGATTATGT
GGAGCACCCCG
GGCACGGTTGCAGGTCTTGTCATTATCACCGGAGGAATACGGGGGACCC
AGATATTATGT
GTTCGCTTTGCTATATGAGGACCTGTGGCATGTTTGTCTACAGTAAGTG
AAAATTATGGG
CAGTGGGTGATAGAGTGGTGGGTTTGGTGTGGTAATTTTTTTTTTAATT
TTTACAGTTTT
GTGGTTTAAAGAATTTTGTATTGTGATTTTTTTAAAAGGTCCTGTGTCT
GAACCTGAGCC
TGAGCCCGAGCCAGAACCGGAGCCTGCAAGACCTACCCGCCGTCCTAAA
ATGGCGCCTGC
TATCCTGAGACGCCCGACATCACCTGTGTCTAGAGAATGCAATAGTAGT
ACGGATAGCTG
TGACTCCGGTCCTTCTAACACACCTCCTGAGATACACCCGGTGGTCCCG
CTGTGCCCCAT
TAAACCAGTTGCCGTGAGAGTTGGTGGGCGTCGCCAGGCTGTGGAATGT
ATCGAGGACTT
GCTTAACGAGCCTGGGCAACCTTTGGACTTGAGCTGTAAACGCCCCAGG
CCATAAGGTGT
AAACCTGTGATTGCGTGTGTGGTTAACGCCTTTGTTTGCTGAATGAGTT
GATGTAAGTTT
AATAAAGGGTGAGATAATGTTTAACTTGCATGGCGTGTTAAATGGGGCG
GGGCTTAAAGG
GTATATAATGCGCCGTGGGCTAATCTTGGTTACATCTGACCTCATGGAG
GCTTGGGAGTG
TTTGGAAGATTTTTCTGCTGTGCGTAACTTGCTGGAACAGAGCTCTAAC
AGTACCTCTTG
GTTTTGGAGGTTTCTGTGGGGCTCATCCCAGGCAAAGTTAGTCTGCAGA
ATTAAGGAGGA
TTACAAGTGGGAATTTGAAGAGCTTTTGAAATCCTGTGGTGAGCTGTTT
GATTCTTGAA
TCTGGGTCACCAGGCGCTTTTCCAAGAGAAGGTCATCAAGACTTTGGAT
TTTTCCACACC
GGGGCGCGCTGCGGCTGCTGTTGCTTTTTTGAGTTTTATAAAGGATAAA
TGGAGCGAAGA
AACCCATCTGAGCGGGGGGTACCTGCTGGATTTTCTGGCCATGCATCTG
TGGAGAGCGGT
TGTGAGACACAAGAATCGCCTGCTACTGTTGTCTTCCGTCCGCCCGGCG
ATAATACCGAC
GGAGGAGCAGCAGCAGCAGCAGGAGGAAGCCAGGCGGCGGCGGCAGGAG
CAGAGCCCATG
GAACCCGAGAGCCGGCCTGGACCCTCGGGAATGAATGTTGTACAGGTGG
CTGAACTGTAT
CCAGAACTGAGACGCATTTTGACAATTACAGAGGATGGGCAGGGGCTAA
AGGGGGTAAAG
AGGGAGCGGGGGGCTTGTGAGGCTACAGAGGAGGCTAGGAATCTAGCTT
TTAGCTTAATG
ACCAGACACCGTCCTGAGTGTATTACTTTTCAACAGATCAAGGATAATT
GCGCTAATGAG
CTTGATCTGCTGGCGCAGAAGTATTCCATAGAGCAGCTGACCACTTACT
GGCTGCAGCCA
GGGGATGATTTTGAGGAGGCTATTAGGGTATATGCAAAGGTGGCACTTA
GGCCAGATTGC
AAGTACAAGATCAGCAAACTTGTAAATATCAGGAATTGTTGCTACATTT
CTGGGAACGGG
GCCGAGGTGGAGATAGATACGGAGGATAGGGTGGCCTTTAGATGTAGCA
TGATAAATATG
TGGCCGGGGGTGCTTGGCATGGACGGGGTGGTTATTATGAATGTAAGGT
TTACTGGCCCC
AATTTTAGCGGTACGGTTTTCCTGGCCAATACCAACCTTATCCTACACG
GTGTAAGCTTC
TATGGGTTTAACAATACCTGTGTGGAAGCCTGGACCGATGTAAGGGTTC
GGGGCTGTGCC
TTTTACTGCTGCTGGAAGGGGGTGGTGTGTCGCCCCAAAAGCAGGGCTT
CAATTAAGAAA
TGCCTCTTTGAAAGGTGTACCTTGGGTATCCTGTCTGAGGGTAACTCCA
GGGTGCGCCAC
AATGTGGCCTCCGACTGTGGTTGCTTCATGCTAGTGAAAAGCGTGGCTG
TGATTAAGCAT
AACATGGTATGTGGCAACTGCGAGGACAGGGCCTCTCAGATGCTGACCT
GCTCGGACGGC
AACTGTCACCTGCTGAAGACCATTCACGTAGCCAGCCACTCTCGCAAGG
CCTGGCCAGTG
TTTGAGCATAACATACTGACCCGCTGTTCCTTGCATTTGGGTAACAGGA
GGGGGGTGTTC
CTACCTTACCAATGCAATTTGAGTCACACTAAGATATTGCTTGAGCCCG
AGAGCATGTCC
AAGGTGAACCTGAACGGGGTGTTTGACATGACCATGAAGATCTGGAAGG
TGCTGAGGTAC
GATGAGACCCGCACCAGGTGCAGACCCTGCGAGTGTGGCGGTAAACATA
TTAGGAACCAG
CCTGTGATGCTGGATGTGACCGAGGAGCTGAGGCCCGATCACTTGGTGC
TGGCCTGCACC
CGCGCTGAGTTTGGCTCTAGCGATGAAGATACAGATTGAGGTACTGAAA
TGTGTGGGCGT
GGCTTAAGGGTGGGAAAGAATATATAAGGTGGGGGTCTTATGTAGTTTT
GTATCTGTTTT
GCAGCAGCCGCCGCCGCCATGAGCACCAACTCGTTTGATGGAAGCATTG
TGAGCTCATAT
TTGACAACGCGCATGCCCCCATGGGCCGGGGTGCGTCAGAATGTGATGG
GCTCCAGCATT
GATGGTCGCCCCGTCCTGCCCGCAAACTCTACTACCTTGACCTACGAGA
CCGTGTCTGGA
ACGCCGTTGGAGACTGCAGCCTCCGCCGCCGCTTCAGCCGCTGCAGCCA
CCGCCCGCGGG
ATTGTGACTGACTTTGCTTTCCTGAGCCCGCTTGCAAGCAGTGCAGCTT
CCCGTTCATCC
GCCCGCGATGACAAGTTGACGGCTCTTTTGGCACAATTGGATTCTTTGA
CCCGGGAACTT
AATGTCGTTTCTCAGCAGCTGTTGGATCTGCGCCAGCAGGTTTCTGCCC
TGAAGGCTTCC
TCCCCTCCCAATGCGGTTTAAAACATAAATAAAAAACCAGACTCTGTTT
GGATTTGGATC
AAGCAAGTGTCTTGCTGTCTTTATTTAGGGGTTTTGCGCGCGCGGTAGG
CCCGGGACCAG
CGGTCTCGGTCGTTGAGGGTCCTGTGTATTTTTTCCAGGACGTGGTAAA
GGTGACTCTGG
ATGTTCAGATACATGGGCATAAGCCCGTCTCTGGGGTGGAGGTAGCACC
ACTGCAGAGCT
TCATGCTGCGGGGTGGTGTTGTAGATGATCCAGTCGTAGCAGGAGCGCT
GGGCGTGGTGC
CTAAAAATGTCTTTCAGTAGCAAGCTGATTGCCAGGGGCAGGCCCTTGG
TGTAAGTGTTT
ACAAAGCGGTTAAGCTGGGATGGGTGCATACGTGGGGATATGAGATGCA
TCTTGGACTGT
ATTTTTAGGTTGGCTATGTTCCCAGCCATATCCCTCCGGGGATTCATGT
TGTGCAGAACC
ACCAGCACAGTGTATCCGGTGCACTTGGGAAATTTGTCATGTAGCTTAG
AAGGAAATGCG
TGGAAGAACTTGGAGACGCCCTTGTGACCTCCAAGATTTTCCATGCATT
CGTCCATAATG
ATGGCAATGGGCCCACGGGCGGCGGCCTGGGCGAAGATATTTCTGGGAT
CACTAACGTCA
TAGTTGTGTTCCAGGATGAGATCGTCATAGGCCATTTTTACAAAGCGCG
GGCGGAGGGTG
CCAGACTGCGGTATAATGGTTCCATCCGGCCCAGGGGCGTAGTTACCCT
CACAGATTTGC
ATTTCCCACGCTTTGAGTTCAGATGGGGGGATCATGTCTACCTGCGGGG
CGATGAAGAAA
ACGGTTTCCGGGGTAGGGGAGATCAGCTGGGAAGAAAGCAGGTTCCTGA
GCAGCTGCGAC
TTACCGCAGCCGGTGGGCCCGTAAATCACACCTATTACCGGGTGCAACT
GGTAGTTAAGA
GAGCTGCAGCTGCCGTCATCCCTGAGCAGGGGGGCCACTTCGTTAAGCA
TGTCCCTGACT
CGCATGTTTTCCCTGACCAAATCCGCCAGAAGGCGCTCGCCGCCCAGCG
ATAGCAGTTCT
TGCAAGGAAGCAAAGTTTTTCAACGGTTTGAGACCGTCCGCCGTAGGCA
TGCTTTTGAGC
GTTTGACCAAGCAGTTCCAGGCGGTCCCACAGCTCGGTCACCTGCTCTA
CGGCATCTCGA
TCCAGCATATCTCCTCGTTTCGCGGGTTGGGGCGGCTTTCGCTGTACGG
CAGTAGTCGGT
GCTCGTCCAGACGGGCCAGGGTCATGTCTTTCCACGGGCGCAGGGTCCT
CGTCAGCGTAG
TCTGGGTCACGGTGAAGGGGTGCGCTCCGGGCTGCGCGCTGGCCAGGGT
GCGCTTGAGGC
TGGTCCTGCTGGTGCTGAAGCGCTGCCGGTCTTCGCCCTGCGCGTCGGC
CAGGTAGCATT
TGACCATGGTGTCATAGTCCAGCCCCTCCGCGGCGTGGCCCTTGGCGCG
CAGCTTGCCCT
TGGAGGAGGCGCCGCACGAGGGGCAGTGCAGACTTTTGAGGGCGTAGAG
CTTGGGCGCGA
GAAATACCGATTCCGGGGAGTAGGCATCCGCGCCGCAGGCCCCGCAGAC
GGTCTCGCATT
CCACGAGCCAGGTGAGCTCTGGCCGTTCGGGGTCAAAAACCAGGTTTCC
CCCATGCTTTT
TGATGCGTTTCTTACCTCTGGTTTCCATGAGCCGGTGTCCACGCTCGGT
GACGAAAAGGC
TGTCCGTGTCCCCGTATACAGACTTGAGAGGCCTGTCCTCGAGCGGTGT
TCCGCGGTCCT
CCTCGTATAGAAACTCGGACCACTCTGAGACAAAGGCTCGCGTCCAGGC
CAGCACGAAGG
AGGCTAAGTGGGAGGGGTAGCGGTCGTTGTCCACTAGGGGGTCCACTCG
CTCCAGGGTGT
GAAGACACATGTCGCCCTCTTCGGCATCAAGGAAGGTGATTGGTTTGTA
GGTGTAGGCCA
CGTGACCGGGTGTTCCTGAAGGGGGGCTATAAAAGGGGGTGGGGGCGCG
TTCGTCCTCAC
TCTCTTCCGCATCGCTGTCTGCGAGGGCCAGCTGTTGGGGTGAGTACTC
CCTCTGAAAAG
CGGGCATGACTTCTGCGCTAAGATTGTCAGTTTCCAAAAACGAGGAGGA
TTTGATATTCA
CCTGGCCCGCGGTGATGCCTTTGAGGGTGGCCGCATCCATCTGGTCAGA
AAAGACAATCT
TTTTGTTGTCAAGCTTGGTGGCAAACGACCCGTAGAGGGCGTTGGACAG
CAACTTGGCGA
TGGAGCGCAGGGTTTGGTTTTTGTCGCGATCGGCGCGCTCCTTGGCCGC
GATGTTTAGCT
GCACGTATTCGCGCGCAACGCACCGCCATTCGGGAAAGACGGTGGTGCG
CTCGTCGGGCA
CCAGGTGCACGCGCCAACCGCGGTTGTGCAGGGTGACAAGGTCAACGCT
GGTGGCTACCT
CTCCGCGTAGGCGCTCGTTGGTCCAGCAGAGGCGGCCGCCCTTGCGCGA
GCAGAATGGCG
GTAGGGGGTCTAGCTGCGTCTCGTCCGGGGGGTCTGCGTCCACGGTAAA
GACCCCGGGCA
GCAGGCGCGCGTCGAAGTAGTCTATCTTGCATCCTTGCAAGTCTAGCGC
CTGCTGCCATG
CGCGGGCGGCAAGCGCGCGCTCGTATGGGTTGAGTGGGGGACCCCATGG
CATGGGGTGGG
TGAGCGCGGAGGCGTACATGCCGCAAATGTCGTAAACGTAGAGGGGCTC
TCTGAGTATTC
CAAGATATGTAGGGTAGCATCTTCCACCGCGGATGCTGGCGCGCACGTA
ATCGTATAGTT
CGTGCGAGGGAGCGAGGAGGTCGGGACCGAGGTTGCTACGGGCGGGCTG
CTCTGCTCGGA
AGACTATCTGCCTGAAGATGGCATGTGAGTTGGATGATATGGTTGGACG
CTGGAAGACGT
TGAAGCTGGCGTCTGTGAGACCTACCGCGTCACGCACGAAGGAGGCGTA
GGAGTCGCGCA
GCTTGTTGACCAGCTCGGCGGTGACCTGCACGTCTAGGGCGCAGTAGTC
CAGGGTTTCCT
TGATGATGTCATACTTATCCTGTCCCTTTTTTTTCCACAGCTCGCGGTT
GAGGACAAACT
CTTCGCGGTCTTTCCAGTACTCTTGGATCGGAAACCCGTCGGCCTCCGA
ACGGTAAGAGC
CTAGCATGTAGAACTGGTTGACGGCCTGGTAGGCGCAGCATCCCTTTTC
TACGGGTAGCG
CGTATGCCTGCGCGGCCTTCCGGAGCGAGGTGTGGGTGAGCGCAAAGGT
GTCCCTGACCA
TGACTTTGAGGTACTGGTATTTGAAGTCAGTGTCGTCGCATCCGCCCTG
CTCCCAGAGCA
AAAAGTCCGTGCGCTTTTTGGAACGCGGATTTGGCAGGGCGAAGGTGAC
ATCGTTGAAGA
GTATCTTTCCCGCGCGAGGCATAAAGTTGCGTGTGATGCGGAAGGGTCC
CGGCACCTCGG
AACGGTTGTTAATTACCTGGGCGGCGAGCACGATCTCGTCAAAGCCGTT
GATGTTGTGGC
CCACAATGTAAAGTTCCAAGAAGCGCGGGATGCCCTTGATGGAAGGCAA
TTTTTTAAGTT
CCTCGTAGGTGAGCTCTTCAGGGGAGCTGAGCCCGTGCTCTGAAAGGGC
CCAGTCTGCAA
GATGAGGGTTGGAAGCGACGAATGAGCTCCACAGGTCACGGGCCATTAG
CATTTGCAGGT
GGTCGCGAAAGGTCCTAAACTGGCGACCTATGGCCATTTTTTCTGGGGT
GATGCAGTAGA
AGGTAAGCGGGTCTTGTTCCCAGCGGTCCCATCCAAGGTTCGCGGCTAG
GTCTCGCGCGG
CAGTCACTAGAGGCTCATCTCCGCCGAACTTCATGACCAGCATGAAGGG
CACGAGCTGCT
TCCCAAAGGCCCCCATCCAAGTATAGGTCTCTACATCGTAGGTGACAAA
GAGACGCTCGG
TGCGAGGATGCGAGCCGATCGGGAAGAACTGGATCTCCCGCCACCAATT
GGAGGAGTGGC
TATTGATGTGGTGAAAGTAGAAGTCCCTGCGACGGGCCGAACACTCGTG
CTGGCTTTTGT
AAAAACGTGCGCAGTACTGGCAGCGGTGCACGGGCTGTACATCCTGCAC
GAGGTTGACCT
GACGACCGCGCACAAGGAAGCAGAGTGGGAATTTGAGCCCCTCGCCTGG
CGGGTTTGGCT
GGTGGTCTTCTACTTCGGCTGCTTGTCCTTGACCGTCTGGCTGCTCGAG
GGGAGTTACGG
TGGATCGGACCACCACGCCGCGCGAGCCCAAAGTCCAGATGTCCGCGCG
CGGCGGTCGGA
GCTTGATGACAACATCGCGCAGATGGGAGCTGTCCATGGTCTGGAGCTC
CCGCGGCGTCA
GGTCAGGCGGGAGCTCCTGCAGGTTTACCTCGCATAGACGGGTCAGGGC
GCGGGCTAGAT
CCAGGTGATACCTAATTTCCAGGGGCTGGTTGGTGGCGGCGTCGATGGC
TTGCAAGAGGC
CGCATCCCCGCGGCGCGACTACGGTACCGCGCGGCGGGCGGTGGGCCGC
GGGGGTGTCCT
TGGATGATGCATCTAAAAGCGGTGACGCGGGCGAGCCCCCGGAGGTAGG
GGGGGCTCCGG
ACCCGCCGGGAGAGGGGGCAGGGGCACGTCGGCGCCGCGCGCGGGCAGG
AGCTGGTGCTG
CGCGCGTAGGTTGCTGGCGAACGCGACGACGCGGCGGTTGATCTCCTGA
ATCTGGCGCCT
CTGCGTGAAGACGACGGGCCCGGTGAGCTTGAGCCTGAAAGAGAGTTCG
ACAGAATCAAT
TTCGGTGTCGTTGACGGCGGCCTGGCGCAAAATCTCCTGCACGTCTCCT
GAGTTGTCTTG
ATAGGCGATCTCGGCCATGAACTGCTCGATCTCTTCCTCCTGGAGATCT
CCGCGTCCGGC
TCGCTCCACGGTGGCGGCGAGGTCGTTGGAAATGCGGGCCATGAGCTGC
GAGAAGGCGTT
GAGGCCTCCCTCGTTCCAGACGCGGCTGTAGACCACGCCCCCTTCGGCA
TCGCGGGCGCG
CATGACCACCTGCGCGAGATTGAGCTCCACGTGCCGGGCGAAGACGGCG
TAGTTTCGCAG
GCGCTGAAAGAGGTAGTTGAGGGTGGTGGCGGTGTGTTCTGCCACGAAG
AAGTACATAAC
CCAGCGTCGCAACGTGGATTCGTTGATATCCCCCAAGGCCTCAAGGCGC
TCCATGGCCTC
GTAGAAGTCCACGGCGAAGTTGAAAAACTGGGAGTTGCGCGCCGACACG
GTTAACTCCTC
CTCCAGAAGACGGATGAGCTCGGCGACAGTGTCGCGCACCTCGCGCTCA
AAGGCTACAGG
GGCCTCTTCTTCTTCTTCAATCTCCTCTTCCATAAGGGCCTCCCCTTCT
TCTTCTTCTGG
CGGCGGTGGGGGAGGGGGGACACGGCGGCGACGACGGCGCACCGGGAGG
CGGTCGACAAA
GCGCTCGATCATCTCCCCGCGGCGACGGCGCATGGTCTCGGTGACGGCG
CGGCCGTTCTC
GCGGGGGCGCAGTTGGAAGACGCCGCCCGTCATGTCCCGGTTATGGGTT
GGCGGGGGGCT
GCCATGCGGCAGGGATACGGCGCTAACGATGCATCTCAACAATTGTTGT
GTAGGTACTCC
GCCGCCGAGGGACCTGAGCGAGTCCGCATCGACCGGATCGGAAAACCTC
TCGAGAAAGGC
GTCTAACCAGTCACAGTCGCAAGGTAGGCTGAGCACCGTGGCGGGCGGC
AGCGGGCGGCG
GTCGGGGTTGTTTCTGGCGGAGGTGCTGCTGATGATGTAATTAAAGTAG
GCGGTCTTGAG
ACGGCGGATGGTCGACAGAAGCACCATGTCCTTGGGTCCGGCCTGCTGA
ATGCGCAGGCG
GTCGGCCATGCCCCAGGCTTCGTTTTGACATCGGCGCAGGTCTTTGTAG
TAGTCTTGCAT
GAGCCTTTCTACCGGCACTTCTTCTTCTCCTTCCTCTTGTCCTGCATCT
CTTGCATCTAT
CGCTGCGGCGGCGGCGGAGTTTGGCCGTAGGTGGCGCCCTCTTCCTCCC
ATGCGTGTGAC
CCCGAAGCCCCTCATCGGCTGAAGCAGGGCTAGGTCGGCGACAACGCGC
TCGGCTAATAT
GGCCTGCTGCACCTGCGTGAGGGTAGACTGGAAGTCATCCATGTCCACA
AAGCGGTGGTA
TGCGCCCGTGTTGATGGTGTAAGTGCAGTTGGCCATAACGGACCAGTTA
ACGGTCTGGTG
ACCCGGCTGCGAGAGCTCGGTGTACCTGAGACGCGAGTAAGCCCTCGAG
TCAAATACGTA
GTCGTTGCAAGTCCGCACCAGGTACTGGTATCCCACCAAAAAGTGCGGC
GGCGGCTGGCG
GTAGAGGGGCCAGCGTAGGGTGGCCGGGGCTCCGGGGGCGAGATCTTCC
AACATAAGGCG
ATGATATCCGTAGATGTACCTGGACATCCAGGTGATGCCGGCGGCGGTG
GTGGAGGCGCG
CGGAAAGTCGCGGACGCGGTTCCAGATGTTGCGCAGCGGCAAAAAGTGC
TCCATGGTCGG
GACGCTCTGGCCGGTCAGGCGCGCGCAATCGTTGACGCTCTAGACCGTG
CAAAAGGAGAG
CCTGTAAGCGGGCACTCTTCCGTGGTCTGGTGGATAAATTCGCAAGGGT
ATCATGGCGGA
CGACCGGGGTTCGAGCCCCGTATCCGGCCGTCCGCCGTGATCCATGCGG
TTACCGCCCGC
GTGTCGAACCCAGGTGTGCGACGTCAGACAACGGGGGAGTGCTCCTTTT
GGCTTCCTTCC
AGGCGCGGCGGCTGCTGCGCTAGCTTTTTTGGCCACTGGCCGCGCGCAG
CGTAAGCGGTT
AGGCTGGAAAGCGAAAGCATTAAGTGGCTCGCTCCCTGTAGCCGGAGGG
TTATTTTCCAA
GGGTTGAGTCGCGGGACCCCCGGTTCGAGTCTCGGACCGGCCGGACTGC
GGCGAACGGGG
GTTTGCCTCCCCGTCATGCAAGACCCCGCTTGCAAATTCCTCCGGAAAC
AGGGACGAGCC
CCTTTTTTGCTTTTCCCAGATGCATCCGGTGCTGCGGCAGATGCGCCCC
CCTCCTCAGCA
GCGGCAAGAGCAAGAGCAGCGGCAGACATGCAGGGCACCCTCCCCTCCT
CCTACCGCGTC
AGGAGGGGCGACATCCGCGGTTGACGCGGCAGCAGATGGTGATTACGAA
CCCCCGCGGCG
CCGGGCCCGGCACTACCTGGACTTGGAGGAGGGCGAGGGCCTGGCGCGG
CTAGGAGCGCC
CTCTCCTGAGCGGTACCCAAGGGTGCAGCTGAAGCGTGATACGCGTGAG
GCGTACGTGCC
GCGGCAGAACCTGTTTCGCGACCGCGAGGGAGAGGAGCCCGAGGAGATG
CGGGATCGAAA
GTTCCACGCAGGGCGCGAGCTGCGGCATGGCCTGAATCGCGAGCGGTTG
CTGCGCGAGGA
GGACTTTGAGCCCGACGCGCGAACCGGGATTAGTCCCGCGCGCGCACAC
GTGGCGGCCGC
CGACCTGGTAACCGCATACGAGCAGACGGTGAACCAGGAGATTAACTTT
CAAAAAAGCTT
TAACAACCACGTGCGTACGCTTGTGGCGCGCGAGGAGGTGGCTATAGGA
CTGATGCATCT
GTGGGACTTTGTAAGCGCGCTGGAGCAAAACCCAAATAGCAAGCCGCTC
ATGGCGCAGCT
GTTCCTTATAGTGCAGCACAGCAGGGACAACGAGGCATTCAGGGATGCG
CTGCTAAACAT
AGTAGAGCCCGAGGGCCGCTGGCTGCTCGATTTGATAAACATCCTGCAG
AGCATAGTGGT
GCAGGAGCGCAGCTTGAGCCTGGCTGACAAGGTGGCCGCCATCAACTAT
TCCATGCTTAG
CCTGGGCAAGTTTTACGCCCGCAAGATATACCATACCCCTTACGTTCCC
ATAGACAAGGA
GGTAAAGATCGAGGGGTTCTACATGCGCATGGCGCTGAAGGTGCTTACC
TTGAGCGACGA
CCTGGGCGTTTATCGCAACGAGCGCATCCACAAGGCCGTGAGCGTGAGC
CGGCGGCGCGA
GCTCAGCGACCGCGAGCTGATGCACAGCCTGCAAAGGGCCCTGGCTGGC
ACGGGCAGCGG
CGATAGAGAGGCCGAGTCCTACTTTGACGCGGGCGCTGACCTGCGCTGG
GCCCCAAGCCG
ACGCGCCCTGGAGGCAGCTGGGGCCGGACCTGGGCTGGCGGTGGCACCC
GCGCGCGCTGG
CAACGTCGGCGGCGTGGAGGAATATGACGAGGACGATGAGTACGAGCCA
GAGGACGGCGA
GTACTAAGCGGTGATGTTTCTGATCAGATGATGCAAGACGCAACGGACC
CGGCGGTGCGG
GCGGCGCTGCAGAGCCAGCCGTCCGGCCTTAACTCCACGGACGACTGGC
GCCAGGTCATG
GACCGCATCATGTCGCTGACTGCGCGCAATCCTGACGCGTTCCGGCAGC
AGCCGCAGGCC
AACCGGCTCTCCGCAATTCTGGAAGCGGTGGTCCCGGCGCGCGCAAACC
CCACGCACGAG
AAGGTGCTGGCGATCGTAAACGCGCTGGCCGAAAACAGGGCCATCCGGC
CCGACGAGGCC
GGCCTGGTCTACGACGCGCTGCTTCAGCGCGTGGCTCGTTACAACAGCG
GCAACGTGCAG
ACCAACCTGGACCGGCTGGTGGGGGATGTGCGCGAGGCCGTGGCGCAGC
GTGAGCGCGCG
CAGCAGCAGGGCAACCTGGGCTCCATGGTTGCACTAAACGCCTTCCTGA
GTACACAGCCC
GCCAACGTGCCGCGGGGACAGGAGGACTACACCAACTTTGTGAGCGCAC
TGCGGCTAATG
GTGACTGAGACACCGCAAAGTGAGGTGTACCAGTCTGGGCCAGACTATT
TTTTCCAGACC
AGTAGACAAGGCCTGCAGACCGTAAACCTGAGCCAGGCTTTCAAAAACT
TGCAGGGGCTG
TGGGGGGTGCGGGCTCCCACAGGCGACCGCGCGACCGTGTCTAGCTTGC
TGACGCCCAAC
TCGCGCCTGTTGCTGCTGCTAATAGCGCCCTTCACGGACAGTGGCAGCG
TGTCCCGGGAC
ACATACCTAGGTCACTTGCTGACACTGTACCGCGAGGCCATAGGTCAGG
CGCATGTGGAC
GAGCATACTTTCCAGGAGATTACAAGTGTCAGCCGCGCGCTGGGGCAGG
AGGACACGGGC
AGCCTGGAGGCAACCCTAAACTACCTGCTGACCAACCGGCGGCAGAAGA
TCCCCTCGTTG
CACAGTTTAAACAGCGAGGAGGAGCGCATTTTGCGCTACGTGCAGCAGA
GCGTGAGCCTT
AACCTGATGCGCGACGGGGTAACGCCCAGCGTGGCGCTGGACATGACCG
CGCGCAACATG
GAACCGGGCATGTATGCCTCAAACCGGCCGTTTATCAACCGCCTAATGG
ACTACTTGCAT
CGCGCGGCCGCCGTGAACCCCGAGTATTTCACCAATGCCATCTTGAACC
CGCACTGGCTA
CCGCCCCCTGGTTTCTACACCGGGGGATTCGAGGTGCCCGAGGGTAACG
ATGGATTCCTC
TGGGACGACATAGACGACAGCGTGTTTTCCCCGCAACCGCAGACCCTGC
TAGAGTTGCAA
CAGCGCGAGCAGGCAGAGGCGGCGCTGCGAAAGGAAAGCTTCCGCAGGC
CAAGCAGCTTG
TCCGATCTAGGCGCTGCGGCCCCGCGGTCAGATGCTAGTAGCCCATTTC
CAAGCTTGATA
GGGTCTCTTACCAGCACTCGCACCACCCGCCCGCGCCTGCTGGGCGAGG
AGGAGTACCTA
AACAACTCGCTGCTGCAGCCGCAGCGCGAAAAAAACCTGCCTCCGGCAT
TTCCCAACAAC
GGGATAGAGAGCCTAGTGGACAAGATGAGTAGATGGAAGACGTACGCGC
AGGAGCACAGG
GACGTGCCAGGCCCGCGCCCGCCCACCCGTCGTCAAAGGCACGACCGTC
AGCGGGGTCTG
GTGTGGGAGGACGATGACTCGGCAGACGACAGCAGCGTCCTGGATTTGG
GAGGGAGTGGC
AACCCGTTTGCGCACCTTCGCCCCAGGCTGGGGAGAATGTTTTAAAAAA
AAAAAAGCATG
ATGCAAAATAAAAAACTCACCAAGGCCATGGCACCGAGCGTTGGTTTTC
TTGTATTCCCC
TTAGTATGCGGCGCGCGGCGATGTATGAGGAAGGTCCTCCTCCCTCCTA
CGAGAGTGTGG
TGAGCGCGGCGCCAGTGGCGGCGGCGCTGGGTTCTCCCTTCGATGCTCC
CCTGGACCCGC
CGTTTGTGCCTCCGCGGTACCTGCGGCCTACCGGGGGGAGAAACAGCAT
CCGTTACTCTG
AGTTGGCACCCCTATTCGACACCACCCGTGTGTACCTGGTGGACAACAA
GTCAACGGATG
TGGCATCCCTGAACTACCAGAACGACCACAGCAACTTTCTGACCACGGT
CATTCAAAACA
ATGACTACAGCCCGGGGGAGGCAAGCACACAGACCATCAATCTTGACGA
CCGGTCGCACT
GGGGCGGCGACCTGAAAACCATCCTGCATACCAACATGCCAAATGTGAA
CGAGTTCATGT
TTACCAATAAGTTTAAGGCGCGGGTGATGGTGTCGCGCTTGCCTACTAA
GGACAATCAGG
TGGAGCTGAAATACGAGTGGGTGGAGTTCACGCTGCCCGAGGGCAACTA
CTCCGAGACCA
TGACCATAGACCTTATGAACAACGCGATCGTGGAGCACTACTTGAAAGT
GGGCAGACAGA
ACGGGGTTCTGGAAAGCGACATCGGGGTAAAGTTTGACACCCGCAACTT
CAGACTGGGGT
TTGACCCCGTCACTGGTCTTGTCATGCCTGGGGTATATACAAACGAAGC
CTTCCATCCAG
ACATCATTTTGCTGCCAGGATGCGGGGTGGACTTCACCCACAGCCGCCT
GAGCAACTTGT
TGGGCATCCGCAAGCGGCAACCCTTCCAGGAGGGCTTTAGGATCACCTA
CGATGATCTGG
AGGGTGGTAACATTCCCGCACTGTTGGATGTGGACGCCTACCAGGCGAG
CTTGAAAGATG
ACACCGAACAGGGCGGGGGTGGCGCAGGCGGCAGCAACAGCAGTGGCAG
CGGCGCGGAAG
AGAACTCCAACGCGGCAGCCGCGGCAATGCAGCCGGTGGAGGACATGAA
CGATCATGCCA
TTCGCGGCGACACCTTTGCCACACGGGCTGAGGAGAAGCGCGCTGAGGC
CGAAGCAGCGG
CCGAAGCTGCCGCCCCCGCTGCGCAACCCGAGGTCGAGAAGCCTCAGAA
GAAACCGGTGA
TCAAACCCCTGACAGAGGACAGCAAGAAACGCAGTTACAACCTAATAAG
CAATGACAGCA
CCTTCACCCAGTACCGCAGCTGGTACCTTGCATACAACTACGGCGACCC
TCAGACCGGAA
TCCGCTCATGGACCCTGCTTTGCACTCCTGACGTAACCTGCGGCTCGGA
GCAGGTCTACT
GGTCGTTGCCAGACATGATGCAAGACCCCGTGACCTTCCGCTCCACGCG
CCAGATCAGCA
ACTTTCCGGTGGTGGGCGCCGAGCTGTTGCCCGTGCACTCCAAGAGCTT
CTACAACGACC
AGGCCGTCTACTCCCAACTCATCCGCCAGTTTACCTCTCTGACCCACGT
GTTCAATCGCT
TTCCCGAGAACCAGATTTTGGCGCGCCCGCCAGCCCCCACCATCACCAC
CGTCAGTGAAA
ACGTTCCTGCTCTCACAGATCACGGGACGCTACCGCTGCGCAACAGCAT
CGGAGGAGTCC
AGCGAGTGACCATTACTGACGCCAGACGCCGCACCTGCCCCTACGTTTA
CAAGGCCCTGG
GCATAGTCTCGCCGCGCGTCCTATCGAGCCGCACTTTTTGAGCAAGCAT
GTCCATCCTTA
TATCGCCCAGCAATAACACAGGCTGGGGCCTGCGCTTCCCAAGCAAGAT
GTTTGGCGGGG
CCAAGAAGCGCTCCGACCAACACCCAGTGCGCGTGCGCGGGCACTACCG
CGCGCCCTGGG
GCGCGCACAAACGCGGCCGCACTGGGCGCACCACCGTCGATGACGCCAT
CGACGCGGTGG
TGGAGGAGGCGCGCAACTACACGCCCACGCCGCCACCAGTGTCCACAGT
GGACGCGGCCA
TTCAGACCGTGGTGCGCGGAGCCCGGCGCTATGCTAAAATGAAGAGACG
GCGGAGGCGCG
TAGCACGTCGCCACCGCCGCCGACCCGGCACTGCCGCCCAACGCGCGGC
GGCGGCCCTGC
TTAACCGCGCACGTCGCACCGGCCGACGGGCGGCCATGCGGGCCGCTCG
AAGGCTGGCCG
CGGGTATTGTCACTGTGCCCCCCAGGTCCAGGCGACGAGCGGCCGCCGC
AGCAGCCGCGG
CCATTAGTGCTATGACTCAGGGTCGCAGGGGCAACGTGTATTGGGTGCG
CGACTCGGTTA
GCGGCCTGCGCGTGCCCGTGCGCACCCGCCCCCCGCGCAACTAGATTGC
AAGAAAAAACT
ACTTAGACTCGTACTGTTGTATGTATCCAGCGGCGGCGGCGCGCAACGA
AGCTATGTCCA
AGCGCAAAATCAAAGAAGAGATGCTCCAGGTCATCGCGCCGGAGATCTA
TGGCCCCCCGA
AGAAGGAAGAGCAGGATTACAAGCCCCGAAAGCTAAAGCGGGTCAAAAA
GAAAAAGAAAG
ATGATGATGATGAACTTGACGACGAGGTGGAACTGCTGCACGCTACCGC
GCCCAGGCGAC
GGGTACAGTGGAAAGGTCGACGCGTAAAACGTGTTTTGCGACCCGGCAC
CACCGTAGTCT
TTACGCCCGGTGAGCGCTCCACCCGCACCTACAAGCGCGTGTATGATGA
GGTGTACGGCG
ACGAGGACCTGCTTGAGCAGGCCAACGAGCGCCTCGGGGAGTTTGCCTA
CGGAAAGCGGC
ATAAGGACATGCTGGCGTTGCCGCTGGACGAGGGCAACCCAACACCTAG
CCTAAAGCCCG
TAACACTGCAGCAGGTGCTGCCCGCGCTTGCACCGTCCGAAGAAAAGCG
CGGCCTAAAGC
GCGAGTCTGGTGACTTGGCACCCACCGTGCAGCTGATGGTACCCAAGCG
CCAGCGACTGG
AAGATGTCTTGGAAAAAATGACCGTGGAACCTGGGCTGGAGCCCGAGGT
CCGCGTGCGGC
CAATCAAGCAGGTGGCGCCGGGACTGGGCGTGCAGACCGTGGACGTTCA
GATACCCACTA
CCAGTAGCACCAGTATTGCCACCGCCACAGAGGGCATGGAGACACAAAC
GTCCCCGGTTG
CCTCAGCGGTGGCGGATGCCGCGGTGCAGGCGGTCGCTGCGGCCGCGTC
CAAGACCTCTA
CGGAGGTGCAAACGGACCCGTGGATGTTTCGCGTTTCAGCCCCCCGGCG
CCCGCGCGGTT
CGAGGAAGTACGGCGCCGCCAGCGCGCTACTGCCCGAATATGCCCTACA
TCCTTCCATTG
CGCCTACCCCCGGCTATCGTGGCTACACCTACCGCCCCAGAAGACGAGC
AACTACCCGAC
GCCGAACCACCACTGGAACCCGCCGCCGCCGTCGCCGTCGCCAGCCCGT
GCTGGCCCCGA
TTTCCGTGCGCAGGGTGGCTCGCGAAGGAGGCAGGACCCTGGTGCTGCC
AACAGCGCGCT
ACCACCCCAGCATCGTTTAAAAGCCGGTCTTTGTGGTTCTTGCAGATAT
GGCCCTCACCT
GCCGCCTCCGTTTCCCGGTGCCGGGATTCCGAGGAAGAATGCACCGTAG
GAGGGGCATGG
CCGGCCACGGCCTGACGGGCGGCATGCGTCGTGCGCACCACCGGCGGCG
GCGCGCGTCGC
ACCGTCGCATGCGCGGCGGTATCCTGCCCCTCCTTATTCCACTGATCGC
CGCGGCGATTG
GCGCCGTGCCCGGAATTGCATCCGTGGCCTTGCAGGCGCAGAGACACTG
ATTAAAAACAA
GTTGCATGTGGAAAAATCAAAATAAAAAGTCTGGACTCTCACGCTCGCT
TGGTCCTGTAA
CTATTTTGTAGAATGGAAGACATCAACTTTGCGTCTCTGGCCCCGCGAC
ACGGCTCGCGC
CCGTTCATGGGAAACTGGCAAGATATCGGCACCAGCAATATGAGCGGTG
GCGCCTTCAGC
TGGGGCTCGCTGTGGAGCGGCATTAAAAATTTCGGTTCCACCGTTAAGA
ACTATGGCAGC
AAGGCCTGGAACAGCAGCACAGGCCAGATGCTGAGGGATAAGTTGAAAG
AGCAAAATTTC
CAACAAAAGGTGGTAGATGGCCTGGCCTCTGGCATTAGCGGGGTGGTGG
ACCTGGCCAAC
CAGGCAGTGCAAAATAAGATTAACAGTAAGCTTGATCCCCGCCCTCCCG
TAGAGGAGCCT
CCACCGGCCGTGGAGACAGTGTCTCCAGAGGGGCGTGGCGAAAAGCGTC
CGCGCCCCGAC
AGGGAAGAAACTCTGGTGACGCAAATAGACGAGCCTCCCTCGTACGAGG
AGGCACTAAAG
CAAGGCCTGCCCACCACCCGTCCCATCGCGCCCATGGCTACCGGAGTGC
TGGGCCAGCAC
ACACCCGTAACGCTGGACCTGCCTCCCCCCGCCGACACCCAGCAGAAAC
CTGTGCTGCCA
GGCCCGACCGCCGTTGTTGTAACCCGTCCTAGCCGCGCGTCCCTGCGCC
GCGCCGCCAGC
GGTCCGCGATCGTTGCGGCCCGTAGCCAGTGGCAACTGGCAAAGCACAC
TGAACAGCATC
GTGGGTCTGGGGGTGCAATCCCTGAAGCGCCGACGATGCTTCTGAATAG
CTAACGTGTCG
TATGTGTGTCATGTATGCGTCCATGTCGCCGCCAGAGGAGCTGCTGAGC
CGCCGCGCGCC
CGCTTTCCAAGATGGCTACCCCTTCGATGATGCCGCAGTGGTCTTACAT
GCACATCTCGG
GCCAGGACGCCTCGGAGTACCTGAGCCCCGGGCTGGTGCAGTTTGCCCG
CGCCACCGAGA
CGTACTTCAGCCTGAATAACAAGTTTAGAAACCCCACGGTGGCGCCTAC
GCACGACGTGA
CCACAGACCGGTCCCAGCGTTTGACGCTGCGGTTCATCCCTGTGGACCG
TGAGGATACTG
CGTACTCGTACAAGGCGCGGTTCACCCTAGCTGTGGGTGATAACCGTGT
GCTGGACATGG
CTTCCACGTACTTTGACATCCGCGGCGTGCTGGACAGGGGCCCTACTTT
TAAGCCCTACT
CTGGCACTGCCTACAACGCCCTGGCTCCCAAGGGTGCCCCAAATCCTTG
CGAATGGGATG
AAGCTGCTACTGCTCTTGAAATAAACCTAGAAGAAGAGGACGATGACAA
CGAAGACGAAG
TAGACGAGCAAGCTGAGCAGCAAAAAACTCACGTATTTGGGCAGGCGCC
TTATTCTGGTA
TAAATATTACAAAGGAGGGTATTCAAATAGGTGTCGAAGGTCAAACACC
TAAATATGCCG
ATAAAACATTTCAACCTGAACCTCAAATAGGAGAATCTCAGTGGTACGA
AACTGAAATTA
ATCATGCAGCTGGGAGAGTCCTTAAAAAGACTACCCCAATGAAACCATG
TTACGGTTCAT
ATGCAAAACCCACAAATGAAAATGGAGGGCAAGGCATTCTTGTAAAGCA
ACAAAATGGAA
AGCTAGAAAGTCAAGTGGAAATGCAATTTTTCTCAACTACTGAGGCGAC
CGCAGGCAATG
GTGATAACTTGACTCCTAAAGTGGTATTGTACAGTGAAGATGTAGATAT
AGAAACCCCAG
ACACTCATATTTCTTACATGCCCACTATTAAGGAAGGTAACTCACGAGA
ACTAATGGGCC
AACAATCTATGCCCAACAGGCCTAATTACATTGCTTTTAGGGACAATTT
TATTGGTCTAA
TGTATTACAACAGCACGGGTAATATGGGTGTTCTGGCGGGCCAAGCATC
GCAGTTGAATG
CTGTTGTAGATTTGCAAGACAGAAACACAGAGCTTTCATACCAGCTTTT
GCTTGATTCCA
TTGGTGATAGAACCAGGTACTTTTCTATGTGGAATCAGGCTGTTGACAG
CTATGATCCAG
ATGTTAGAATTATTGAAAATCATGGAACTGAAGATGAACTTCCAAATTA
CTGCTTTCCAC
TGGGAGGTGTGATTAATACAGAGACTCTTACCAAGGTAAAACCTAAAAC
AGGTCAGGAAA
ATGGATGGGAAAAAGATGCTACAGAATTTTCAGATAAAAATGAAATAAG
AGTTGGAAATA
ATTTTGCCATGGAAATCAATCTAAATGCCAACCTGTGGAGAAATTTCCT
GTACTCCAACA
TAGCGCTGTATTTGCCCGACAAGCTAAAGTACAGTCCTTCCAACGTAAA
AATTTCTGATA
ACCCAAACACCTACGACTACATGAACAAGCGAGTGGTGGCTCCCGGGTT
AGTGGACTGCT
ACATTAACCTTGGAGCACGCTGGTCCCTTGACTATATGGACAACGTCAA
CCCATTTAACC
ACCACCGCAATGCTGGCCTGCGCTACCGCTCAATGTTGCTGGGCAATGG
TCGCTATGTGC
CCTTCCACATCCAGGTGCCTCAGAAGTTCTTTGCCATTAAAAACCTCCT
TCTCCTGCCGG
GCTCATACACCTACGAGTGGAACTTCAGGAAGGATGTTAACATGGTTCT
GCAGAGCTCCC
TAGGAAATGACCTAAGGGTTGACGGAGCCAGCATTAAGTTTGATAGCAT
TTGCCTTTACG
CCACCTTCTTCCCCATGGCCCACAACACCGCCTCCACGCTTGAGGCCAT
GCTTAGAAACG
ACACCAACGACCAGTCCTTTAACGACTATCTCTCCGCCGCCAACATGCT
CTACCCTATAC
CCGCCAACGCTACCAACGTGCCCATATCCATCCCCTCCCGCAACTGGGC
GGCTTTCCGCG
GCTGGGCCTTCACGCGCCTTAAGACTAAGGAAACCCCATCACTGGGCTC
GGGCTACGACC
CTTATTACACCTACTCTGGCTCTATACCCTACCTAGATGGAACCTTTTA
CCTCAACCACA
CCTTTAAGAAGGTGGCCATTACCTTTGACTCTTCTGTCAGCTGGCCTGG
CAATGACCGCC
TGCTTACCCCCAACGAGTTTGAAATTAAGCGCTCAGTTGACGGGGAGGG
TTACAACGTTG
CCCAGTGTAACATGACCAAAGACTGGTTCCTGGTACAAATGCTAGCTAA
CTACAACATTG
GCTACCAGGGCTTCTATATCCCAGAGAGCTACAAGGACCGCATGTACTC
CTTCTTTAGAA
ACTTCCAGCCCATGAGCCGTCAGGTGGTGGATGATACTAAATACAAGGA
CTACCAACAGG
TGGGCATCCTACACCAACACAACAACTCTGGATTTGTTGGCTACCTTGC
CCCCACCATGC
GCGAAGGACAGGCCTACCCTGCTAACTTCCCCTATCCGCTTATAGGCAA
GACCGCAGTTG
ACAGCATTACCCAGAAAAAGTTTCTTTGCGATCGCACCCTTTGGCGCAT
CCCATTCTCCA
GTAACTTTATGTCCATGGGCGCACTCACAGACCTGGGCCAAAACCTTCT
CTACGCCAACT
CCGCCCACGCGCTAGACATGACTTTTGAGGTGGATCCCATGGACGAGCC
CACCCTTCTTT
ATGTTTTGTTTGAAGTCTTTGACGTGGTCCGTGTGCACCGGCCGCACCG
CGGCGTCATCG
AAACCGTGTACCTGCGCACGCCCTTCTCGGCCGGCAACGCCACAACATA
AAGAAGCAAGC
AACATCAACAACAGCTGCCGCCATGGGCTCCAGTGAGCAGGAACTGAAA
GCCATTGTCAA
AGATCTTGGTTGTGGGCCATATTTTTTGGGCACCTATGACAAGCGCTTT
CCAGGCTTTGT
TTCTCCACACAAGCTCGCCTGCGCCATAGTCAATACGGCCGGTCGCGAG
ACTGGGGGCGT
ACACTGGATGGCCTTTGCCTGGAACCCGCACTCAAAAACATGCTACCTC
TTTGAGCCCTT
TGGCTTTTCTGACCAGCGACTCAAGCAGGTTTACCAGTTTGAGTACGAG
TCACTCCTGCG
CCGTAGCGCCATTGCTTCTTCCCCCGACCGCTGTATAACGCTGGAAAAG
TCCACCCAAAG
CGTACAGGGGCCCAACTCGGCCGCCTGTGGACTATTCTGCTGCATGTTT
CTCCACGCCTT
TGCCAACTGGCCCCAAACTCCCATGGATCACAACCCCACCATGAACCTT
ATTACCGGGGT
ACCCAACTCCATGCTCAACAGTCCCCAGGTACAGCCCACCCTGCGTCGC
AACCAGGAACA
GCTCTACAGCTTCCTGGAGCGCCACTCGCCCTACTTCCGCAGCCACAGT
GCGCAGATTAG
GAGCGCCACTTCTTTTTGTCACTTGAAAAACATGTAAAAATAATGTACT
AGAGACACTTT
CAATAAAGGCAAATGCTTTTATTTGTACACTCTCGGGTGATTATTTACC
CCCACCCTTGC
CGTCTGCGCCGTTTAAAAATCAAAGGGGTTCTGCCGCGCATCGCTATGC
GCCACTGGCAG
GGACACGTTGCGATACTGGTGTTTAGTGCTCCACTTAAACTCAGGCACA
ACCATCCGCGG
CAGCTCGGTGAAGTTTTCACTCCACAGGCTGCGCACCATCACCAACGCG
TTTAGCAGGTC
GGGCGCCGATATCTTGAAGTCGCAGTTGGGGCCTCCGCCCTGCGCGCGC
GAGTTGCGATA
CACAGGGTTGCAGCACTGGAACACTATCAGCGCCGGGTGGTGCACGCTG
GCCAGCACGCT
CTTGTCGGAGATCAGATCCGCGTCCAGGTCCTCCGCGTTGCTCAGGGCG
AACGGAGTCAA
CTTTGGTAGCTGCCTTCCCAAAAAGGGCGCGTGCCCAGGCTTTGAGTTG
CACTCGCACCG
TAGTGGCATCAAAAGGTGACCGTGCCCGGTCTGGGCGTTAGGATACAGC
GCCTGCATAAA
AGCCTTGATCTGCTTAAAAGCCACCTGAGCCTTTGCGCCTTCAGAGAAG
AACATGCCGCA
AGACTTGCCGGAAAACTGATTGGCCGGACAGGCCGCGTCGTGCACGCAG
CACCTTGCGTC
GGTGTTGGAGATCTGCACCACATTTCGGCCCCACCGGTTCTTCACGATC
TTGGCCTTGCT
AGACTGCTCCTTCAGCGCGCGCTGCCCGTTTTCGCTCGTCACATCCATT
TCAATCACGTG
CTCCTTATTTATCATAATGCTTCCGTGTAGACACTTAAGCTCGCCTTCG
ATCTCAGCGCA
GCGGTGCAGCCACAACGCGCAGCCCGTGGGCTCGTGATGCTTGTAGGTC
ACCTCTGCAAA
CGACTGCAGGTACGCCTGCAGGAATCGCCCCATCATCGTCACAAAGGTC
TTGTTGCTGGT
GAAGGTCAGCTGCAACCCGCGGTGCTCCTCGTTCAGCCAGGTCTTGCAT
ACGGCCGCCAG
AGCTTCCACTTGGTCAGGCAGTAGTTTGAAGTTCGCCTTTAGATCGTTA
TCCACGTGGTA
CTTGTCCATCAGCGCGCGCGCAGCCTCCATGCCCTTCTCCCACGCAGAC
ACGATCGGCAC
ACTCAGCGGGTTCATCACCGTAATTTCACTTTCCGCTTCGCTGGGCTCT
TCCTCTTCCTC
TTGCGTCCGCATACCACGCGCCACTGGGTCGTCTTCATTCAGCCGCCGC
ACTGTGCGCTT
ACCTCCTTTGCCATGCTTGATTAGCACCGGTGGGTTGCTGAAACCCACC
ATTTGTAGCGC
CACATCTTCTCTTTCTTCCTCGCTGTCCACGATTACCTCTGGTGATGGC
GGGCGCTCGGG
CTTGGGAGAAGGGCGCTTCTTTTTCTTCTTGGGCGCAATGGCCAAATCC
GCCGCCGAGGT
CGATGGCCGCGGGCTGGGTGTGCGCGGCACCAGCGCGTCTTGTGATGAG
TCTTCCTCGTC
CTCGGACTCGATACGCCGCCTCATCCGCTTTTTTGGGGGCGCCCGGGGA
GGCGGCGGCGA
CGGGGACGGGGACGACACGTCCTCCATGGTTGGGGGACGTCGCGCCGCA
CCGCGTCCGCG
CTCGGGGGTGGTTTCGCGCTGCTCCTCTTCCCGACTGGCCATTTCCTTC
TCCTATAGGCA
GAAAAAGATCATGGAGTCAGTCGAGAAGAAGGACAGCCTAACCGCCCCC
TCTGAGTTCGC
CACCACCGCCTCCACCGATGCCGCCAACGCGCCTACCACCTTCCCCGTC
GAGGCACCCCC
GCTTGAGGAGGAGGAAGTGATTATCGAGCAGGACCCAGGTTTTGTAAGC
GAAGACGACGA
GGACCGCTCAGTACCAACAGAGGATAAAAAGCAAGACCAGGACAACGCA
GAGGCAAACGA
GGAACAAGTCGGGCGGGGGGACGAAAGGCATGGCGACTACCTAGATGTG
GGAGACGACGT
GCTGTTGAAGCATCTGCAGCGCCAGTGCGCCATTATCTGCGACGCGTTG
CAAGAGCGCAG
CGATGTGCCCCTCGCCATAGCGGATGTCAGCCTTGCCTACGAACGCCAC
CTATTCTCACC
GCGCGTACCCCCCAAACGCCAAGAAAACGGCACATGCGAGCCCAACCCG
CGCCTCAACTT
CTACCCCGTATTTGCCGTGCCAGAGGTGCTTGCCACCTATCACATCTTT
TTCCAAAACTG
CAAGATACCCCTATCCTGCCGTGCCAACCGCAGCCGAGCGGACAAGCAG
CTGGCCTTGCG
GCAGGGCGCTGTCATACCTGATATCGCCTCGCTCAACGAAGTGCCAAAA
ATCTTTGAGGG
TCTTGGACGCGACGAGAAGCGCGCGGCAAACGCTCTGCAACAGGAAAAC
AGCGAAAATGA
AAGTCACTCTGGAGTGTTGGTGGAACTCGAGGGTGACAACGCGCGCCTA
GCCGTACTAAA
ACGCAGCATCGAGGTCACCCACTTTGCCTACCCGGCACTTAACCTACCC
CCCAAGGTCAT
GAGCACAGTCATGAGTGAGCTGATCGTGCGCCGTGCGCAGCCCCTGGAG
AGGGATGCAAA
TTTGCAAGAACAAACAGAGGAGGGCCTACCCGCAGTTGGCGACGAGCAG
CTAGCGCGCTG
GCTTCAAACGCGCGAGCCTGCCGACTTGGAGGAGCGACGCAAACTAATG
ATGGCCGCAGT
GCTCGTTACCGTGGAGCTTGAGTGCATGCAGCGGTTCTTTGCTGACCCG
GAGATGCAGCG
CAAGCTAGAGGAAACATTGCACTACACCTTTCGACAGGGCTACGTACGC
CAGGCCTGCAA
GATCTCCAACGTGGAGCTCTGCAACCTGGTCTCCTACCTTGGAATTTTG
CACGAAAACCG
CCTTGGGCAAAACGTGCTTCATTCCACGCTCAAGGGCGAGGCGCGCCGC
GACTACGTCCG
CGACTGCGTTTACTTATTTCTATGCTACACCTGGCAGACGGCCATGGGC
GTTTGGCAGCA
GTGCTTGGAGGAGTGCAACCTCAAGGAGCTGCAGAAACTGCTAAAGCAA
AACTTGAAGGA
CCTATGGACGGCCTTCAACGAGCGCTCCGTGGCCGCGCACCTGGCGGAC
ATCATTTTCCC
CGAACGCCTGCTTAAAACCCTGCAACAGGGTCTGCCAGACTTCACCAGT
CAAAGCATGTT
GCAGAACTTTAGGAACTTTATCCTAGAGCGCTCAGGAATCTTGCCCGCC
ACCTGCTGTGC
ACTTCCTAGCGACTTTGTGCCCATTAAGTACCGCGAATGCCCTCCGCCG
CTTTGGGGCCA
CTGCTACCTTCTGCAGCTAGCCAACTACCTTGCCTACCACTCTGACATA
ATGGAAGACGT
GAGCGGTGACGGTCTACTGGAGTGTCACTGTCGCTGCAACCTATGCACC
CCGCACCGCTC
CCTGGTTTGCAATTCGCAGCTGCTTAACGAAAGTCAAATTATCGGTACC
TTTGAGCTGCA
GGGTCCCTCGCCTGACGAAAAGTCCGCGGCTCCGGGGTTGAAACTCACT
CCGGGGCTGTG
GACGTCGGCTTACCTTCGCAAATTTGTACCTGAGGACTACCACGCCCAC
GAGATTAGGTT
CTACGAAGACCAATCCCGCCCGCCAAATGCGGAGCTTACCGCCTGCGTC
ATTACCCAGGG
CCACATTCTTGGCCAATTGCAAGCCATCAACAAAGCCCGCCAAGAGTTT
CTGCTACGAAA
GGGACGGGGGGTTTACTTGGACCCCCAGTCCGGCGAGGAGCTCAACCCA
ATCCCCCCGCC
GCCGCAGCCCTATCAGCAGCAGCCGCGGGCCCTTGCTTCCCAGGATGGC
ACCCAAAAAGA
AGCTGCAGCTGCCGCCGCCACCCACGGACGAGGAGGAATACTGGGACAG
TCAGGCAGAGG
AGGTTTTGGACGAGGAGGAGGAGGACATGATGGAAGACTGGGAGAGCCT
AGACGAGGAAG
CTTCCGAGGTCGAAGAGGTGTCAGACGAAACACCGTCACCCTCGGTCGC
ATTCCCCTCGC
CGGCGCCCCAGAAATCGGCAACCGGTTCCAGCATGGCTACAACCTCCGC
TCCTCAGGCGC
CGCCGGCACTGCCCGTTCGCCGACCCAACCGTAGATGGGACACCACTGG
AACCAGGGCCG
GTAAGTCCAAGCAGCCGCCGCCGTTAGCCCAAGAGCAACAACAGCGCCA
AGGCTACCGCT
CATGGCGCGGGCACAAGAACGCCATAGTTGCTTGCTTGCAAGACTGTGG
GGGCAACATCT
CCTTCGCCCGCCGCTTTCTTCTCTACCATCACGGCGTGGCCTTCCCCCG
TAACATCCTGC
ATTACTACCGTCATCTCTACAGCCCATACTGCACCGGCGGCAGCGGCAG
CGGCAGCAACA
GCAGCGGCCACACAGAAGCAAAGGCGACCGGATAGCAAGACTCTGACAA
AGCCCAAGAAA
TCCACAGCGGCGGCAGCAGCAGGAGGAGGAGCGCTGCGTCTGGCGCCCA
ACGAACCCGTA
TCGACCCGCGAGCTTAGAAACAGGATTTTTCCCACTCTGTATGCTATAT
TTCAACAGAGC
AGGGGCCAAGAACAAGAGCTGAAAATAAAAAACAGGTCTCTGCGATCCC
TCACCCGCAGC
TGCCTGTATCACAAAAGCGAAGATCAGCTTCGGCGCACGCTGGAAGACG
CGGAGGCTCTC
TTCAGTAAATACTGCGCGCTGACTCTTAAGGACTAGTTTCGCGCCCTTT
CTCAAATTTAA
GCGCGAAAACTACGTCATCTCCAGCGGCCACACCCGGCGCCAGCACCTG
TCGTCAGCGCC
ATTATGAGCAAGGAAATTCCCACGCCCTACATGTGGAGTTACCAGCCAC
AAATGGGACTT
GCGGCTGGAGCTGCCCAAGACTACTCAACCCGAATAAACTACATGAGCG
CGGGACCCCAC
ATGATATCCCGGGTCAACGGAATCCGCGCCCACCGAAACCGAATTCTCT
TGGAACAGGCG
GCTATTACCACCACACCTCGTAATAACCTTAATCCCCGTAGTTGGCCCG
CTGCCCTGGTG
TACCAGGAAAGTCCCGCTCCCACCACTGTGGTACTTCCCAGAGACGCCC
AGGCCGAAGTT
CAGATGACTAACTCAGGGGCGCAGCTTGCGGGCGGCTTTCGTCACAGGG
TGCGGTCGCCC
GGGCAGGGTATAACTCACCTGACAATCAGAGGGCGAGGTATTCAGCTCA
ACGACGAGTCG
GTGAGCTCCTCGCTTGGTCTCCGTCCGGACGGGACATTTCAGATCGGCG
GCGCCGGCCGT
CCTTCATTCACGCCTCGTCAGGCAATCCTAACTCTGCAGACCTCGTCCT
CTGAGCCGCGC
TCTGGAGGCATTGGAACTCTGCAATTTATTGAGGAGTTTGTGCCATCGG
TCTACTTTAAC
CCCTTCTCGGGACCTCCCGGCCACTATCCGGATCAATTTATTCCTAACT
TTGACGCGGTA
AAGGACTCGGCGGACGGCTACGACTGAATGTTAAGTGGAGAGGCAGAGC
AACTGCGCCTG
AAACACCTGGTCCACTGTCGCCGCCACAAGTGCTTTGCCCGCGACTCCG
GTGAGTTTTGC
TACTTTGAATTGCCCGAGGATCATATCGAGGGCCCGGCGCACGGCGTCC
GGCTTACCGCC
CAGGGAGAGCTTGCCCGTAGCCTGATTCGGGAGTTTACCCAGCGCCCCC
TGCTAGTTGAG
CGGGACAGGGGACCCTGTGTTCTCACTGTGATTTGCAACTGTCCTAACC
TTGGATTACAT
CAAGATCTTTGTTGCCATCTCTGTGCTGAGTATAATAAATACAGAAATT
AAAATATACTG
GGGCTCCTATCGCCATCCTGTAAACGCCACCGTCTTCACCCGCCCAAGC
AAACCAAGGCG
AACCTTACCTGGTACTTTTAACATCTCTCCCTCTGTGATTTACAACAGT
TTCAACCCAGA
CGGAGTGAGTCTACGAGAGAACCTCTCCGAGCTCAGCTACTCCATCAGA
AAAAACACCAC
CCTCCTTACCTGCCGGGAACGTACGAGTGCGTCACCGGCCGCTGCACCA
CACCTACCGCC
TGACCGTAAACCAGACTTTTTCCGGACAGACCTCAATAACTCTGTTTAC
CAGAACAGGAG
GTGAGCTTAGAAAACCCTTAGGGTATTAGGCCAAAGGCGCAGCTACTGT
GGGGTTTATGA
ACAATTCAAGCAACTCTACGGGCTATTCTAATTCAGGTTTCTCTAGAAT
CGGGGTTGGGG
TTATTCTCTGTCTTGTGATTCTCTTTATTCTTATACTAACGCTTCTCTG
CCTAAGGCTCG
CCGCCTGCTGTGTGCACATTTGCATTTATTGTCAGCTTTTTAAACGCTG
GGGTCGCCACC
CAAGATGATTAGGTACATAATCCTAGGTTTACTCACCCTTGCGTCAGCC
CACGGTACCAC
CCAAAAGGTGGATTTTAAGGAGCCAGCCTGTAATGTTACATTCGCAGCT
GAAGCTAATGA
GTGCACCACTCTTATAAAATGCACCACAGAACATGAAAAGCTGCTTATT
CGCCACAAAAA
CAAAATTGGCAAGTATGCTGTTTATGCTATTTGGCAGCCAGGTGACACT
ACAGAGTATAA
TGTTACAGTTTTCCAGGGTAAAAGTCATAAAACTTTTATGTATACTTTT
CCATTTTATGA
AATGTGCGACATTACCATGTACATGAGCAAACAGTATAAGTTGTGGCCC
CCACAAAATTG
TGTGGAAAACACTGGCACTTTCTGCTGCACTGCTATGCTAATTACAGTG
CTCGCTTTGGT
CTGTACCCTACTCTATATTAAATACAAAAGCAGACGCAGCTTTATTGAG
GAAAAGAAAAT
GCCTTAATTTACTAAGTTACAAAGCTAATGTCACCACTAACTGCTTTAC
TCGCTGCTTGC
AAAACAAATTCAAAAAGTTAGCATTATAATTAGAATAGGATTTAAACCC
CCCGGTCATTT
CCTGCTCAATACCATTCCCCTGAACAATTGACTCTATGTGGGATATGCT
CCAGCGCTACA
ACCTTGAAGTCAGGCTTCCTGGATGTCAGCATCTGACTTTGGCCAGCAC
CTGTCCCGCGG
ATTTGTTCCAGTCCAACTACAGCGACCCACCCTAACAGAGATGACCAAC
ACAACCAACGC
GGCCGCCGCTACCGGACTTACATCTACCACAAATACACCCCAAGTTTCT
GCCTTTGTCAA
TAACTGGGATAACTTGGGCATGTGGTGGTTCTCCATAGCGCTTATGTTT
GTATGCCTTAT
TATTATGTGGCTCATCTGCTGCCTAAAGCGCAAACGCGCCCGACCACCC
ATCTATAGTCC
CATCATTGTGCTACACCCAAACAATGATGGAATCCATAGATTGGACGGA
CTGAAACACAT
GTTCTTTTCTCTTACAGTATGATTAAATGAGACATGATTCCTCGAGTTT
TTATATTACTG
ACCCTTGTTGCGCTTTTTTGTGCGTGCTCCACATTGGCTGCGGTTTCTC
ACATCGAAGTA
GACTGCATTCCAGCCTTCACAGTCTATTTGCTTTACGGATTTGTCACCC
TCACGCTCATC
TGCAGCCTCATCACTGTGGTCATCGCCTTTATCCAGTGCATTGACTGGG
TCTGTGTGCGC
TTTGCATATCTCAGACACCATCCCCAGTACAGGGACAGGACTATAGCTG
AGCTTCTTAGA
ATTCTTTAATTATGAAATTTACTGTGACTTTTCTGCTGATTATTTGCAC
CCTATCTGCGT
TTTGTTCCCCGACCTCCAAGCCTCAAAGACATATATCATGCAGATTCAC
TCGTATATGGA
ATATTCCAAGTTGCTACAATGAAAAAAGCGATCTTTCCGAAGCCTGGTT
ATATGCAATCA
TCTCTGTTATGGTGTTCTGCAGTACCATCTTAGCCCTAGCTATATATCC
CTACCTTGACA
TTGGCTGGAACGCAATAGATGCCATGAACCACCCAACTTTCCCCGCGCC
CGCTATGCTTC
CACTGCAACAAGTTGTTGCCGGCGGCTTTGTCCCAGCCAATCAGCCTCG
CCCACCTTCTC
CCACCCCCACTGAAATCAGCTACTTTAATCTAACAGGAGGAGATGACTG
ACACCCTAGAT
CTAGAAATGGACGGAATTATTACAGAGCAGCGCCTGCTAGAAAGACGCA
GGGCAGCGGCC
GAGCAACAGCGCATGAATCAAGAGCTCCAAGACATGGTTAACTTGCACC
AGTGCAAAAGG
GGTATCTTTTGTCTGGTAAAGCAGGCCAAAGTCACCTACGACAGTAATA
CCACCGGACAC
CGCCTTAGCTACAAGTTGCCAACCAAGCGTCAGAAATTGGTGGTCATGG
TGGGAGAAAAG
CCCATTACCATAACTCAGCACTCGGTAGAAACCGAAGGCTGCATTCACT
CACCTTGTCAA
GGACCTGAGGATCTCTGCACCCTTATTAAGACCCTGTGCGGTCTCAAAG
ATCTTATTCCC
TTTAACTAATAAAAAAAAATAATAAAGCATCACTTACTTAAAATCAGTT
AGCAAATTTCT
GTCCAGTTTATTCAGCAGCACCTCCTTGCCCTCCTCCCAGCTCTGGTAT
TGCAGCTTCCT
CCTGGCTGCAAACTTTCTCCACAATCTAAATGGAATGTCAGTTTCCTCC
TGTTCCTGTCC
ATCCGCACCCACTATCTTCATGTTGTTGCAGATGAAGCGCGCAAGACCG
TCTGAAGATAC
CTTCAACCCCGTGTATCCATATGACACGGAAACCGGTCCTCCAACTGTG
CCTTTTCTTAC
TCCTCCCTTTGTATCCCCCAATGGGTTTCAAGAGAGTCCCCCTGGGGTA
CTCTCTTTGCG
CCTATCCGAACCTCTAGTTACCTCCAATGGCATGCTTGCGCTCAAAATG
GGCAACGGCCT
CTCTCTGGACGAGGCCGGCAACCTTACCTCCCAAAATGTAACCACTGTG
AGCCCACCTCT
CAAAAAAACCAAGTCAAACATAAACCTGGAAATATCTGCACCCCTCACA
GTTACCTCAGA
AGCCCTAACTGTGGCTGCCGCCGCACCTCTAATGGTCGCGGGCAACACA
CTCACCATGCA
ATCACAGGCCCCGCTAACCGTGCACGACTCCAAACTTAGCATTGCCACC
CAAGGACCCCT
CACAGTGTCAGAAGGAAAGCTAGCCCTGCAAACATCAGGCCCCCTCACC
ACCACCGATAG
CAGTACCCTTACTATCACTGCCTCACCCCCTCTAACTACTGCCACTGGT
AGCTTGGGCAT
TGACTTGAAAGAGCCCATTTATACACAAAATGGAAAACTAGGACTAAAG
TACGGGGCTCC
TTTGCATGTAACAGACGACCTAAACACTTTGACCGTAGCAACTGGTCCA
GGTGTGACTAT
TAATAATACTTCCTTGCAAACTAAAGTTACTGGAGCCTTGGGTTTTGAT
TCACAAGGCAA
TATGCAACTTAATGTAGCAGGAGGACTAAGGATTGATTCTCAAAACAGA
CGCCTTATACT
TGATGTTAGTTATCCGTTTGATGCTCAAAACCAACTAAATCTAAGACTA
GGACAGGGCCC
TCTTTTTATAAACTCAGCCCACAACTTGGATATTAACTACAACAAAGGC
CTTTACTTGTT
TACAGCTTCAAACAATTCCAAAAAGCTTGAGGTTAACCTAAGCACTGCC
AAGGGGTTGAT
GTTTGACGCTACAGCCATAGCCATTAATGCAGGAGATGGGCTTGAATTT
GGTTCACCTAA
TGCACCAAACACAAATCCCCTCAAAACAAAAATTGGCCATGGCCTAGAA
TTTGATTCAAA
CAAGGCTATGGTTCCTAAACTAGGAACTGGCCTTAGTTTTGACAGCACA
GGTGCCATTAC
AGTAGGAAACAAAAATAATGATAAGCTAACTTTGTGGACCACACCAGCT
CCATCTCCTAA
CTGTAGACTAAATGCAGAGAAAGATGCTAAACTCACTTTGGTCTTAACA
AAATGTGGCAG
TCAAATACTTGCTACAGTTTCAGTTTTGGCTGTTAAAGGCAGTTTGGCT
CCAATATCTGG
AACAGTTCAAAGTGCTCATCTTATTATAAGATTTGACGAAAATGGAGTG
CTACTAAACAA
TTCCTTCCTGGACCCAGAATATTGGAACTTTAGAAATGGAGATCTTACT
GAAGGCACAGC
CTATACAAACGCTGTTGGATTTATGCCTAACCTATCAGCTTATCCAAAA
TCTCACGGTAA
AACTGCCAAAAGTAACATTGTCAGTCAAGTTTACTTAAACGGAGACAAA
ACTAAACCTGT
AACACTAACCATTACACTAAACGGTACACAGGAAACAGGAGACACAACT
CCAAGTGCATA
CTCTATGTCATTTTCATGGGACTGGTCTGGCCACAACTACATTAATGAA
ATATTTGCCAC
ATCCTCTTACACTTTTTCATACATTGCCCAAGAATAAAGAATCGTTTGT
GTTATGTTTCA
ACGTGTTTATTTTTCAATTGCAGAAAATTTCAAGTCATTTTTCATTCAG
TAGTATAGCCC
CACCACCACATAGCTTATACAGATCACCGTACCTTAATCAAACTCACAG
AACCCTAGTAT
TCAACCTGCCACCTCCCTCCCAACACACAGAGTACACAGTCCTTTCTCC
CCGGCTGGCCT
TAAAAAGCATCATATCATGGGTAACAGACATATTCTTAGGTGTTATATT
CCACACGGTTT
CCTGTCGAGCCAAACGCTCATCAGTGATATTAATAAACTCCCCGGGCAG
CTCACTTAAGT
TCATGTCGCTGTCCAGCTGCTGAGCCACAGGCTGCTGTCCAACTTGCGG
TTGCTTAACGG
GCGGCGAAGGAGAAGTCCACGCCTACATGGGGGTAGAGTCATAATCGTG
CATCAGGATAG
GGCGGTGGTGCTGCAGCAGCGCGCGAATAAACTGCTGCCGCCGCCGCTC
CGTCCTGCAGG
AATACAACATGGCAGTGGTCTCCTCAGCGATGATTCGCACCGCCCGCAG
CATAAGGCGCC
TTGTCCTCCGGGCACAGCAGCGCACCCTGATCTCACTTAAATCAGCACA
GTAACTGCAGC
ACAGCACCACAATATTGTTCAAAATCCCACAGTGCAAGGCGCTGTATCC
AAAGCTCATGG
CGGGGACCACAGAACCCACGTGGCCATCATACCACAAGCGCAGGTAGAT
TAAGTGGCGAC
CCCTCATAAACACGCTGGACATAAACATTACCTCTTTTGGCATGTTGTA
ATTCACCACCT
CCCGGTACCATATAAACCTCTGATTAAACATGGCGCCATCCACCACCAT
CCTAAACCAGC
TGGCCAAAACCTGCCCGCCGGCTATACACTGCAGGGAACCGGGACTGGA
ACAATGACAGT
GGAGAGCCCAGGACTCGTAACCATGGATCATCATGCTCGTCATGATATC
AATGTTGGCAC
AACACAGGCACACGTGCATACACTTCCTCAGGATTACAAGCTCCTCCCG
CGTTAGAACCA
TATCCCAGGGAACAACCCATTCCTGAATCAGCGTAAATCCCACACTGCA
GGGAAGACCTC
GCACGTAACTCACGTTGTGCATTGTCAAAGTGTTACATTCGGGCAGCAG
CGGATGATCCT
CCAGTATGGTAGCGCGGGTTTCTGTCTCAAAAGGAGGTAGACGATCCCT
ACTGTACGGAG
TGCGCCGAGACAACCGAGATCGTGTTGGTCGTAGTGTCATGCCAAATGG
AACGCCGGACG
TAGTCATATTTCCTGAAGCAAAACCAGGTGCGGGCGTGACAAACAGATC
TGCGTCTCCGG
TCTCGCCGCTTAGATCGCTCTGTGTAGTAGTTGTAGTATATCCACTCTC
TCAAAGCATCC
AGGCGCCCCCTGGCTTCGGGTTCTATGTAAACTCCTTCATGCGCCGCTG
CCCTGATAACA
TCCACCACCGCAGAATAAGCCACACCCAGCCAACCTACACATTCGTTCT
GCGAGTCACAC
ACGGGAGGAGCGGGAAGAGCTGGAAGAACCATGTTTTTTTTTTTATTCC
AAAAGATTATC
CAAAACCTCAAAATGAAGATCTATTAAGTGAACGCGCTCCCCTCCGGTG
GCGTGGTCAAA
CTCTACAGCCAAAGAACAGATAATGGCATTTGTAAGATGTTGCACAATG
GCTTCCAAAAG
GCAAACGGCCCTCACGTCCAAGTGGACGTAAAGGCTAAACCCTTCAGGG
TGAATCTCCTC
TATAAACATTCCAGCACCTTCAACCATGCCCAAATAATTCTCATCTCGC
CACCTTCTCAA
TATATCTCTAAGCAAATCCCGAATATTAAGTCCGGCCATTGTAAAAATC
TGCTCCAGAGC
GCCCTCCACCTTCAGCCTCAAGCAGCGAATCATGATTGCAAAAATTCAG
GTTCCTCACAG
ACCTGTATAAGATTCAAAAGCGGAACATTAACAAAAATACCGCGATCCC
GTAGGTCCCTT
CGCAGGGCCAGCTGAACATAATCGTGCAGGTCTGCACGGACCAGCGCGG
CCACTTCCCCG
CCAGGAACCTTGACAAAAGAACCCACACTGATTATGACACGCATACTCG
GAGCTATGCTA
ACCAGCGTAGCCCCGATGTAAGCTTTGTTGCATGGGCGGCGATATAAAA
TGCAAGGTGCT
GCTCAAAAAATCAGGCAAAGCCTCGCGCAAAAAAGAAAGCACATCGTAG
TCATGCTCATG
CAGATAAAGGCAGGTAAGCTCCGGAACCACCACAGAAAAAGACACCATT
TTTCTCTCAAA
CATGTCTGCGGGTTTCTGCATAAACACAAAATAAAATAACAAAAAAACA
TTTAAACATTA
GAAGCCTGTCTTACAACAGGAAAAACAACCCTTATAAGCATAAGACGGA
CTACGGCCATG
CCGGCGTGACCGTAAAAAAACTGGTCACCGTGATTAAAAAGCACCACCG
ACAGCTCCTCG
GTCATGTCCGGAGTCATAATGTAAGACTCGGTAAACACATCAGGTTGAT
TCATCGGTCAG
TGCTAAAAAGCGACCGAAATAGCCCGGGGGAATACATACCCGCAGGCGT
AGAGACAACAT
TACAGCCCCCATAGGAGGTATAACAAAATTAATAGGAGAGAAAAACACA
TAAACACCTGA
AAAACCCTCCTGCCTAGGCAAAATAGCACCCTCCCGCTCCAGAACAACA
TACAGCGCTTC
CACAGCGGCAGCCATAACAGTCAGCCTTACCAGTAAAAAAGAAAACCTA
TTAAAAAAACA
CCACTCGACACGGCACCAGCTCAATCAGTCACAGTGTAAAAAAGGGCCA
AGTGCAGAGCG
AGTATATATAGGACTAAAAAATGACGTAACGGTTAAAGTCCACAAAAAA
CACCCAGAAAA
CCGCACGCGAACCTACGCCCAGAAACGAAAGCCAAAAAACCCACAACTT
CCTCAAATCGT
CACTTCCGTTTTCCCACGTTACGTCACTTCCCATTTTAATTAAGAAAAC
TACAATTCCCA
ACACATACAAGTTACTCCGCCCTAAAACCTACGTCACCCGCCCCGTTCC
CACGCCCCGCG
CCACGTCACAAACTCCACCCCCTCATTATCATATTGGCTTCAATCCAAA
ATAAGGTATAT
TATTGATGATGATTACCCTGTTAT
SEQ ID NO: 50 is the OV1164 sequence
CAGGGTAATCATCATCAATAATATACCTTATTTTGGATTGAAGCCAATA
TGATAATGAGG
GGGTGGAGTTTGTGACGTGGCGCGGGGCGTGGGAACGGGGCGGGTGACG
TAGTAGTGTGG
CGGAAGTGTGATGTTGCAAGTGTGGCGGAACACATGTAAGCGACGGATG
TGGCAAAAGTG
ACGTTTTTGGTGTGCGCCGGTGTACACAGGAAGTGACAATTTTCGCGCG
GTTTTAGGCGG
ATGTTGTAGTAAATTTGGGCGTAACCGAGTAAGATTTGGCCATTTTCGC
GGGAAAACTGA
ATAAGAGGAAGTGAAATCTGAATAATTTTGTGTTACTCATAGCGCGTAA
TATTTGTCTAG
GGCCGGGATCTCTGCAGGAATTTGATATCAAGCTTATCGATACCGTCGA
AACTTGTTTAT
TGCAGCTTATAATGGTTACAAATAAAGCAATAGCATCACAAATTTCACA
AATAAAGCATT
TTTTTCACTGCATTCTAGTTGTGGTTTGTCCAAACTCATCAATGTATCT
TATCATGTCTG
GATCCGCTAGCGGCGCGCCGTTTCATCCGGACAAAGCCTGCGCGCGCCC
CGCCCCGCCAT
TGGCCGTACCGCCCCGCGCCGCCGCCCCATCTCGCCCCTCGCCGCCGGG
TCCGGCGCGTT
AAAGCCAATAGGAACCGCCGCCGTTGTTCCCGTCACGGCCGGGGCAGCC
AATTGTGGCGG
CGCTCGGCGGCTCGTGGCTCTTTCGCGGCAAAAAGGATTTGGCGCGTAA
AAGTGGCCGGG
ACTTTGCAGGCAGCGGCGGCCGGGGGCGGAGCGGGATCGAGCCCTCGAT
GATATCAGATC
AAACGATATCACCGGTCGACTGAAAATGAGACATATTATCTGCCACGGA
GGTGTTATTAC
CGAAGAAATGGCCGCCAGTCTTTTGGACCAGCTGATCGAAGAGGTACTG
GCTGATAATCT
TCCACCTCCTAGCCATTTTGAACCACCTACCCTTCACGAACTGTATGAT
TTAGACGTGAC
GGCCCCCGAAGATCCCAACGAGGAGGCGGTTTCGCAGATTTTTCCCGAC
TCTGTAATGTT
GGCGGTGCAGGAAGGGATTGACTTACTCACTTTTCCGCCGGCGCCCGGT
TCTCCGGAGCC
GCCTCACCTTTCCCGGCAGCCCGAGCAGCCGGAGCAGAGAGCCTTGGGT
CCGGTTTCTAT
GCCAAACCTTGTACCGGAGGTGATCGATCTTACCTGCCACGAGGCTGGC
TTTCCACCCAG
TGACGACGAGGATGAAGAGGGTGAGGAGTTTGTGTTAGATTATGTGGAG
CACCCCGGGCA
CGGTTGCAGGTCTTGTCATTATCACCGGAGGAATACGGGGGACCCAGAT
ATTATGTGTTC
GCTTTGCTATATGAGGACCTGTGGCATGTTTGTCTACAGTAAGTGAAAA
TTATGGGCAGT
GGGTGATAGAGTGGTGGGTTTGGTGTGGTAATTTTTTTTTTAATTTTTA
CAGTTTTGTGG
TTTAAAGAATTTTGTATTGTGATTTTTTTAAAAGGTCCTGTGTCTGAAC
CTGAGCCTGAG
CCCGAGCCAGAACCGGAGCCTGCAAGACCTACCCGCCGTCCTAAAATGG
CGCCTGCTATC
CTGAGACGCCCGACATCACCTGTGTCTAGAGAATGCAATAGTAGTACGG
ATAGCTGTGAC
TCCGGTCCTTCTAACACACCTCCTGAGATACACCCGGTGGTCCCGCTGT
GCCCCATTAAA
CCAGTTGCCGTGAGAGTTGGTGGGCGTCGCCAGGCTGTGGAATGTATCG
AGGACTTGCTT
AACGAGCCTGGGCAACCTTTGGACTTGAGCTGTAAACGCCCCAGGCCAT
AAGGTGTAAAC
CTGTGATTGCGTGTGTGGTTAACGCCTTTGTTTGCTGAATGAGTTGATG
TAAGTTTAATA
AAGGGTGAGATAATGTTTAACTTGCATGGCGTGTTAAATGGGGCGGGGC
TTAAAGGGTAT
ATAATGCGCCGTGGGCTAATCTTGGTTACATCTGACCTCATGGAGGCTT
GGGAGTGTTTG
GAAGATTTTTCTGCTGTGCGTAACTTGCTGGAACAGAGCTCTAACAGTA
CCTCTTGGTTT
TGGAGGTTTCTGTGGGGCTCATCCCAGGCAAAGTTAGTCTGCAGAATTA
AGGAGGATTAC
AAGTGGGAATTTGAAGAGCTTTTGAAATCCTGTGGTGAGCTGTTTGATT
CTTTGAATCTG
GGTCACCAGGCGCTTTTCCAAGAGAAGGTCATCAAGACTTTGGATTTTT
CCACACCGGGG
CGCGCTGCGGCTGCTGTTGCTTTTTTGAGTTTTATAAAGGATAAATGGA
GCGAAGAAACC
CATCTGAGCGGGGGGTACCTGCTGGATTTTCTGGCCATGCATCTGTGGA
GAGCGGTTGTG
AGACACAAGAATCGCCTGCTACTGTTGTCTTCCGTCCGCCCGGCGATAA
TACCGACGGAG
GAGCAGCAGCAGCAGCAGGAGGAAGCCAGGCGGCGGCGGCAGGAGCAGA
GCCCATGGAAC
CCGAGAGCCGGCCTGGACCCTCGGGAATGAATGTTGTACAGGTGGCTGA
ACTGTATCCAG
AACTGAGACGCATTTTGACAATTACAGAGGATGGGCAGGGGCTAAAGGG
GGTAAAGAGGG
AGCGGGGGGCTTGTGAGGCTACAGAGGAGGCTAGGAATCTAGCTTTTAG
CTTAATGACCA
GACACCGTCCTGAGTGTATTACTTTTCAACAGATCAAGGATAATTGCGC
TAATGAGCTTG
ATCTGCTGGCGCAGAAGTATTCCATAGAGCAGCTGACCACTTACTGGCT
GCAGCCAGGGG
ATGATTTTGAGGAGGCTATTAGGGTATATGCAAAGGTGGCACTTAGGCC
AGATTGCAAGT
ACAAGATCAGCAAACTTGTAAATATCAGGAATTGTTGCTACATTTCTGG
GAACGGGGCCG
AGGTGGAGATAGATACGGAGGATAGGGTGGCCTTTAGATGTAGCATGAT
AAATATGTGGC
CGGGGGTGCTTGGCATGGACGGGGTGGTTATTATGAATGTAAGGTTTAC
TGGCCCCAATT
TTAGCGGTACGGTTTTCCTGGCCAATACCAACCTTATCCTACACGGTGT
AAGCTTCTATG
GGTTTAACAATACCTGTGTGGAAGCCTGGACCGATGTAAGGGTTCGGGG
CTGTGCCTTTT
ACTGCTGCTGGAAGGGGGTGGTGTGTCGCCCCAAAAGCAGGGCTTCAAT
TAAGAAATGCC
TCTTTGAAAGGTGTACCTTGGGTATCCTGTCTGAGGGTAACTCCAGGGT
GCGCCACAATG
TGGCCTCCGACTGTGGTTGCTTCATGCTAGTGAAAAGCGTGGCTGTGAT
TAAGCATAACA
TGGTATGTGGCAACTGCGAGGACAGGGCCTCTCAGATGCTGACCTGCTC
GGACGGCAACT
GTCACCTGCTGAAGACCATTCACGTAGCCAGCCACTCTCGCAAGGCCTG
GCCAGTGTTTG
AGCATAACATACTGACCCGCTGTTCCTTGCATTTGGGTAACAGGAGGGG
GGTGTTCCTAC
CTTACCAATGCAATTTGAGTCACACTAAGATATTGCTTGAGCCCGAGAG
CATGTCCAAGG
TGAACCTGAACGGGGTGTTTGACATGACCATGAAGATCTGGAAGGTGCT
GAGGTACGATG
AGACCCGCACCAGGTGCAGACCCTGCGAGTGTGGCGGTAAACATATTAG
GAACCAGCCTG
TGATGCTGGATGTGACCGAGGAGCTGAGGCCCGATCACTTGGTGCTGGC
CTGCACCCGCG
CTGAGTTTGGCTCTAGCGATGAAGATACAGATTGAGGTACTGAAATGTG
TGGGCGTGGCT
TAAGGGTGGGAAAGAATATATAAGGTGGGGGTCTTATGTAGTTTTGTAT
CTGTTTTGCAG
CAGCCGCCGCCGCCATGAGCACCAACTCGTTTGATGGAAGCATTGTGAG
CTCATATTTGA
CAACGCGCATGCCCCCATGGGCCGGGGTGCGTCAGAATGTGATGGGCTC
CAGCATTGATG
GTCGCCCCGTCCTGCCCGCAAACTCTACTACCTTGACCTACGAGACCGT
GTCTGGAACGC
CGTTGGAGACTGCAGCCTCCGCCGCCGCTTCAGCCGCTGCAGCCACCGC
CCGCGGGATTG
TGACTGACTTTGCTTTCCTGAGCCCGCTTGCAAGCAGTGCAGCTTCCCG
TTCATCCGCCC
GCGATGACAAGTTGACGGCTCTTTTGGCACAATTGGATTCTTTGACCCG
GGAACTTAATG
TCGTTTCTCAGCAGCTGTTGGATCTGCGCCAGCAGGTTTCTGCCCTGAA
GGCTTCCTCCC
CTCCCAATGCGGTTTAAAACATAAATAAAAAACCAGACTCTGTTTGGAT
TTGGATCAAGC
AAGTGTCTTGCTGTCTTTATTTAGGGGTTTTGCGCGCGCGGTAGGCCCG
GGACCAGCGGT
CTCGGTCGTTGAGGGTCCTGTGTATTTTTTCCAGGACGTGGTAAAGGTG
ACTCTGGATGT
TCAGATACATGGGCATAAGCCCGTCTCTGGGGTGGAGGTAGCACCACTG
CAGAGCTTCAT
GCTGCGGGGTGGTGTTGTAGATGATCCAGTCGTAGCAGGAGCGCTGGGC
GTGGTGCCTAA
AAATGTCTTTCAGTAGCAAGCTGATTGCCAGGGGCAGGCCCTTGGTGTA
AGTGTTTACAA
AGCGGTTAAGCTGGGATGGGTGCATACGTGGGGATATGAGATGCATCTT
GGACTGTATTT
TTAGGTTGGCTATGTTCCCAGCCATATCCCTCCGGGGATTCATGTTGTG
CAGAACCACCA
GCACAGTGTATCCGGTGCACTTGGGAAATTTGTCATGTAGCTTAGAAGG
AAATGCGTGGA
AGAACTTGGAGACGCCCTTGTGACCTCCAAGATTTTCCATGCATTCGTC
CATAATGATGG
CAATGGGCCCACGGGCGGCGGCCTGGGCGAAGATATTTCTGGGATCACT
AACGTCATAGT
TGTGTTCCAGGATGAGATCGTCATAGGCCATTTTTACAAAGCGCGGGCG
GAGGGTGCCAG
ACTGCGGTATAATGGTTCCATCCGGCCCAGGGGCGTAGTTACCCTCACA
GATTTGCATTT
CCCACGCTTTGAGTTCAGATGGGGGGATCATGTCTACCTGCGGGGCGAT
GAAGAAAACGG
TTTCCGGGGTAGGGGAGATCAGCTGGGAAGAAAGCAGGTTCCTGAGCAG
CTGCGACTTAC
CGCAGCCGGTGGGCCCGTAAATCACACCTATTACCGGGTGCAACTGGTA
GTTAAGAGAGC
TGCAGCTGCCGTCATCCCTGAGCAGGGGGGCCACTTCGTTAAGCATGTC
CCTGACTCGCA
TGTTTTCCCTGACCAAATCCGCCAGAAGGCGCTCGCCGCCCAGCGATAG
CAGTTCTTGCA
AGGAAGCAAAGTTTTTCAACGGTTTGAGACCGTCCGCCGTAGGCATGCT
TTTGAGCGTTT
GACCAAGCAGTTCCAGGCGGTCCCACAGCTCGGTCACCTGCTCTACGGC
ATCTCGATCCA
GCATATCTCCTCGTTTCGCGGGTTGGGGCGGCTTTCGCTGTACGGCAGT
AGTCGGTGCTC
GTCCAGACGGGCCAGGGTCATGTCTTTCCACGGGCGCAGGGTCCTCGTC
AGCGTAGTCTG
GGTCACGGTGAAGGGGTGCGCTCCGGGCTGCGCGCTGGCCAGGGTGCGC
TTGAGGCTGGT
CCTGCTGGTGCTGAAGCGCTGCCGGTCTTCGCCCTGCGCGTCGGCCAGG
TAGCATTTGAC
CATGGTGTCATAGTCCAGCCCCTCCGCGGCGTGGCCCTTGGCGCGCAGC
TTGCCCTTGGA
GGAGGCGCCGCACGAGGGGCAGTGCAGACTTTTGAGGGCGTAGAGCTTG
GGCGCGAGAAA
TACCGATTCCGGGGAGTAGGCATCCGCGCCGCAGGCCCCGCAGACGGTC
TCGCATTCCAC
GAGCCAGGTGAGCTCTGGCCGTTCGGGGTCAAAAACCAGGTTTCCCCCA
TGCTTTTTGAT
GCGTTTCTTACCTCTGGTTTCCATGAGCCGGTGTCCACGCTCGGTGACG
AAAAGGCTGTC
CGTGTCCCCGTATACAGACTTGAGAGGCCTGTCCTCGAGCGGTGTTCCG
CGGTCCTCCTC
GTATAGAAACTCGGACCACTCTGAGACAAAGGCTCGCGTCCAGGCCAGC
ACGAAGGAGGC
TAAGTGGGAGGGGTAGCGGTCGTTGTCCACTAGGGGGTCCACTCGCTCC
AGGGTGTGAAG
ACACATGTCGCCCTCTTCGGCATCAAGGAAGGTGATTGGTTTGTAGGTG
TAGGCCACGTG
ACCGGGTGTTCCTGAAGGGGGGCTATAAAAGGGGGTGGGGGCGCGTTCG
TCCTCACTCTC
TTCCGCATCGCTGTCTGCGAGGGCCAGCTGTTGGGGTGAGTACTCCCTC
TGAAAAGCGGG
CATGACTTCTGCGCTAAGATTGTCAGTTTCCAAAAACGAGGAGGATTTG
ATATTCACCTG
GCCCGCGGTGATGCCTTTGAGGGTGGCCGCATCCATCTGGTCAGAAAAG
ACAATCTTTTT
GTTGTCAAGCTTGGTGGCAAACGACCCGTAGAGGGCGTTGGACAGCAAC
TTGGCGATGGA
GCGCAGGGTTTGGTTTTTGTCGCGATCGGCGCGCTCCTTGGCCGCGATG
TTTAGCTGCAC
GTATTCGCGCGCAACGCACCGCCATTCGGGAAAGACGGTGGTGCGCTCG
TCGGGCACCAG
GTGCACGCGCCAACCGCGGTTGTGCAGGGTGACAAGGTCAACGCTGGTG
GCTACCTCTCC
GCGTAGGCGCTCGTTGGTCCAGCAGAGGCGGCCGCCCTTGCGCGAGCAG
AATGGCGGTAG
GGGGTCTAGCTGCGTCTCGTCCGGGGGGTCTGCGTCCACGGTAAAGACC
CCGGGCAGCAG
GCGCGCGTCGAAGTAGTCTATCTTGCATCCTTGCAAGTCTAGCGCCTGC
TGCCATGCGCG
GGCGGCAAGCGCGCGCTCGTATGGGTTGAGTGGGGGACCCCATGGCATG
GGGTGGGTGAG
CGCGGAGGCGTACATGCCGCAAATGTCGTAAACGTAGAGGGGCTCTCTG
AGTATTCCAAG
ATATGTAGGGTAGCATCTTCCACCGCGGATGCTGGCGCGCACGTAATCG
TATAGTTCGTG
CGAGGGAGCGAGGAGGTCGGGACCGAGGTTGCTACGGGCGGGCTGCTCT
GCTCGGAAGAC
TATCTGCCTGAAGATGGCATGTGAGTTGGATGATATGGTTGGACGCTGG
AAGACGTTGAA
GCTGGCGTCTGTGAGACCTACCGCGTCACGCACGAAGGAGGCGTAGGAG
TCGCGCAGCTT
GTTGACCAGCTCGGCGGTGACCTGCACGTCTAGGGCGCAGTAGTCCAGG
GTTTCCTTGAT
GATGTCATACTTATCCTGTCCCTTTTTTTTCCACAGCTCGCGGTTGAGG
ACAAACTCTTC
GCGGTCTTTCCAGTACTCTTGGATCGGAAACCCGTCGGCCTCCGAACGG
TAAGAGCCTAG
CATGTAGAACTGGTTGACGGCCTGGTAGGCGCAGCATCCCTTTTCTACG
GGTAGCGCGTA
TGCCTGCGCGGCCTTCCGGAGCGAGGTGTGGGTGAGCGCAAAGGTGTCC
CTGACCATGAC
TTTGAGGTACTGGTATTTGAAGTCAGTGTCGTCGCATCCGCCCTGCTCC
CAGAGCAAAAA
GTCCGTGCGCTTTTTGGAACGCGGATTTGGCAGGGCGAAGGTGACATCG
TTGAAGAGTAT
CTTTCCCGCGCGAGGCATAAAGTTGCGTGTGATGCGGAAGGGTCCCGGC
ACCTCGGAACG
GTTGTTAATTACCTGGGCGGCGAGCACGATCTCGTCAAAGCCGTTGATG
TTGTGGCCCAC
AATGTAAAGTTCCAAGAAGCGCGGGATGCCCTTGATGGAAGGCAATTTT
TTAAGTTCCTC
GTAGGTGAGCTCTTCAGGGGAGCTGAGCCCGTGCTCTGAAAGGGCCCAG
TCTGCAAGATG
AGGGTTGGAAGCGACGAATGAGCTCCACAGGTCACGGGCCATTAGCATT
TGCAGGTGGTC
GCGAAAGGTCCTAAACTGGCGACCTATGGCCATTTTTTCTGGGGTGATG
CAGTAGAAGGT
AAGCGGGTCTTGTTCCCAGCGGTCCCATCCAAGGTTCGCGGCTAGGTCT
CGCGCGGCAGT
CACTAGAGGCTCATCTCCGCCGAACTTCATGACCAGCATGAAGGGCACG
AGCTGCTTCCC
AAAGGCCCCCATCCAAGTATAGGTCTCTACATCGTAGGTGACAAAGAGA
CGCTCGGTGCG
AGGATGCGAGCCGATCGGGAAGAACTGGATCTCCCGCCACCAATTGGAG
GAGTGGCTATT
GATGTGGTGAAAGTAGAAGTCCCTGCGACGGGCCGAACACTCGTGCTGG
CTTTTGTAAAA
ACGTGCGCAGTACTGGCAGCGGTGCACGGGCTGTACATCCTGCACGAGG
TTGACCTGACG
ACCGCGCACAAGGAAGCAGAGTGGGAATTTGAGCCCCTCGCCTGGCGGG
TTTGGCTGGTG
GTCTTCTACTTCGGCTGCTTGTCCTTGACCGTCTGGCTGCTCGAGGGGA
GTTACGGTGGA
TCGGACCACCACGCCGCGCGAGCCCAAAGTCCAGATGTCCGCGCGCGGC
GGTCGGAGCTT
GATGACAACATCGCGCAGATGGGAGCTGTCCATGGTCTGGAGCTCCCGC
GGCGTCAGGTC
AGGCGGGAGCTCCTGCAGGTTTACCTCGCATAGACGGGTCAGGGCGCGG
GCTAGATCCAG
GTGATACCTAATTTCCAGGGGCTGGTTGGTGGCGGCGTCGATGGCTTGC
AAGAGGCCGCA
TCCCCGCGGCGCGACTACGGTACCGCGCGGCGGGCGGTGGGCCGCGGGG
GTGTCCTTGGA
TGATGCATCTAAAAGCGGTGACGCGGGCGAGCCCCCGGAGGTAGGGGGG
GCTCCGGACCC
GCCGGGAGAGGGGGCAGGGGCACGTCGGCGCCGCGCGCGGGCAGGAGCT
GGTGCTGCGCG
CGTAGGTTGCTGGCGAACGCGACGACGCGGCGGTTGATCTCCTGAATCT
GGCGCCTCTGC
GTGAAGACGACGGGCCCGGTGAGCTTGAGCCTGAAAGAGAGTTCGACAG
AATCAATTTCG
GTGTCGTTGACGGCGGCCTGGCGCAAAATCTCCTGCACGTCTCCTGAGT
TGTCTTGATAG
GCGATCTCGGCCATGAACTGCTCGATCTCTTCCTCCTGGAGATCTCCGC
GTCCGGCTCGC
TCCACGGTGGCGGCGAGGTCGTTGGAAATGCGGGCCATGAGCTGCGAGA
AGGCGTTGAGG
CCTCCCTCGTTCCAGACGCGGCTGTAGACCACGCCCCCTTCGGCATCGC
GGGCGCGCATG
ACCACCTGCGCGAGATTGAGCTCCACGTGCCGGGCGAAGACGGCGTAGT
TTCGCAGGCGC
TGAAAGAGGTAGTTGAGGGTGGTGGCGGTGTGTTCTGCCACGAAGAAGT
ACATAACCCAG
CGTCGCAACGTGGATTCGTTGATATCCCCCAAGGCCTCAAGGCGCTCCA
TGGCCTCGTAG
AAGTCCACGGCGAAGTTGAAAAACTGGGAGTTGCGCGCCGACACGGTTA
ACTCCTCCTCC
AGAAGACGGATGAGCTCGGCGACAGTGTCGCGCACCTCGCGCTCAAAGG
CTACAGGGGCC
TCTTCTTCTTCTTCAATCTCCTCTTCCATAAGGGCCTCCCCTTCTTCTT
CTTCTGGCGGC
GGTGGGGGAGGGGGGACACGGCGGCGACGACGGCGCACCGGGAGGCGGT
CGACAAAGCGC
TCGATCATCTCCCCGCGGCGACGGCGCATGGTCTCGGTGACGGCGCGGC
CGTTCTCGCGG
GGGCGCAGTTGGAAGACGCCGCCCGTCATGTCCCGGTTATGGGTTGGCG
GGGGGCTGCCA
TGCGGCAGGGATACGGCGCTAACGATGCATCTCAACAATTGTTGTGTAG
GTACTCCGCCG
CCGAGGGACCTGAGCGAGTCCGCATCGACCGGATCGGAAAACCTCTCGA
GAAAGGCGTCT
AACCAGTCACAGTCGCAAGGTAGGCTGAGCACCGTGGCGGGCGGCAGCG
GGCGGCGGTCG
GGGTTGTTTCTGGCGGAGGTGCTGCTGATGATGTAATTAAAGTAGGCGG
TCTTGAGACGG
CGGATGGTCGACAGAAGCACCATGTCCTTGGGTCCGGCCTGCTGAATGC
GCAGGCGGTCG
GCCATGCCCCAGGCTTCGTTTTGACATCGGCGCAGGTCTTTGTAGTAGT
CTTGCATGAGC
CTTTCTACCGGCACTTCTTCTTCTCCTTCCTCTTGTCCTGCATCTCTTG
CATCTATCGCT
GCGGCGGCGGCGGAGTTTGGCCGTAGGTGGCGCCCTCTTCCTCCCATGC
GTGTGACCCCG
AAGCCCCTCATCGGCTGAAGCAGGGCTAGGTCGGCGACAACGCGCTCGG
CTAATATGGCC
TGCTGCACCTGCGTGAGGGTAGACTGGAAGTCATCCATGTCCACAAAGC
GGTGGTATGCG
CCCGTGTTGATGGTGTAAGTGCAGTTGGCCATAACGGACCAGTTAACGG
TCTGGTGACCC
GGCTGCGAGAGCTCGGTGTACCTGAGACGCGAGTAAGCCCTCGAGTCAA
ATACGTAGTCG
TTGCAAGTCCGCACCAGGTACTGGTATCCCACCAAAAAGTGCGGCGGCG
GCTGGCGGTAG
AGGGGCCAGCGTAGGGTGGCCGGGGCTCCGGGGGCGAGATCTTCCAACA
TAAGGCGATGA
TATCCGTAGATGTACCTGGACATCCAGGTGATGCCGGCGGCGGTGGTGG
AGGCGCGCGGA
AAGTCGCGGACGCGGTTCCAGATGTTGCGCAGCGGCAAAAAGTGCTCCA
TGGTCGGGACG
CTCTGGCCGGTCAGGCGCGCGCAATCGTTGACGCTCTAGACCGTGCAAA
AGGAGAGCCTG
TAAGCGGGCACTCTTCCGTGGTCTGGTGGATAAATTCGCAAGGGTATCA
TGGCGGACGAC
CGGGGTTCGAGCCCCGTATCCGGCCGTCCGCCGTGATCCATGCGGTTAC
CGCCCGCGTGT
CGAACCCAGGTGTGCGACGTCAGACAACGGGGGAGTGCTCCTTTTGGCT
TCCTTCCAGGC
GCGGCGGCTGCTGCGCTAGCTTTTTTGGCCACTGGCCGCGCGCAGCGTA
AGCGGTTAGGC
TGGAAAGCGAAAGCATTAAGTGGCTCGCTCCCTGTAGCCGGAGGGTTAT
TTTCCAAGGGT
TGAGTCGCGGGACCCCCGGTTCGAGTCTCGGACCGGCCGGACTGCGGCG
AACGGGGGTTT
GCCTCCCCGTCATGCAAGACCCCGCTTGCAAATTCCTCCGGAAACAGGG
ACGAGCCCCTT
TTTTGCTTTTCCCAGATGCATCCGGTGCTGCGGCAGATGCGCCCCCCTC
CTCAGCAGCGG
CAAGAGCAAGAGCAGCGGCAGACATGCAGGGCACCCTCCCCTCCTCCTA
CCGCGTCAGGA
GGGGCGACATCCGCGGTTGACGCGGCAGCAGATGGTGATTACGAACCCC
CGCGGCGCCGG
GCCCGGCACTACCTGGACTTGGAGGAGGGCGAGGGCCTGGCGCGGCTAG
GAGCGCCCTCT
CCTGAGCGGTACCCAAGGGTGCAGCTGAAGCGTGATACGCGTGAGGCGT
ACGTGCCGCGG
CAGAACCTGTTTCGCGACCGCGAGGGAGAGGAGCCCGAGGAGATGCGGG
ATCGAAAGTTC
CACGCAGGGCGCGAGCTGCGGCATGGCCTGAATCGCGAGCGGTTGCTGC
GCGAGGAGGAC
TTTGAGCCCGACGCGCGAACCGGGATTAGTCCCGCGCGCGCACACGTGG
CGGCCGCCGAC
CTGGTAACCGCATACGAGCAGACGGTGAACCAGGAGATTAACTTTCAAA
AAAGCTTTAAC
AACCACGTGCGTACGCTTGTGGCGCGCGAGGAGGTGGCTATAGGACTGA
TGCATCTGTGG
GACTTTGTAAGCGCGCTGGAGCAAAACCCAAATAGCAAGCCGCTCATGG
CGCAGCTGTTC
CTTATAGTGCAGCACAGCAGGGACAACGAGGCATTCAGGGATGCGCTGC
TAAACATAGTA
GAGCCCGAGGGCCGCTGGCTGCTCGATTTGATAAACATCCTGCAGAGCA
TAGTGGTGCAG
GAGCGCAGCTTGAGCCTGGCTGACAAGGTGGCCGCCATCAACTATTCCA
TGCTTAGCCTG
GGCAAGTTTTACGCCCGCAAGATATACCATACCCCTTACGTTCCCATAG
ACAAGGAGGTA
AAGATCGAGGGGTTCTACATGCGCATGGCGCTGAAGGTGCTTACCTTGA
GCGACGACCTG
GGCGTTTATCGCAACGAGCGCATCCACAAGGCCGTGAGCGTGAGCCGGC
GGCGCGAGCTC
AGCGACCGCGAGCTGATGCACAGCCTGCAAAGGGCCCTGGCTGGCACGG
GCAGCGGCGAT
AGAGAGGCCGAGTCCTACTTTGACGCGGGCGCTGACCTGCGCTGGGCCC
CAAGCCGACGC
GCCCTGGAGGCAGCTGGGGCCGGACCTGGGCTGGCGGTGGCACCCGCGC
GCGCTGGCAAC
GTCGGCGGCGTGGAGGAATATGACGAGGACGATGAGTACGAGCCAGAGG
ACGGCGAGTAC
TAAGCGGTGATGTTTCTGATCAGATGATGCAAGACGCAACGGACCCGGC
GGTGCGGGCGG
CGCTGCAGAGCCAGCCGTCCGGCCTTAACTCCACGGACGACTGGCGCCA
GGTCATGGACC
GCATCATGTCGCTGACTGCGCGCAATCCTGACGCGTTCCGGCAGCAGCC
GCAGGCCAACC
GGCTCTCCGCAATTCTGGAAGCGGTGGTCCCGGCGCGCGCAAACCCCAC
GCACGAGAAGG
TGCTGGCGATCGTAAACGCGCTGGCCGAAAACAGGGCCATCCGGCCCGA
CGAGGCCGGCC
TGGTCTACGACGCGCTGCTTCAGCGCGTGGCTCGTTACAACAGCGGCAA
CGTGCAGACCA
ACCTGGACCGGCTGGTGGGGGATGTGCGCGAGGCCGTGGCGCAGCGTGA
GCGCGCGCAGC
AGCAGGGCAACCTGGGCTCCATGGTTGCACTAAACGCCTTCCTGAGTAC
ACAGCCCGCCA
ACGTGCCGCGGGGACAGGAGGACTACACCAACTTTGTGAGCGCACTGCG
GCTAATGGTGA
CTGAGACACCGCAAAGTGAGGTGTACCAGTCTGGGCCAGACTATTTTTT
CCAGACCAGTA
GACAAGGCCTGCAGACCGTAAACCTGAGCCAGGCTTTCAAAAACTTGCA
GGGGCTGTGGG
GGGTGCGGGCTCCCACAGGCGACCGCGCGACCGTGTCTAGCTTGCTGAC
GCCCAACTCGC
GCCTGTTGCTGCTGCTAATAGCGCCCTTCACGGACAGTGGCAGCGTGTC
CCGGGACACAT
ACCTAGGTCACTTGCTGACACTGTACCGCGAGGCCATAGGTCAGGCGCA
TGTGGACGAGC
ATACTTTCCAGGAGATTACAAGTGTCAGCCGCGCGCTGGGGCAGGAGGA
CACGGGCAGCC
TGGAGGCAACCCTAAACTACCTGCTGACCAACCGGCGGCAGAAGATCCC
CTCGTTGCACA
GTTTAAACAGCGAGGAGGAGCGCATTTTGCGCTACGTGCAGCAGAGCGT
GAGCCTTAACC
TGATGCGCGACGGGGTAACGCCCAGCGTGGCGCTGGACATGACCGCGCG
CAACATGGAAC
CGGGCATGTATGCCTCAAACCGGCCGTTTATCAACCGCCTAATGGACTA
CTTGCATCGCG
CGGCCGCCGTGAACCCCGAGTATTTCACCAATGCCATCTTGAACCCGCA
CTGGCTACCGC
CCCCTGGTTTCTACACCGGGGGATTCGAGGTGCCCGAGGGTAACGATGG
ATTCCTCTGGG
ACGACATAGACGACAGCGTGTTTTCCCCGCAACCGCAGACCCTGCTAGA
GTTGCAACAGC
GCGAGCAGGCAGAGGCGGCGCTGCGAAAGGAAAGCTTCCGCAGGCCAAG
CAGCTTGTCCG
ATCTAGGCGCTGCGGCCCCGCGGTCAGATGCTAGTAGCCCATTTCCAAG
CTTGATAGGGT
CTCTTACCAGCACTCGCACCACCCGCCCGCGCCTGCTGGGCGAGGAGGA
GTACCTAAACA
ACTCGCTGCTGCAGCCGCAGCGCGAAAAAAACCTGCCTCCGGCATTTCC
CAACAACGGGA
TAGAGAGCCTAGTGGACAAGATGAGTAGATGGAAGACGTACGCGCAGGA
GCACAGGGACG
TGCCAGGCCCGCGCCCGCCCACCCGTCGTCAAAGGCACGACCGTCAGCG
GGGTCTGGTGT
GGGAGGACGATGACTCGGCAGACGACAGCAGCGTCCTGGATTTGGGAGG
GAGTGGCAACC
CGTTTGCGCACCTTCGCCCCAGGCTGGGGAGAATGTTTTAAAAAAAAAA
AAGCATGATGC
AAAATAAAAAACTCACCAAGGCCATGGCACCGAGCGTTGGTTTTCTTGT
ATTCCCCTTAG
TATGCGGCGCGCGGCGATGTATGAGGAAGGTCCTCCTCCCTCCTACGAG
AGTGTGGTGAG
CGCGGCGCCAGTGGCGGCGGCGCTGGGTTCTCCCTTCGATGCTCCCCTG
GACCCGCCGTT
TGTGCCTCCGCGGTACCTGCGGCCTACCGGGGGGAGAAACAGCATCCGT
TACTCTGAGTT
GGCACCCCTATTCGACACCACCCGTGTGTACCTGGTGGACAACAAGTCA
ACGGATGTGGC
ATCCCTGAACTACCAGAACGACCACAGCAACTTTCTGACCACGGTCATT
CAAAACAATGA
CTACAGCCCGGGGGAGGCAAGCACACAGACCATCAATCTTGACGACCGG
TCGCACTGGGG
CGGCGACCTGAAAACCATCCTGCATACCAACATGCCAAATGTGAACGAG
TTCATGTTTAC
CAATAAGTTTAAGGCGCGGGTGATGGTGTCGCGCTTGCCTACTAAGGAC
AATCAGGTGGA
GCTGAAATACGAGTGGGTGGAGTTCACGCTGCCCGAGGGCAACTACTCC
GAGACCATGAC
CATAGACCTTATGAACAACGCGATCGTGGAGCACTACTTGAAAGTGGGC
AGACAGAACGG
GGTTCTGGAAAGCGACATCGGGGTAAAGTTTGACACCCGCAACTTCAGA
CTGGGGTTTGA
CCCCGTCACTGGTCTTGTCATGCCTGGGGTATATACAAACGAAGCCTTC
CATCCAGACAT
CATTTTGCTGCCAGGATGCGGGGTGGACTTCACCCACAGCCGCCTGAGC
AACTTGTTGGG
CATCCGCAAGCGGCAACCCTTCCAGGAGGGCTTTAGGATCACCTACGAT
GATCTGGAGGG
TGGTAACATTCCCGCACTGTTGGATGTGGACGCCTACCAGGCGAGCTTG
AAAGATGACAC
CGAACAGGGCGGGGGTGGCGCAGGCGGCAGCAACAGCAGTGGCAGCGGC
GCGGAAGAGAA
CTCCAACGCGGCAGCCGCGGCAATGCAGCCGGTGGAGGACATGAACGAT
CATGCCATTCG
CGGCGACACCTTTGCCACACGGGCTGAGGAGAAGCGCGCTGAGGCCGAA
GCAGCGGCCGA
AGCTGCCGCCCCCGCTGCGCAACCCGAGGTCGAGAAGCCTCAGAAGAAA
CCGGTGATCAA
ACCCCTGACAGAGGACAGCAAGAAACGCAGTTACAACCTAATAAGCAAT
GACAGCACCTT
CACCCAGTACCGCAGCTGGTACCTTGCATACAACTACGGCGACCCTCAG
ACCGGAATCCG
CTCATGGACCCTGCTTTGCACTCCTGACGTAACCTGCGGCTCGGAGCAG
GTCTACTGGTC
GTTGCCAGACATGATGCAAGACCCCGTGACCTTCCGCTCCACGCGCCAG
ATCAGCAACTT
TCCGGTGGTGGGCGCCGAGCTGTTGCCCGTGCACTCCAAGAGCTTCTAC
AACGACCAGGC
CGTCTACTCCCAACTCATCCGCCAGTTTACCTCTCTGACCCACGTGTTC
AATCGCTTTCC
CGAGAACCAGATTTTGGCGCGCCCGCCAGCCCCCACCATCACCACCGTC
AGTGAAAACGT
TCCTGCTCTCACAGATCACGGGACGCTACCGCTGCGCAACAGCATCGGA
GGAGTCCAGCG
AGTGACCATTACTGACGCCAGACGCCGCACCTGCCCCTACGTTTACAAG
GCCCTGGGCAT
AGTCTCGCCGCGCGTCCTATCGAGCCGCACTTTTTGAGCAAGCATGTCC
ATCCTTATATC
GCCCAGCAATAACACAGGCTGGGGCCTGCGCTTCCCAAGCAAGATGTTT
GGCGGGGCCAA
GAAGCGCTCCGACCAACACCCAGTGCGCGTGCGCGGGCACTACCGCGCG
CCCTGGGGCGC
GCACAAACGCGGCCGCACTGGGCGCACCACCGTCGATGACGCCATCGAC
GCGGTGGTGGA
GGAGGCGCGCAACTACACGCCCACGCCGCCACCAGTGTCCACAGTGGAC
GCGGCCATTCA
GACCGTGGTGCGCGGAGCCCGGCGCTATGCTAAAATGAAGAGACGGCGG
AGGCGCGTAGC
ACGTCGCCACCGCCGCCGACCCGGCACTGCCGCCCAACGCGCGGCGGCG
GCCCTGCTTAA
CCGCGCACGTCGCACCGGCCGACGGGCGGCCATGCGGGCCGCTCGAAGG
CTGGCCGCGGG
TATTGTCACTGTGCCCCCCAGGTCCAGGCGACGAGCGGCCGCCGCAGCA
GCCGCGGCCAT
TAGTGCTATGACTCAGGGTCGCAGGGGCAACGTGTATTGGGTGCGCGAC
TCGGTTAGCGG
CCTGCGCGTGCCCGTGCGCACCCGCCCCCCGCGCAACTAGATTGCAAGA
AAAAACTACTT
AGACTCGTACTGTTGTATGTATCCAGCGGCGGCGGCGCGCAACGAAGCT
ATGTCCAAGCG
CAAAATCAAAGAAGAGATGCTCCAGGTCATCGCGCCGGAGATCTATGGC
CCCCCGAAGAA
GGAAGAGCAGGATTACAAGCCCCGAAAGCTAAAGCGGGTCAAAAAGAAA
AAGAAAGATGA
TGATGATGAACTTGACGACGAGGTGGAACTGCTGCACGCTACCGCGCCC
AGGCGACGGGT
ACAGTGGAAAGGTCGACGCGTAAAACGTGTTTTGCGACCCGGCACCACC
GTAGTCTTTAC
GCCCGGTGAGCGCTCCACCCGCACCTACAAGCGCGTGTATGATGAGGTG
TACGGCGACGA
GGACCTGCTTGAGCAGGCCAACGAGCGCCTCGGGGAGTTTGCCTACGGA
AAGCGGCATAA
GGACATGCTGGCGTTGCCGCTGGACGAGGGCAACCCAACACCTAGCCTA
AAGCCCGTAAC
ACTGCAGCAGGTGCTGCCCGCGCTTGCACCGTCCGAAGAAAAGCGCGGC
CTAAAGCGCGA
GTCTGGTGACTTGGCACCCACCGTGCAGCTGATGGTACCCAAGCGCCAG
CGACTGGAAGA
TGTCTTGGAAAAAATGACCGTGGAACCTGGGCTGGAGCCCGAGGTCCGC
GTGCGGCCAAT
CAAGCAGGTGGCGCCGGGACTGGGCGTGCAGACCGTGGACGTTCAGATA
CCCACTACCAG
TAGCACCAGTATTGCCACCGCCACAGAGGGCATGGAGACACAAACGTCC
CCGGTTGCCTC
AGCGGTGGCGGATGCCGCGGTGCAGGCGGTCGCTGCGGCCGCGTCCAAG
ACCTCTACGGA
GGTGCAAACGGACCCGTGGATGTTTCGCGTTTCAGCCCCCCGGCGCCCG
CGCGGTTCGAG
GAAGTACGGCGCCGCCAGCGCGCTACTGCCCGAATATGCCCTACATCCT
TCCATTGCGCC
TACCCCCGGCTATCGTGGCTACACCTACCGCCCCAGAAGACGAGCAACT
ACCCGACGCCG
AACCACCACTGGAACCCGCCGCCGCCGTCGCCGTCGCCAGCCCGTGCTG
GCCCCGATTTC
CGTGCGCAGGGTGGCTCGCGAAGGAGGCAGGACCCTGGTGCTGCCAACA
GCGCGCTACCA
CCCCAGCATCGTTTAAAAGCCGGTCTTTGTGGTTCTTGCAGATATGGCC
CTCACCTGCCG
CCTCCGTTTCCCGGTGCCGGGATTCCGAGGAAGAATGCACCGTAGGAGG
GGCATGGCCGG
CCACGGCCTGACGGGCGGCATGCGTCGTGCGCACCACCGGCGGCGGCGC
GCGTCGCACCG
TCGCATGCGCGGCGGTATCCTGCCCCTCCTTATTCCACTGATCGCCGCG
GCGATTGGCGC
CGTGCCCGGAATTGCATCCGTGGCCTTGCAGGCGCAGAGACACTGATTA
AAAACAAGTTG
CATGTGGAAAAATCAAAATAAAAAGTCTGGACTCTCACGCTCGCTTGGT
CCTGTAACTAT
TTTGTAGAATGGAAGACATCAACTTTGCGTCTCTGGCCCCGCGACACGG
CTCGCGCCCGT
TCATGGGAAACTGGCAAGATATCGGCACCAGCAATATGAGCGGTGGCGC
CTTCAGCTGGG
GCTCGCTGTGGAGCGGCATTAAAAATTTCGGTTCCACCGTTAAGAACTA
TGGCAGCAAGG
CCTGGAACAGCAGCACAGGCCAGATGCTGAGGGATAAGTTGAAAGAGCA
AAATTTCCAAC
AAAAGGTGGTAGATGGCCTGGCCTCTGGCATTAGCGGGGTGGTGGACCT
GGCCAACCAGG
CAGTGCAAAATAAGATTAACAGTAAGCTTGATCCCCGCCCTCCCGTAGA
GGAGCCTCCAC
CGGCCGTGGAGACAGTGTCTCCAGAGGGGCGTGGCGAAAAGCGTCCGCG
CCCCGACAGGG
AAGAAACTCTGGTGACGCAAATAGACGAGCCTCCCTCGTACGAGGAGGC
ACTAAAGCAAG
GCCTGCCCACCACCCGTCCCATCGCGCCCATGGCTACCGGAGTGCTGGG
CCAGCACACAC
CCGTAACGCTGGACCTGCCTCCCCCCGCCGACACCCAGCAGAAACCTGT
GCTGCCAGGCC
CGACCGCCGTTGTTGTAACCCGTCCTAGCCGCGCGTCCCTGCGCCGCGC
CGCCAGCGGTC
CGCGATCGTTGCGGCCCGTAGCCAGTGGCAACTGGCAAAGCACACTGAA
CAGCATCGTGG
GTCTGGGGGTGCAATCCCTGAAGCGCCGACGATGCTTCTGAATAGCTAA
CGTGTCGTATG
TGTGTCATGTATGCGTCCATGTCGCCGCCAGAGGAGCTGCTGAGCCGCC
GCGCGCCCGCT
TTCCAAGATGGCTACCCCTTCGATGATGCCGCAGTGGTCTTACATGCAC
ATCTCGGGCCA
GGACGCCTCGGAGTACCTGAGCCCCGGGCTGGTGCAGTTTGCCCGCGCC
ACCGAGACGTA
CTTCAGCCTGAATAACAAGTTTAGAAACCCCACGGTGGCGCCTACGCAC
GACGTGACCAC
AGACCGGTCCCAGCGTTTGACGCTGCGGTTCATCCCTGTGGACCGTGAG
GATACTGCGTA
CTCGTACAAGGCGCGGTTCACCCTAGCTGTGGGTGATAACCGTGTGCTG
GACATGGCTTC
CACGTACTTTGACATCCGCGGCGTGCTGGACAGGGGCCCTACTTTTAAG
CCCTACTCTGG
CACTGCCTACAACGCCCTGGCTCCCAAGGGTGCCCCAAATCCTTGCGAA
TGGGATGAAGC
TGCTACTGCTCTTGAAATAAACCTAGAAGAAGAGGACGATGACAACGAA
GACGAAGTAGA
CGAGCAAGCTGAGCAGCAAAAAACTCACGTATTTGGGCAGGCGCCTTAT
TCTGGTATAAA
TATTACAAAGGAGGGTATTCAAATAGGTGTCGAAGGTCAAACACCTAAA
TATGCCGATAA
AACATTTCAACCTGAACCTCAAATAGGAGAATCTCAGTGGTACGAAACT
GAAATTAATCA
TGCAGCTGGGAGAGTCCTTAAAAAGACTACCCCAATGAAACCATGTTAC
GGTTCATATGC
AAAACCCACAAATGAAAATGGAGGGCAAGGCATTCTTGTAAAGCAACAA
AATGGAAAGCT
AGAAAGTCAAGTGGAAATGCAATTTTTCTCAACTACTGAGGCGACCGCA
GGCAATGGTGA
TAACTTGACTCCTAAAGTGGTATTGTACAGTGAAGATGTAGATATAGAA
ACCCCAGACAC
TCATATTTCTTACATGCCCACTATTAAGGAAGGTAACTCACGAGAACTA
ATGGGCCAACA
ATCTATGCCCAACAGGCCTAATTACATTGCTTTTAGGGACAATTTTATT
GGTCTAATGTA
TTACAACAGCACGGGTAATATGGGTGTTCTGGCGGGCCAAGCATCGCAG
TTGAATGCTGT
TGTAGATTTGCAAGACAGAAACACAGAGCTTTCATACCAGCTTTTGCTT
GATTCCATTGG
TGATAGAACCAGGTACTTTTCTATGTGGAATCAGGCTGTTGACAGCTAT
GATCCAGATGT
TAGAATTATTGAAAATCATGGAACTGAAGATGAACTTCCAAATTACTGC
TTTCCACTGGG
AGGTGTGATTAATACAGAGACTCTTACCAAGGTAAAACCTAAAACAGGT
CAGGAAAATGG
ATGGGAAAAAGATGCTACAGAATTTTCAGATAAAAATGAAATAAGAGTT
GGAAATAATTT
TGCCATGGAAATCAATCTAAATGCCAACCTGTGGAGAAATTTCCTGTAC
TCCAACATAGC
GCTGTATTTGCCCGACAAGCTAAAGTACAGTCCTTCCAACGTAAAAATT
TCTGATAACCC
AAACACCTACGACTACATGAACAAGCGAGTGGTGGCTCCCGGGTTAGTG
GACTGCTACAT
TAACCTTGGAGCACGCTGGTCCCTTGACTATATGGACAACGTCAACCCA
TTTAACCACCA
CCGCAATGCTGGCCTGCGCTACCGCTCAATGTTGCTGGGCAATGGTCGC
TATGTGCCCTT
CCACATCCAGGTGCCTCAGAAGTTCTTTGCCATTAAAAACCTCCTTCTC
CTGCCGGGCTC
ATACACCTACGAGTGGAACTTCAGGAAGGATGTTAACATGGTTCTGCAG
AGCTCCCTAGG
AAATGACCTAAGGGTTGACGGAGCCAGCATTAAGTTTGATAGCATTTGC
CTTTACGCCAC
CTTCTTCCCCATGGCCCACAACACCGCCTCCACGCTTGAGGCCATGCTT
AGAAACGACAC
CAACGACCAGTCCTTTAACGACTATCTCTCCGCCGCCAACATGCTCTAC
CCTATACCCGC
CAACGCTACCAACGTGCCCATATCCATCCCCTCCCGCAACTGGGCGGCT
TTCCGCGGCTG
GGCCTTCACGCGCCTTAAGACTAAGGAAACCCCATCACTGGGCTCGGGC
TACGACCCTTA
TTACACCTACTCTGGCTCTATACCCTACCTAGATGGAACCTTTTACCTC
AACCACACCTT
TAAGAAGGTGGCCATTACCTTTGACTCTTCTGTCAGCTGGCCTGGCAAT
GACCGCCTGCT
TACCCCCAACGAGTTTGAAATTAAGCGCTCAGTTGACGGGGAGGGTTAC
AACGTTGCCCA
GTGTAACATGACCAAAGACTGGTTCCTGGTACAAATGCTAGCTAACTAC
AACATTGGCTA
CCAGGGCTTCTATATCCCAGAGAGCTACAAGGACCGCATGTACTCCTTC
TTTAGAAACTT
CCAGCCCATGAGCCGTCAGGTGGTGGATGATACTAAATACAAGGACTAC
CAACAGGTGGG
CATCCTACACCAACACAACAACTCTGGATTTGTTGGCTACCTTGCCCCC
ACCATGCGCGA
AGGACAGGCCTACCCTGCTAACTTCCCCTATCCGCTTATAGGCAAGACC
GCAGTTGACAG
CATTACCCAGAAAAAGTTTCTTTGCGATCGCACCCTTTGGCGCATCCCA
TTCTCCAGTAA
CTTTATGTCCATGGGCGCACTCACAGACCTGGGCCAAAACCTTCTCTAC
GCCAACTCCGC
CCACGCGCTAGACATGACTTTTGAGGTGGATCCCATGGACGAGCCCACC
CTTCTTTATGT
TTTGTTTGAAGTCTTTGACGTGGTCCGTGTGCACCGGCCGCACCGCGGC
GTCATCGAAAC
CGTGTACCTGCGCACGCCCTTCTCGGCCGGCAACGCCACAACATAAGCG
ATCGCAGCAGG
TTTCCCCAACTGACACAAAACGTGCAACTTGAAACTCCGCCTGGTCTTT
CCAGGTCTAGA
GGGGTAACACTTTGTACTGCGTTTGGCTCCACGCTCGATCCACTGGCGA
GTGTTAGTAAC
AGCACTGTTGCTTCGTAGCGGAGCATGACGGCCGTGGGAACTCCTCCTT
GGTAACAAGGA
CCCACGGGGCCAAAAGCCACGCCCACACGGGCCCGTCATGTGTGCAACC
CCAGCACGGCG
ACTTTACTGCGAAACCCACTTTAAAGTGACATTGAAACTGGTACCCACA
CACTGGTGACA
GGCTAAGGATGCCCTTCAGGTACCCCGAGGTAACACGCGACACTCGGGA
TCTGAGAAGGG
GACTGGGGCTTCTATAAAAGCGCTCGGTTTAAAAAGCTTCTATGCCTGA
ATANGTGACCG
GANGTCGGCACCTTTCCTTTGCAATTAATGACCCTGTATACGCCACCAT
GGCTATGATGG
AGGTCCAGGGGGGACCCAGCCTGGGACAGACCTGCGTGCTGATCGTGAT
CTTTACAGTGC
TCCTGCAGTCTCTCTGTGTGGCTGTAACTTACGTGTACTTTACCAACGA
GCTGAAGCAGA
TGCAGGACAAGTACTCCAAAAGTGGCATTGCTTGTTTCTTAAAAGAAGA
TGACAGTTATT
GGGACCCCAATGACGAAGAGAGTATGAACAGCCCCTGCTGGCAAGTCAA
GTGGCAACTCC
GTCAGCTCGTTAGAAAGATGATTTTGAGAACCTCTGAGGAAACCATTTC
TACAGTTCAAG
AAAAGCAACAAAATATTTCTCCCCTAGTGAGAGAAAGAGGTCCTCAGAG
AGTAGCAGCTC
ACATAACTGGGACCAGAGGAAGAAGCAACACATTGTCTTCTCCAAACTC
CAAGAATGAAA
AGGCTCTGGGCCGCAAAATAAACTCCTGGGAATCATCAAGGAGTGGGCA
TTCATTCCTGA
GCAACTTGCACTTGAGGAATGGTGAACTGGTCATCCATGAAAAAGGGTT
TTACTACATCT
ATTCCCAAACATACTTTCGATTTCAGGAGGAAATAAAAGAAAACACAAA
GAACGACAAAC
AAATGGTCCAATATATTTACAAATACACAAGTTATCCTGACCCTATATT
GTTGATGAAAA
GTGCTAGAAATAGTTGTTGGTCTAAAGATGCAGAATATGGACTCTATTC
CATCTATCAAG
GGGGAATATTTGAGCTTAAGGAAAATGACAGAATTTTTGTTTCTGTAAC
AAATGAGCACT
TAATAGACATGGACCATGAAGCCAGTTTTTTCGGGGCCTTTTTAGTTGG
CTAAGTATACT
TCGAATGCATGCGATCGCAGAAGCAAGCAACATCAACAACAGCTGCCGC
CATGGGCTCCA
GTGAGCAGGAACTGAAAGCCATTGTCAAAGATCTTGGTTGTGGGCCATA
TTTTTTGGGCA
CCTATGACAAGCGCTTTCCAGGCTTTGTTTCTCCACACAAGCTCGCCTG
CGCCATAGTCA
ATACGGCCGGTCGCGAGACTGGGGGCGTACACTGGATGGCCTTTGCCTG
GAACCCGCACT
CAAAAACATGCTACCTCTTTGAGCCCTTTGGCTTTTCTGACCAGCGACT
CAAGCAGGTTT
ACCAGTTTGAGTACGAGTCACTCCTGCGCCGTAGCGCCATTGCTTCTTC
CCCCGACCGCT
GTATAACGCTGGAAAAGTCCACCCAAAGCGTACAGGGGCCCAACTCGGC
CGCCTGTGGAC
TATTCTGCTGCATGTTTCTCCACGCCTTTGCCAACTGGCCCCAAACTCC
CATGGATCACA
ACCCCACCATGAACCTTATTACCGGGGTACCCAACTCCATGCTCAACAG
TCCCCAGGTAC
AGCCCACCCTGCGTCGCAACCAGGAACAGCTCTACAGCTTCCTGGAGCG
CCACTCGCCCT
ACTTCCGCAGCCACAGTGCGCAGATTAGGAGCGCCACTTCTTTTTGTCA
CTTGAAAAACA
TGTAAAAAATAATGTACTAGAGACACTTTCAATAAAGGCAAATGCTTTT
ATTTGTACACT
CTCGGGTGATTATTTACCCCCACCCTTGCCGTCTGCGCCGTTTAAAAAT
CAAAGGGGTTC
TGCCGCGCATCGCTATGCGCCACTGGCAGGGACACGTTGCGATACTGGT
GTTTAGTGCTC
CACTTAAACTCAGGCACAACCATCCGCGGCAGCTCGGTGAAGTTTTCAC
TCCACAGGCTG
CGCACCATCACCAACGCGTTTAGCAGGTCGGGCGCCGATATCTTGAAGT
CGCAGTTGGGG
CCTCCGCCCTGCGCGCGCGAGTTGCGATACACAGGGTTGCAGCACTGGA
ACACTATCAGC
GCCGGGTGGTGCACGCTGGCCAGCACGCTCTTGTCGGAGATCAGATCCG
CGTCCAGGTCC
TCCGCGTTGCTCAGGGCGAACGGAGTCAACTTTGGTAGCTGCCTTCCCA
AAAAGGGCGCG
TGCCCAGGCTTTGAGTTGCACTCGCACCGTAGTGGCATCAAAAGGTGAC
CGTGCCCGGTC
TGGGCGTTAGGATACAGCGCCTGCATAAAAGCCTTGATCTGCTTAAAAG
CCACCTGAGCC
TTTGCGCCTTCAGAGAAGAACATGCCGCAAGACTTGCCGGAAAACTGAT
TGGCCGGACAG
GCCGCGTCGTGCACGCAGCACCTTGCGTCGGTGTTGGAGATCTGCACCA
CATTTCGGCCC
CACCGGTTCTTCACGATCTTGGCCTTGCTAGACTGCTCCTTCAGCGCGC
GCTGCCCGTTT
TCGCTCGTCACATCCATTTCAATCACGTGCTCCTTATTTATCATAATGC
TTCCGTGTAGA
CACTTAAGCTCGCCTTCGATCTCAGCGCAGCGGTGCAGCCACAACGCGC
AGCCCGTGGGC
TCGTGATGCTTGTAGGTCACCTCTGCAAACGACTGCAGGTACGCCTGCA
GGAATCGCCCC
ATCATCGTCACAAAGGTCTTGTTGCTGGTGAAGGTCAGCTGCAACCCGC
GGTGCTCCTCG
TTCAGCCAGGTCTTGCATACGGCCGCCAGAGCTTCCACTTGGTCAGGCA
GTAGTTTGAAG
TTCGCCTTTAGATCGTTATCCACGTGGTACTTGTCCATCAGCGCGCGCG
CAGCCTCCATG
CCCTTCTCCCACGCAGACACGATCGGCACACTCAGCGGGTTCATCACCG
TAATTTCACTT
TCCGCTTCGCTGGGCTCTTCCTCTTCCTCTTGCGTCCGCATACCACGCG
CCACTGGGTCG
TCTTCATTCAGCCGCCGCACTGTGCGCTTACCTCCTTTGCCATGCTTGA
TTAGCACCGGT
GGGTTGCTGAAACCCACCATTTGTAGCGCCACATCTTCTCTTTCTTCCT
CGCTGTCCACG
ATTACCTCTGGTGATGGCGGGCGCTCGGGCTTGGGAGAAGGGCGCTTCT
TTTTCTTCTTG
GGCGCAATGGCCAAATCCGCCGCCGAGGTCGATGGCCGCGGGCTGGGTG
TGCGCGGCACC
AGCGCGTCTTGTGATGAGTCTTCCTCGTCCTCGGACTCGATACGCCGCC
TCATCCGCTTT
TTTGGGGGCGCCCGGGGAGGCGGCGGCGACGGGGACGGGGACGACACGT
CCTCCATGGTT
GGGGGACGTCGCGCCGCACCGCGTCCGCGCTCGGGGGTGGTTTCGCGCT
GCTCCTCTTCC
CGACTGGCCATTTCCTTCTCCTATAGGCAGAAAAAGATCATGGAGTCAG
TCGAGAAGAAG
GACAGCCTAACCGCCCCCTCTGAGTTCGCCACCACCGCCTCCACCGATG
CCGCCAACGCG
CCTACCACCTTCCCCGTCGAGGCACCCCCGCTTGAGGAGGAGGAAGTGA
TTATCGAGCAG
GACCCAGGTTTTGTAAGCGAAGACGACGAGGACCGCTCAGTACCAACAG
AGGATAAAAAG
CAAGACCAGGACAACGCAGAGGCAAACGAGGAACAAGTCGGGCGGGGGG
ACGAAAGGCAT
GGCGACTACCTAGATGTGGGAGACGACGTGCTGTTGAAGCATCTGCAGC
GCCAGTGCGCC
ATTATCTGCGACGCGTTGCAAGAGCGCAGCGATGTGCCCCTCGCCATAG
CGGATGTCAGC
CTTGCCTACGAACGCCACCTATTCTCACCGCGCGTACCCCCCAAACGCC
AAGAAAACGGC
ACATGCGAGCCCAACCCGCGCCTCAACTTCTACCCCGTATTTGCCGTGC
CAGAGGTGCTT
GCCACCTATCACATCTTTTTCCAAAACTGCAAGATACCCCTATCCTGCC
GTGCCAACCGC
AGCCGAGCGGACAAGCAGCTGGCCTTGCGGCAGGGCGCTGTCATACCTG
ATATCGCCTCG
CTCAACGAAGTGCCAAAAATCTTTGAGGGTCTTGGACGCGACGAGAAGC
GCGCGGCAAAC
GCTCTGCAACAGGAAAACAGCGAAAATGAAAGTCACTCTGGAGTGTTGG
TGGAACTCGAG
GGTGACAACGCGCGCCTAGCCGTACTAAAACGCAGCATCGAGGTCACCC
ACTTTGCCTAC
CCGGCACTTAACCTACCCCCCAAGGTCATGAGCACAGTCATGAGTGAGC
TGATCGTGCGC
CGTGCGCAGCCCCTGGAGAGGGATGCAAATTTGCAAGAACAAACAGAGG
AGGGCCTACCC
GCAGTTGGCGACGAGCAGCTAGCGCGCTGGCTTCAAACGCGCGAGCCTG
CCGACTTGGAG
GAGCGACGCAAACTAATGATGGCCGCAGTGCTCGTTACCGTGGAGCTTG
AGTGCATGCAG
CGGTTCTTTGCTGACCCGGAGATGCAGCGCAAGCTAGAGGAAACATTGC
ACTACACCTTT
CGACAGGGCTACGTACGCCAGGCCTGCAAGATCTCCAACGTGGAGCTCT
GCAACCTGGTC
TCCTACCTTGGAATTTTGCACGAAAACCGCCTTGGGCAAAACGTGCTTC
ATTCCACGCTC
AAGGGCGAGGCGCGCCGCGACTACGTCCGCGACTGCGTTTACTTATTTC
TATGCTACACC
TGGCAGACGGCCATGGGCGTTTGGCAGCAGTGCTTGGAGGAGTGCAACC
TCAAGGAGCTG
CAGAAACTGCTAAAGCAAAACTTGAAGGACCTATGGACGGCCTTCAACG
AGCGCTCCGTG
GCCGCGCACCTGGCGGACATCATTTTCCCCGAACGCCTGCTTAAAACCC
TGCAACAGGGT
CTGCCAGACTTCACCAGTCAAAGCATGTTGCAGAACTTTAGGAACTTTA
TCCTAGAGCGC
TCAGGAATCTTGCCCGCCACCTGCTGTGCACTTCCTAGCGACTTTGTGC
CCATTAAGTAC
CGCGAATGCCCTCCGCCGCTTTGGGGCCACTGCTACCTTCTGCAGCTAG
CCAACTACCTT
GCCTACCACTCTGACATAATGGAAGACGTGAGCGGTGACGGTCTACTGG
AGTGTCACTGT
CGCTGCAACCTATGCACCCCGCACCGCTCCCTGGTTTGCAATTCGCAGC
TGCTTAACGAA
AGTCAAATTATCGGTACCTTTGAGCTGCAGGGTCCCTCGCCTGACGAAA
AGTCCGCGGCT
CCGGGGTTGAAACTCACTCCGGGGCTGTGGACGTCGGCTTACCTTCGCA
AATTTGTACCT
GAGGACTACCACGCCCACGAGATTAGGTTCTACGAAGACCAATCCCGCC
CGCCAAATGCG
GAGCTTACCGCCTGCGTCATTACCCAGGGCCACATTCTTGGCCAATTGC
AAGCCATCAAC
AAAGCCCGCCAAGAGTTTCTGCTACGAAAGGGACGGGGGGTTTACTTGG
ACCCCCAGTCC
GGCGAGGAGCTCAACCCAATCCCCCCGCCGCCGCAGCCCTATCAGCAGC
AGCCGCGGGCC
CTTGCTTCCCAGGATGGCACCCAAAAAGAAGCTGCAGCTGCCGCCGCCA
CCCACGGACGA
GGAGGAATACTGGGACAGTCAGGCAGAGGAGGTTTTGGACGAGGAGGAG
GAGGACATGAT
GGAAGACTGGGAGAGCCTAGACGAGGAAGCTTCCGAGGTCGAAGAGGTG
TCAGACGAAAC
ACCGTCACCCTCGGTCGCATTCCCCTCGCCGGCGCCCCAGAAATCGGCA
ACCGGTTCCAG
CATGGCTACAACCTCCGCTCCTCAGGCGCCGCCGGCACTGCCCGTTCGC
CGACCCAACCG
TAGATGGGACACCACTGGAACCAGGGCCGGTAAGTCCAAGCAGCCGCCG
CCGTTAGCCCA
AGAGCAACAACAGCGCCAAGGCTACCGCTCATGGCGCGGGCACAAGAAC
GCCATAGTTGC
TTGCTTGCAAGACTGTGGGGGCAACATCTCCTTCGCCCGCCGCTTTCTT
CTCTACCATCA
CGGCGTGGCCTTCCCCCGTAACATCCTGCATTACTACCGTCATCTCTAC
AGCCCATACTG
CACCGGCGGCAGCGGCAGCGGCAGCAACAGCAGCGGCCACACAGAAGCA
AAGGCGACCGG
ATAGCAAGACTCTGACAAAGCCCAAGAAATCCACAGCGGCGGCAGCAGC
AGGAGGAGGAG
CGCTGCGTCTGGCGCCCAACGAACCCGTATCGACCCGCGAGCTTAGAAA
CAGGATTTTTC
CCACTCTGTATGCTATATTTCAACAGAGCAGGGGCCAAGAACAAGAGCT
GAAAATAAAAA
ACAGGTCTCTGCGATCCCTCACCCGCAGCTGCCTGTATCACAAAAGCGA
AGATCAGCTTC
GGCGCACGCTGGAAGACGCGGAGGCTCTCTTCAGTAAATACTGCGCGCT
GACTCTTAAGG
ACTAGTTTCGCGCCCTTTCTCAAATTTAAGCGCGAAAACTACGTCATCT
CCAGCGGCCAC
ACCCGGCGCCAGCACCTGTCGTCAGCGCCATTATGAGCAAGGAAATTCC
CACGCCCTACA
TGTGGAGTTACCAGCCACAAATGGGACTTGCGGCTGGAGCTGCCCAAGA
CTACTCAACCC
GAATAAACTACATGAGCGCGGGACCCCACATGATATCCCGGGTCAACGG
AATCCGCGCCC
ACCGAAACCGAATTCTCTTGGAACAGGCGGCTATTACCACCACACCTCG
TAATAACCTTA
ATCCCCGTAGTTGGCCCGCTGCCCTGGTGTACCAGGAAAGTCCCGCTCC
CACCACTGTGG
TACTTCCCAGAGACGCCCAGGCCGAAGTTCAGATGACTAACTCAGGGGC
GCAGCTTGCGG
GCGGCTTTCGTCACAGGGTGCGGTCGCCCGGGCAGGGTATAACTCACCT
GACAATCAGAG
GGCGAGGTATTCAGCTCAACGACGAGTCGGTGAGCTCCTCGCTTGGTCT
CCGTCCGGACG
GGACATTTCAGATCGGCGGCGCCGGCCGTCCTTCATTCACGCCTCGTCA
GGCAATCCTAA
CTCTGCAGACCTCGTCCTCTGAGCCGCGCTCTGGAGGCATTGGAACTCT
GCAATTTATTG
AGGAGTTTGTGCCATCGGTCTACTTTAACCCCTTCTCGGGACCTCCCGG
CCACTATCCGG
ATCAATTTATTCCTAACTTTGACGCGGTAAAGGACTCGGCGGACGGCTA
CGACTGAATGT
TAAGTGGAGAGGCAGAGCAACTGCGCCTGAAACACCTGGTCCACTGTCG
CCGCCACAAGT
GCTTTGCCCGCGACTCCGGTGAGTTTTGCTACTTTGAATTGCCCGAGGA
TCATATCGAGG
GCCCGGCGCACGGCGTCCGGCTTACCGCCCAGGGAGAGCTTGCCCGTAG
CCTGATTCGGG
AGTTTACCCAGCGCCCCCTGCTAGTTGAGCGGGACAGGGGACCCTGTGT
TCTCACTGTGA
TTTGCAACTGTCCTAACCTTGGATTACATCAAGATCTTTGTTGCCATCT
CTGTGCTGAGT
ATAATAAATACAGAAATTAAAATATACTGGGGCTCCTATCGCCATCCTG
TAAACGCCACC
GTCTTCACCCGCCCAAGCAAACCAAGGCGAACCTTACCTGGTACTTTTA
ACATCTCTCCC
TCTGTGATTTACAACAGTTTCAACCCAGACGGAGTGAGTCTACGAGAGA
ACCTCTCCGAG
CTCAGCTACTCCATCAGAAAAAACACCACCCTCCTTACCTGCCGGGAAC
GTACGAGTGCG
TCACCGGCCGCTGCACCACACCTACCGCCTGACCGTAAACCAGACTTTT
TCCGGACAGAC
CTCAATAACTCTGTTTACCAGAACAGGAGGTGAGCTTAGAAAACCCTTA
GGGTATTAGGC
CAAAGGCGCAGCTACTGTGGGGTTTATGAACAATTCAAGCAACTCTACG
GGCTATTCTAA
TTCAGGTTTCTCTAGAATCGGGGTTGGGGTTATTCTCTGTCTTGTGATT
CTCTTTATTCT
TATACTAACGCTTCTCTGCCTAAGGCTCGCCGCCTGCTGTGTGCACATT
TGCATTTATTG
TCAGCTTTTTAAACGCTGGGGTCGCCACCCAAGATGATTAGGTACATAA
TCCTAGGTTTA
CTCACCCTTGCGTCAGCCCACGGTACCACCCAAAAGGTGGATTTTAAGG
AGCCAGCCTGT
AATGTTACATTCGCAGCTGAAGCTAATGAGTGCACCACTCTTATAAAAT
GCACCACAGAA
CATGAAAAGCTGCTTATTCGCCACAAAAACAAAATTGGCAAGTATGCTG
TTTATGCTATT
TGGCAGCCAGGTGACACTACAGAGTATAATGTTACAGTTTTCCAGGGTA
AAAGTCATAAA
ACTTTTATGTATACTTTTCCATTTTATGAAATGTGCGACATTACCATGT
ACATGAGCAAA
CAGTATAAGTTGTGGCCCCCACAAAATTGTGTGGAAAACACTGGCACTT
TCTGCTGCACT
GCTATGCTAATTACAGTGCTCGCTTTGGTCTGTACCCTACTCTATATTA
AATACAAAAGC
AGACGCAGCTTTATTGAGGAAAAGAAAATGCCTTAATTTACTAAGTTAC
AAAGCTAATGT
CACCACTAACTGCTTTACTCGCTGCTTGCAAAACAAATTCAAAAAGTTA
GCATTATAATT
AGAATAGGATTTAAACCCCCCGGTCATTTCCTGCTCAATACCATTCCCC
TGAACAATTGA
CTCTATGTGGGATATGCTCCAGCGCTACAACCTTGAAGTCAGGCTTCCT
GGATGTCAGCA
TCTGACTTTGGCCAGCACCTGTCCCGCGGATTTGTTCCAGTCCAACTAC
AGCGACCCACC
CTAACAGAGATGACCAACACAACCAACGCGGCCGCCGCTACCGGACTTA
CATCTACCACA
AATACACCCCAAGTTTCTGCCTTTGTCAATAACTGGGATAACTTGGGCA
TGTGGTGGTTC
TCCATAGCGCTTATGTTTGTATGCCTTATTATTATGTGGCTCATCTGCT
GCCTAAAGCGC
AAACGCGCCCGACCACCCATCTATAGTCCCATCATTGTGCTACACCCAA
ACAATGATGGA
ATCCATAGATTGGACGGACTGAAACACATGTTCTTTTCTCTTACAGTAT
GATTAAATGAG
ACATGATTCCTCGAGTTTTTATATTACTGACCCTTGTTGCGCTTTTTTG
TGCGTGCTCCA
CATTGGCTGCGGTTTCTCACATCGAAGTAGACTGCATTCCAGCCTTCAC
AGTCTATTTGC
TTTACGGATTTGTCACCCTCACGCTCATCTGCAGCCTCATCACTGTGGT
CATCGCCTTTA
TCCAGTGCATTGACTGGGTCTGTGTGCGCTTTGCATATCTCAGACACCA
TCCCCAGTACA
GGGACAGGACTATAGCTGAGCTTCTTAGAATTCTTTAATTATGAAATTT
ACTGTGACTTT
TCTGCTGATTATTTGCACCCTATCTGCGTTTTGTTCCCCGACCTCCAAG
CCTCAAAGACA
TATATCATGCAGATTCACTCGTATATGGAATATTCCAAGTTGCTACAAT
GAAAAAAGCGA
TCTTTCCGAAGCCTGGTTATATGCAATCATCTCTGTTATGGTGTTCTGC
AGTACCATCTT
AGCCCTAGCTATATATCCCTACCTTGACATTGGCTGGAACGCAATAGAT
GCCATGAACCA
CCCAACTTTCCCCGCGCCCGCTATGCTTCCACTGCAACAAGTTGTTGCC
GGCGGCTTTGT
CCCAGCCAATCAGCCTCGCCCACCTTCTCCCACCCCCACTGAAATCAGC
TACTTTAATCT
AACAGGAGGAGATGACTGACACCCTAGATCTAGAAATGGACGGAATTAT
TACAGAGCAGC
GCCTGCTAGAAAGACGCAGGGCAGCGGCCGAGCAACAGCGCATGAATCA
AGAGCTCCAAG
ACATGGTTAACTTGCACCAGTGCAAAAGGGGTATCTTTTGTCTGGTAAA
GCAGGCCAAAG
TCACCTACGACAGTAATACCACCGGACACCGCCTTAGCTACAAGTTGCC
AACCAAGCGTC
AGAAATTGGTGGTCATGGTGGGAGAAAAGCCCATTACCATAACTCAGCA
CTCGGTAGAAA
CCGAAGGCTGCATTCACTCACCTTGTCAAGGACCTGAGGATCTCTGCAC
CCTTATTAAGA
CCCTGTGCGGTCTCAAAGATCTTATTCCCTTTAACTAATAAAAAAAAAT
AATAAAGCATC
ACTTACTTAAAATCAGTTAGCAAATTTCTGTCCAGTTTATTCAGCAGCA
CCTCCTTGCCC
TCCTCCCAGCTCTGGTATTGCAGCTTCCTCCTGGCTGCAAACTTTCTCC
ACAATCTAAAT
GGAATGTCAGTTTCCTCCTGTTCCTGTCCATCCGCACCCACTATCTTCA
TGTTGTTGCAG
ATGAAGCGCGCAAGACCGTCTGAAGATACCTTCAACCCCGTGTATCCAT
ATGACACGGAA
ACCGGTCCTCCAACTGTGCCTTTTCTTACTCCTCCCTTTGTATCCCCCA
ATGGGTTTCAA
GAGAGTCCCCCTGGGGTACTCTCTTTGCGCCTATCCGAACCTCTAGTTA
CCTCCAATGGC
ATGCTTGCGCTCAAAATGGGCAACGGCCTCTCTCTGGACGAGGCCGGCA
ACCTTACCTCC
CAAAATGTAACCACTGTGAGCCCACCTCTCAAAAAAACCAAGTCAAACA
TAAACCTGGAA
ATATCTGCACCCCTCACAGTTACCTCAGAAGCCCTAACTGTGGCTGCCG
CCGCACCTCTA
ATGGTCGCGGGCAACACACTCACCATGCAATCACAGGCCCCGCTAACCG
TGCACGACTCC
AAACTTAGCATTGCCACCCAAGGACCCCTCACAGTGTCAGAAGGAAAGC
TAGCCCTGCAA
ACATCAGGCCCCCTCACCACCACCGATAGCAGTACCCTTACTATCACTG
CCTCACCCCCT
CTAACTACTGCCACTGGTAGCTTGGGCATTGACTTGAAAGAGCCCATTT
ATACACAAAAT
GGAAAACTAGGACTAAAGTACGGGGCTCCTTTGCATGTAACAGACGACC
TAAACACTTTG
ACCGTAGCAACTGGTCCAGGTGTGACTATTAATAATACTTCCTTGCAAA
CTAAAGTTACT
GGAGCCTTGGGTTTTGATTCACAAGGCAATATGCAACTTAATGTAGCAG
GAGGACTAAGG
ATTGATTCTCAAAACAGACGCCTTATACTTGATGTTAGTTATCCGTTTG
ATGCTCAAAAC
CAACTAAATCTAAGACTAGGACAGGGCCCTCTTTTTATAAACTCAGCCC
ACAACTTGGAT
ATTAACTACAACAAAGGCCTTTACTTGTTTACAGCTTCAAACAATTCCA
AAAAGCTTGAG
GTTAACCTAAGCACTGCCAAGGGGTTGATGTTTGACGCTACAGCCATAG
CCATTAATGCA
GGAGATGGGCTTGAATTTGGTTCACCTAATGCACCAAACACAAATCCCC
TCAAAACAAAA
ATTGGCCATGGCCTAGAATTTGATTCAAACAAGGCTATGGTTCCTAAAC
TAGGAACTGGC
CTTAGTTTTGACAGCACAGGTGCCATTACAGTAGGAAACAAAAATAATG
ATAAGCTAACT
TTGTGGACCACACCAGCTCCATCTCCTAACTGTAGACTAAATGCAGAGA
AAGATGCTAAA
CTCACTTTGGTCTTAACAAAATGTGGCAGTCAAATACTTGCTACAGTTT
CAGTTTTGGCT
GTTAAAGGCAGTTTGGCTCCAATATCTGGAACAGTTCAAAGTGCTCATC
TTATTATAAGA
TTTGACGAAAATGGAGTGCTACTAAACAATTCCTTCCTGGACCCAGAAT
ATTGGAACTTT
AGAAATGGAGATCTTACTGAAGGCACAGCCTATACAAACGCTGTTGGAT
TTATGCCTAAC
CTATCAGCTTATCCAAAATCTCACGGTAAAACTGCCAAAAGTAACATTG
TCAGTCAAGTT
TACTTAAACGGAGACAAAACTAAACCTGTAACACTAACCATTACACTAA
ACGGTACACAG
GAAACAGGAGACACAACTCCAAGTGCATACTCTATGTCATTTTCATGGG
ACTGGTCTGGC
CACAACTACATTAATGAAATATTTGCCACATCCTCTTACACTTTTTCAT
ACATTGCCCAA
GAATAAAGAATCGTTTGTGTTATGTTTCAACGTGTTTATTTTTCAATTG
CAGAAAATTTC
AAGTCATTTTTCATTCAGTAGTATAGCCCCACCACCACATAGCTTATAC
AGATCACCGTA
CCTTAATCAAACTCACAGAACCCTAGTATTCAACCTGCCACCTCCCTCC
CAACACACAGA
GTACACAGTCCTTTCTCCCCGGCTGGCCTTAAAAAGCATCATATCATGG
GTAACAGACAT
ATTCTTAGGTGTTATATTCCACACGGTTTCCTGTCGAGCCAAACGCTCA
TCAGTGATATT
AATAAACTCCCCGGGCAGCTCACTTAAGTTCATGTCGCTGTCCAGCTGC
TGAGCCACAGG
CTGCTGTCCAACTTGCGGTTGCTTAACGGGCGGCGAAGGAGAAGTCCAC
GCCTACATGGG
GGTAGAGTCATAATCGTGCATCAGGATAGGGCGGTGGTGCTGCAGCAGC
GCGCGAATAAA
CTGCTGCCGCCGCCGCTCCGTCCTGCAGGAATACAACATGGCAGTGGTC
TCCTCAGCGAT
GATTCGCACCGCCCGCAGCATAAGGCGCCTTGTCCTCCGGGCACAGCAG
CGCACCCTGAT
CTCACTTAAATCAGCACAGTAACTGCAGCACAGCACCACAATATTGTTC
AAAATCCCACA
GTGCAAGGCGCTGTATCCAAAGCTCATGGCGGGGACCACAGAACCCACG
TGGCCATCATA
CCACAAGCGCAGGTAGATTAAGTGGCGACCCCTCATAAACACGCTGGAC
ATAAACATTAC
CTCTTTTGGCATGTTGTAATTCACCACCTCCCGGTACCATATAAACCTC
TGATTAAACAT
GGCGCCATCCACCACCATCCTAAACCAGCTGGCCAAAACCTGCCCGCCG
GCTATACACTG
CAGGGAACCGGGACTGGAACAATGACAGTGGAGAGCCCAGGACTCGTAA
CCATGGATCAT
CATGCTCGTCATGATATCAATGTTGGCACAACACAGGCACACGTGCATA
CACTTCCTCAG
GATTACAAGCTCCTCCCGCGTTAGAACCATATCCCAGGGAACAACCCAT
TCCTGAATCAG
CGTAAATCCCACACTGCAGGGAAGACCTCGCACGTAACTCACGTTGTGC
ATTGTCAAAGT
GTTACATTCGGGCAGCAGCGGATGATCCTCCAGTATGGTAGCGCGGGTT
TCTGTCTCAAA
AGGAGGTAGACGATCCCTACTGTACGGAGTGCGCCGAGACAACCGAGAT
CGTGTTGGTCG
TAGTGTCATGCCAAATGGAACGCCGGACGTAGTCATATTTCCTGAAGCA
AAACCAGGTGC
GGGCGTGACAAACAGATCTGCGTCTCCGGTCTCGCCGCTTAGATCGCTC
TGTGTAGTAGT
TGTAGTATATCCACTCTCTCAAAGCATCCAGGCGCCCCCTGGCTTCGGG
TTCTATGTAAA
CTCCTTCATGCGCCGCTGCCCTGATAACATCCACCACCGCAGAATAAGC
CACACCCAGCC
AACCTACACATTCGTTCTGCGAGTCACACACGGGAGGAGCGGGAAGAGC
TGGAAGAACCA
TGTTTTTTTTTTTATTCCAAAAGATTATCCAAAACCTCAAAATGAAGAT
CTATTAAGTGA
ACGCGCTCCCCTCCGGTGGCGTGGTCAAACTCTACAGCCAAAGAACAGA
TAATGGCATTT
GTAAGATGTTGCACAATGGCTTCCAAAAGGCAAACGGCCCTCACGTCCA
AGTGGACGTAA
AGGCTAAACCCTTCAGGGTGAATCTCCTCTATAAACATTCCAGCACCTT
CAACCATGCCC
AAATAATTCTCATCTCGCCACCTTCTCAATATATCTCTAAGCAAATCCC
GAATATTAAGT
CCGGCCATTGTAAAAATCTGCTCCAGAGCGCCCTCCACCTTCAGCCTCA
AGCAGCGAATC
ATGATTGCAAAAATTCAGGTTCCTCACAGACCTGTATAAGATTCAAAAG
CGGAACATTAA
CAAAAATACCGCGATCCCGTAGGTCCCTTCGCAGGGCCAGCTGAACATA
ATCGTGCAGGT
CTGCACGGACCAGCGCGGCCACTTCCCCGCCAGGAACCTTGACAAAAGA
ACCCACACTGA
TTATGACACGCATACTCGGAGCTATGCTAACCAGCGTAGCCCCGATGTA
AGCTTTGTTGC
ATGGGCGGCGATATAAAATGCAAGGTGCTGCTCAAAAAATCAGGCAAAG
CCTCGCGCAAA
AAAGAAAGCACATCGTAGTCATGCTCATGCAGATAAAGGCAGGTAAGCT
CCGGAACCACC
ACAGAAAAAGACACCATTTTTCTCTCAAACATGTCTGCGGGTTTCTGCA
TAAACACAAAA
TAAAATAACAAAAAAACATTTAAACATTAGAAGCCTGTCTTACAACAGG
AAAAACAACCC
TTATAAGCATAAGACGGACTACGGCCATGCCGGCGTGACCGTAAAAAAA
CTGGTCACCGT
GATTAAAAAGCACCACCGACAGCTCCTCGGTCATGTCCGGAGTCATAAT
GTAAGACTCGG
TAAACACATCAGGTTGATTCATCGGTCAGTGCTAAAAAGCGACCGAAAT
AGCCCGGGGGA
ATACATACCCGCAGGCGTAGAGACAACATTACAGCCCCCATAGGAGGTA
TAACAAAATTA
ATAGGAGAGAAAAACACATAAACACCTGAAAAACCCTCCTGCCTAGGCA
AAATAGCACCC
TCCCGCTCCAGAACAACATACAGCGCTTCCACAGCGGCAGCCATAACAG
TCAGCCTTACC
AGTAAAAAAGAAAACCTATTAAAAAAACACCACTCGACACGGCACCAGC
TCAATCAGTCA
CAGTGTAAAAAAGGGCCAAGTGCAGAGCGAGTATATATAGGACTAAAAA
ATGACGTAACG
GTTAAAGTCCACAAAAAACACCCAGAAAACCGCACGCGAACCTACGCCC
AGAAACGAAAG
CCAAAAAACCCACAACTTCCTCAAATCGTCACTTCCGTTTTCCCACGTT
ACGTCACTTCC
CATTTTAATTAAGAAAACTACAATTCCCAACACATACAAGTTACTCCGC
CCTAAAACCTA
CGTCACCCGCCCCGTTCCCACGCCCCGCGCCACGTCACAAACTCCACCC
CCTCATTATCA
TATTGGCTTCAATCCAAAATAAGGTATATTATTGATGATGATTACCCTG
TTAT
SEQ ID NO: 51 is the OV1160 sequence
AGGGTAATCATCATCAATAATATACCTTATTTTGGATTGAAGCCAATAT
GATAATGAGGG
GGTGGAGTTTGTGACGTGGCGCGGGGCGTGGGAACGGGGCGGGTGACGT
AGTAGTGTGGC
GGAAGTGTGATGTTGCAAGTGTGGCGGAACACATGTAAGCGACGGATGT
GGCAAAAGTGA
CGTTTTTGGTGTGCGCCGGTGTACACAGGAAGTGACAATTTTCGCGCGG
TTTTAGGCGGA
TGTTGTAGTAAATTTGGGCGTAACCGAGTAAGATTTGGCCATTTTCGCG
GGAAAACTGAA
TAAGAGGAAGTGAAATCTGAATAATTTTGTGTTACTCATAGCGCGTAAT
ATTTGTCTAGG
GCCGGGATCTCTGCAGGAATTTGATATCAAGCTTATCGATACCGTCGAA
ACTTGTTTATT
GCAGCTTATAATGGTTACAAATAAAGCAATAGCATCACAAATTTCACAA
ATAAAGCATTT
TTTTCACTGCATTCTAGTTGTGGTTTGTCCAAACTCATCAATGTATCTT
ATCATGTCTGG
ATCCGCTAGCGGCGCGCCGTTTCATCCGGACAAAGCCTGCGCGCGCCCC
GCCCCGCCATT
GGCCGTACCGCCCCGCGCCGCCGCCCCATCTCGCCCCTCGCCGCCGGGT
CCGGCGCGTTA
AAGCCAATAGGAACCGCCGCCGTTGTTCCCGTCACGGCCGGGGCAGCCA
ATTGTGGCGGC
GCTCGGCGGCTCGTGGCTCTTTCGCGGCAAAAAGGATTTGGCGCGTAAA
AGTGGCCGGGA
CTTTGCAGGCAGCGGCGGCCGGGGGCGGAGCGGGATCGAGCCCTCGATG
ATATCAGATCA
AACGATATCACCGGTCGACTGAAAATGAGACATATTATCTGCCACGGAG
GTGTTATTACC
GAAGAAATGGCCGCCAGTCTTTTGGACCAGCTGATCGAAGAGGTACTGG
CTGATAATCTT
CCACCTCCTAGCCATTTTGAACCACCTACCCTTCACGAACTGTATGATT
TAGACGTGACG
GCCCCCGAAGATCCCAACGAGGAGGCGGTTTCGCAGATTTTTCCCGACT
CTGTAATGTTG
GCGGTGCAGGAAGGGATTGACTTACTCACTTTTCCGCCGGCGCCCGGTT
CTCCGGAGCCG
CCTCACCTTTCCCGGCAGCCCGAGCAGCCGGAGCAGAGAGCCTTGGGTC
CGGTTTCTATG
CCAAACCTTGTACCGGAGGTGATCGATCTTACCTGCCACGAGGCTGGCT
TTCCACCCAGT
GACGACGAGGATGAAGAGGGTGAGGAGTTTGTGTTAGATTATGTGGAGC
ACCCCGGGCAC
GGTTGCAGGTCTTGTCATTATCACCGGAGGAATACGGGGGACCCAGATA
TTATGTGTTCG
CTTTGCTATATGAGGACCTGTGGCATGTTTGTCTACAGTAAGTGAAAAT
TATGGGCAGTG
GGTGATAGAGTGGTGGGTTTGGTGTGGTAATTTTTTTTTTAATTTTTAC
AGTTTTGTGGT
TTAAAGAATTTTGTATTGTGATTTTTTTAAAAGGTCCTGTGTCTGAACC
TGAGCCTGAGC
CCGAGCCAGAACCGGAGCCTGCAAGACCTACCCGCCGTCCTAAAATGGC
GCCTGCTATCC
TGAGACGCCCGACATCACCTGTGTCTAGAGAATGCAATAGTAGTACGGA
TAGCTGTGACT
CCGGTCCTTCTAACACACCTCCTGAGATACACCCGGTGGTCCCGCTGTG
CCCCATTAAAC
CAGTTGCCGTGAGAGTTGGTGGGCGTCGCCAGGCTGTGGAATGTATCGA
GGACTTGCTTA
ACGAGCCTGGGCAACCTTTGGACTTGAGCTGTAAACGCCCCAGGCCATA
AGGTGTAAACC
TGTGATTGCGTGTGTGGTTAACGCCTTTGTTTGCTGAATGAGTTGATGT
AAGTTTAATAA
AGGGTGAGATAATGTTTAACTTGCATGGCGTGTTAAATGGGGCGGGGCT
TAAAGGGTATA
TAATGCGCCGTGGGCTAATCTTGGTTACATCTGACCTCATGGAGGCTTG
GGAGTGTTTGG
AAGATTTTTCTGCTGTGCGTAACTTGCTGGAACAGAGCTCTAACAGTAC
CTCTTGGTTTT
GGAGGTTTCTGTGGGGCTCATCCCAGGCAAAGTTAGTCTGCAGAATTAA
GGAGGATTACA
AGTGGGAATTTGAAGAGCTTTTGAAATCCTGTGGTGAGCTGTTTGATTC
TTTGAATCTGG
GTCACCAGGCGCTTTTCCAAGAGAAGGTCATCAAGACTTTGGATTTTTC
CACACCGGGGC
GCGCTGCGGCTGCTGTTGCTTTTTTGAGTTTTATAAAGGATAAATGGAG
CGAAGAAACCC
ATCTGAGCGGGGGGTACCTGCTGGATTTTCTGGCCATGCATCTGTGGAG
AGCGGTTGTGA
GACACAAGAATCGCCTGCTACTGTTGTCTTCCGTCCGCCCGGCGATAAT
ACCGACGGAGG
AGCAGCAGCAGCAGCAGGAGGAAGCCAGGCGGCGGCGGCAGGAGCAGAG
CCCATGGAACC
CGAGAGCCGGCCTGGACCCTCGGGAATGAATGTTGTACAGGTGGCTGAA
CTGTATCCAGA
ACTGAGACGCATTTTGACAATTACAGAGGATGGGCAGGGGCTAAAGGGG
GTAAAGAGGGA
GCGGGGGGCTTGTGAGGCTACAGAGGAGGCTAGGAATCTAGCTTTTAGC
TTAATGACCAG
ACACCGTCCTGAGTGTATTACTTTTCAACAGATCAAGGATAATTGCGCT
AATGAGCTTGA
TCTGCTGGCGCAGAAGTATTCCATAGAGCAGCTGACCACTTACTGGCTG
CAGCCAGGGGA
TGATTTTGAGGAGGCTATTAGGGTATATGCAAAGGTGGCACTTAGGCCA
GATTGCAAGTA
CAAGATCAGCAAACTTGTAAATATCAGGAATTGTTGCTACATTTCTGGG
AACGGGGCCGA
GGTGGAGATAGATACGGAGGATAGGGTGGCCTTTAGATGTAGCATGATA
AATATGTGGCC
GGGGGTGCTTGGCATGGACGGGGTGGTTATTATGAATGTAAGGTTTACT
GGCCCCAATTT
TAGCGGTACGGTTTTCCTGGCCAATACCAACCTTATCCTACACGGTGTA
AGCTTCTATGG
GTTTAACAATACCTGTGTGGAAGCCTGGACCGATGTAAGGGTTCGGGGC
TGTGCCTTTTA
CTGCTGCTGGAAGGGGGTGGTGTGTCGCCCCAAAAGCAGGGCTTCAATT
AAGAAATGCCT
CTTTGAAAGGTGTACCTTGGGTATCCTGTCTGAGGGTAACTCCAGGGTG
CGCCACAATGT
GGCCTCCGACTGTGGTTGCTTCATGCTAGTGAAAAGCGTGGCTGTGATT
AAGCATAACAT
GGTATGTGGCAACTGCGAGGACAGGGCCTCTCAGATGCTGACCTGCTCG
GACGGCAACTG
TCACCTGCTGAAGACCATTCACGTAGCCAGCCACTCTCGCAAGGCCTGG
CCAGTGTTTGA
GCATAACATACTGACCCGCTGTTCCTTGCATTTGGGTAACAGGAGGGGG
GTGTTCCTACC
TTACCAATGCAATTTGAGTCACACTAAGATATTGCTTGAGCCCGAGAGC
ATGTCCAAGGT
GAACCTGAACGGGGTGTTTGACATGACCATGAAGATCTGGAAGGTGCTG
AGGTACGATGA
GACCCGCACCAGGTGCAGACCCTGCGAGTGTGGCGGTAAACATATTAGG
AACCAGCCTGT
GATGCTGGATGTGACCGAGGAGCTGAGGCCCGATCACTTGGTGCTGGCC
TGCACCCGCGC
TGAGTTTGGCTCTAGCGATGAAGATACAGATTGAGGTACTGAAATGTGT
GGGCGTGGCTT
AAGGGTGGGAAAGAATATATAAGGTGGGGGTCTTATGTAGTTTTGTATC
TGTTTTGCAGC
AGCCGCCGCCGCCATGAGCACCAACTCGTTTGATGGAAGCATTGTGAGC
TCATATTTGAC
AACGCGCATGCCCCCATGGGCCGGGGTGCGTCAGAATGTGATGGGCTCC
AGCATTGATGG
TCGCCCCGTCCTGCCCGCAAACTCTACTACCTTGACCTACGAGACCGTG
TCTGGAACGCC
GTTGGAGACTGCAGCCTCCGCCGCCGCTTCAGCCGCTGCAGCCACCGCC
CGCGGGATTGT
GACTGACTTTGCTTTCCTGAGCCCGCTTGCAAGCAGTGCAGCTTCCCGT
TCATCCGCCCG
CGATGACAAGTTGACGGCTCTTTTGGCACAATTGGATTCTTTGACCCGG
GAACTTAATGT
CGTTTCTCAGCAGCTGTTGGATCTGCGCCAGCAGGTTTCTGCCCTGAAG
GCTTCCTCCCC
TCCCAATGCGGTTTAAAACATAAATAAAAAACCAGACTCTGTTTGGATT
TGGATCAAGCA
AGTGTCTTGCTGTCTTTATTTAGGGGTTTTGCGCGCGCGGTAGGCCCGG
GACCAGCGGTC
TCGGTCGTTGAGGGTCCTGTGTATTTTTTCCAGGACGTGGTAAAGGTGA
CTCTGGATGTT
CAGATACATGGGCATAAGCCCGTCTCTGGGGTGGAGGTAGCACCACTGC
AGAGCTTCATG
CTGCGGGGTGGTGTTGTAGATGATCCAGTCGTAGCAGGAGCGCTGGGCG
TGGTGCCTAAA
AATGTCTTTCAGTAGCAAGCTGATTGCCAGGGGCAGGCCCTTGGTGTAA
GTGTTTACAAA
GCGGTTAAGCTGGGATGGGTGCATACGTGGGGATATGAGATGCATCTTG
GACTGTATTTT
TAGGTTGGCTATGTTCCCAGCCATATCCCTCCGGGGATTCATGTTGTGC
AGAACCACCAG
CACAGTGTATCCGGTGCACTTGGGAAATTTGTCATGTAGCTTAGAAGGA
AATGCGTGGAA
GAACTTGGAGACGCCCTTGTGACCTCCAAGATTTTCCATGCATTCGTCC
ATAATGATGGC
AATGGGCCCACGGGCGGCGGCCTGGGCGAAGATATTTCTGGGATCACTA
ACGTCATAGTT
GTGTTCCAGGATGAGATCGTCATAGGCCATTTTTACAAAGCGCGGGCGG
AGGGTGCCAGA
CTGCGGTATAATGGTTCCATCCGGCCCAGGGGCGTAGTTACCCTCACAG
ATTTGCATTTC
CCACGCTTTGAGTTCAGATGGGGGGATCATGTCTACCTGCGGGGCGATG
AAGAAAACGGT
TTCCGGGGTAGGGGAGATCAGCTGGGAAGAAAGCAGGTTCCTGAGCAGC
TGCGACTTACC
GCAGCCGGTGGGCCCGTAAATCACACCTATTACCGGGTGCAACTGGTAG
TTAAGAGAGCT
GCAGCTGCCGTCATCCCTGAGCAGGGGGGCCACTTCGTTAAGCATGTCC
CTGACTCGCAT
GTTTTCCCTGACCAAATCCGCCAGAAGGCGCTCGCCGCCCAGCGATAGC
AGTTCTTGCAA
GGAAGCAAAGTTTTTCAACGGTTTGAGACCGTCCGCCGTAGGCATGCTT
TTGAGCGTTTG
ACCAAGCAGTTCCAGGCGGTCCCACAGCTCGGTCACCTGCTCTACGGCA
TCTCGATCCAG
CATATCTCCTCGTTTCGCGGGTTGGGGCGGCTTTCGCTGTACGGCAGTA
GTCGGTGCTCG
TCCAGACGGGCCAGGGTCATGTCTTTCCACGGGCGCAGGGTCCTCGTCA
GCGTAGTCTGG
GTCACGGTGAAGGGGTGCGCTCCGGGCTGCGCGCTGGCCAGGGTGCGCT
TGAGGCTGGTC
CTGCTGGTGCTGAAGCGCTGCCGGTCTTCGCCCTGCGCGTCGGCCAGGT
AGCATTTGACC
ATGGTGTCATAGTCCAGCCCCTCCGCGGCGTGGCCCTTGGCGCGCAGCT
TGCCCTTGGAG
GAGGCGCCGCACGAGGGGCAGTGCAGACTTTTGAGGGCGTAGAGCTTGG
GCGCGAGAAAT
ACCGATTCCGGGGAGTAGGCATCCGCGCCGCAGGCCCCGCAGACGGTCT
CGCATTCCACG
AGCCAGGTGAGCTCTGGCCGTTCGGGGTCAAAAACCAGGTTTCCCCCAT
GCTTTTTGATG
CGTTTCTTACCTCTGGTTTCCATGAGCCGGTGTCCACGCTCGGTGACGA
AAAGGCTGTCC
GTGTCCCCGTATACAGACTTGAGAGGCCTGTCCTCGAGCGGTGTTCCGC
GGTCCTCCTCG
TATAGAAACTCGGACCACTCTGAGACAAAGGCTCGCGTCCAGGCCAGCA
CGAAGGAGGCT
AAGTGGGAGGGGTAGCGGTCGTTGTCCACTAGGGGGTCCACTCGCTCCA
GGGTGTGAAGA
CACATGTCGCCCTCTTCGGCATCAAGGAAGGTGATTGGTTTGTAGGTGT
AGGCCACGTGA
CCGGGTGTTCCTGAAGGGGGGCTATAAAAGGGGGTGGGGGCGCGTTCGT
CCTCACTCTCT
TCCGCATCGCTGTCTGCGAGGGCCAGCTGTTGGGGTGAGTACTCCCTCT
GAAAAGCGGGC
ATGACTTCTGCGCTAAGATTGTCAGTTTCCAAAAACGAGGAGGATTTGA
TATTCACCTGG
CCCGCGGTGATGCCTTTGAGGGTGGCCGCATCCATCTGGTCAGAAAAGA
CAATCTTTTTG
TTGTCAAGCTTGGTGGCAAACGACCCGTAGAGGGCGTTGGACAGCAACT
TGGCGATGGAG
CGCAGGGTTTGGTTTTTGTCGCGATCGGCGCGCTCCTTGGCCGCGATGT
TTAGCTGCACG
TATTCGCGCGCAACGCACCGCCATTCGGGAAAGACGGTGGTGCGCTCGT
CGGGCACCAGG
TGCACGCGCCAACCGCGGTTGTGCAGGGTGACAAGGTCAACGCTGGTGG
CTACCTCTCCG
CGTAGGCGCTCGTTGGTCCAGCAGAGGCGGCCGCCCTTGCGCGAGCAGA
ATGGCGGTAGG
GGGTCTAGCTGCGTCTCGTCCGGGGGGTCTGCGTCCACGGTAAAGACCC
CGGGCAGCAGG
CGCGCGTCGAAGTAGTCTATCTTGCATCCTTGCAAGTCTAGCGCCTGCT
GCCATGCGCGG
GCGGCAAGCGCGCGCTCGTATGGGTTGAGTGGGGGACCCCATGGCATGG
GGTGGGTGAGC
GCGGAGGCGTACATGCCGCAAATGTCGTAAACGTAGAGGGGCTCTCTGA
GTATTCCAAGA
TATGTAGGGTAGCATCTTCCACCGCGGATGCTGGCGCGCACGTAATCGT
ATAGTTCGTGC
GAGGGAGCGAGGAGGTCGGGACCGAGGTTGCTACGGGCGGGCTGCTCTG
CTCGGAAGACT
ATCTGCCTGAAGATGGCATGTGAGTTGGATGATATGGTTGGACGCTGGA
AGACGTTGAAG
CTGGCGTCTGTGAGACCTACCGCGTCACGCACGAAGGAGGCGTAGGAGT
CGCGCAGCTTG
TTGACCAGCTCGGCGGTGACCTGCACGTCTAGGGCGCAGTAGTCCAGGG
TTTCCTTGATG
ATGTCATACTTATCCTGTCCCTTTTTTTTCCACAGCTCGCGGTTGAGGA
CAAACTCTTCG
CGGTCTTTCCAGTACTCTTGGATCGGAAACCCGTCGGCCTCCGAACGGT
AAGAGCCTAGC
ATGTAGAACTGGTTGACGGCCTGGTAGGCGCAGCATCCCTTTTCTACGG
GTAGCGCGTAT
GCCTGCGCGGCCTTCCGGAGCGAGGTGTGGGTGAGCGCAAAGGTGTCCC
TGACCATGACT
TTGAGGTACTGGTATTTGAAGTCAGTGTCGTCGCATCCGCCCTGCTCCC
AGAGCAAAAAG
TCCGTGCGCTTTTTGGAACGCGGATTTGGCAGGGCGAAGGTGACATCGT
TGAAGAGTATC
TTTCCCGCGCGAGGCATAAAGTTGCGTGTGATGCGGAAGGGTCCCGGCA
CCTCGGAACGG
TTGTTAATTACCTGGGCGGCGAGCACGATCTCGTCAAAGCCGTTGATGT
TGTGGCCCACA
ATGTAAAGTTCCAAGAAGCGCGGGATGCCCTTGATGGAAGGCAATTTTT
TAAGTTCCTCG
TAGGTGAGCTCTTCAGGGGAGCTGAGCCCGTGCTCTGAAAGGGCCCAGT
CTGCAAGATGA
GGGTTGGAAGCGACGAATGAGCTCCACAGGTCACGGGCCATTAGCATTT
GCAGGTGGTCG
CGAAAGGTCCTAAACTGGCGACCTATGGCCATTTTTTCTGGGGTGATGC
AGTAGAAGGTA
AGCGGGTCTTGTTCCCAGCGGTCCCATCCAAGGTTCGCGGCTAGGTCTC
GCGCGGCAGTC
ACTAGAGGCTCATCTCCGCCGAACTTCATGACCAGCATGAAGGGCACGA
GCTGCTTCCCA
AAGGCCCCCATCCAAGTATAGGTCTCTACATCGTAGGTGACAAAGAGAC
GCTCGGTGCGA
GGATGCGAGCCGATCGGGAAGAACTGGATCTCCCGCCACCAATTGGAGG
AGTGGCTATTG
ATGTGGTGAAAGTAGAAGTCCCTGCGACGGGCCGAACACTCGTGCTGGC
TTTTGTAAAAA
CGTGCGCAGTACTGGCAGCGGTGCACGGGCTGTACATCCTGCACGAGGT
TGACCTGACGA
CCGCGCACAAGGAAGCAGAGTGGGAATTTGAGCCCCTCGCCTGGCGGGT
TTGGCTGGTGG
TCTTCTACTTCGGCTGCTTGTCCTTGACCGTCTGGCTGCTCGAGGGGAG
TTACGGTGGAT
CGGACCACCACGCCGCGCGAGCCCAAAGTCCAGATGTCCGCGCGCGGCG
GTCGGAGCTTG
ATGACAACATCGCGCAGATGGGAGCTGTCCATGGTCTGGAGCTCCCGCG
GCGTCAGGTCA
GGCGGGAGCTCCTGCAGGTTTACCTCGCATAGACGGGTCAGGGCGCGGG
CTAGATCCAGG
TGATACCTAATTTCCAGGGGCTGGTTGGTGGCGGCGTCGATGGCTTGCA
AGAGGCCGCAT
CCCCGCGGCGCGACTACGGTACCGCGCGGCGGGCGGTGGGCCGCGGGGG
TGTCCTTGGAT
GATGCATCTAAAAGCGGTGACGCGGGCGAGCCCCCGGAGGTAGGGGGGG
CTCCGGACCCG
CCGGGAGAGGGGGCAGGGGCACGTCGGCGCCGCGCGCGGGCAGGAGCTG
GTGCTGCGCGC
GTAGGTTGCTGGCGAACGCGACGACGCGGCGGTTGATCTCCTGAATCTG
GCGCCTCTGCG
TGAAGACGACGGGCCCGGTGAGCTTGAGCCTGAAAGAGAGTTCGACAGA
ATCAATTTCGG
TGTCGTTGACGGCGGCCTGGCGCAAAATCTCCTGCACGTCTCCTGAGTT
GTCTTGATAGG
CGATCTCGGCCATGAACTGCTCGATCTCTTCCTCCTGGAGATCTCCGCG
TCCGGCTCGCT
CCACGGTGGCGGCGAGGTCGTTGGAAATGCGGGCCATGAGCTGCGAGAA
GGCGTTGAGGC
CTCCCTCGTTCCAGACGCGGCTGTAGACCACGCCCCCTTCGGCATCGCG
GGCGCGCATGA
CCACCTGCGCGAGATTGAGCTCCACGTGCCGGGCGAAGACGGCGTAGTT
TCGCAGGCGCT
GAAAGAGGTAGTTGAGGGTGGTGGCGGTGTGTTCTGCCACGAAGAAGTA
CATAACCCAGC
GTCGCAACGTGGATTCGTTGATATCCCCCAAGGCCTCAAGGCGCTCCAT
GGCCTCGTAGA
AGTCCACGGCGAAGTTGAAAAACTGGGAGTTGCGCGCCGACACGGTTAA
CTCCTCCTCCA
GAAGACGGATGAGCTCGGCGACAGTGTCGCGCACCTCGCGCTCAAAGGC
TACAGGGGCCT
CTTCTTCTTCTTCAATCTCCTCTTCCATAAGGGCCTCCCCTTCTTCTTC
TTCTGGCGGCG
GTGGGGGAGGGGGGACACGGCGGCGACGACGGCGCACCGGGAGGCGGTC
GACAAAGCGCT
CGATCATCTCCCCGCGGCGACGGCGCATGGTCTCGGTGACGGCGCGGCC
GTTCTCGCGGG
GGCGCAGTTGGAAGACGCCGCCCGTCATGTCCCGGTTATGGGTTGGCGG
GGGGCTGCCAT
GCGGCAGGGATACGGCGCTAACGATGCATCTCAACAATTGTTGTGTAGG
TACTCCGCCGC
CGAGGGACCTGAGCGAGTCCGCATCGACCGGATCGGAAAACCTCTCGAG
AAAGGCGTCTA
ACCAGTCACAGTCGCAAGGTAGGCTGAGCACCGTGGCGGGCGGCAGCGG
GCGGCGGTCGG
GGTTGTTTCTGGCGGAGGTGCTGCTGATGATGTAATTAAAGTAGGCGGT
CTTGAGACGGC
GGATGGTCGACAGAAGCACCATGTCCTTGGGTCCGGCCTGCTGAATGCG
CAGGCGGTCGG
CCATGCCCCAGGCTTCGTTTTGACATCGGCGCAGGTCTTTGTAGTAGTC
TTGCATGAGCC
TTTCTACCGGCACTTCTTCTTCTCCTTCCTCTTGTCCTGCATCTCTTGC
ATCTATCGCTG
CGGCGGCGGCGGAGTTTGGCCGTAGGTGGCGCCCTCTTCCTCCCATGCG
TGTGACCCCGA
AGCCCCTCATCGGCTGAAGCAGGGCTAGGTCGGCGACAACGCGCTCGGC
TAATATGGCCT
GCTGCACCTGCGTGAGGGTAGACTGGAAGTCATCCATGTCCACAAAGCG
GTGGTATGCGC
CCGTGTTGATGGTGTAAGTGCAGTTGGCCATAACGGACCAGTTAACGGT
CTGGTGACCCG
GCTGCGAGAGCTCGGTGTACCTGAGACGCGAGTAAGCCCTCGAGTCAAA
TACGTAGTCGT
TGCAAGTCCGCACCAGGTACTGGTATCCCACCAAAAAGTGCGGCGGCGG
CTGGCGGTAGA
GGGGCCAGCGTAGGGTGGCCGGGGCTCCGGGGGCGAGATCTTCCAACAT
AAGGCGATGAT
ATCCGTAGATGTACCTGGACATCCAGGTGATGCCGGCGGCGGTGGTGGA
GGCGCGCGGAA
AGTCGCGGACGCGGTTCCAGATGTTGCGCAGCGGCAAAAAGTGCTCCAT
GGTCGGGACGC
TCTGGCCGGTCAGGCGCGCGCAATCGTTGACGCTCTAGACCGTGCAAAA
GGAGAGCCTGT
AAGCGGGCACTCTTCCGTGGTCTGGTGGATAAATTCGCAAGGGTATCAT
GGCGGACGACC
GGGGTTCGAGCCCCGTATCCGGCCGTCCGCCGTGATCCATGCGGTTACC
GCCCGCGTGTC
GAACCCAGGTGTGCGACGTCAGACAACGGGGGAGTGCTCCTTTTGGCTT
CCTTCCAGGCG
CGGCGGCTGCTGCGCTAGCTTTTTTGGCCACTGGCCGCGCGCAGCGTAA
GCGGTTAGGCT
GGAAAGCGAAAGCATTAAGTGGCTCGCTCCCTGTAGCCGGAGGGTTATT
TTCCAAGGGTT
GAGTCGCGGGACCCCCGGTTCGAGTCTCGGACCGGCCGGACTGCGGCGA
ACGGGGGTTTG
CCTCCCCGTCATGCAAGACCCCGCTTGCAAATTCCTCCGGAAACAGGGA
CGAGCCCCTTT
TTTGCTTTTCCCAGATGCATCCGGTGCTGCGGCAGATGCGCCCCCCTCC
TCAGCAGCGGC
AAGAGCAAGAGCAGCGGCAGACATGCAGGGCACCCTCCCCTCCTCCTAC
CGCGTCAGGAG
GGGCGACATCCGCGGTTGACGCGGCAGCAGATGGTGATTACGAACCCCC
GCGGCGCCGGG
CCCGGCACTACCTGGACTTGGAGGAGGGCGAGGGCCTGGCGCGGCTAGG
AGCGCCCTCTC
CTGAGCGGTACCCAAGGGTGCAGCTGAAGCGTGATACGCGTGAGGCGTA
CGTGCCGCGGC
AGAACCTGTTTCGCGACCGCGAGGGAGAGGAGCCCGAGGAGATGCGGGA
TCGAAAGTTCC
ACGCAGGGCGCGAGCTGCGGCATGGCCTGAATCGCGAGCGGTTGCTGCG
CGAGGAGGACT
TTGAGCCCGACGCGCGAACCGGGATTAGTCCCGCGCGCGCACACGTGGC
GGCCGCCGACC
TGGTAACCGCATACGAGCAGACGGTGAACCAGGAGATTAACTTTCAAAA
AAGCTTTAACA
ACCACGTGCGTACGCTTGTGGCGCGCGAGGAGGTGGCTATAGGACTGAT
GCATCTGTGGG
ACTTTGTAAGCGCGCTGGAGCAAAACCCAAATAGCAAGCCGCTCATGGC
GCAGCTGTTCC
TTATAGTGCAGCACAGCAGGGACAACGAGGCATTCAGGGATGCGCTGCT
AAACATAGTAG
AGCCCGAGGGCCGCTGGCTGCTCGATTTGATAAACATCCTGCAGAGCAT
AGTGGTGCAGG
AGCGCAGCTTGAGCCTGGCTGACAAGGTGGCCGCCATCAACTATTCCAT
GCTTAGCCTGG
GCAAGTTTTACGCCCGCAAGATATACCATACCCCTTACGTTCCCATAGA
CAAGGAGGTAA
AGATCGAGGGGTTCTACATGCGCATGGCGCTGAAGGTGCTTACCTTGAG
CGACGACCTGG
GCGTTTATCGCAACGAGCGCATCCACAAGGCCGTGAGCGTGAGCCGGCG
GCGCGAGCTCA
GCGACCGCGAGCTGATGCACAGCCTGCAAAGGGCCCTGGCTGGCACGGG
CAGCGGCGATA
GAGAGGCCGAGTCCTACTTTGACGCGGGCGCTGACCTGCGCTGGGCCCC
AAGCCGACGCG
CCCTGGAGGCAGCTGGGGCCGGACCTGGGCTGGCGGTGGCACCCGCGCG
CGCTGGCAACG
TCGGCGGCGTGGAGGAATATGACGAGGACGATGAGTACGAGCCAGAGGA
CGGCGAGTACT
AAGCGGTGATGTTTCTGATCAGATGATGCAAGACGCAACGGACCCGGCG
GTGCGGGCGGC
GCTGCAGAGCCAGCCGTCCGGCCTTAACTCCACGGACGACTGGCGCCAG
GTCATGGACCG
CATCATGTCGCTGACTGCGCGCAATCCTGACGCGTTCCGGCAGCAGCCG
CAGGCCAACCG
GCTCTCCGCAATTCTGGAAGCGGTGGTCCCGGCGCGCGCAAACCCCACG
CACGAGAAGGT
GCTGGCGATCGTAAACGCGCTGGCCGAAAACAGGGCCATCCGGCCCGAC
GAGGCCGGCCT
GGTCTACGACGCGCTGCTTCAGCGCGTGGCTCGTTACAACAGCGGCAAC
GTGCAGACCAA
CCTGGACCGGCTGGTGGGGGATGTGCGCGAGGCCGTGGCGCAGCGTGAG
CGCGCGCAGCA
GCAGGGCAACCTGGGCTCCATGGTTGCACTAAACGCCTTCCTGAGTACA
CAGCCCGCCAA
CGTGCCGCGGGGACAGGAGGACTACACCAACTTTGTGAGCGCACTGCGG
CTAATGGTGAC
TGAGACACCGCAAAGTGAGGTGTACCAGTCTGGGCCAGACTATTTTTTC
CAGACCAGTAG
ACAAGGCCTGCAGACCGTAAACCTGAGCCAGGCTTTCAAAAACTTGCAG
GGGCTGTGGGG
GGTGCGGGCTCCCACAGGCGACCGCGCGACCGTGTCTAGCTTGCTGACG
CCCAACTCGCG
CCTGTTGCTGCTGCTAATAGCGCCCTTCACGGACAGTGGCAGCGTGTCC
CGGGACACATA
CCTAGGTCACTTGCTGACACTGTACCGCGAGGCCATAGGTCAGGCGCAT
GTGGACGAGCA
TACTTTCCAGGAGATTACAAGTGTCAGCCGCGCGCTGGGGCAGGAGGAC
ACGGGCAGCCT
GGAGGCAACCCTAAACTACCTGCTGACCAACCGGCGGCAGAAGATCCCC
TCGTTGCACAG
TTTAAACAGCGAGGAGGAGCGCATTTTGCGCTACGTGCAGCAGAGCGTG
AGCCTTAACCT
GATGCGCGACGGGGTAACGCCCAGCGTGGCGCTGGACATGACCGCGCGC
AACATGGAACC
GGGCATGTATGCCTCAAACCGGCCGTTTATCAACCGCCTAATGGACTAC
TTGCATCGCGC
GGCCGCCGTGAACCCCGAGTATTTCACCAATGCCATCTTGAACCCGCAC
TGGCTACCGCC
CCCTGGTTTCTACACCGGGGGATTCGAGGTGCCCGAGGGTAACGATGGA
TTCCTCTGGGA
CGACATAGACGACAGCGTGTTTTCCCCGCAACCGCAGACCCTGCTAGAG
TTGCAACAGCG
CGAGCAGGCAGAGGCGGCGCTGCGAAAGGAAAGCTTCCGCAGGCCAAGC
AGCTTGTCCGA
TCTAGGCGCTGCGGCCCCGCGGTCAGATGCTAGTAGCCCATTTCCAAGC
TTGATAGGGTC
TCTTACCAGCACTCGCACCACCCGCCCGCGCCTGCTGGGCGAGGAGGAG
TACCTAAACAA
CTCGCTGCTGCAGCCGCAGCGCGAAAAAAACCTGCCTCCGGCATTTCCC
AACAACGGGAT
AGAGAGCCTAGTGGACAAGATGAGTAGATGGAAGACGTACGCGCAGGAG
CACAGGGACGT
GCCAGGCCCGCGCCCGCCCACCCGTCGTCAAAGGCACGACCGTCAGCGG
GGTCTGGTGTG
GGAGGACGATGACTCGGCAGACGACAGCAGCGTCCTGGATTTGGGAGGG
AGTGGCAACCC
GTTTGCGCACCTTCGCCCCAGGCTGGGGAGAATGTTTTAAAAAAAAAAA
AGCATGATGCA
AAATAAAAAACTCACCAAGGCCATGGCACCGAGCGTTGGTTTTCTTGTA
TTCCCCTTAGT
ATGCGGCGCGCGGCGATGTATGAGGAAGGTCCTCCTCCCTCCTACGAGA
GTGTGGTGAGC
GCGGCGCCAGTGGCGGCGGCGCTGGGTTCTCCCTTCGATGCTCCCCTGG
ACCCGCCGTTT
GTGCCTCCGCGGTACCTGCGGCCTACCGGGGGGAGAAACAGCATCCGTT
ACTCTGAGTTG
GCACCCCTATTCGACACCACCCGTGTGTACCTGGTGGACAACAAGTCAA
CGGATGTGGCA
TCCCTGAACTACCAGAACGACCACAGCAACTTTCTGACCACGGTCATTC
AAAACAATGAC
TACAGCCCGGGGGAGGCAAGCACACAGACCATCAATCTTGACGACCGGT
CGCACTGGGGC
GGCGACCTGAAAACCATCCTGCATACCAACATGCCAAATGTGAACGAGT
TCATGTTTACC
AATAAGTTTAAGGCGCGGGTGATGGTGTCGCGCTTGCCTACTAAGGACA
ATCAGGTGGAG
CTGAAATACGAGTGGGTGGAGTTCACGCTGCCCGAGGGCAACTACTCCG
AGACCATGACC
ATAGACCTTATGAACAACGCGATCGTGGAGCACTACTTGAAAGTGGGCA
GACAGAACGGG
GTTCTGGAAAGCGACATCGGGGTAAAGTTTGACACCCGCAACTTCAGAC
TGGGGTTTGAC
CCCGTCACTGGTCTTGTCATGCCTGGGGTATATACAAACGAAGCCTTCC
ATCCAGACATC
ATTTTGCTGCCAGGATGCGGGGTGGACTTCACCCACAGCCGCCTGAGCA
ACTTGTTGGGC
ATCCGCAAGCGGCAACCCTTCCAGGAGGGCTTTAGGATCACCTACGATG
ATCTGGAGGGT
GGTAACATTCCCGCACTGTTGGATGTGGACGCCTACCAGGCGAGCTTGA
AAGATGACACC
GAACAGGGCGGGGGTGGCGCAGGCGGCAGCAACAGCAGTGGCAGCGGCG
CGGAAGAGAAC
TCCAACGCGGCAGCCGCGGCAATGCAGCCGGTGGAGGACATGAACGATC
ATGCCATTCGC
GGCGACACCTTTGCCACACGGGCTGAGGAGAAGCGCGCTGAGGCCGAAG
CAGCGGCCGAA
GCTGCCGCCCCCGCTGCGCAACCCGAGGTCGAGAAGCCTCAGAAGAAAC
CGGTGATCAAA
CCCCTGACAGAGGACAGCAAGAAACGCAGTTACAACCTAATAAGCAATG
ACAGCACCTTC
ACCCAGTACCGCAGCTGGTACCTTGCATACAACTACGGCGACCCTCAGA
CCGGAATCCGC
TCATGGACCCTGCTTTGCACTCCTGACGTAACCTGCGGCTCGGAGCAGG
TCTACTGGTCG
TTGCCAGACATGATGCAAGACCCCGTGACCTTCCGCTCCACGCGCCAGA
TCAGCAACTTT
CCGGTGGTGGGCGCCGAGCTGTTGCCCGTGCACTCCAAGAGCTTCTACA
ACGACCAGGCC
GTCTACTCCCAACTCATCCGCCAGTTTACCTCTCTGACCCACGTGTTCA
ATCGCTTTCCC
GAGAACCAGATTTTGGCGCGCCCGCCAGCCCCCACCATCACCACCGTCA
GTGAAAACGTT
CCTGCTCTCACAGATCACGGGACGCTACCGCTGCGCAACAGCATCGGAG
GAGTCCAGCGA
GTGACCATTACTGACGCCAGACGCCGCACCTGCCCCTACGTTTACAAGG
CCCTGGGCATA
GTCTCGCCGCGCGTCCTATCGAGCCGCACTTTTTGAGCAAGCATGTCCA
TCCTTATATCG
CCCAGCAATAACACAGGCTGGGGCCTGCGCTTCCCAAGCAAGATGTTTG
GCGGGGCCAAG
AAGCGCTCCGACCAACACCCAGTGCGCGTGCGCGGGCACTACCGCGCGC
CCTGGGGCGCG
CACAAACGCGGCCGCACTGGGCGCACCACCGTCGATGACGCCATCGACG
CGGTGGTGGAG
GAGGCGCGCAACTACACGCCCACGCCGCCACCAGTGTCCACAGTGGACG
CGGCCATTCAG
ACCGTGGTGCGCGGAGCCCGGCGCTATGCTAAAATGAAGAGACGGCGGA
GGCGCGTAGCA
CGTCGCCACCGCCGCCGACCCGGCACTGCCGCCCAACGCGCGGCGGCGG
CCCTGCTTAAC
CGCGCACGTCGCACCGGCCGACGGGCGGCCATGCGGGCCGCTCGAAGGC
TGGCCGCGGGT
ATTGTCACTGTGCCCCCCAGGTCCAGGCGACGAGCGGCCGCCGCAGCAG
CCGCGGCCATT
AGTGCTATGACTCAGGGTCGCAGGGGCAACGTGTATTGGGTGCGCGACT
CGGTTAGCGGC
CTGCGCGTGCCCGTGCGCACCCGCCCCCCGCGCAACTAGATTGCAAGAA
AAAACTACTTA
GACTCGTACTGTTGTATGTATCCAGCGGCGGCGGCGCGCAACGAAGCTA
TGTCCAAGCGC
AAAATCAAAGAAGAGATGCTCCAGGTCATCGCGCCGGAGATCTATGGCC
CCCCGAAGAAG
GAAGAGCAGGATTACAAGCCCCGAAAGCTAAAGCGGGTCAAAAAGAAAA
AGAAAGATGAT
GATGATGAACTTGACGACGAGGTGGAACTGCTGCACGCTACCGCGCCCA
GGCGACGGGTA
CAGTGGAAAGGTCGACGCGTAAAACGTGTTTTGCGACCCGGCACCACCG
TAGTCTTTACG
CCCGGTGAGCGCTCCACCCGCACCTACAAGCGCGTGTATGATGAGGTGT
ACGGCGACGAG
GACCTGCTTGAGCAGGCCAACGAGCGCCTCGGGGAGTTTGCCTACGGAA
AGCGGCATAAG
GACATGCTGGCGTTGCCGCTGGACGAGGGCAACCCAACACCTAGCCTAA
AGCCCGTAACA
CTGCAGCAGGTGCTGCCCGCGCTTGCACCGTCCGAAGAAAAGCGCGGCC
TAAAGCGCGAG
TCTGGTGACTTGGCACCCACCGTGCAGCTGATGGTACCCAAGCGCCAGC
GACTGGAAGAT
GTCTTGGAAAAAATGACCGTGGAACCTGGGCTGGAGCCCGAGGTCCGCG
TGCGGCCAATC
AAGCAGGTGGCGCCGGGACTGGGCGTGCAGACCGTGGACGTTCAGATAC
CCACTACCAGT
AGCACCAGTATTGCCACCGCCACAGAGGGCATGGAGACACAAACGTCCC
CGGTTGCCTCA
GCGGTGGCGGATGCCGCGGTGCAGGCGGTCGCTGCGGCCGCGTCCAAGA
CCTCTACGGAG
GTGCAAACGGACCCGTGGATGTTTCGCGTTTCAGCCCCCCGGCGCCCGC
GCGGTTCGAGG
AAGTACGGCGCCGCCAGCGCGCTACTGCCCGAATATGCCCTACATCCTT
CCATTGCGCCT
ACCCCCGGCTATCGTGGCTACACCTACCGCCCCAGAAGACGAGCAACTA
CCCGACGCCGA
ACCACCACTGGAACCCGCCGCCGCCGTCGCCGTCGCCAGCCCGTGCTGG
CCCCGATTTCC
GTGCGCAGGGTGGCTCGCGAAGGAGGCAGGACCCTGGTGCTGCCAACAG
CGCGCTACCAC
CCCAGCATCGTTTAAAAGCCGGTCTTTGTGGTTCTTGCAGATATGGCCC
TCACCTGCCGC
CTCCGTTTCCCGGTGCCGGGATTCCGAGGAAGAATGCACCGTAGGAGGG
GCATGGCCGGC
CACGGCCTGACGGGCGGCATGCGTCGTGCGCACCACCGGCGGCGGCGCG
CGTCGCACCGT
CGCATGCGCGGCGGTATCCTGCCCCTCCTTATTCCACTGATCGCCGCGG
CGATTGGCGCC
GTGCCCGGAATTGCATCCGTGGCCTTGCAGGCGCAGAGACACTGATTAA
AAACAAGTTGC
ATGTGGAAAAATCAAAATAAAAAGTCTGGACTCTCACGCTCGCTTGGTC
CTGTAACTATT
TTGTAGAATGGAAGACATCAACTTTGCGTCTCTGGCCCCGCGACACGGC
TCGCGCCCGTT
CATGGGAAACTGGCAAGATATCGGCACCAGCAATATGAGCGGTGGCGCC
TTCAGCTGGGG
CTCGCTGTGGAGCGGCATTAAAAATTTCGGTTCCACCGTTAAGAACTAT
GGCAGCAAGGC
CTGGAACAGCAGCACAGGCCAGATGCTGAGGGATAAGTTGAAAGAGCAA
AATTTCCAACA
AAAGGTGGTAGATGGCCTGGCCTCTGGCATTAGCGGGGTGGTGGACCTG
GCCAACCAGGC
AGTGCAAAATAAGATTAACAGTAAGCTTGATCCCCGCCCTCCCGTAGAG
GAGCCTCCACC
GGCCGTGGAGACAGTGTCTCCAGAGGGGCGTGGCGAAAAGCGTCCGCGC
CCCGACAGGGA
AGAAACTCTGGTGACGCAAATAGACGAGCCTCCCTCGTACGAGGAGGCA
CTAAAGCAAGG
CCTGCCCACCACCCGTCCCATCGCGCCCATGGCTACCGGAGTGCTGGGC
CAGCACACACC
CGTAACGCTGGACCTGCCTCCCCCCGCCGACACCCAGCAGAAACCTGTG
CTGCCAGGCCC
GACCGCCGTTGTTGTAACCCGTCCTAGCCGCGCGTCCCTGCGCCGCGCC
GCCAGCGGTCC
GCGATCGTTGCGGCCCGTAGCCAGTGGCAACTGGCAAAGCACACTGAAC
AGCATCGTGGG
TCTGGGGGTGCAATCCCTGAAGCGCCGACGATGCTTCTGAATAGCTAAC
GTGTCGTATGT
GTGTCATGTATGCGTCCATGTCGCCGCCAGAGGAGCTGCTGAGCCGCCG
CGCGCCCGCTT
TCCAAGATGGCTACCCCTTCGATGATGCCGCAGTGGTCTTACATGCACA
TCTCGGGCCAG
GACGCCTCGGAGTACCTGAGCCCCGGGCTGGTGCAGTTTGCCCGCGCCA
CCGAGACGTAC
TTCAGCCTGAATAACAAGTTTAGAAACCCCACGGTGGCGCCTACGCACG
ACGTGACCACA
GACCGGTCCCAGCGTTTGACGCTGCGGTTCATCCCTGTGGACCGTGAGG
ATACTGCGTAC
TCGTACAAGGCGCGGTTCACCCTAGCTGTGGGTGATAACCGTGTGCTGG
ACATGGCTTCC
ACGTACTTTGACATCCGCGGCGTGCTGGACAGGGGCCCTACTTTTAAGC
CCTACTCTGGC
ACTGCCTACAACGCCCTGGCTCCCAAGGGTGCCCCAAATCCTTGCGAAT
GGGATGAAGCT
GCTACTGCTCTTGAAATAAACCTAGAAGAAGAGGACGATGACAACGAAG
ACGAAGTAGAC
GAGCAAGCTGAGCAGCAAAAAACTCACGTATTTGGGCAGGCGCCTTATT
CTGGTATAAAT
ATTACAAAGGAGGGTATTCAAATAGGTGTCGAAGGTCAAACACCTAAAT
ATGCCGATAAA
ACATTTCAACCTGAACCTCAAATAGGAGAATCTCAGTGGTACGAAACTG
AAATTAATCAT
GCAGCTGGGAGAGTCCTTAAAAAGACTACCCCAATGAAACCATGTTACG
GTTCATATGCA
AAACCCACAAATGAAAATGGAGGGCAAGGCATTCTTGTAAAGCAACAAA
ATGGAAAGCTA
GAAAGTCAAGTGGAAATGCAATTTTTCTCAACTACTGAGGCGACCGCAG
GCAATGGTGAT
AACTTGACTCCTAAAGTGGTATTGTACAGTGAAGATGTAGATATAGAAA
CCCCAGACACT
CATATTTCTTACATGCCCACTATTAAGGAAGGTAACTCACGAGAACTAA
TGGGCCAACAA
TCTATGCCCAACAGGCCTAATTACATTGCTTTTAGGGACAATTTTATTG
GTCTAATGTAT
TACAACAGCACGGGTAATATGGGTGTTCTGGCGGGCCAAGCATCGCAGT
TGAATGCTGTT
GTAGATTTGCAAGACAGAAACACAGAGCTTTCATACCAGCTTTTGCTTG
ATTCCATTGGT
GATAGAACCAGGTACTTTTCTATGTGGAATCAGGCTGTTGACAGCTATG
ATCCAGATGTT
AGAATTATTGAAAATCATGGAACTGAAGATGAACTTCCAAATTACTGCT
TTCCACTGGGA
GGTGTGATTAATACAGAGACTCTTACCAAGGTAAAACCTAAAACAGGTC
AGGAAAATGGA
TGGGAAAAAGATGCTACAGAATTTTCAGATAAAAATGAAATAAGAGTTG
GAAATAATTTT
GCCATGGAAATCAATCTAAATGCCAACCTGTGGAGAAATTTCCTGTACT
CCAACATAGCG
CTGTATTTGCCCGACAAGCTAAAGTACAGTCCTTCCAACGTAAAAATTT
CTGATAACCCA
AACACCTACGACTACATGAACAAGCGAGTGGTGGCTCCCGGGTTAGTGG
ACTGCTACATT
AACCTTGGAGCACGCTGGTCCCTTGACTATATGGACAACGTCAACCCAT
TTAACCACCAC
CGCAATGCTGGCCTGCGCTACCGCTCAATGTTGCTGGGCAATGGTCGCT
ATGTGCCCTTC
CACATCCAGGTGCCTCAGAAGTTCTTTGCCATTAAAAACCTCCTTCTCC
TGCCGGGCTCA
TACACCTACGAGTGGAACTTCAGGAAGGATGTTAACATGGTTCTGCAGA
GCTCCCTAGGA
AATGACCTAAGGGTTGACGGAGCCAGCATTAAGTTTGATAGCATTTGCC
TTTACGCCACC
TTCTTCCCCATGGCCCACAACACCGCCTCCACGCTTGAGGCCATGCTTA
GAAACGACACC
AACGACCAGTCCTTTAACGACTATCTCTCCGCCGCCAACATGCTCTACC
CTATACCCGCC
AACGCTACCAACGTGCCCATATCCATCCCCTCCCGCAACTGGGCGGCTT
TCCGCGGCTGG
GCCTTCACGCGCCTTAAGACTAAGGAAACCCCATCACTGGGCTCGGGCT
ACGACCCTTAT
TACACCTACTCTGGCTCTATACCCTACCTAGATGGAACCTTTTACCTCA
ACCACACCTTT
AAGAAGGTGGCCATTACCTTTGACTCTTCTGTCAGCTGGCCTGGCAATG
ACCGCCTGCTT
ACCCCCAACGAGTTTGAAATTAAGCGCTCAGTTGACGGGGAGGGTTACA
ACGTTGCCCAG
TGTAACATGACCAAAGACTGGTTCCTGGTACAAATGCTAGCTAACTACA
ACATTGGCTAC
CAGGGCTTCTATATCCCAGAGAGCTACAAGGACCGCATGTACTCCTTCT
TTAGAAACTTC
CAGCCCATGAGCCGTCAGGTGGTGGATGATACTAAATACAAGGACTACC
AACAGGTGGGC
ATCCTACACCAACACAACAACTCTGGATTTGTTGGCTACCTTGCCCCCA
CCATGCGCGAA
GGACAGGCCTACCCTGCTAACTTCCCCTATCCGCTTATAGGCAAGACCG
CAGTTGACAGC
ATTACCCAGAAAAAGTTTCTTTGCGATCGCACCCTTTGGCGCATCCCAT
TCTCCAGTAAC
TTTATGTCCATGGGCGCACTCACAGACCTGGGCCAAAACCTTCTCTACG
CCAACTCCGCC
CACGCGCTAGACATGACTTTTGAGGTGGATCCCATGGACGAGCCCACCC
TTCTTTATGTT
TTGTTTGAAGTCTTTGACGTGGTCCGTGTGCACCGGCCGCACCGCGGCG
TCATCGAAACC
GTGTACCTGCGCACGCCCTTCTCGGCCGGCAACGCCACAACATAAAGAA
GCAAGCAACAT
CAACAACAGCTGCCGCCATGGGCTCCAGTGAGCAGGAACTGAAAGCCAT
TGTCAAAGATC
TTGGTTGTGGGCCATATTTTTTGGGCACCTATGACAAGCGCTTTCCAGG
CTTTGTTTCTC
CACACAAGCTCGCCTGCGCCATAGTCAATACGGCCGGTCGCGAGACTGG
GGGCGTACACT
GGATGGCCTTTGCCTGGAACCCGCACTCAAAAACATGCTACCTCTTTGA
GCCCTTTGGCT
TTTCTGACCAGCGACTCAAGCAGGTTTACCAGTTTGAGTACGAGTCACT
CCTGCGCCGTA
GCGCCATTGCTTCTTCCCCCGACCGCTGTATAACGCTGGAAAAGTCCAC
CCAAAGCGTAC
AGGGGCCCAACTCGGCCGCCTGTGGACTATTCTGCTGCATGTTTCTCCA
CGCCTTTGCCA
ACTGGCCCCAAACTCCCATGGATCACAACCCCACCATGAACCTTATTAC
CGGGGTACCCA
ACTCCATGCTCAACAGTCCCCAGGTACAGCCCACCCTGCGTCGCAACCA
GGAACAGCTCT
ACAGCTTCCTGGAGCGCCACTCGCCCTACTTCCGCAGCCACAGTGCGCA
GATTAGGAGCG
CCACTTCTTTTTGTCACTTGAAAAACATGTAAAAATAATTACTTATGAC
TCGTACTATTG
TTATTCATCCAGGCGGTAGGAGGGCCATCATGGCTATGATGGAGGTCCA
GGGGGGACCCA
GCCTGGGACAGACCTGCGTGCTGATCGTGATCTTTACAGTGCTCCTGCA
GTCTCTCTGTG
TGGCTGTAACTTACGTGTACTTTACCAACGAGCTGAAGCAGATGCAGGA
CAAGTACTCCA
AAAGTGGCATTGCTTGTTTCTTAAAAGAAGATGACAGTTATTGGGACCC
CAATGACGAAG
AGAGTATGAACAGCCCCTGCTGGCAAGTCAAGTGGCAACTCCGTCAGCT
CGTTAGAAAGA
TGATTTTGAGAACCTCTGAGGAAACCATTTCTACAGTTCAAGAAAAGCA
ACAAAATATTT
CTCCCCTAGTGAGAGAAAGAGGTCCTCAGAGAGTAGCAGCTCACATAAC
TGGGACCAGAG
GAAGAAGCAACACATTGTCTTCTCCAAACTCCAAGAATGAAAAGGCTCT
GGGCCGCAAAA
TAAACTCCTGGGAATCATCAAGGAGTGGGCATTCATTCCTGAGCAACTT
GCACTTGAGGA
ATGGTGAACTGGTCATCCATGAAAAAGGGTTTTACTACATCTATTCCCA
AACATACTTTC
GATTTCAGGAGGAAATAAAAGAAAACACAAAGAACGACAAACAAATGGT
CCAATATATTT
ACAAATACACAAGTTATCCTGACCCTATATTGTTGATGAAAAGTGCTAG
AAATAGTTGTT
GGTCTAAAGATGCAGAATATGGACTCTATTCCATCTATCAAGGGGGAAT
ATTTGAGCTTA
AGGAAAATGACAGAATTTTTGTTTCTGTAACAAATGAGCACTTAATAGA
CATGGACCATG
AAGCCAGTTTTTTCGGGGCCTTTTTAGTTGGCTAAGCTAGCTACTAGAG
ACACTTTCAAT
AAAGGCAAATGCTTTTATTTGTACACTCTCGGGTGATTATTTACCCCCA
CCCTTGCCGTC
TGCGCCGTTTAAAAATCAAAGGGGTTCTGCCGCGCATCGCTATGCGCCA
CTGGCAGGGAC
ACGTTGCGATACTGGTGTTTAGTGCTCCACTTAAACTCAGGCACAACCA
TCCGCGGCAGC
TCGGTGAAGTTTTCACTCCACAGGCTGCGCACCATCACCAACGCGTTTA
GCAGGTCGGGC
GCCGATATCTTGAAGTCGCAGTTGGGGCCTCCGCCCTGCGCGCGCGAGT
TGCGATACACA
GGGTTGCAGCACTGGAACACTATCAGCGCCGGGTGGTGCACGCTGGCCA
GCACGCTCTTG
TCGGAGATCAGATCCGCGTCCAGGTCCTCCGCGTTGCTCAGGGCGAACG
GAGTCAACTTT
GGTAGCTGCCTTCCCAAAAAGGGCGCGTGCCCAGGCTTTGAGTTGCACT
CGCACCGTAGT
GGCATCAAAAGGTGACCGTGCCCGGTCTGGGCGTTAGGATACAGCGCCT
GCATAAAAGCC
TTGATCTGCTTAAAAGCCACCTGAGCCTTTGCGCCTTCAGAGAAGAACA
TGCCGCAAGAC
TTGCCGGAAAACTGATTGGCCGGACAGGCCGCGTCGTGCACGCAGCACC
TTGCGTCGGTG
TTGGAGATCTGCACCACATTTCGGCCCCACCGGTTCTTCACGATCTTGG
CCTTGCTAGAC
TGCTCCTTCAGCGCGCGCTGCCCGTTTTCGCTCGTCACATCCATTTCAA
TCACGTGCTCC
TTATTTATCATAATGCTTCCGTGTAGACACTTAAGCTCGCCTTCGATCT
CAGCGCAGCGG
TGCAGCCACAACGCGCAGCCCGTGGGCTCGTGATGCTTGTAGGTCACCT
CTGCAAACGAC
TGCAGGTACGCCTGCAGGAATCGCCCCATCATCGTCACAAAGGTCTTGT
TGCTGGTGAAG
GTCAGCTGCAACCCGCGGTGCTCCTCGTTCAGCCAGGTCTTGCATACGG
CCGCCAGAGCT
TCCACTTGGTCAGGCAGTAGTTTGAAGTTCGCCTTTAGATCGTTATCCA
CGTGGTACTTG
TCCATCAGCGCGCGCGCAGCCTCCATGCCCTTCTCCCACGCAGACACGA
TCGGCACACTC
AGCGGGTTCATCACCGTAATTTCACTTTCCGCTTCGCTGGGCTCTTCCT
CTTCCTCTTGC
GTCCGCATACCACGCGCCACTGGGTCGTCTTCATTCAGCCGCCGCACTG
TGCGCTTACCT
CCTTTGCCATGCTTGATTAGCACCGGTGGGTTGCTGAAACCCACCATTT
GTAGCGCCACA
TCTTCTCTTTCTTCCTCGCTGTCCACGATTACCTCTGGTGATGGCGGGC
GCTCGGGCTTG
GGAGAAGGGCGCTTCTTTTTCTTCTTGGGCGCAATGGCCAAATCCGCCG
CCGAGGTCGAT
GGCCGCGGGCTGGGTGTGCGCGGCACCAGCGCGTCTTGTGATGAGTCTT
CCTCGTCCTCG
GACTCGATACGCCGCCTCATCCGCTTTTTTGGGGGCGCCCGGGGAGGCG
GCGGCGACGGG
GACGGGGACGACACGTCCTCCATGGTTGGGGGACGTCGCGCCGCACCGC
GTCCGCGCTCG
GGGGTGGTTTCGCGCTGCTCCTCTTCCCGACTGGCCATTTCCTTCTCCT
ATAGGCAGAAA
AAGATCATGGAGTCAGTCGAGAAGAAGGACAGCCTAACCGCCCCCTCTG
AGTTCGCCACC
ACCGCCTCCACCGATGCCGCCAACGCGCCTACCACCTTCCCCGTCGAGG
CACCCCCGCTT
GAGGAGGAGGAAGTGATTATCGAGCAGGACCCAGGTTTTGTAAGCGAAG
ACGACGAGGAC
CGCTCAGTACCAACAGAGGATAAAAAGCAAGACCAGGACAACGCAGAGG
CAAACGAGGAA
CAAGTCGGGCGGGGGGACGAAAGGCATGGCGACTACCTAGATGTGGGAG
ACGACGTGCTG
TTGAAGCATCTGCAGCGCCAGTGCGCCATTATCTGCGACGCGTTGCAAG
AGCGCAGCGAT
GTGCCCCTCGCCATAGCGGATGTCAGCCTTGCCTACGAACGCCACCTAT
TCTCACCGCGC
GTACCCCCCAAACGCCAAGAAAACGGCACATGCGAGCCCAACCCGCGCC
TCAACTTCTAC
CCCGTATTTGCCGTGCCAGAGGTGCTTGCCACCTATCACATCTTTTTCC
AAAACTGCAAG
ATACCCCTATCCTGCCGTGCCAACCGCAGCCGAGCGGACAAGCAGCTGG
CCTTGCGGCAG
GGCGCTGTCATACCTGATATCGCCTCGCTCAACGAAGTGCCAAAAATCT
TTGAGGGTCTT
GGACGCGACGAGAAGCGCGCGGCAAACGCTCTGCAACAGGAAAACAGCG
AAAATGAAAGT
CACTCTGGAGTGTTGGTGGAACTCGAGGGTGACAACGCGCGCCTAGCCG
TACTAAAACGC
AGCATCGAGGTCACCCACTTTGCCTACCCGGCACTTAACCTACCCCCCA
AGGTCATGAGC
ACAGTCATGAGTGAGCTGATCGTGCGCCGTGCGCAGCCCCTGGAGAGGG
ATGCAAATTTG
CAAGAACAAACAGAGGAGGGCCTACCCGCAGTTGGCGACGAGCAGCTAG
CGCGCTGGCTT
CAAACGCGCGAGCCTGCCGACTTGGAGGAGCGACGCAAACTAATGATGG
CCGCAGTGCTC
GTTACCGTGGAGCTTGAGTGCATGCAGCGGTTCTTTGCTGACCCGGAGA
TGCAGCGCAAG
CTAGAGGAAACATTGCACTACACCTTTCGACAGGGCTACGTACGCCAGG
CCTGCAAGATC
TCCAACGTGGAGCTCTGCAACCTGGTCTCCTACCTTGGAATTTTGCACG
AAAACCGCCTT
GGGCAAAACGTGCTTCATTCCACGCTCAAGGGCGAGGCGCGCCGCGACT
ACGTCCGCGAC
TGCGTTTACTTATTTCTATGCTACACCTGGCAGACGGCCATGGGCGTTT
GGCAGCAGTGC
TTGGAGGAGTGCAACCTCAAGGAGCTGCAGAAACTGCTAAAGCAAAACT
TGAAGGACCTA
TGGACGGCCTTCAACGAGCGCTCCGTGGCCGCGCACCTGGCGGACATCA
TTTTCCCCGAA
CGCCTGCTTAAAACCCTGCAACAGGGTCTGCCAGACTTCACCAGTCAAA
GCATGTTGCAG
AACTTTAGGAACTTTATCCTAGAGCGCTCAGGAATCTTGCCCGCCACCT
GCTGTGCACTT
CCTAGCGACTTTGTGCCCATTAAGTACCGCGAATGCCCTCCGCCGCTTT
GGGGCCACTGC
TACCTTCTGCAGCTAGCCAACTACCTTGCCTACCACTCTGACATAATGG
AAGACGTGAGC
GGTGACGGTCTACTGGAGTGTCACTGTCGCTGCAACCTATGCACCCCGC
ACCGCTCCCTG
GTTTGCAATTCGCAGCTGCTTAACGAAAGTCAAATTATCGGTACCTTTG
AGCTGCAGGGT
CCCTCGCCTGACGAAAAGTCCGCGGCTCCGGGGTTGAAACTCACTCCGG
GGCTGTGGACG
TCGGCTTACCTTCGCAAATTTGTACCTGAGGACTACCACGCCCACGAGA
TTAGGTTCTAC
GAAGACCAATCCCGCCCGCCAAATGCGGAGCTTACCGCCTGCGTCATTA
CCCAGGGCCAC
ATTCTTGGCCAATTGCAAGCCATCAACAAAGCCCGCCAAGAGTTTCTGC
TACGAAAGGGA
CGGGGGGTTTACTTGGACCCCCAGTCCGGCGAGGAGCTCAACCCAATCC
CCCCGCCGCCG
CAGCCCTATCAGCAGCAGCCGCGGGCCCTTGCTTCCCAGGATGGCACCC
AAAAAGAAGCT
GCAGCTGCCGCCGCCACCCACGGACGAGGAGGAATACTGGGACAGTCAG
GCAGAGGAGGT
TTTGGACGAGGAGGAGGAGGACATGATGGAAGACTGGGAGAGCCTAGAC
GAGGAAGCTTC
CGAGGTCGAAGAGGTGTCAGACGAAACACCGTCACCCTCGGTCGCATTC
CCCTCGCCGGC
GCCCCAGAAATCGGCAACCGGTTCCAGCATGGCTACAACCTCCGCTCCT
CAGGCGCCGCC
GGCACTGCCCGTTCGCCGACCCAACCGTAGATGGGACACCACTGGAACC
AGGGCCGGTAA
GTCCAAGCAGCCGCCGCCGTTAGCCCAAGAGCAACAACAGCGCCAAGGC
TACCGCTCATG
GCGCGGGCACAAGAACGCCATAGTTGCTTGCTTGCAAGACTGTGGGGGC
AACATCTCCTT
CGCCCGCCGCTTTCTTCTCTACCATCACGGCGTGGCCTTCCCCCGTAAC
ATCCTGCATTA
CTACCGTCATCTCTACAGCCCATACTGCACCGGCGGCAGCGGCAGCGGC
AGCAACAGCAG
CGGCCACACAGAAGCAAAGGCGACCGGATAGCAAGACTCTGACAAAGCC
CAAGAAATCCA
CAGCGGCGGCAGCAGCAGGAGGAGGAGCGCTGCGTCTGGCGCCCAACGA
ACCCGTATCGA
CCCGCGAGCTTAGAAACAGGATTTTTCCCACTCTGTATGCTATATTTCA
ACAGAGCAGGG
GCCAAGAACAAGAGCTGAAAATAAAAAACAGGTCTCTGCGATCCCTCAC
CCGCAGCTGCC
TGTATCACAAAAGCGAAGATCAGCTTCGGCGCACGCTGGAAGACGCGGA
GGCTCTCTTCA
GTAAATACTGCGCGCTGACTCTTAAGGACTAGTTTCGCGCCCTTTCTCA
AATTTAAGCGC
GAAAACTACGTCATCTCCAGCGGCCACACCCGGCGCCAGCACCTGTCGT
CAGCGCCATTA
TGAGCAAGGAAATTCCCACGCCCTACATGTGGAGTTACCAGCCACAAAT
GGGACTTGCGG
CTGGAGCTGCCCAAGACTACTCAACCCGAATAAACTACATGAGCGCGGG
ACCCCACATGA
TATCCCGGGTCAACGGAATCCGCGCCCACCGAAACCGAATTCTCTTGGA
ACAGGCGGCTA
TTACCACCACACCTCGTAATAACCTTAATCCCCGTAGTTGGCCCGCTGC
CCTGGTGTACC
AGGAAAGTCCCGCTCCCACCACTGTGGTACTTCCCAGAGACGCCCAGGC
CGAAGTTCAGA
TGACTAACTCAGGGGCGCAGCTTGCGGGCGGCTTTCGTCACAGGGTGCG
GTCGCCCGGGC
AGGGTATAACTCACCTGACAATCAGAGGGCGAGGTATTCAGCTCAACGA
CGAGTCGGTGA
GCTCCTCGCTTGGTCTCCGTCCGGACGGGACATTTCAGATCGGCGGCGC
CGGCCGTCCTT
CATTCACGCCTCGTCAGGCAATCCTAACTCTGCAGACCTCGTCCTCTGA
GCCGCGCTCTG
GAGGCATTGGAACTCTGCAATTTATTGAGGAGTTTGTGCCATCGGTCTA
CTTTAACCCCT
TCTCGGGACCTCCCGGCCACTATCCGGATCAATTTATTCCTAACTTTGA
CGCGGTAAAGG
ACTCGGCGGACGGCTACGACTGAATGTTAAGTGGAGAGGCAGAGCAACT
GCGCCTGAAAC
ACCTGGTCCACTGTCGCCGCCACAAGTGCTTTGCCCGCGACTCCGGTGA
GTTTTGCTACT
TTGAATTGCCCGAGGATCATATCGAGGGCCCGGCGCACGGCGTCCGGCT
TACCGCCCAGG
GAGAGCTTGCCCGTAGCCTGATTCGGGAGTTTACCCAGCGCCCCCTGCT
AGTTGAGCGGG
ACAGGGGACCCTGTGTTCTCACTGTGATTTGCAACTGTCCTAACCTTGG
ATTACATCAAG
ATCTTTGTTGCCATCTCTGTGCTGAGTATAATAAATACAGAAATTAAAA
TATACTGGGGC
TCCTATCGCCATCCTGTAAACGCCACCGTCTTCACCCGCCCAAGCAAAC
CAAGGCGAACC
TTACCTGGTACTTTTAACATCTCTCCCTCTGTGATTTACAACAGTTTCA
ACCCAGACGGA
GTGAGTCTACGAGAGAACCTCTCCGAGCTCAGCTACTCCATCAGAAAAA
ACACCACCCTC
CTTACCTGCCGGGAACGTACGAGTGCGTCACCGGCCGCTGCACCACACC
TACCGCCTGAC
CGTAAACCAGACTTTTTCCGGACAGACCTCAATAACTCTGTTTACCAGA
ACAGGAGGTGA
GCTTAGAAAACCCTTAGGGTATTAGGCCAAAGGCGCAGCTACTGTGGGG
TTTATGAACAA
TTCAAGCAACTCTACGGGCTATTCTAATTCAGGTTTCTCTAGAATCGGG
GTTGGGGTTAT
TCTCTGTCTTGTGATTCTCTTTATTCTTATACTAACGCTTCTCTGCCTA
AGGCTCGCCGC
CTGCTGTGTGCACATTTGCATTTATTGTCAGCTTTTTAAACGCTGGGGT
CGCCACCCAAG
ATGATTAGGTACATAATCCTAGGTTTACTCACCCTTGCGTCAGCCCACG
GTACCACCCAA
AAGGTGGATTTTAAGGAGCCAGCCTGTAATGTTACATTCGCAGCTGAAG
CTAATGAGTGC
ACCACTCTTATAAAATGCACCACAGAACATGAAAAGCTGCTTATTCGCC
ACAAAAACAAA
ATTGGCAAGTATGCTGTTTATGCTATTTGGCAGCCAGGTGACACTACAG
AGTATAATGTT
ACAGTTTTCCAGGGTAAAAGTCATAAAACTTTTATGTATACTTTTCCAT
TTTATGAAATG
TGCGACATTACCATGTACATGAGCAAACAGTATAAGTTGTGGCCCCCAC
AAAATTGTGTG
GAAAACACTGGCACTTTCTGCTGCACTGCTATGCTAATTACAGTGCTCG
CTTTGGTCTGT
ACCCTACTCTATATTAAATACAAAAGCAGACGCAGCTTTATTGAGGAAA
AGAAAATGCCT
TAATTTACTAAGTTACAAAGCTAATGTCACCACTAACTGCTTTACTCGC
TGCTTGCAAAA
CAAATTCAAAAAGTTAGCATTATAATTAGAATAGGATTTAAACCCCCCG
GTCATTTCCTG
CTCAATACCATTCCCCTGAACAATTGACTCTATGTGGGATATGCTCCAG
CGCTACAACCT
TGAAGTCAGGCTTCCTGGATGTCAGCATCTGACTTTGGCCAGCACCTGT
CCCGCGGATTT
GTTCCAGTCCAACTACAGCGACCCACCCTAACAGAGATGACCAACACAA
CCAACGCGGCC
GCCGCTACCGGACTTACATCTACCACAAATACACCCCAAGTTTCTGCCT
TTGTCAATAAC
TGGGATAACTTGGGCATGTGGTGGTTCTCCATAGCGCTTATGTTTGTAT
GCCTTATTATT
ATGTGGCTCATCTGCTGCCTAAAGCGCAAACGCGCCCGACCACCCATCT
ATAGTCCCATC
ATTGTGCTACACCCAAACAATGATGGAATCCATAGATTGGACGGACTGA
AACACATGTTC
TTTTCTCTTACAGTATGATTAAATGAGACATGATTCCTCGAGTTTTTAT
ATTACTGACCC
TTGTTGCGCTTTTTTGTGCGTGCTCCACATTGGCTGCGGTTTCTCACAT
CGAAGTAGACT
GCATTCCAGCCTTCACAGTCTATTTGCTTTACGGATTTGTCACCCTCAC
GCTCATCTGCA
GCCTCATCACTGTGGTCATCGCCTTTATCCAGTGCATTGACTGGGTCTG
TGTGCGCTTTG
CATATCTCAGACACCATCCCCAGTACAGGGACAGGACTATAGCTGAGCT
TCTTAGAATTC
TTTAATTATGAAATTTACTGTGACTTTTCTGCTGATTATTTGCACCCTA
TCTGCGTTTTG
TTCCCCGACCTCCAAGCCTCAAAGACATATATCATGCAGATTCACTCGT
ATATGGAATAT
TCCAAGTTGCTACAATGAAAAAAGCGATCTTTCCGAAGCCTGGTTATAT
GCAATCATCTC
TGTTATGGTGTTCTGCAGTACCATCTTAGCCCTAGCTATATATCCCTAC
CTTGACATTGG
CTGGAACGCAATAGATGCCATGAACCACCCAACTTTCCCCGCGCCCGCT
ATGCTTCCACT
GCAACAAGTTGTTGCCGGCGGCTTTGTCCCAGCCAATCAGCCTCGCCCA
CCTTCTCCCAC
CCCCACTGAAATCAGCTACTTTAATCTAACAGGAGGAGATGACTGACAC
CCTAGATCTAG
AAATGGACGGAATTATTACAGAGCAGCGCCTGCTAGAAAGACGCAGGGC
AGCGGCCGAGC
AACAGCGCATGAATCAAGAGCTCCAAGACATGGTTAACTTGCACCAGTG
CAAAAGGGGTA
TCTTTTGTCTGGTAAAGCAGGCCAAAGTCACCTACGACAGTAATACCAC
CGGACACCGCC
TTAGCTACAAGTTGCCAACCAAGCGTCAGAAATTGGTGGTCATGGTGGG
AGAAAAGCCCA
TTACCATAACTCAGCACTCGGTAGAAACCGAAGGCTGCATTCACTCACC
TTGTCAAGGAC
CTGAGGATCTCTGCACCCTTATTAAGACCCTGTGCGGTCTCAAAGATCT
TATTCCCTTTA
ACTAATAAAAAAAAATAATAAAGCATCACTTACTTAAAATCAGTTAGCA
AATTTCTGTCC
AGTTTATTCAGCAGCACCTCCTTGCCCTCCTCCCAGCTCTGGTATTGCA
GCTTCCTCCTG
GCTGCAAACTTTCTCCACAATCTAAATGGAATGTCAGTTTCCTCCTGTT
CCTGTCCATCC
GCACCCACTATCTTCATGTTGTTGCAGATGAAGCGCGCAAGACCGTCTG
AAGATACCTTC
AACCCCGTGTATCCATATGACACGGAAACCGGTCCTCCAACTGTGCCTT
TTCTTACTCCT
CCCTTTGTATCCCCCAATGGGTTTCAAGAGAGTCCCCCTGGGGTACTCT
CTTTGCGCCTA
TCCGAACCTCTAGTTACCTCCAATGGCATGCTTGCGCTCAAAATGGGCA
ACGGCCTCTCT
CTGGACGAGGCCGGCAACCTTACCTCCCAAAATGTAACCACTGTGAGCC
CACCTCTCAAA
AAAACCAAGTCAAACATAAACCTGGAAATATCTGCACCCCTCACAGTTA
CCTCAGAAGCC
CTAACTGTGGCTGCCGCCGCACCTCTAATGGTCGCGGGCAACACACTCA
CCATGCAATCA
CAGGCCCCGCTAACCGTGCACGACTCCAAACTTAGCATTGCCACCCAAG
GACCCCTCACA
GTGTCAGAAGGAAAGCTAGCCCTGCAAACATCAGGCCCCCTCACCACCA
CCGATAGCAGT
ACCCTTACTATCACTGCCTCACCCCCTCTAACTACTGCCACTGGTAGCT
TGGGCATTGAC
TTGAAAGAGCCCATTTATACACAAAATGGAAAACTAGGACTAAAGTACG
GGGCTCCTTTG
CATGTAACAGACGACCTAAACACTTTGACCGTAGCAACTGGTCCAGGTG
TGACTATTAAT
AATACTTCCTTGCAAACTAAAGTTACTGGAGCCTTGGGTTTTGATTCAC
AAGGCAATATG
CAACTTAATGTAGCAGGAGGACTAAGGATTGATTCTCAAAACAGACGCC
TTATACTTGAT
GTTAGTTATCCGTTTGATGCTCAAAACCAACTAAATCTAAGACTAGGAC
AGGGCCCTCTT
TTTATAAACTCAGCCCACAACTTGGATATTAACTACAACAAAGGCCTTT
ACTTGTTTACA
GCTTCAAACAATTCCAAAAAGCTTGAGGTTAACCTAAGCACTGCCAAGG
GGTTGATGTTT
GACGCTACAGCCATAGCCATTAATGCAGGAGATGGGCTTGAATTTGGTT
CACCTAATGCA
CCAAACACAAATCCCCTCAAAACAAAAATTGGCCATGGCCTAGAATTTG
ATTCAAACAAG
GCTATGGTTCCTAAACTAGGAACTGGCCTTAGTTTTGACAGCACAGGTG
CCATTACAGTA
GGAAACAAAAATAATGATAAGCTAACTTTGTGGACCACACCAGCTCCAT
CTCCTAACTGT
AGACTAAATGCAGAGAAAGATGCTAAACTCACTTTGGTCTTAACAAAAT
GTGGCAGTCAA
ATACTTGCTACAGTTTCAGTTTTGGCTGTTAAAGGCAGTTTGGCTCCAA
TATCTGGAACA
GTTCAAAGTGCTCATCTTATTATAAGATTTGACGAAAATGGAGTGCTAC
TAAACAATTCC
TTCCTGGACCCAGAATATTGGAACTTTAGAAATGGAGATCTTACTGAAG
GCACAGCCTAT
ACAAACGCTGTTGGATTTATGCCTAACCTATCAGCTTATCCAAAATCTC
ACGGTAAAACT
GCCAAAAGTAACATTGTCAGTCAAGTTTACTTAAACGGAGACAAAACTA
AACCTGTAACA
CTAACCATTACACTAAACGGTACACAGGAAACAGGAGACACAACTCCAA
GTGCATACTCT
ATGTCATTTTCATGGGACTGGTCTGGCCACAACTACATTAATGAAATAT
TTGCCACATCC
TCTTACACTTTTTCATACATTGCCCAAGAATAAAGAATCGTTTGTGTTA
TGTTTCAACGT
GTTTATTTTTCAATTGCAGAAAATTTCAAGTCATTTTTCATTCAGTAGT
ATAGCCCCACC
ACCACATAGCTTATACAGATCACCGTACCTTAATCAAACTCACAGAACC
CTAGTATTCAA
CCTGCCACCTCCCTCCCAACACACAGAGTACACAGTCCTTTCTCCCCGG
CTGGCCTTAAA
AAGCATCATATCATGGGTAACAGACATATTCTTAGGTGTTATATTCCAC
ACGGTTTCCTG
TCGAGCCAAACGCTCATCAGTGATATTAATAAACTCCCCGGGCAGCTCA
CTTAAGTTCAT
GTCGCTGTCCAGCTGCTGAGCCACAGGCTGCTGTCCAACTTGCGGTTGC
TTAACGGGCGG
CGAAGGAGAAGTCCACGCCTACATGGGGGTAGAGTCATAATCGTGCATC
AGGATAGGGCG
GTGGTGCTGCAGCAGCGCGCGAATAAACTGCTGCCGCCGCCGCTCCGTC
CTGCAGGAATA
CAACATGGCAGTGGTCTCCTCAGCGATGATTCGCACCGCCCGCAGCATA
AGGCGCCTTGT
CCTCCGGGCACAGCAGCGCACCCTGATCTCACTTAAATCAGCACAGTAA
CTGCAGCACAG
CACCACAATATTGTTCAAAATCCCACAGTGCAAGGCGCTGTATCCAAAG
CTCATGGCGGG
GACCACAGAACCCACGTGGCCATCATACCACAAGCGCAGGTAGATTAAG
TGGCGACCCCT
CATAAACACGCTGGACATAAACATTACCTCTTTTGGCATGTTGTAATTC
ACCACCTCCCG
GTACCATATAAACCTCTGATTAAACATGGCGCCATCCACCACCATCCTA
AACCAGCTGGC
CAAAACCTGCCCGCCGGCTATACACTGCAGGGAACCGGGACTGGAACAA
TGACAGTGGAG
AGCCCAGGACTCGTAACCATGGATCATCATGCTCGTCATGATATCAATG
TTGGCACAACA
CAGGCACACGTGCATACACTTCCTCAGGATTACAAGCTCCTCCCGCGTT
AGAACCATATC
CCAGGGAACAACCCATTCCTGAATCAGCGTAAATCCCACACTGCAGGGA
AGACCTCGCAC
GTAACTCACGTTGTGCATTGTCAAAGTGTTACATTCGGGCAGCAGCGGA
TGATCCTCCAG
TATGGTAGCGCGGGTTTCTGTCTCAAAAGGAGGTAGACGATCCCTACTG
TACGGAGTGCG
CCGAGACAACCGAGATCGTGTTGGTCGTAGTGTCATGCCAAATGGAACG
CCGGACGTAGT
CATATTTCCTGAAGCAAAACCAGGTGCGGGCGTGACAAACAGATCTGCG
TCTCCGGTCTC
GCCGCTTAGATCGCTCTGTGTAGTAGTTGTAGTATATCCACTCTCTCAA
AGCATCCAGGC
GCCCCCTGGCTTCGGGTTCTATGTAAACTCCTTCATGCGCCGCTGCCCT
GATAACATCCA
CCACCGCAGAATAAGCCACACCCAGCCAACCTACACATTCGTTCTGCGA
GTCACACACGG
GAGGAGCGGGAAGAGCTGGAAGAACCATGTTTTTTTTTTTATTCCAAAA
GATTATCCAAA
ACCTCAAAATGAAGATCTATTAAGTGAACGCGCTCCCCTCCGGTGGCGT
GGTCAAACTCT
ACAGCCAAAGAACAGATAATGGCATTTGTAAGATGTTGCACAATGGCTT
CCAAAAGGCAA
ACGGCCCTCACGTCCAAGTGGACGTAAAGGCTAAACCCTTCAGGGTGAA
TCTCCTCTATA
AACATTCCAGCACCTTCAACCATGCCCAAATAATTCTCATCTCGCCACC
TTCTCAATATA
TCTCTAAGCAAATCCCGAATATTAAGTCCGGCCATTGTAAAAATCTGCT
CCAGAGCGCCC
TCCACCTTCAGCCTCAAGCAGCGAATCATGATTGCAAAAATTCAGGTTC
CTCACAGACCT
GTATAAGATTCAAAAGCGGAACATTAACAAAAATACCGCGATCCCGTAG
GTCCCTTCGCA
GGGCCAGCTGAACATAATCGTGCAGGTCTGCACGGACCAGCGCGGCCAC
TTCCCCGCCAG
GAACCTTGACAAAAGAACCCACACTGATTATGACACGCATACTCGGAGC
TATGCTAACCA
GCGTAGCCCCGATGTAAGCTTTGTTGCATGGGCGGCGATATAAAATGCA
AGGTGCTGCTC
AAAAAATCAGGCAAAGCCTCGCGCAAAAAAGAAAGCACATCGTAGTCAT
GCTCATGCAGA
TAAAGGCAGGTAAGCTCCGGAACCACCACAGAAAAAGACACCATTTTTC
TCTCAAACATG
TCTGCGGGTTTCTGCATAAACACAAAATAAAATAACAAAAAAACATTTA
AACATTAGAAG
CCTGTCTTACAACAGGAAAAACAACCCTTATAAGCATAAGACGGACTAC
GGCCATGCCGG
CGTGACCGTAAAAAAACTGGTCACCGTGATTAAAAAGCACCACCGACAG
CTCCTCGGTCA
TGTCCGGAGTCATAATGTAAGACTCGGTAAACACATCAGGTTGATTCAT
CGGTCAGTGCT
AAAAAGCGACCGAAATAGCCCGGGGGAATACATACCCGCAGGCGTAGAG
ACAACATTACA
GCCCCCATAGGAGGTATAACAAAATTAATAGGAGAGAAAAACACATAAA
CACCTGAAAAA
CCCTCCTGCCTAGGCAAAATAGCACCCTCCCGCTCCAGAACAACATACA
GCGCTTCCACA
GCGGCAGCCATAACAGTCAGCCTTACCAGTAAAAAAGAAAACCTATTAA
AAAAACACCAC
TCGACACGGCACCAGCTCAATCAGTCACAGTGTAAAAAAGGGCCAAGTG
CAGAGCGAGTA
TATATAGGACTAAAAAATGACGTAACGGTTAAAGTCCACAAAAAACACC
CAGAAAACCGC
ACGCGAACCTACGCCCAGAAACGAAAGCCAAAAAACCCACAACTTCCTC
AAATCGTCACT
TCCGTTTTCCCACGTTACGTCACTTCCCATTTTAATTAAGAAAACTACA
ATTCCCAACAC
ATACAAGTTACTCCGCCCTAAAACCTACGTCACCCGCCCCGTTCCCACG
CCCCGCGCCAC
GTCACAAACTCCACCCCCTCATTATCATATTGGCTTCAATCCAAAATAA
GGTATATTATT
GATGATGATTACCCT
SEQ ID NO: 52 is the sequence for oligonucleotide
primer 1618.95.1
5′ TACCGGGGTACCCAACTCCA
SEQ ID NO: 53 is the sequence for oligonucleotide
primer 1618.95.6
5′ GACGCGGCCTGTCCGGCC
SEQ ID NO: 54 is the sequence for oligonucleotide
primer 1618.97.1
5′ TACTTATGACTCGTACTATTGTTATTCATCCAGGCGGTAGGAGGGC
CATCATGAA
SEQ ID NO: 55 is the sequence for oligonucleotide
primer 1618.97.2
5′ CCTTTATTGAAAGTGTCTCTAGTAGCTAGCGGGAGGGAGGTCC
SEQ ID NO: 56 is the sequence for oligonucleotide
primer 1618.95.5
5′ TACTAGAGACACTTTCAATAAAGG
SEQ ID NO: 57 is the sequence for oligonucleotide
primer 1706.83.1
5′ GTT AAC ATG GTT CTG CAG AGC
SEQ ID NO: 58 is the sequence for oligonucleotide
primer 1706.83.2
5′ GGC TCG TCC ATG GGA TCC ACC TCA AAA GTC
SEQ ID NO: 59 is the sequence for oligonucleotide
primer 1706.95.1
5′ GGA TCC CAT GGA CGA GCC CA
SEQ ID NO: 60 is the sequence for oligonucleotide
primer 1618.116.3
5′ CT TAT TAC CGG GGT ACC CAA CTC CTC GAG TAT TT
SEQ ID NO: 61 is the sequence for oligonucleotide
primer 1619.144.1
5′ TACAA GTA TAC GCC ACC ATG GCT ATG ATG GAG GTC
CAG
SEQ ID NO: 62 is the sequence for oligonucleotide
primer 1619.144.3
5′ TTGTA GTATAC TTA GCC AAC TAA AAA GGC CCC

1. A modified adenovirus fiber, comprising a modification to the fiber protein shaft, wherein the modification comprises a modification selected from among:
a modification of a last full repeat;
a modification of a β-repeat corresponding to a third β-repeat and a modification of a last full repeat; and
a modification of one or both of a modification of a last full repeat and a modification of at least one amino acid in a contiguous sequence of amino acids corresponding to the amino acid sequence TTVT/S set forth in SEQ ID No. 44 in a third β-repeat, whereby binding of the fiber or of a viral particle containing such fiber to the Coxsackie-Adenovirus Receptor (CAR) is reduced compared to the unmodified fiber.

2. A modified adenovirus fiber, comprising a modification to the fiber protein shaft, whereby binding of the modified fiber to Coxsackie-Adenovirus Receptor (CAR) is reduced or eliminated, wherein:
the unmodified fiber binds the Coxsackie-Adenovirus Receptor (CAR); and
the modification comprises a modification of a repeat corresponding to the last full β-repeat, or the third β-repeat and the last full β-repeat of the shaft or one or both of the last full β-repeat and a portion of the third β-repeat that comprises the TTVT/S motif (SEQ ID No. 44} or a corresponding motif.

3. A modified fiber of claim 1 or 2, wherein the modified fiber binds to CAR with less than 50%, 40%, 30%, 20%, 10%, 5%, 1% of the binding affinity of the unmodified fiber.

4. A modified adenovirus fiber of any of claims 1-3, wherein the modified fiber is more rigid than the unmodified fiber.

5. A modified adenovirus fiber of any or claims 1-4, wherein the modification is a mutation, deletion, insertion or replacement of at least one amino acid in the fiber shaft repeat corresponding to the third repeat.

6. The modified adenovirus fiber of any of claims 1-5, wherein the unmodified fiber is a fiber of a serotype C adenovirus.

7. The modified adenovirus fiber of claim 6, wherein the serotype C adenovirus is Ad2 or Ad5.

8. The modified adenovirus fiber of any of claims 1-7, wherein the modified fiber is more rigid and shorter than the unmodified fiber.

9. The modified adenovirus fiber of any of claims 1-8, wherein at least one amino acid in the contiguous sequence of amino acids corresponding to the amino acid sequence set forth in SEQ ID No. 42 or 43 is modified.

10. The modified adenovirus fiber of any of claims 1-6, wherein the third β-repeat is modified by replacing it with a corresponding repeat from a serotype D fiber shaft repeat sequence.

11. The modified adenovirus fiber of claim 10, wherein the serotype D adenovirus is selected from the group consisting of Ad8, Ad9, Ad15, Ad19p and Ad37.

12. The modified adenovirus fiber of any of claims 1-6, wherein the third β-repeat is modified by replacing it with a corresponding repeat selected from the group consisting of SEQ ID NOS: 58, 66, 67 and 68.

13. The modified adenovirus fiber of any of claims 1-12, wherein the modification is a mutation, deletion, insertion or replacement of at least one amino acid in a fiber shaft β-repeat corresponding to the last full β repeat and/or corresponding to the third β-repeat.

14. The modified adenovirus fiber of claim 13, wherein the unmodified fiber is a serotype C adenovirus fiber.

15. The modified adenovirus fiber of claim 12, wherein the serotype C adenovirus is Ad2 or Ad5.

16. The modified adenovirus fiber of claim 15, wherein the modification is a modification of at least one amino acid in a contiguous sequence of amino acids corresponding to those set forth in SEQ ID No. 46 or SEQ ID No. 47.

17. The modified adenovirus fiber of any of claims 1-15, wherein the modification comprises replacement of the last full β-repeat with a corresponding repeat sequence from a serotype D adenovirus fiber shaft.

18. The modified adenovirus fiber of claim 17, wherein the serotype D adenovirus is selected from the group consisting of Ad8, Ad9, Ad15, Ad19p and Ad37.

19. The modified adenovirus fiber of any of claims 13-18, wherein the modification is in the last full repeat; and the last full repeat comprises a change of at least one amino acid in the repeat at contiguous amino acids corresponding to the amino acid sequence set forth in SEQ ID No. 49.

20. The modified adenovirus fiber of any of claims 13-18, wherein the modification is in the last full repeat; and the last full repeat comprises a sequence of amino acids selected from the group consisting of SEQ ID NOS: 48, 59, 60 and 61.

21. The modified adenovirus fiber of any of claims 1-7, wherein a contiguous sequence of amino acids corresponding to the third repeat of the fiber shaft is deleted.

22. The modified adenovirus fiber of any of claims 1-7, wherein a contiguous sequence of amino acids corresponding to the last full repeat of the fiber shaft is deleted.

23. The modified adenovirus fiber of any of claims 1-7, wherein a contiguous sequence of amino acids corresponding to the third repeat and the contiguous sequence of amino acids corresponding to the last full repeat are modified.

24. The modified adenovirus fiber of claim 22 or claim 23, wherein the modification is a mutation, deletion, insertion or replacement of at least one amino acid in a fiber shaft repeat corresponding to the third repeat and/or the last full repeat.

25. The modified adenovirus fiber of claim 24, wherein the unmodified fiber shaft is from a serotype C adenovirus.

26. The modified adenovirus fiber of claim 25, wherein the serotype C adenovirus is Ad2 or Ad5.

27. The modified adenovirus fiber of claim 25, wherein the modified repeats corresponding to the third repeat and the last full repeat are from a serotype D adenovirus.

28. The modified adenovirus fiber of claim 27, wherein the serotype D adenovirus is selected from the group consisting of Ad8, Ad9, Ad15, Ad19p and Ad37.

29. The modified adenovirus fiber of claim 25, wherein the third repeat comprises a sequence selected from the group consisting of SEQ ID NOs. 58, 66, 67 and 68 and the last full repeat comprises an amino acid sequence selected from the group consisting of SEQ ID NOs. 48, 59, 60 and 61.

30. The modified adenovirus fiber of claim 25, wherein the third repeat sequence is selected from a corresponding repeat sequence of a fiber protein from Ad8, Ad9, Ad15, Ad19p or Ad37; and the last full repeat is selected from a corresponding repeat sequence of a fiber protein from Ad8, Ad9, Ad15, Ad19p or Ad37.

31. The modified adenovirus fiber of any of claims 1-30, wherein the modified adenovirus fiber further comprises at least one additional modification in the fiber protein, whereby the modified fiber binds to a receptor other than CAR with greater affinity than the unmodified fiber binds to such receptor.

32. The modified adenovirus fiber of any of claims 1-30, wherein the modified adenovirus fiber further comprises at least one additional modification in the fiber protein; and the modification is a modification in the fiber knob that further reduces or eliminates any binding of the modified fiber to CAR.

33. The modified adenovirus fiber of claim 31, wherein an additional modification is a modification of the Heparin Sulfate Proteoglycans (HSP) binding site in the fiber shaft.

34. The modified adenovirus fiber of claim 31 or claim 32, wherein an additional modification is a modification in the fiber knob.

35. The modified fiber of any of claims 1-34, wherein the fiber is shortened or its flexibility is reduced compared to the unmodified fiber.

36. The modified adenovirus fiber of claim 34, wherein the fiber knob is replaced with fiber knobs from an adenovirus that does not interact with CAR.

37. The modified adenovirus fiber of claim 36, wherein the adenovirus fiber knob that does not interact with CAR is Ad3 fiber knob, Ad41 short fiber knob, or Ad35 fiber knob.

38. The modified adenovirus fiber of claim 34, wherein fiber knob mutations are mutations in the AB loop or CD loop.

39. The modified adenovirus fiber of claim 38, wherein fiber knob mutations are mutations in the AB loop or CD loop selected from KO1 and KO12.

40. A modified adenovirus fiber, comprising a fiber protein, wherein:
the unmodified fiber binds the Coxsackie-Adenovirus Receptor (CAR);
the fiber protein comprises a modification to the fiber protein shaft such that binding of the modified fiber to CAR is substantially reduced or eliminated;
the modified fiber comprises repeats corresponding to the third repeat and the last full repeat; and at least one repeat of the fiber shaft is deleted.

41. The modified adenovirus fiber of claim 40, wherein the repeats corresponding to repeats 4-17 are deleted.

42. The modified adenovirus fiber of claim 40 or claim 41, wherein the fiber is from a serotype C adenovirus.

43. The modified adenovirus fiber of claim 42, wherein the serotype C adenovirus is Ad2 or Ad5.

44. The modified adenovirus fiber of claim 43, wherein the amino acids corresponding to positions 95-316 are deleted.

45. The modified adenovirus fiber of any of claims claim 1-39, wherein the fiber protein is from a serotype A, B, C or F adenovirus; and at least one amino acid corresponding to the consensus repeat sequence as set forth in SEQ ID No. 49 is modified in the repeat corresponding to either the third repeat or the last full repeat.

46. A nucleic acid molecule, comprising a sequence of nucleotides that encodes a modified fiber of any of claims 1-45.

47. The nucleic acid molecule of claim 46 that comprises a vector.

48. The nucleic acid molecule of claim 46 or claim 47 that comprises heterologous nucleic acid encoding a gene product.

49. The nucleic acid molecule of any of claims 46-4B that is an adenovirus vector.

50. The nucleic acid molecule of claim 49 that is an adenoviral vector from a serotype C adenovirus.

51. The nucleic acid molecule of claim 49 or claim 50, wherein the heterologous nucleic acid encodes a therapeutic product.

52. The nucleic acid molecule of any of claims 46-51 that is an early generation adenoviral vector, a gutless adenoviral vector or a replication-conditional adenoviral vector.

53. The nucleic acid molecule of claim 52, wherein the replication-conditional adenoviral vector is an oncolytic adenoviral vector.

54. A cell, comprising the nucleic acid of any of claims claim 46-53.

55. The cell of claim 54 that is a eukaryotic cell.

56. The cell of claim 54 that is a prokaryotic cell.

57. A cell of claim 54 that is in a packaging cell line.

58. An adenovirus particle, comprising the modified fiber of any of claims 1-45.

59. The adenovirus particle of claim 58, wherein the capsid further comprises a penton modification.

60. The adenovirus particle of claim 58 or claim 59, wherein the modified fiber includes an N-terminal portion from a fiber of a serotype C Ad virus, wherein the N-terminal portion is sufficient to increase incorporation into the particle compared to in its absence.

61. The adenovirus particle of any of claims 58-60, that comprises a modified serotype C genome, wherein the N-terminal portion of the modified fiber comprises at least the N-terminal 15, 16 or 17 amino acids of a serotype C fiber.

62. The particle of claim 61 wherein the serotype C virus is an Ad2 or Ad5 virus.

63. The adenoviral particle of any of claims 58-62 that further comprises a targeting ligand in the capsid.

64. The adenovirus particle of any of claims 58-63 further, comprising a heterologous nucleic acid in the genome thereof.

65. The adenovirus particle of claim 64, wherein the heterologous nucleic acid encodes a therapeutically effective product.

66. The adenoviral particle of any of claims 58-65 that includes a modification to the capsid whereby binding of the viral particle to HSP is altered compared to a particle that expresses an unmodified capsid.

67. The adenoviral particle of claim 66, wherein the capsid modification that alters HSP binding is in the fiber.

68. An adenoviral particle of any of claims 58-67, comprising a mutation in the αᵥ integrin-binding region of the capsid, whereby binding to the integrin is eliminated or reduced.

69. The adenoviral particle of any of claims 58-68, wherein the fiber further comprises a modification in the fiber knob to further reduce any CAR binding.

70. The adenoviral particle of claim 69, wherein the fiber knob modification is in the AB loop or CD loop.

71. The adenoviral particle of claim 70, wherein the fiber knob modification is selected from the group consisting of K01 and K012.

72. A composition formulated for administration to a subject, comprising the adenovirus particle of any of claims 58-71.

73. A method of detargeting an adenoviral vector, comprising reducing or eliminating the binding of an adenoviral particle to CAR by producing an adenoviral particle that expresses a modified fiber of any of claims 1-45.

74. The method of claim 73, wherein the modified fiber increases the binding to the particular cell type compared to the unmodified fiber.
